SEQUENCE LISTING (42 sequences)

SEQ ID NO: 1 - DNA, 49 bp, Artificial Sequence
  Description of Artificial Sequence Synthetic primer
gacaaattaatacgactcactataggaaacctgatcatgtagatcgaac

SEQ ID NO: 2 - DNA, 21 bp, Artificial Sequence
  Description of Artificial Sequence Synthetic primer
ccccaggctttacactttatg

SEQ ID NO: 3 - DNA, 39 bp, Artificial Sequence
  Description of Artificial Sequence Synthetic primer
tggcggaaaccccgggaatctaacccggctgaacggatt

SEQ ID NO: 4 - DNA, 21 bp, Artificial Sequence
  Description of Artificial Sequence Synthetic primer
tccacgccgaacctcccgatc

SEQ ID NO: 5 - DNA, 24 bp, Artificial Sequence
  Description of Artificial Sequence Synthetic primer
tcccggcttcgctgcatttattgc

SEQ ID NO: 6 - DNA, 29 bp, Artificial Sequence
  Description of Artificial Sequence Synthetic primer
aaaatcacggcagacaaacaaaagaatgg

SEQ ID NO: 7 - DNA, 40 bp, Artificial Sequence
  Description of Artificial Sequence Synthetic primer
atgggtctcacacaaactcgagtacaactttaactcacac

SEQ ID NO: 8 - DNA, 33 bp, Artificial Sequence
  Description of Artificial Sequence Synthetic primer
atgggtctcgattccattcttttgtttgtctgc

SEQ ID NO: 9 - DNA, 19 bp, Artificial Sequence
  Description of Artificial Sequence Synthetic primer
taatacgactcactatagg

SEQ ID NO: 10 - DNA, 63 bp, Artificial Sequence
  Description of Artificial Sequence Synthetic oligonucleotide
ctcgagtacaactttaactcacacaatgtatacatcacggcagacaaacaaaagaatggaatc

SEQ ID NO: 11 - DNA, 63 bp, Artificial Sequence
  Description of Artificial Sequence Synthetic oligonucleotide
ctcgagtacaactttaactcacacaatgtagtaatcacggcagacaaacaaaagaatggaatc

SEQ ID NO: 12 - DNA, 63 bp, Artificial Sequence
  Description of Artificial Sequence Synthetic oligonucleotide
  modified_base (32)..(32): Nicotinamide modified nucleotide
ctcgagtacaactttaactcacacaatgtaancatcacggcagacaaacaaaagaatggaatc

SEQ ID NO: 13 - DNA, 63 bp, Artificial Sequence; identical to SEQ ID NO: 12
  Description of Artificial Sequence Synthetic oligonucleotide
  modified_base (32)..(32): TPT3 modified nucleotide

SEQ ID NO: 14 - DNA, 63 bp, Artificial Sequence
  Description of Artificial Sequence Synthetic oligonucleotide
  modified_base (32)..(32): Nicotinamide modified nucleotide
ctcgagtacaactttaactcacacaatgtagncatcacggcagacaaacaaaagaatggaatc

SEQ ID NO: 15 - DNA, 63 bp, Artificial Sequence; identical to SEQ ID NO: 14
  Description of Artificial Sequence Synthetic oligonucleotide
  modified_base (32)..(32): TPT3 modified nucleotide

SEQ ID NO: 16 - DNA, 63 bp, Artificial Sequence
  Description of Artificial Sequence Synthetic oligonucleotide
  modified_base (32)..(32): Nicotinamide modified nucleotide
ctcgagtacaactttaactcacacaatgtagntatcacggcagacaaacaaaagaatggaatc

SEQ ID NO: 17 - DNA, 63 bp, Artificial Sequence; identical to SEQ ID NO: 16
  Description of Artificial Sequence Synthetic oligonucleotide
  modified_base (32)..(32): TPT3 modified nucleotide

SEQ ID NO: 18 - DNA, 63 bp, Artificial Sequence
  Description of Artificial Sequence Synthetic oligonucleotide
  modified_base (33)..(33): Nicotinamide modified nucleotide
ctcgagtacaactttaactcacacaatgtaagnatcacggcagacaaacaaaagaatggaatc

SEQ ID NO: 19 - DNA, 63 bp, Artificial Sequence
  Description of Artificial Sequence Synthetic oligonucleotide
  modified_base (31)..(31): Nicotinamide modified nucleotide
ctcgagtacaactttaactcacacaatgtanccatcacggcagacaaacaaaagaatggaatc

SEQ ID NO: 20 - DNA, 63 bp, Artificial Sequence; identical to SEQ ID NO: 19
  Description of Artificial Sequence Synthetic oligonucleotide
  modified_base (31)..(31): TPT3 modified nucleotide

SEQ ID NO: 21 - DNA, 52 bp, Artificial Sequence
  Description of Artificial Sequence Synthetic oligonucleotide
cctgatcatgtagatcgaacggactgtaaatccgttcagccgggttagattc

SEQ ID NO: 22 - DNA, 52 bp, Artificial Sequence
  Description of Artificial Sequence Synthetic oligonucleotide
cctgatcatgtagatcgaacggactctaaatccgttcagccgggttagattc

SEQ ID NO: 23 - DNA, 52 bp, Artificial Sequence
  Description of Artificial Sequence Synthetic oligonucleotide
  modified_base (27)..(27): TPT3 modified nucleotide
cctgatcatgtagatcgaacggactgntaatccgttcagccgggttagattc

SEQ ID NO: 24 - DNA, 52 bp, Artificial Sequence; identical to SEQ ID NO: 23
  Description of Artificial Sequence Synthetic oligonucleotide
  modified_base (27)..(27): Nicotinamide modified nucleotide

SEQ ID NO: 25 - DNA, 52 bp, Artificial Sequence
  Description of Artificial Sequence Synthetic oligonucleotide
  modified_base (27)..(27): TPT3 modified nucleotide
cctgatcatgtagatcgaacggactgncaatccgttcagccgggttagattc

SEQ ID NO: 26 - DNA, 52 bp, Artificial Sequence; identical to SEQ ID NO: 25
  Description of Artificial Sequence Synthetic oligonucleotide
  modified_base (27)..(27): Nicotinamide modified nucleotide

SEQ ID NO: 27 - DNA, 52 bp, Artificial Sequence
  Description of Artificial Sequence Synthetic oligonucleotide
  modified_base (27)..(27): TPT3 modified nucleotide
cctgatcatgtagatcgaacggactancaatccgttcagccgggttagattc

SEQ ID NO: 28 - DNA, 52 bp, Artificial Sequence; identical to SEQ ID NO: 27
  Description of Artificial Sequence Synthetic oligonucleotide
  modified_base (27)..(27): Nicotinamide modified nucleotide

SEQ ID NO: 29 - DNA, 52 bp, Artificial Sequence
  Description of Artificial Sequence Synthetic oligonucleotide
  modified_base (26)..(26): TPT3 modified nucleotide
cctgatcatgtagatcgaacggactnctaatccgttcagccgggttagattc

SEQ ID NO: 30 - DNA, 52 bp, Artificial Sequence; identical to SEQ ID NO: 29
  Description of Artificial Sequence Synthetic oligonucleotide
  modified_base (26)..(26): Nicotinamide modified nucleotide

SEQ ID NO: 31 - DNA, 52 bp, Artificial Sequence
  Description of Artificial Sequence Synthetic oligonucleotide
  modified_base (28)..(28): TPT3 modified nucleotide
cctgatcatgtagatcgaacggactggnaatccgttcagccgggttagattc

SEQ ID NO: 32 - DNA, 52 bp, Artificial Sequence; identical to SEQ ID NO: 31
  Description of Artificial Sequence Synthetic oligonucleotide
  modified_base (28)..(28): Nicotinamide modified nucleotide

SEQ ID NO: 33 - DNA, 604 bp, Artificial Sequence
  Description of Artificial Sequence Synthetic polynucleotide
catctagggcggccaattccgcccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccac

SEQ ID NO: 34 - DNA, 711 bp, Artificial Sequence
  Description of Artificial Sequence Synthetic polynucleotide
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 35 - DNA, 1260 bp, Artificial Sequence
  Description of Artificial Sequence Synthetic polynucleotide
atggataaaaaaccgctggacgttctgatctccgctacgggtctgtggatgagccgcacgggtacgctgcataaaatcaagcactatgagatttctcgttctaaaatctacatcgaaatggcgtgtggtgaccatctggttgtgaacaactctcgttcttgtcgtccggcacgtgcattccgttatcataaataccgtaaaacctgcaaacgttgtcgtgtttctgacgaagatatcaacaacttcctgacccgttctaccgaaggcaaaacctctgttaaagttaaagttgtttctgaaccgaaagtgaaaaaagcgatgccgaaatctgtttctcgtgcgccgaaaccgctggaaaatccggtttctgcgaaagcgtctaccgacacctctcgttctgttccgtctccggcgaaatctaccccgaactctccggttccgacctctgcaagtgcccccgcacttacgaagagccagactgacaggcttgaagtcctgttaaacccaaaagatgagatttccctgaattccggcaagcctttcagggagcttgagtccgaattgctctctcgcagaaaaaaagacctgcagcagatctacgcggaagaaagggagaattatctggggaaactcgagcgtgaaattaccaggttctttgtggacaggggttttctggaaataaaatccccgatcctgatccctcttgagtatatcgaaaggatgggcattgataatgataccgaactttcaaaacagatcttcagggttgacaagaacttctgcctgagacccatgcttgctccaaacctttacaactacctgcgcaagcttgacagggccctgcctgatccaataaaaatttttgaaataggcccatgctacagaaaagagtccgacggcaaagaacacctcgaagagtttaccatgctgaacttctgccagatgggatcgggatgcacacgggaaaatcttgaaagcataattacggacttcctgaaccacctgggaattgatttcaagatcgtaggcgattcctgcatggtctatggggatacccttgatgtaatgcacggagacctggaactttcctctgcagtagtcggacccataccgcttgaccgggaatggggtattgataaaccctggataggggcaggtttcggactcgaacgccttctaaaggttaaacacgactttaaaaatatcaagagagctgcacgctcggaatcgtattacaacggcatctcaaccaatctgtaa

SEQ ID NO: 36 - DNA, 39 bp, Artificial Sequence
  Description of Artificial Sequence Synthetic oligonucleotide
gaatacaagctacttgttctttttgcaggatccgccacc

SEQ ID NO: 37 - DNA, 141 bp, Artificial Sequence
  Description of Artificial Sequence Synthetic polynucleotide
aagcttaattagctgagcttggactcctaagcatgcaagcttggcgtaatcatggtcatagctgtttcctgtgtgaaattgttatccgctcacaattccacacaacatacgagccggaagcataaagtgtaaagcctgggg

SEQ ID NO: 38 - DNA, 36 bp, Artificial Sequence
  Description of Artificial Sequence Synthetic oligonucleotide
cgcgcctagcagtgtcccagccgggttcgtgtcgcc

SEQ ID NO: 39 - DNA, 64 bp, Artificial Sequence
  Description of Artificial Sequence Synthetic oligonucleotide
cctcgccccggacctgccctcccgccaggtgcacccacctgcaataaatgcagcgaagccggga

SEQ ID NO: 40 - DNA, 782 bp, Artificial Sequence
  Description of Artificial Sequence Synthetic polynucleotide
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagagaccctcgagaatattctcgagggtctcggaatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagaagctttggagccacccgcagttcgagaaaggtggaggttccggaggtggatcgggaggttcggcgtggagccacccgcagttcgaaaaataa

SEQ ID NO: 41 - DNA, 1272 bp, Artificial Sequence
  Description of Artificial Sequence Synthetic polynucleotide
atgccacaatttgatatattatgtaaaacaccacctaaggtgcttgttcgtcagtttgtggaaaggtttgaaagaccttcaggtgagaaaatagcattatgtgctgctgaactaacctatttatgttggatgattacacataacggaacagcaatcaagagagccacattcatgagctataatactatcataagcaattcgctgagtttggatattgtcaacaagtcactgcagtttaaatacaagacgcaaaaagcaacaattctggaagcctcattaaagaaattgattcctgcttgggaatttacaattattccttactatggacaaaaacatcaatctgatatcactgatattgtaagtagtttgcaattacagttcgaatcatcggaagaagcagataagggaaatagccacagtaaaaaaatgcttaaagcacttctaagtgagggtgaaagcatctgggagatcactgagaaaatactaaattcgtttgagtatacttcgagatttacaaaaacaaaaactttataccaattcctcttcctagctactttcatcaattgtggaagattcagcgatattaagaacgttgatccgaaatcatttaaattagtccaaaataagtatctgggagtaataatccagtgtttagtgacagagacaaagacaagcgttagtaggcacatatacttctttagcgcaaggggtaggatcgatccacttgtatatttggatgaatttttgaggaattctgaaccagtcctaaaacgagtaaataggaccggcaattcttcaagcaacaagcaggaataccaattattaaaagataacttagtcagatcgtacaacaaagctttgaagaaaaatgcgccttattcaatctttgctataaaaaatggcccaaaatctcacattggaagacatttgatgacctcatttctttcaatgaagggcctaacggagttgactaatgttgtgggaaattggagcgataagcgtgcttctgccgtggccaggacaacgtatactcatcagataacagcaatacctgatcactacttcgcactagtttctcggtactatgcatatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataa

SEQ ID NO: 42 - DNA, 48 bp, Artificial Sequence
  Description of Artificial Sequence Synthetic oligonucleotide
gaagttcctattccgaagttcctattctctagaaagtataggaacttc

(19) United States
(12) Patent Application Publication
(10) Pub. No.: US 2022/0228148 A1
(43) Pub. Date: Jul. 21, 2022

(54) EUKARYOTIC SEMI-SYNTHETIC ORGANISMS

(71) Applicant: The Scripps Research Institute, La Jolla, CA (US)

(72) Inventors: Floyd E. ROMESBERG, La Jolla, CA (US); Anne Xiaozhou ZHOU, La Jolla, CA (US); Kai SHENG, La Jolla, CA (US)

(21) Appl. No.: 17/709,041

(22) Filed: Mar. 30, 2022

Related U.S. Application Data
(63) Continuation of application No. PCT/US2020/053339, filed on Sep. 29, 2020.
(60) Provisional application No. 62/908,421, filed on Sep. 30, 2019.

(51) Int. Cl. C12N 15/113; C12P 21/02

(52) U.S. Cl. CPC C12N 15/113; C12P 21/02

(57) ABSTRACT

Provided herein are eukaryotic semi-synthetic organisms and their methods of use and manufacture.

### CROSS-REFERENCE TO RELATED APPLICATIONS

This application is a continuation of International Application No. PCT/US2020/053339, filed Sep. 29, 2020, which claims priority to U.S. Provisional Application No. 62/908,421, filed on Sep. 30, 2019, which is herein incorporated by reference in its entirety.

### STATEMENT AS TO FEDERALLY SPONSORED RESEARCH

This invention was made with government support under Grant No. GM 118178 awarded by the National Institutes of Health (NIH). The government has certain rights in this invention.

### SEQUENCE LISTING

The instant application contains a Sequence Listing which has been submitted electronically in ASCII format and is hereby incorporated by reference in its entirety. Said ASCII copy, created on Sep. 24, 2020, is named 36271-810_301_SL.txt and is 19,000 bytes in size.

### BACKGROUND OF THE INVENTION

Every protein ever produced in a cell has been encoded with a four-letter, two-base-pair genetic alphabet. This generally restricts the amino acids from which proteins may be built to the canonical 20 proteogenic amino acids. While this has allowed for the diversity of life, many potential functionalities are not available, and thus an expansion to include non-canonical amino acids (ncAAs), including ones selected to provide a desired activity, might allow for the creation of novel proteins with improved properties, for applications ranging from materials to therapeutics. Efforts to incorporate ncAAs have mostly relied on expansion of the genetic alphabet via stop (UAG) or four-letter codon (quadruplet codons) suppression, although in these cases incorporation of the ncAA must compete with the codons' natural functions. To circumvent this limitation, efforts have been focused on the synthesis of genomes with natural stop or rare codons eliminated, thus liberating them for reassignment to ncAAs. However, rare codons may potentially play important roles in the regulation of translation and protein folding, and genome synthesis is impractical as a general strategy, especially with large eukaryotic genomes.

An alternative approach relies on the use of an unnatural base pair (UBP), which in principle, from a practical perspective, would allow for the creation of a virtually unlimited number of new entirely new codons unencumbered by any natural function. By pursuing a medicinal chemistry-like, a family of UBPs have been developed, typified by dNaM-dTPT3 (FIG. 1B), which have been used as the basis of an E. coli semi-synthetic organism (SSO). The E. coli SSO stores the UBP in its genome or on a plasmid, transcribes it into mRNA and tRNA, and with the tRNA charged with a ncAA by an orthogonal synthetase, translates proteins containing the ncAA. The E. coli SSO has important practical applications as it is currently being used to produce novel therapeutics.

The breadth of ncAAs and resulting unnatural polypeptides that may be produced is dictated, at least in part, on the SSO used. To date, use of the UBPs, such as dNAM-dTPT3, has not been shown in eukaryotic SSO or system. Proof-of-concept of the approach summarized herein in eukaryotic cells would enable the production of a wider range of ncAAs and resulting unnatural polypeptides, that may be useful for important practical applications such as to produce novel therapeutics.

### SUMMARY OF THE INVENTION

Provided herein, in some embodiments, are eukaryotic semi-synthetic organisms (SSOs) that were generated by exploring the translation of unnatural codons. Protein production was characterized after direct, transient, triple transfection with mRNA containing an unnatural codon, tRNA containing a cognate unnatural codon, and DNA encoding an appropriate synthetase to charge the tRNA with a non-canonical amino acid (ncAA).

Aspects disclosed herein provide eukaryotic cells comprising (a) a messenger RNA (mRNA) with a codon comprising a first unnatural base and (b) a transfer RNA (tRNA) with an anticodon comprising a second unnatural base, wherein the first and second unnatural bases form an unnatural base pair (UBP) in the eukaryotic cell, and wherein the mRNA is capable of being translated in the cell to produce a polypeptide comprising at least one unnatural amino acid. In some embodiments, the tRNA is charged with an unnatural amino acid. In some embodiments, the eukaryotic cell further comprises a polypeptide translated from the mRNA, wherein the polypeptide comprises at least one unnatural amino acid. In some embodiments, eukaryotic cell further comprises a ribosome that is capable of translating a polypeptide comprising the at least one unnatural amino acid from the mRNA using the tRNA.

Aspects disclosed herein also provide eukaryotic cells comprising an unnatural base pair (UBP) comprising: (a) a first unnatural ribonucleotide comprising a first unnatural base; (b) a second unnatural ribonucleotide comprising a second unnatural base, wherein the first and second unnatural bases form an unnatural base pair (UBP) in the eukaryotic cell.

In some embodiments, the first unnatural base or the second unnatural base is selected from the group consisting of: (i) 2-thiouracil, 2-thio-thymine, 2′-deoxyuridine, 4-thio-uracil, 4-thio-thymine, uracil-5-yl, hypoxanthin-9-yl (I), 5-halouracil; 5-propynyl-uracil, 6-azo-thymine, 6-azo-uracil, 5-methylaminomethyluracil, 5-methoxyaminomethyl-2-thiouracil, pseudouracil, uracil-5-oxacetic acid methylester, uracil-5-oxacetic acid, 5-methyl-2-thiouracil, 3-(3-amino-3-N-2-carboxypropyl) uracil, 5-methyl-2-thiouracil, 4-thiouracil, 5-methyluracil, 5′-methoxycarboxymethyluracil, 5-methoxyuracil, uracil-5-oxyacetic acid, 5-(carboxyhydroxylmethyl) uracil, 5-carboxymethylaminomethyl-2-thiouridine, 5-carboxymethylaminomethyluracil, or dihydrouracil; (ii) 5-hydroxymethyl cytosine, 5-trifluoromethyl cytosine, 5-halocytosine, 5-propynyl cytosine, 5-hydroxycytosine, cyclocytosine, cytosine arabinoside, 5,6-dihydrocytosine, 5-nitrocytosine, 6-azo cytosine, azacytosine, N4-ethylcytosine, 3-methylcytosine, 5-methylcytosine, 4-acetylcytosine, 2-thiocytosine, phenoxazine cytidine([5,4-b][1,4]benzoxazin-2(3H)-one), phenothiazine cytidine (1H-pyrimido[5,4-b][1,4]benzothiazin-2(3H)-one), phenoxazine cytidine (9-(2-aminoethoxy)-H-pyrimido[5,4-b][1,4]benzoxazin-2(3H)-one), carbazole cytidine (2H-pyrimido[4,5-b]indol-2-one), or pyridoindole cytidine (H-pyrido [3′,2′:4,5]pyrrolo [2,3-d]pyrimidin-2-one); (iii) 2-aminoadenine, 2-propyl adenine, 2-amino-adenine, 2-F-adenine, 2-amino-propyl-adenine, 2-amino-2′-deoxyadenosine, 3-deazaadenine, 7-methyladenine, 7-deaza-adenine, 8-azaadenine, 8-halo, 8-amino, 8-thiol, 8-thioalkyl, and 8-hydroxyl substituted adenines, N6-isopentenyladenine, 2-methyladenine, 2,6-diaminopurine, 2-methythio-N6-isopentenyladenine, or 6-aza-adenine; (iv) 2-methylguanine, 2-propyl and alkyl derivatives of guanine, 3-deazaguanine, 6-thio-guanine, 7-methylguanine, 7-deazaguanine, 7-deazaguanosine, 7-deaza-8-azaguanine, 8-azaguanine, 8-halo, 8-amino, 8-thiol, 8-thioalkyl, and 8-hydroxyl substituted guanines, 1-methylguanine, 2,2-dimethylguanine, 7-methylguanine, or 6-aza-guanine; and (v) hypoxanthine, xanthine, 1-methylinosine, queosine, beta-D-galactosylqueosine, inosine, beta-D-mannosylqueosine, wybutoxosine, hydroxyurea, (acp3)w, 2-aminopyridine, or 2-pyridone. In some embodiments, the first unnatural base and the second unnatural base are each, independently, selected from the group consisting of

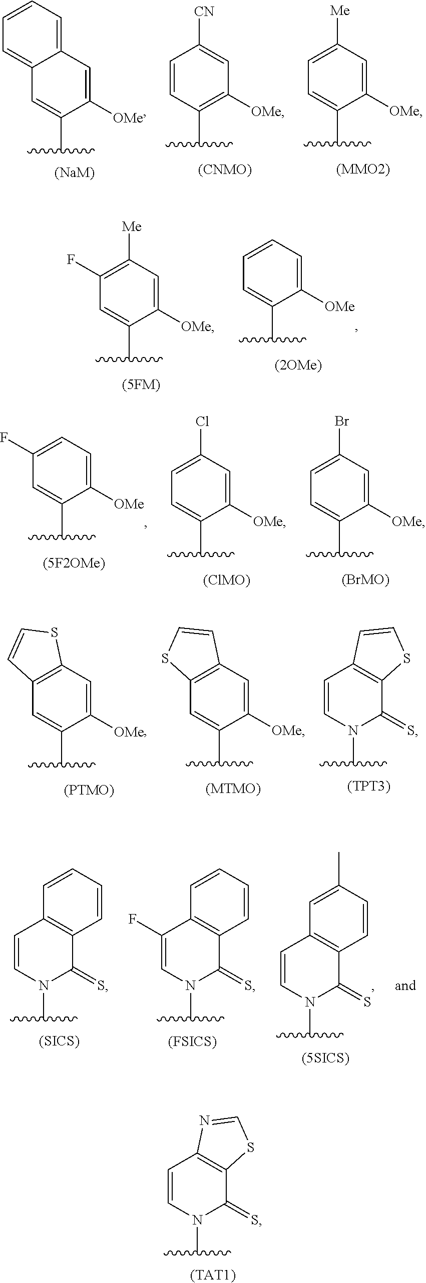

wherein the wavy line indicates a bond to a ribosyl moiety. In some embodiments, when the first unnatural base is

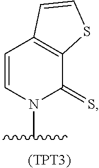

the second unnatural base is

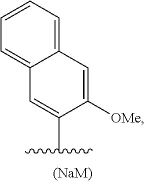

and when the first unnatural base is

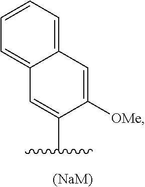

the second unnatural base is

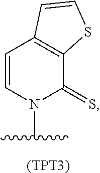

wherein the wavy line indicates a bond to a ribosyl moiety. In some embodiments, when the first unnatural base is

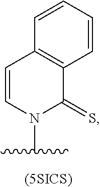

the second unnatural base is

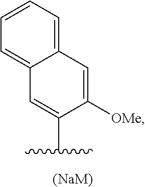

and when the first unnatural base is

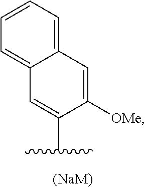

the second unnatural base is

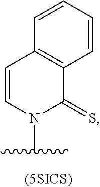

wherein the wavy line indicates a bond to a ribosyl moiety. In some embodiments, when the first unnatural base is

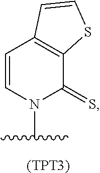

the second unnatural base is

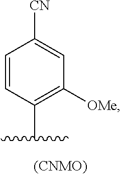

and when the first unnatural base is

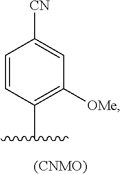

the second unnatural base is

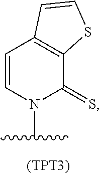

wherein the wavy line indicates a bond to a ribosyl moiety. In some embodiments, when the first unnatural base is

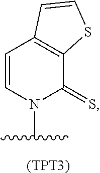

the second unnatural base is

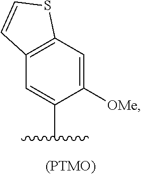

and when the first unnatural base is

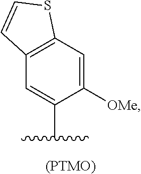

the second unnatural base is

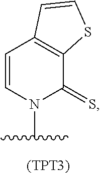

wherein the wavy line indicates a bond to a ribosyl moiety. In some embodiments, when the first unnatural base is

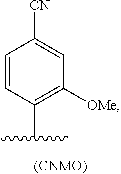

the second unnatural base is

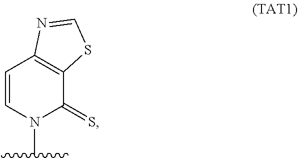 [SMILES: *n1ccc2ncsc2c1=S]

and when the first unnatural base is

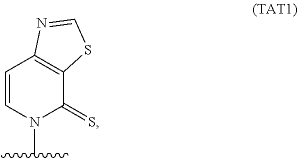 [SMILES: *n1ccc2ncsc2c1=S]

the second unnatural base is

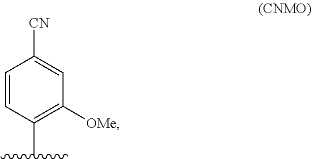 [SMILES: *c1ccc(C#N)cc1OC]

wherein the wavy line indicates a bond to a ribosyl moiety. In some embodiments, when the first unnatural base is

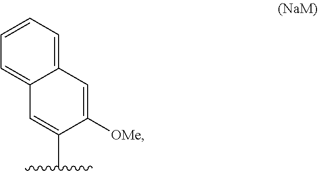 [SMILES: *c1cc2ccccc2cc1OC]

the second unnatural base is

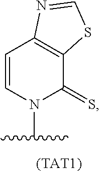

and when the first unnatural base is

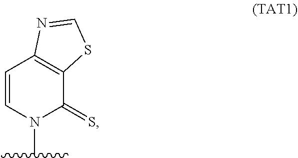 [SMILES: *n1ccc2ncsc2c1=S]

the second unnatural base is

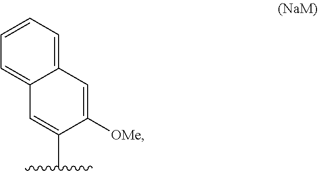 [SMILES: *c1cc2ccccc2cc1OC]

wherein the wavy line indicates a bond to a ribosyl moiety. In some embodiments, the first unnatural base or the second unnatural base comprise a modified sugar moiety selected from the group consisting of: a modification at the 2′ position:
OH, substituted lower alkyl, alkaryl, aralkyl, O-alkaryl or O-aralkyl, SH, SCH₃, OCN, Cl,
Br, CN, CF₃, OCF₃, SOCH₃, SO₂CH₃, ONO₂, NO₂, N₃, NH₂F;
O-alkyl, S-alkyl, N-alkyl;
O-alkenyl, S-alkenyl, N-alkenyl;
O-alkynyl, S-alkynyl, N-alkynyl;
O-alkyl-O-alkyl, 2′-F, 2′—OCH₃, 2′—O(CH₂)₂OCH₃ wherein the alkyl, alkenyl and alkynyl may be substituted or unsubstituted C₁-C₁₀, alkyl, C₂-C₁₀ alkenyl, C₂-C₁₀ alkynyl, —
O[(CH₂)ₙO]ₘCH₃, —O(CH₂)ₙOCH₃, —O(CH₂)ₙNH₂, —O(CH₂)ₙCH₃, —O(CH₂)ₙ—NH₂, and —
O(CH₂)ₙON[(CH₂)ₙCH₃)]₂, wherein n and m are from 1 to about 10;
and/or a modification at the 5′ position:
5′-vinyl, 5′-methyl (R or S);
a modification at the 4′ position:
4′-S, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and any combination thereof.

In some embodiments, the eukaryotic cell further comprises: (a) a transfer RNA (tRNA) with an anticodon comprising the first unnatural base; (b) a messenger RNA (mRNA) with a codon comprising the second unnatural base, wherein the first and second unnatural bases are capable of forming an unnatural base pair (UBP) in the eukaryotic cell. In some embodiments, the eukaryotic cell further comprises: (a) a transfer RNA (tRNA) with an anticodon comprising the second unnatural base; (b) a messenger RNA (mRNA) with a codon comprising the first unnatural base, wherein the first and second unnatural bases are capable of forming an unnatural base pair (UBP) in the eukaryotic cell. In some embodiments, the codon of the mRNA comprises three contiguous nucleobases (N—N—N); and wherein the first unnatural base (X) is located at the first position (X—N—N) in the codon of the mRNA. In some embodiments, the codon of the mRNA comprises three contiguous nucleobases (N—N—N); and wherein the first unnatural base (X) is located at the middle position (N—X—N) in the codon of the mRNA. In some embodiments, the codon of the mRNA comprises three contiguous nucleobases (N—N—N); and wherein the first unnatural base (X) is located at the last position (N—N—X) in the codon of the mRNA. In some embodiments, the eukaryotic cell further comprises a polypeptide translated from the mRNA, wherein the polypeptide comprises at least one unnatural amino acid. In some embodiments, the at least one unnatural amino acid: (a) is a lysine analogue; (b) comprises an aromatic side chain; (c) comprises an azido group; (d) comprises an alkyne group; or (e) comprises an aldehyde or ketone group. In some embodiments, the one or more unnatural amino acid is selected from the group consisting of N6-((azidoethoxy)-carbonyl)-L-lysine (AzK), N6-((propargylethoxy)-carbonyl)-L-lysine (PraK), BCN-L-lysine, norbomene lysine, TCO-lysine, methyltetrazine lysine, allyloxycarbonyllysine, 2-amino-8-oxononanoic acid, 2-amino-8-oxooctanoic acid, p-acetyl-L-phenylalanine, p-azidomethyl-L-phenylalanine (pAMF), p-iodo-L-phenylalanine, m-acetylphenylalanine, 2-amino-8-oxononanoic acid, p-propargyloxyphenylalanine, p-propargyl-phenylalanine, 3-methyl-phenylalanine, L-Dopa, fluorinated phenylalanine, isopropyl-L-phenylalanine, p-azido-L-phenylalanine, p-acyl-L-phenylalanine, p-benzoyl-L-phenylalanine, p-bromophenylalanine, p-amino-L-phenylalanine, isopropyl-L-phenylalanine, O-allyltyrosine, O-methyl-L-tyrosine, O-4-allyl-L-tyrosine, 4-propyl-L-tyrosine, phosphonotyrosine, tri-O-acetyl-GlcNAcp-serine, L-phosphoserine, phosphonoserine, L-3-(2-naphthyl)alanine, 2-amino-3-((2-((3-(benzyloxy)-3-oxopropyl)amino)ethyl)selanyl)propanoic acid, 2-amino-3-(phenylselanyl)propanoic, selenocysteine, N6-(((2-azidobenzyl)oxy)carbonyl)-L-lysine, N6-(((3-azidobenzyl)oxy)carbonyl)-L-lysine, and N6-(((4-azidobenzyl)oxy)carbonyl)-L-lysine. In some embodiments, the at least one unnatural amino acid is N6-((azidoethoxy)-carbonyl)-L-lysine (AzK). In some embodiments, the at least one unnatural amino acid is N6-(((2-azidobenzyl)oxy)carbonyl)-L-lysine. In some embodiments, the at least one unnatural amino acid is N6-(((3-azidobenzyl)oxy)carbonyl)-L-lysine. In some embodiments, the at least one unnatural amino acid is N6-(((4-azidobenzyl)oxy)carbonyl)-L-lysine. In some embodiments, the eukaryotic cell is a human cell. In some embodiments, the human cell is a HEK293T cell. In some embodiments, the cell is a hamster cell. In some embodiments, the hamster cell is a Chinese hamster ovary (CHO) cell. In some embodiments, the cell is isolated and purified. In some embodiments, the mRNA and the tRNA are stabilized to degradation in the eukaryotic cell.

Aspects disclosed herein provide semi-synthetic organisms comprising the eukaryotic cell described herein.

Aspects disclosed herein provide eukaryotic cell lines comprising a plurality of eukaryotic cells of the present disclosure.

Aspects disclosed herein provide methods of producing a polypeptide comprising one or more unnatural amino acids in a eukaryotic cell, comprising: (a) introducing into the cell: (i) a messenger RNA (mRNA) with a codon comprising a first unnatural base; and (ii) a transfer RNA (tRNA) with an anticodon comprising a second unnatural base in the eukaryotic cell, wherein the first and second unnatural bases form an unnatural base pair (UBP) in the eukaryotic cell; and (b) translating the polypeptide comprising the one or more unnatural amino acids from the mRNA using the tRNA. In some embodiments, the tRNA is charged with an unnatural amino acid.

Aspects disclosed herein also provide methods of producing a polypeptide comprising one or more unnatural amino acids in a eukaryotic cell, comprising: (a) providing a eukaryotic cell comprising: (i) a messenger RNA (mRNA) with a codon comprising a first unnatural base; (ii) a transfer RNA (tRNA) with an anticodon comprising a second unnatural base, wherein the first and second unnatural bases form an unnatural base pair (UBP) in the eukaryotic cell; (b) translating the polypeptide comprising the one or more unnatural amino acids from the mRNA using the tRNA by a ribosome that is endogenous to the eukaryotic cell. In some embodiments, the polypeptide comprises a eukaryotic glycosylation pattern. The glycosylation pattern may correspond to the cell in which it is produced (e.g., be a mammalian glycosylation pattern when the cell is mammalian, a human glycosylation pattern when the cell is human, etc.).

Aspects disclosed herein also provide methods of producing a polypeptide in a eukaryotic cell, wherein the polypeptide comprises one or more unnatural amino acids, the method comprising, the method comprising: (a) providing a eukaryotic cell, the eukaryotic cell comprising: (i) an mRNA comprising a codon, wherein the codon comprises a first unnatural base; (ii) a tRNA comprising an anti-codon, wherein the anti-codon comprises a second unnatural base, and wherein the first and second unnatural bases form a complimentary base pair; and (iii) a tRNA synthetase, wherein the tRNA synthetase preferentially aminoacylates the tRNA with the one or more unnatural amino acids compared to a natural amino acid; and (b) providing the one more unnatural amino acids to the eukaryotic cell, wherein the eukaryotic cell produces the polypeptide comprising the one or more unnatural amino acids.

Aspects disclosed herein also provide methods of producing a polypeptide comprising one or more unnatural amino acids in a eukaryotic cell, comprising: (a) providing a eukaryotic cell comprising: (i) a transfer RNA (tRNA) with an anticodon comprising a first unnatural base; (ii) a messenger RNA (mRNA) with a codon comprising a second unnatural base, wherein the first and second unnatural bases form an unnatural base pair (UBP) in the eukaryotic cell; and (c) translating the polypeptide comprising the one or more unnatural amino acids from the mRNA using the tRNA by a ribosome that is endogenous to the eukaryotic cell.

In some embodiments, the codon of the mRNA comprises three contiguous nucleobases (N—N—N); and wherein the first unnatural base (X) is located at the first position (X—N—N) in the codon of the mRNA. In some embodiments, the codon of the mRNA comprises three contiguous nucleobases (N—N—N); and wherein the first unnatural base (X) is located at the middle position (N—X—N) in the codon of the mRNA. In some embodiments, the codon of the mRNA comprises three contiguous nucleobases (N—N—N); and wherein the first unnatural base (X) is located at the last position (N—N—X) in the codon of the mRNA. In some embodiments, the first unnatural base or the second unnatural base is selected from the group consisting of: (a) 2-thiouracil, 2-thio-thymine, 2′-deoxyuridine, 4-thio-uracil, 4-thio-thymine, uracil-5-yl, hypoxanthin-9-yl (I), 5-halouracil; 5-propynyl-uracil, 6-azo-thymine, 6-azo-uracil, 5-methylaminomethyluracil, 5-methoxyaminomethyl-2-thiouracil, pseudouracil, uracil-5-oxacetic acid methylester, uracil-5-oxacetic acid, 5-methyl-2-thiouracil, 3-(3-amino-3-N-2-carboxypropyl) uracil, 5-methyl-2-thiouracil, 4-thiouracil, 5-methyluracil, 5′-methoxycarboxymethyluracil, 5-methoxyuracil, uracil-5-oxyacetic acid, 5-(carboxyhydroxylmethyl) uracil, 5-carboxymethylaminomethyl-2-thiouridine, 5-carboxymethylaminomethyluracil, or dihydrouracil; (b) 5-hydroxymethyl cytosine, 5-trifluoromethyl cytosine, 5-halocytosine, 5-propynyl cytosine, 5-hydroxycytosine, cyclocytosine, cytosine arabinoside, 5,6-dihydrocytosine, 5-nitrocytosine, 6-azo cytosine, azacytosine, N4-ethylcytosine, 3-methylcytosine, 5-methylcytosine, 4-acetylcytosine, 2-thiocytosine, phenoxazine cytidine([5,4-b][1,4]benzoxazin-2(3H)-one), phenothiazine cytidine (1H-pyrimido[5,4-b][1,4]benzothiazin-2(3H)-one), phenoxazine cytidine (9-(2-aminoethoxy)-H-pyrimido[5,4-b][1,4]benzoxazin-2(3H)-one), carbazole cytidine (2H-pyrimido[4,5-b]indol-2-one), or pyridoindole cytidine (H-pyrido [3′,2′:4,5]pyrrolo [2,3-d]pyrimidin-2-one); (c) 2-aminoadenine, 2-propyl adenine, 2-amino-adenine, 2-F-adenine, 2-amino-propyl-adenine, 2-amino-2′-deoxyadenosine, 3-deazaadenine, 7-methyladenine, 7-deaza-adenine, 8-azaadenine, 8-halo, 8-amino, 8-thiol, 8-thioalkyl, and 8-hydroxyl substituted adenines, N6-isopentenyladenine, 2-methyladenine, 2,6-diaminopurine, 2-methythio-N6-isopentenyladenine, or 6-aza-adenine; (d) 2-methylguanine, 2-propyl and alkyl derivatives of guanine, 3-deazaguanine, 6-thio-guanine, 7-methylguanine, 7-deazaguanine, 7-deazaguanosine, 7-deaza-8-azaguanine, 8-azaguanine, 8-halo, 8-amino, 8-thiol, 8-thioalkyl, and 8-hydroxyl substituted guanines, 1-methylguanine, 2,2-dimethylguanine, 7-methylguanine, or 6-aza-guanine; and (e) hypoxanthine, xanthine, 1-methylinosine, queosine, beta-D-galactosylqueosine, inosine, beta-D-mannosylqueosine, wybutoxosine, hydroxyurea, (acp3)w, 2-aminopyridine, or 2-pyridone. In some embodiments, the first unnatural base or the second unnatural base is selected from the group consisting of

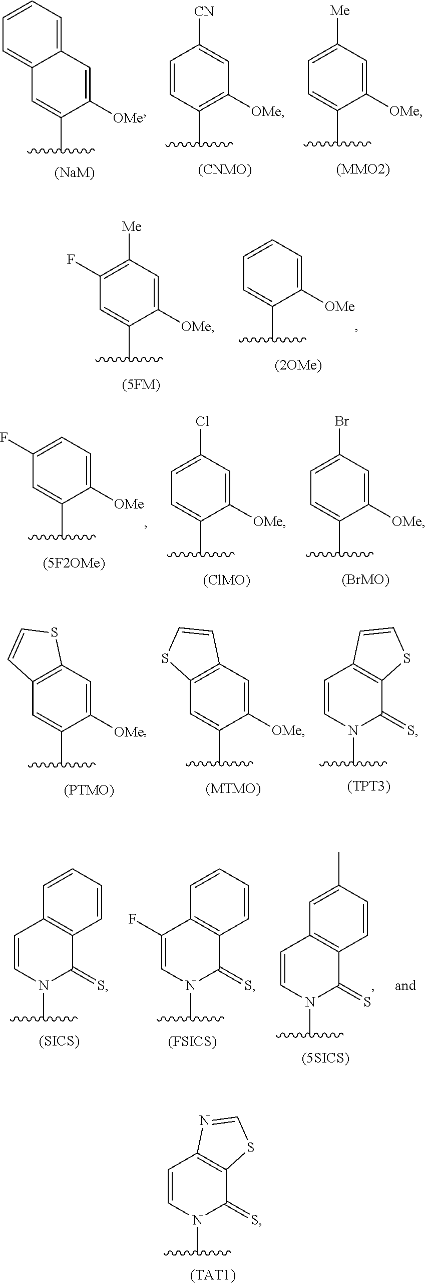

wherein the wavy line indicates a bond to a ribosyl moiety. In some embodiments, the first unnatural base is

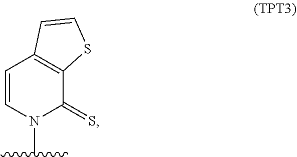 [SMILES: *n1ccc2ccsc2c1=S]

the second unnatural base is

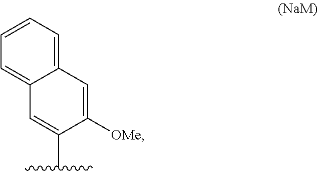 [SMILES: *c1cc2ccccc2cc1OC]

and when the first unnatural base is

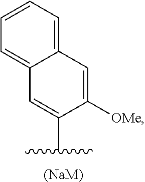

the second unnatural base is

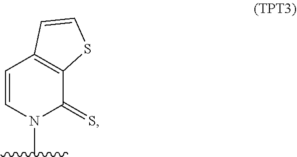 [SMILES: *n1ccc2ccsc2c1=S]

wherein the wavy line indicates a bond to a ribosyl moiety. In some embodiments, when the first unnatural base is

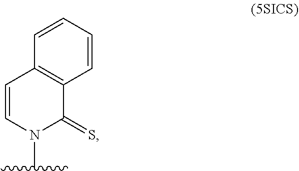 [SMILES: *n1ccc2ccccc2c1=S]

the second unnatural base is

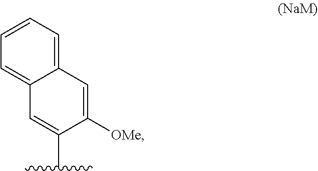 [SMILES: *c1cc2ccccc2cc1OC]

and when the first unnatural base is

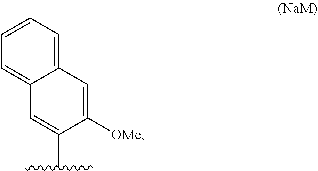 [SMILES: *c1cc2ccccc2cc1OC]

the second unnatural base is

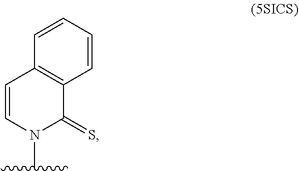 [SMILES: *n1ccc2ccccc2c1=S]

wherein the wavy line indicates a bond to a ribosyl moiety. In some embodiments, when the first unnatural base is

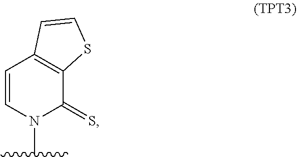 [SMILES: *n1ccc2ccsc2c1=S]

the second unnatural base is

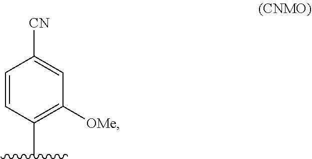 [SMILES: *c1ccc(C#N)cc1OC]

and when the first unnatural base is

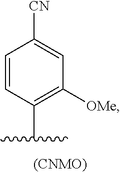 [SMILES: *c1ccc(C#N)cc1OC]

the second unnatural base is

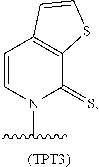

wherein the wavy line indicates a bond to a ribosyl moiety. In some embodiments, when the first unnatural base is

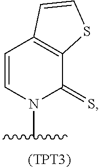

the second unnatural base is

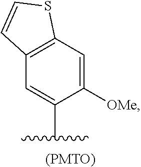

and when the first unnatural base is

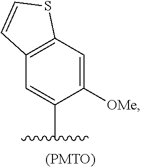

the second unnatural base is

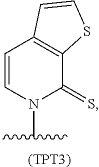

wherein the wavy line indicates a bond to a ribosyl moiety. In some embodiments, when the first unnatural base is

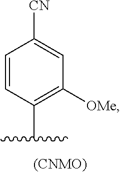 [SMILES: *c1ccc(C#N)cc1OC]

the second unnatural base is

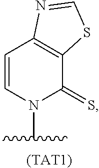

and when the first unnatural base is

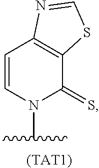

the second unnatural base is

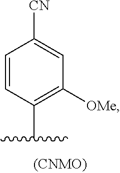 [SMILES: *c1ccc(C#N)cc1OC]

wherein the wavy line indicates a bond to a ribosyl moiety. In some embodiments when the first unnatural base is

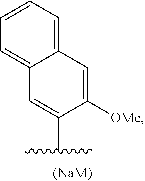

the second unnatural base is

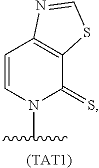

and when the first unnatural base is

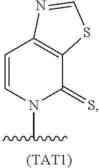

the second unnatural base is

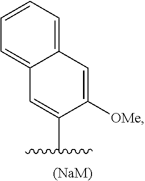

wherein the wavy line indicates a bond to a ribosyl moiety. In some embodiments, the first unnatural base or the second unnatural base comprise a modified sugar moiety selected from the group consisting of: a modification at the 2′ position:
OH, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N3, NH₂F;
O-alkyl, S-alkyl, N-alkyl;
O-alkenyl, S-alkenyl, N-alkenyl;
O-alkynyl, S-alkynyl, N-alkynyl;
O-alkyl-O-alkyl, 2′-F, 2′—OCH₃, 2′—O(CH₂)₂OCH₃ wherein the alkyl, alkenyl and alkynyl may be substituted or unsubstituted C₁-C₁₀, alkyl, C₂-C₁₀ alkenyl, C₂-C₁₀ alkynyl, —
O[(CH₂)ₙO]ₘCH₃, —O(CH₂)ₙOCH₃, —O(CH₂)ₙNH₂, —O(CH₂)ₙCH₃, —O(CH₂)ₙ—NH₂, and —O(CH₂)ₙON[(CH₂)ₙCH₃)]₂, wherein n and m are from 1 to about 10;
and/or a modification at the 5′ position:
5′-vinyl, 5′-methyl (R or S);
a modification at the 4′ position:
4′-S, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and any combination thereof.

In some embodiments, the eukaryotic cell is a human cell. In some embodiments, the human cell is a HEK293T cell. In some embodiments, the cell is a hamster cell. In some embodiments, the hamster cell is a Chinese hamster ovary (CHO) cell. In some embodiments, the unnatural amino acid: (a) is a lysine analogue; (b) comprises an aromatic side chain; (c) comprises an azido group; (d) comprises an alkyne group; or (e) comprises an aldehyde or ketone group. In some embodiments, the unnatural amino acid is selected from the group consisting of N6-((azidoethoxy)-carbonyl)-L-lysine (AzK), N6-((propargylethoxy)-carbonyl)-L-lysine (PraK), BCN-L-lysine, norbomene lysine, TCO-lysine, methyltetrazine lysine, allyloxycarbonyllysine, 2-amino-8-oxononanoic acid, 2-amino-8-oxooctanoic acid, p-acetyl-L-phenylalanine, p-azidomethyl-L-phenylalanine (pAMF), p-iodo-L-phenylalanine, m-acetylphenylalanine, 2-amino-8-oxononanoic acid, p-propargyloxyphenylalanine, p-propargyl-phenylalanine, 3-methyl-phenylalanine, L-Dopa, fluorinated phenylalanine, isopropyl-L-phenylalanine, p-azido-L-phenylalanine, p-acyl-L-phenylalanine, p-benzoyl-L-phenylalanine, p-bromophenylalanine, p-amino-L-phenylalanine, isopropyl-L-phenylalanine, O-allyltyrosine, O-methyl-L-tyrosine, O-4-allyl-L-tyrosine, 4-propyl-L-tyrosine, phosphonotyrosine, tri-O-acetyl-GlcNAcp-serine, L-phosphoserine, phosphonoserine, L-3-(2-naphthyl)alanine, 2-amino-3-((2-((3-(benzyloxy)-3-oxopropyl)amino)ethyl)selanyl)propanoic acid, 2-amino-3-(phenylselanyl)propanoic, selenocysteine, N6-(((2-azidobenzyl)oxy)carbonyl)-L-lysine, N6-(((3-azidobenzyl)oxy)carbonyl)-L-lysine, and N6-(((4-azidobenzyl)oxy)carbonyl)-L-lysine. In some embodiments, the unnatural amino acid is N6-((azidoethoxy)-carbonyl)-L-lysine (AzK). In some embodiments, the one or more unnatural amino acids is N6-(((2-azidobenzyl)oxy)carbonyl)-L-lysine. In some embodiments, the one or more unnatural amino acids is N6-(((3-azidobenzyl)oxy)carbonyl)-L-lysine. In some embodiments, the one or more unnatural amino acids is N6-(((4-azidobenzyl)oxy)carbonyl)-L-lysine.

Aspects disclosed herein provide methods of producing a polypeptide in a eukaryotic cell, wherein the polypeptide comprises one or more unnatural amino acids, the method comprising: (a) providing a eukaryotic cell, the eukaryotic cell comprising: (i) an mRNA comprising a codon, wherein the codon comprises one or more unnatural bases; (ii) a tRNA comprising an anti-codon, wherein the anti-codon comprises one or more unnatural bases, and wherein the one or more unnatural bases comprising the codon in the mRNA and the one or more unnatural bases comprising the anti-codon in the tRNA form a complimentary base pair; and (iii) a tRNA synthetase, wherein the tRNA synthetase preferentially aminoacylates the tRNA with the one or more unnatural amino acids compared to a natural amino acid; and (b) providing the one more unnatural amino acids to the eukaryotic cell, wherein the eukaryotic cell produces the polypeptide comprising the one or more unnatural amino acids. In some embodiments, the codon of the mRNA comprises three contiguous nucleobases (N—N—N); and wherein the first unnatural base (X) is located at the first position (X—N—N) in the codon of the mRNA. In some embodiments, the codon of the mRNA comprises three contiguous nucleobases (N—N—N); and wherein the first unnatural base (X) is located at the middle position (N—X—N) in the codon of the mRNA. In some embodiments, the codon of the mRNA comprises three contiguous nucleobases (N—N—N); and wherein the first unnatural base (X) is located at the last position (N—N—X) in the codon of the mRNA. In some embodiments, the one or more unnatural bases comprising the codon in the mRNA is of the formula

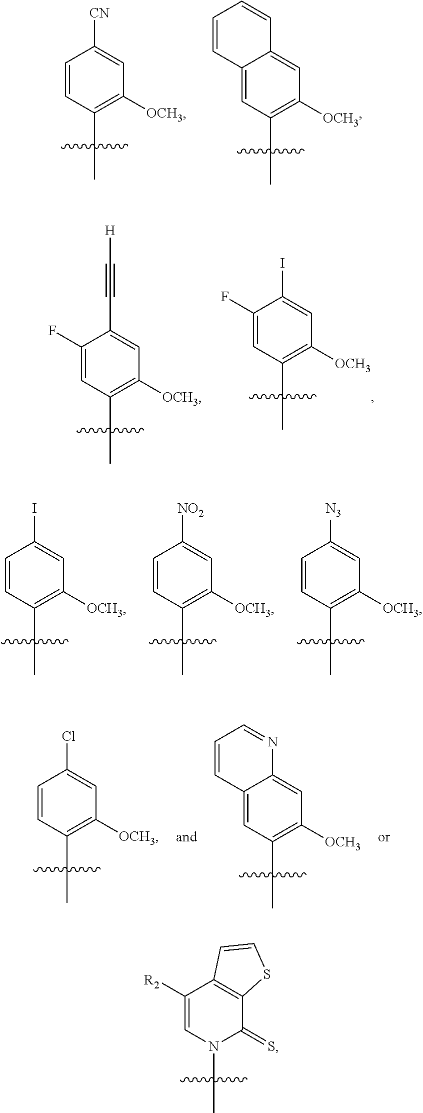

wherein R2 is selected from the group consisting of hydrogen, alkyl, alkenyl, alkynyl, methoxy, methanethiol, methaneseleno, halogen, cyano, and azido, and the wavy line indicates a bond to a ribosyl moiety. In some embodiments, the first unnatural base or the second unnatural base is selected from the group consisting of

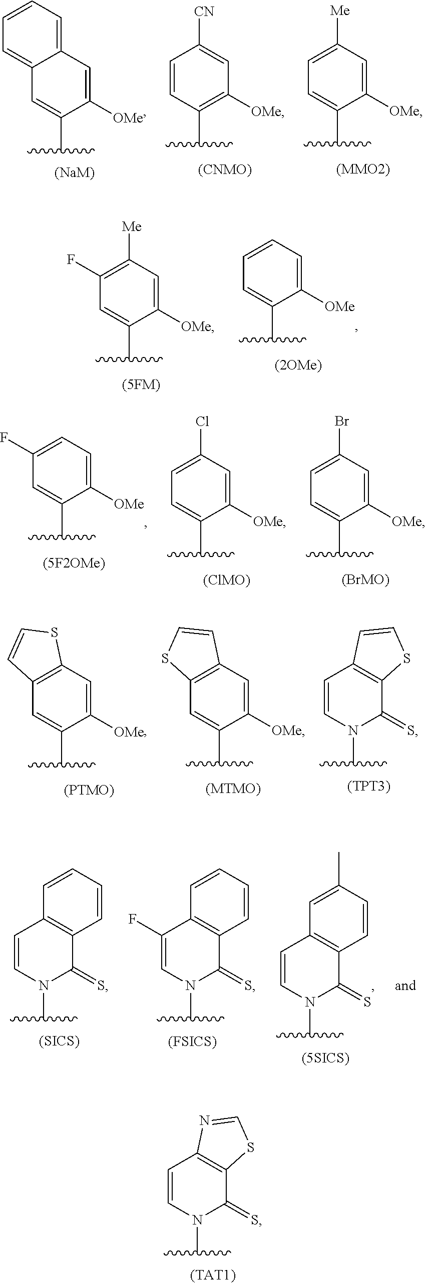

wherein the wavy line indicates a bond to a ribosyl moiety. In some embodiments, when the first unnatural base is

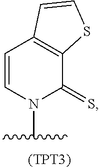

the second unnatural base is

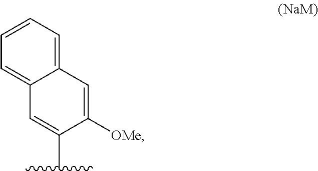 [SMILES: *c1cc2ccccc2cc1OC]

and when the first unnatural base is

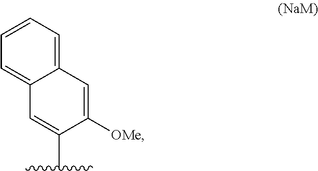 [SMILES: *c1cc2ccccc2cc1OC]

the second unnatural base is

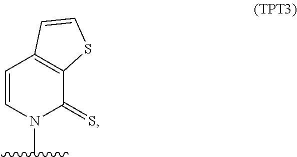 [SMILES: *n1ccc2ccsc2c1=S]

wherein the wavy line indicates a bond to a ribosyl moiety. In some embodiments, when the first unnatural base is

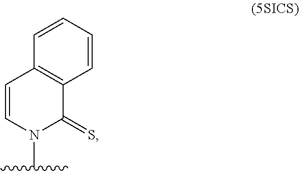 [SMILES: *n1ccc2ccccc2c1=S]

the second unnatural base is

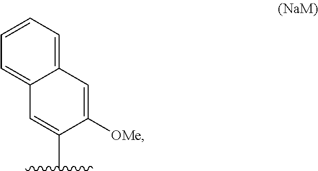 [SMILES: *c1cc2ccccc2cc1OC]

and when the first unnatural base is

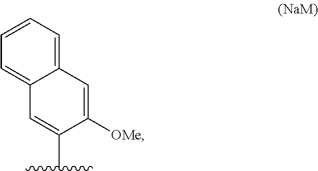 [SMILES: *c1cc2ccccc2cc1OC]

the second unnatural base is

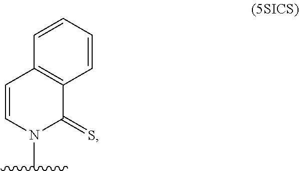 [SMILES: *n1ccc2ccccc2c1=S]

wherein the wavy line indicates a bond to a ribosyl moiety. In some embodiments, when the first unnatural base is

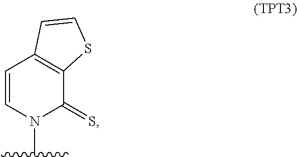 [SMILES: *n1ccc2ccsc2c1=S]

the second unnatural base is

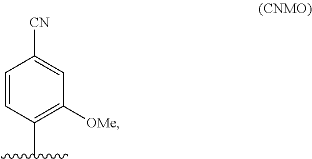 [SMILES: *c1ccc(C#N)cc1OC]

and when the first unnatural base is

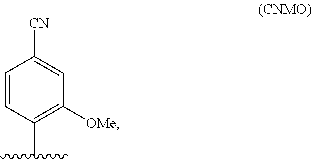 [SMILES: *c1ccc(C#N)cc1OC]

the second unnatural base is

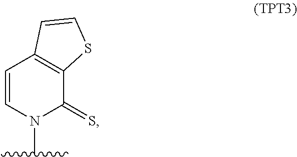 [SMILES: *n1ccc2ccsc2c1=S]

wherein the wavy line indicates a bond to a ribosyl moiety. In some embodiments, when the first unnatural base is

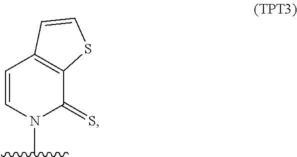 [SMILES: *n1ccc2ccsc2c1=S]

the second unnatural base is

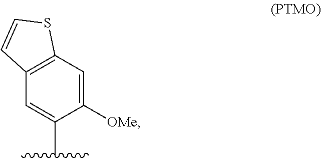 [SMILES: *c1cc2ccsc2cc1OC]

and when the first unnatural base is

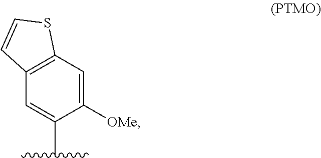 [SMILES: *c1cc2ccsc2cc1OC]

the second unnatural base is

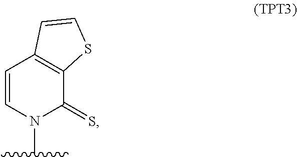 [SMILES: *n1ccc2ccsc2c1=S]

wherein the wavy line indicates a bond to a ribosyl moiety. In some embodiments, when the first unnatural base is

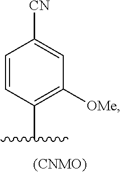 [SMILES: *c1ccc(C#N)cc1OC]

the second unnatural base is

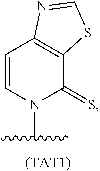

and when the first unnatural base is

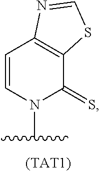

the second unnatural base is

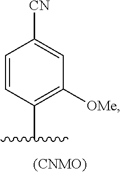 [SMILES: *c1ccc(C#N)cc1OC]

wherein the wavy line indicates a bond to a ribosyl moiety. In some embodiments, when the first unnatural base is

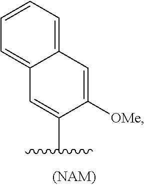 [SMILES: *c1cc2ccccc2cc1OC]

the second unnatural base is

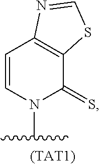

and when the first unnatural base is

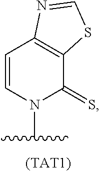

the second unnatural base is

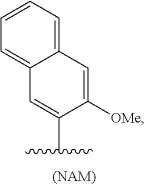 [SMILES: *c1cc2ccccc2cc1OC]

wherein the wavy line indicates a bond to a ribosyl moiety. In some embodiments, when the first unnatural base is

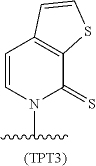

and the second unnatural base is

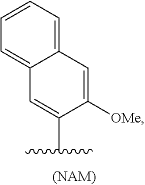 [SMILES: *c1cc2ccccc2cc1OC]

wherein the wavy line indicates a bond to a ribosyl moiety. In some embodiments, wherein the unnatural nucleotide comprising the codon in the mRNA is selected from

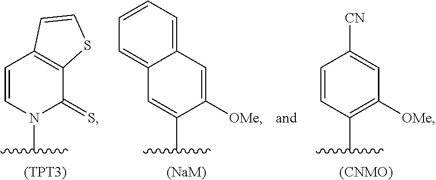

wherein the wavy line indicates a bond to a ribosyl moiety. In some embodiments, the unnatural nucleotide comprising the codon in the mRNA is

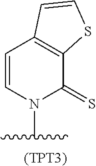

wherein the wavy line indicates a bond to a ribosyl moiety. In some embodiments, the unnatural nucleotide comprising the codon in the mRNA is

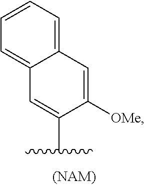 [SMILES: *c1cc2ccccc2cc1OC]

wherein the wavy line indicates a bond to a ribosyl moiety. In some embodiments, the unnatural nucleotide comprising the codon in the mRNA is

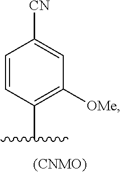 [SMILES: *c1ccc(C#N)cc1OC]

wherein the wavy line indicates a bond to a ribosyl moiety. In some embodiments, the codon of the mRNA comprises three contiguous nucleobases (N—N—N), wherein the unnatural base (X) is located at the first position (X—N—N) in the codon of the mRNA, wherein the unnatural base is selected from

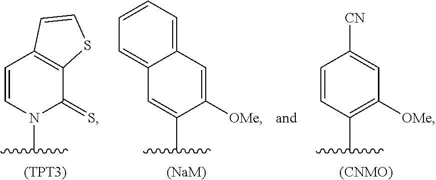

and wherein the wavy line indicates a bond to a ribosyl moiety. In some embodiments, the unnatural base is

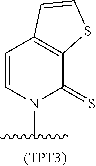

wherein the wavy line indicates a bond to a ribosyl moiety. In some embodiments, the unnatural base is

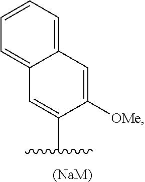

wherein the wavy line indicates a bond to a ribosyl moiety. In some embodiments, the unnatural base is

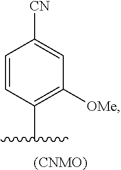

wherein the wavy line indicates a bond to a ribosyl moiety. In some embodiments, the codon of the mRNA comprises three contiguous nucleobases (N—N—N), wherein the unnatural base (X) is located at the middle position (N—X—N) in the codon of the mRNA, wherein the unnatural base is selected from

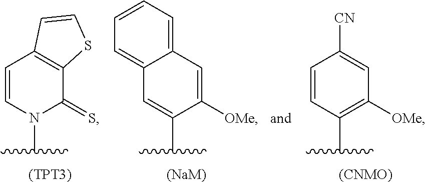

and wherein the wavy line indicates a bond to a ribosyl moiety. In some embodiments, the unnatural base is

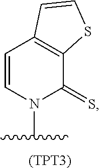

wherein the wavy line indicates a bond to a ribosyl moiety. In some embodiments, the unnatural base is

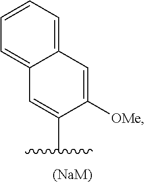

wherein the wavy line indicates a bond to a ribosyl moiety. In some embodiments, the unnatural base is

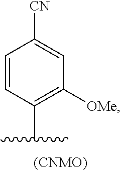

wherein the wavy line indicates a bond to a ribosyl moiety. In some embodiments, the codon of the mRNA comprises three contiguous nucleobases (N—N—N), wherein the unnatural base (X) is located at the last position (N—N—X) in the codon of the mRNA, wherein the unnatural base is selected from

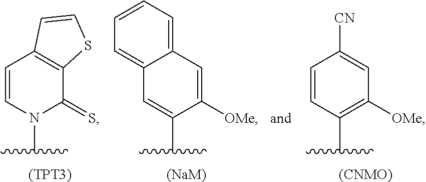

and wherein the wavy line indicates a bond to a ribosyl moiety. In some embodiments, the unnatural base is

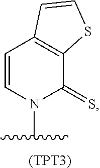

wherein the wavy line indicates a bond to a ribosyl moiety. In some embodiments, the unnatural base is

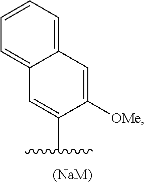

wherein the wavy line indicates a bond to a ribosyl moiety. In some embodiments, the unnatural base is

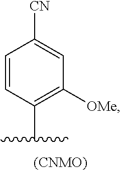

wherein the wavy line indicates a bond to a ribosyl moiety. In some embodiments, the anticodon of the tRNA comprises three contiguous nucleobases (N—N—N); and wherein the first unnatural base (X) is located at the first position (X—N—N) in the anticodon of the tRNA. In some embodiments, the unnatural base is selected from

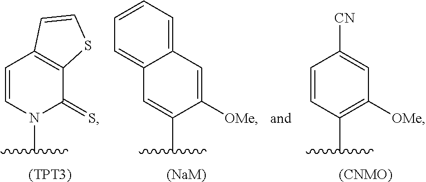

and wherein the wavy line indicates a bond to a ribosyl moiety. In some embodiments, the unnatural base is

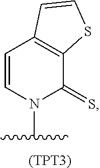

wherein the wavy line indicates a bond to a ribosyl moiety. In some embodiments, the unnatural base is

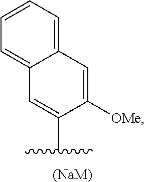

wherein the wavy line indicates a bond to a ribosyl moiety. In some embodiments, the unnatural base is

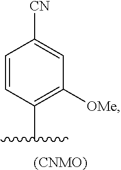

wherein the wavy line indicates a bond to a ribosyl moiety. In some embodiments, the anticodon of the tRNA comprises three contiguous nucleobases (N—N—N); and wherein the first unnatural base (X) is located at the middle position (N—X—N) in the anticodon of the tRNA. In some embodiments, the unnatural base is selected from

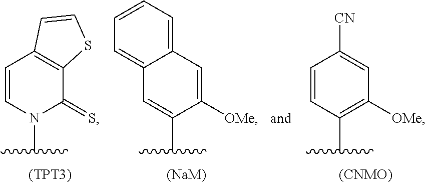

and wherein the wavy line indicates a bond to a ribosyl moiety. In some embodiments, the unnatural base is

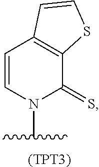

wherein the wavy line indicates a bond to a ribosyl moiety. In some embodiments, the unnatural base is

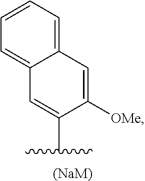

wherein the wavy line indicates a bond to a ribosyl moiety. In some embodiments, the unnatural base is

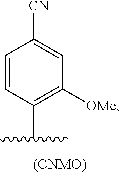 [SMILES: *c1ccc(C#N)cc1OC]

wherein the wavy line indicates a bond to a ribosyl moiety. In some embodiments, the anticodon of the tRNA comprises three contiguous nucleobases (N—N—N); and wherein the first unnatural base (X) is located at the last position (N—N—X) in the anticodon of the tRNA. In some embodiments, the unnatural base is selected from

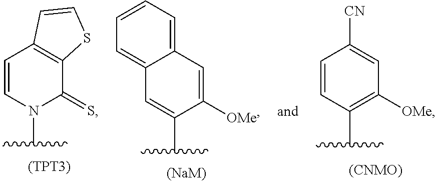

and wherein the wavy line indicates a bond to a ribosyl moiety. In some embodiments, the unnatural base is

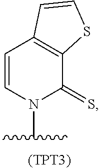

wherein the wavy line indicates a bond to a ribosyl moiety. In some embodiments, the unnatural base is

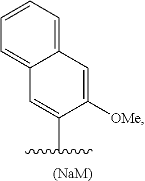

wherein the wavy line indicates a bond to a ribosyl moiety. In some embodiments, the unnatural base is

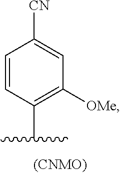 [SMILES: *c1ccc(C#N)cc1OC]

wherein the wavy line indicates a bond to a ribosyl moiety. In some embodiments, the codon and the anticodon each comprise three contiguous nucleobases (N—N—N), wherein the codon in the mRNA comprises the first unnatural base (X) located at a first position (X—N—N) of the codon, and the anticodon in the tRNA comprises the second unnatural base (Y) located at the last position (N—N—Y) of the anticodon. In some embodiments, the first unnatural base (X) located in the codon of the mRNA and the second unnatural base (Y) located in the anticodon of the tRNA are the same or are different. In some embodiments, the first unnatural base (X) located in the codon of the mRNA and the second unnatural base (Y) located in the anticodon of the tRNA are the same. In some embodiments, the first unnatural base (X) located in the codon of the mRNA and the second unnatural base (Y) located in the anticodon of the tRNA are different. In some embodiments, the first unnatural base (X) located in the codon of the mRNA and the second unnatural base (Y) located in the anticodon of the tRNA are selected from the group consisting of

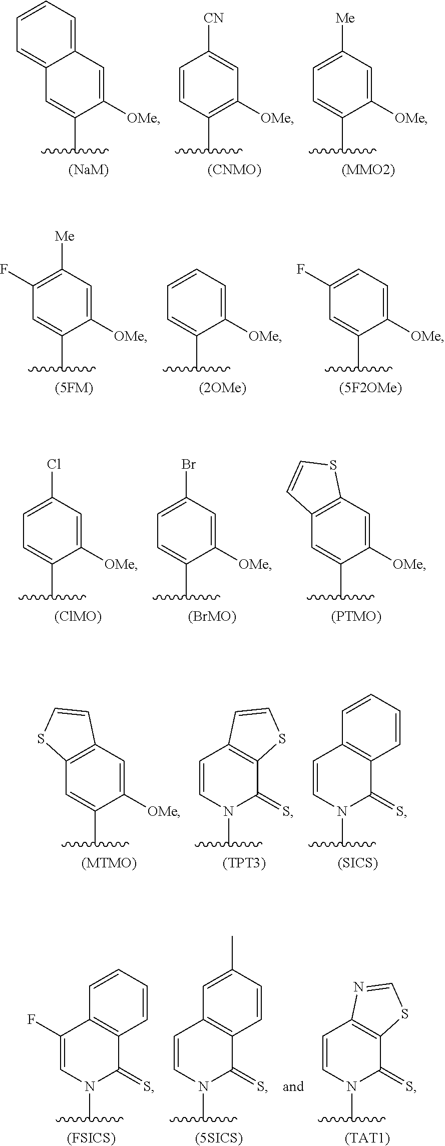

wherein the wavy line indicates a bond to a ribosyl moiety. In some embodiments, the first unnatural base (X) located in the codon of the mRNA and the second unnatural base (Y) located in the anticodon of the tRNA are selected from the group consisting of

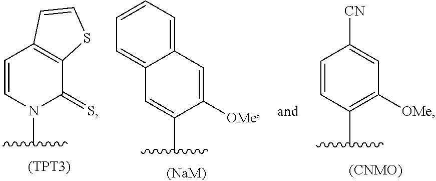

wherein the wavy line indicates a bond to a ribosyl moiety. In some embodiments, the first unnatural base (X) located in the codon of the mRNA and the second unnatural base (Y) located in the anticodon of the tRNA are both

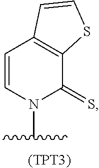

wherein the wavy line indicates a bond to a ribosyl moiety. In some embodiments, the first unnatural base (X) located in the codon of the mRNA and the second unnatural base (Y) located in the anticodon of the tRNA are both

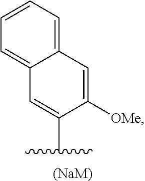

wherein the wavy line indicates a bond to a ribosyl moiety. In some embodiments, the first unnatural base (X) located in the codon of the mRNA and the second unnatural base (Y) located in the anticodon of the tRNA are both

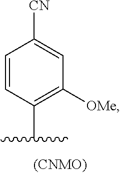 [SMILES: *c1ccc(C#N)cc1OC]

wherein the wavy line indicates a bond to a ribosyl moiety. In some embodiments, the first unnatural base (X) located in the codon of the mRNA is selected from

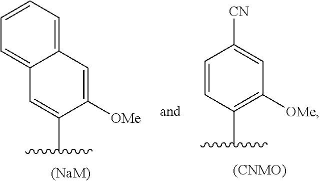

and the second unnatural base (Y) located in the anticodon of the tRNA is

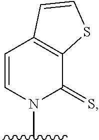 [SMILES: *n1ccc2ccsc2c1=S]

wherein in each case the wavy line indicates a bond to a ribosyl moiety. In some embodiments, the first unnatural base (X) located in the codon of the mRNA is

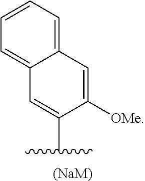

In some embodiments, the first unnatural base (X) located in the codon of the mRNA is

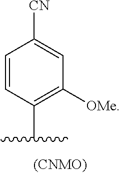 [SMILES: *c1ccc(C#N)cc1OC]

In some embodiments, the codon and the anticodon each comprise three contiguous nucleobases (N—N—N), wherein the codon in the mRNA comprises a first unnatural base (X) located at the middle position (N—X—N) of the codon, and the anticodon in the tRNA comprises a second unnatural base (Y) located at the middle position (N—Y—N) of the anticodon. In some embodiments, the first unnatural base (X) located in the codon of the mRNA and the second unnatural base (Y) located in the anticodon of the tRNA are the same or are different. In some embodiments, the first unnatural base (X) located in the codon of the mRNA and the second unnatural base (Y) located in the anticodon of the tRNA are the same. In some embodiments, the first unnatural base (X) located in the codon of the mRNA and the second unnatural base (Y) located in the anticodon of the tRNA are different. In some embodiments, the first unnatural base (X) located in the codon of the mRNA and the second unnatural base (Y) located in the anticodon of the tRNA are selected from the group consisting of

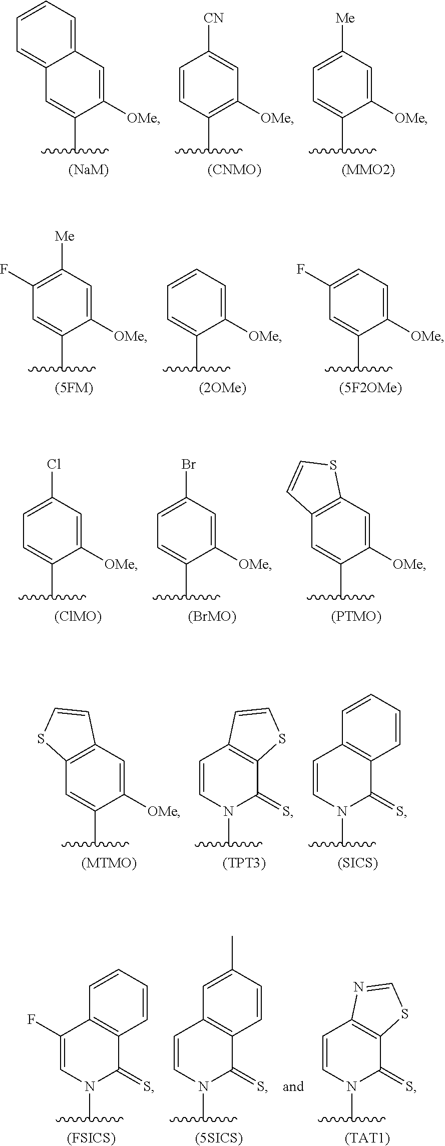

wherein the wavy line indicates a bond to a ribosyl moiety. In some embodiments, the first unnatural base (X) located in the codon of the mRNA and the second unnatural base located in the anticodon of the tRNA are selected from the group consisting of

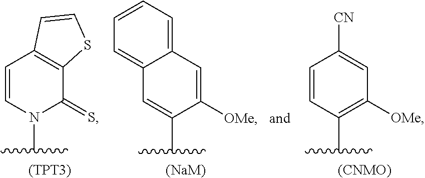

wherein the wavy line indicates a bond to a ribosyl moiety. In some embodiments, the first unnatural base (X) located in the codon of the mRNA and the second unnatural base (Y) located in the anticodon of the tRNA are both

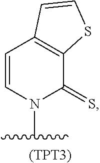

wherein the wavy line indicates a bond to a ribosyl moiety. In some embodiments, the first unnatural base (X) located in the codon of the mRNA and the second unnatural base (Y) located in the anticodon of the tRNA are both

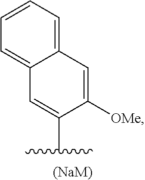

wherein the wavy line indicates a bond to a ribosyl moiety. In some embodiments, the first unnatural base (X) located in the codon of the mRNA and the second unnatural base (Y) located in the anticodon of the tRNA are both

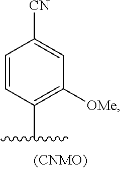 [SMILES: *c1ccc(C#N)cc1OC]

wherein the wavy line indicates a bond to a ribosyl moiety. In some embodiments, the first unnatural base (X) located in the codon of the mRNA is selected from

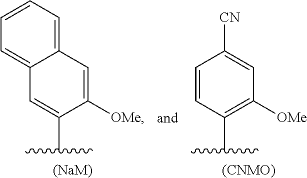

and the second unnatural base (Y) located in the anticodon of the tRNA is

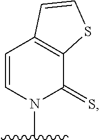 [SMILES: *n1ccc2ccsc2c1=S]

wherein in each case the wavy line indicates a bond to a ribosyl moiety. In some embodiments, the first unnatural base (X) located in the codon of the mRNA is

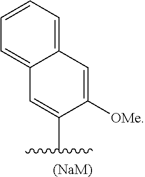

In some embodiments, the first unnatural base (X) located in the codon of the mRNA is

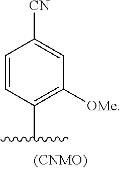 [SMILES: *c1ccc(C#N)cc1OC]

In some embodiments, the codon and the anticodon each comprise three contiguous nucleobases (N—N—N), wherein the codon in the mRNA comprises a first unnatural base (X) located at the last position (N—N—X) of the codon, and the anticodon in the tRNA comprises a second unnatural base (Y) located at the first position (Y—N—N) of the anticodon. In some embodiments, the first unnatural base (X) located in the codon of the mRNA and the second unnatural base (Y) located in the anticodon of the tRNA are the same or are different. In some embodiments, the first unnatural base (X) located in the codon of the mRNA and the second unnatural base (Y) located in the anticodon of the tRNA are the same. In some embodiments, the first unnatural base (X) located in the codon of the mRNA and the second unnatural base (Y) located in the anticodon of the tRNA are different. In some embodiments, the first unnatural base (X) located in the codon of the mRNA and the second unnatural base (Y) located in the anticodon of the tRNA are selected from the group consisting of

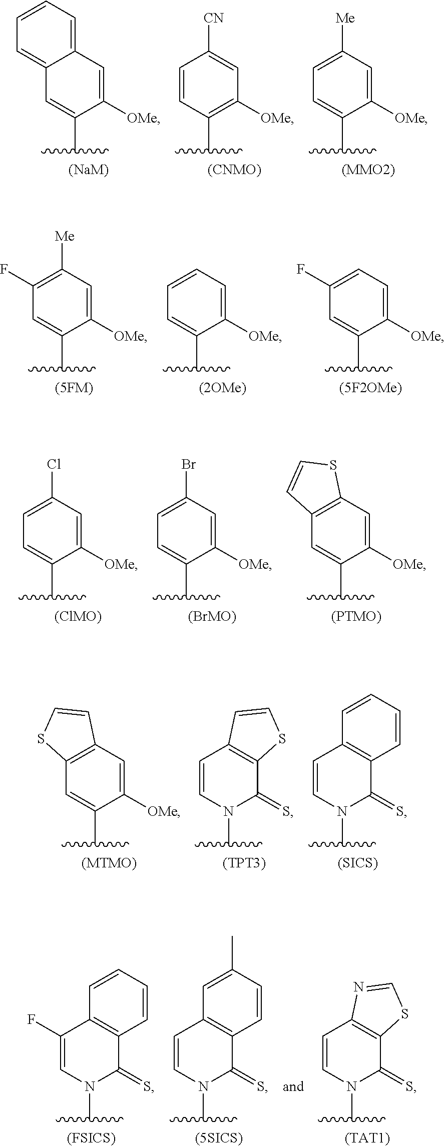

wherein the wavy line indicates a bond to a ribosyl moiety. In some embodiments, the first unnatural base (X) located in the codon of the mRNA and the second unnatural base (Y) located in the anticodon of the tRNA are selected from the group consisting of

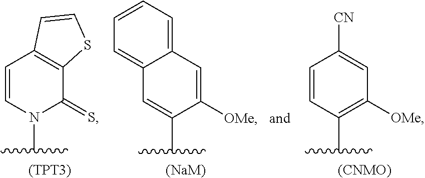

wherein the wavy line indicates a bond to a ribosyl moiety. In some embodiments, the first unnatural base (X) located in the codon of the mRNA and the second unnatural base (Y) located in the anticodon of the tRNA are both

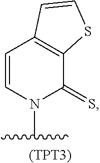

wherein the wavy line indicates a bond to a ribosyl moiety. In some embodiments, the first unnatural base (X) located in the codon of the mRNA and the second unnatural base (Y) located in the anticodon of the tRNA are both

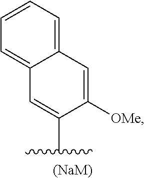

wherein the wavy line indicates a bond to a ribosyl moiety. In some embodiments, the first unnatural base (X) located in the codon of the mRNA and the second unnatural base (Y) located in the anticodon of the tRNA are both

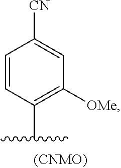 [SMILES: *c1ccc(C#N)cc1OC]

wherein the wavy line indicates a bond to a ribosyl moiety. In some embodiments the first unnatural base (X) located in the codon of the mRNA is selected from

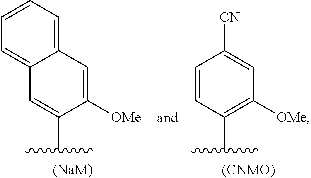

and the second unnatural base (Y) located in the anticodon of the tRNA is

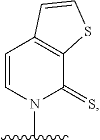 [SMILES: *n1ccc2ccsc2c1=S]

wherein in each case the wavy line indicates a bond to a ribosyl moiety. In some embodiments, the first unnatural base (X) located in the codon of the mRNA is

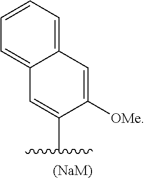

In some embodiments, the first unnatural base (X) located in the codon of the mRNA is

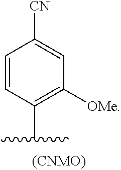 [SMILES: *c1ccc(C#N)cc1OC]

In some embodiments, the codon in the mRNA is selected from AXC, GXC or GXU, wherein X is the unnatural base. In some embodiments, the codon in the mRNA is AXC, wherein X is the unnatural base. In some embodiments, the codon in the mRNA is GXC, wherein X is the unnatural base. In some embodiments, the codon in the mRNA is GXU, wherein X is the unnatural base. In some embodiments, the codon in the mRNA is selected from AXC, GXC or GXU, wherein the anticodon in the tRNA is selected from GYU, GYC, and AYC, wherein X is a first unnatural base and Y is a second unnatural base. In some embodiments, X and Y are the same or are different. In some embodiments, X and Y are the same. In some embodiments, X and Y are different. In some embodiments, the codon in the mRNA is AXC and the anticodon in the tRNA is GYU. In some embodiments, X and Y are the same or are different. In some embodiments, X and Y are the same. In some embodiments, X and Y are different. In some embodiments, the codon in the mRNA is GXC and the anticodon in the tRNA is GYC. In some embodiments, X and Y are the same or are different. In some embodiments, X and Y are the same. In some embodiments, X and Y are different. In some embodiments, the codon in the mRNA is GXU and the anticodon is AYC. In some embodiments, X and Y are the same or are different. In some embodiments, X and Y are the same. In some embodiments, X and Y are different. In some embodiments, the tRNA is derived from Methanococcus jannaschii, Methanosarcina barkeri, Methanosarcina mazei, or Methanosarcina acetivorans. In some embodiments, the amino acyl tRNA synthetase (also referred to herein simply as a tRNA synthetase) is derived from Methanococcus jannaschii, Methanosarcina barkeri, Methanosarcina mazei, or Methanosarcina acetivorans. In some embodiments, the tRNA and the tRNA synthetase are derived from Methanococcus jannaschii. In some embodiments, the tRNA and the tRNA synthetase are derived from Methanosarcina barkeri. In some embodiments, the tRNA and the tRNA synthetase are derived from Methanosarcina mazei. In some embodiments, the tRNA and the tRNA synthetase are derived from Methanosarcina acetivorans. In some embodiments, the tRNA is derived from Methanococcus jannaschii and tRNA synthetase is derived from Methanosarcina barkeri, Methanosarcina mazei, or Methanosarcina acetivorans. In some embodiments, the tRNA is derived from Methanosarcina barkeri and tRNA synthetase is derived from Methanococcus jannaschii, Methanosarcina mazei, or Methanosarcina acetivorans. In some embodiments, the tRNA is derived from Methanosarcina mazei and tRNA synthetase is derived from Methanococcus jannaschii. Methanosarcina barkeri, or Methanosarcina acetivorans. In some embodiments, the tRNA is derived from Methanosarcina acetivorans and tRNA synthetase is derived from Methanococcus jannaschii, Methanosarcina barkeri, or Methanosarcina mazei. In some embodiments, the tRNA is derived from Methanosarcina mazei and tRNA synthetase is derived from Methanosarcina barkeri. In some embodiments, the cell is a human cell. In some embodiments, the human cell is a HEK293T cell. In some embodiments, the cell is a hamster cell. In some embodiments, the hamster cell is a Chinese hamster ovary (CHO) cell. In some embodiments, the unnatural amino acid: (a) is a lysine analogue; (b) comprises an aromatic side chain; (c) comprises an azido group; (d) comprises an alkyne group; or (e) comprises an aldehyde or ketone group. In some embodiments, the unnatural amino acid is selected from the group consisting of N6-((azidoethoxy)-carbonyl)-L-lysine (AzK), N6-((propargylethoxy)-carbonyl)-L-lysine (PraK), BCN-L-lysine, norbomene lysine, TCO-lysine, methyltetrazine lysine, allyloxycarbonyllysine, 2-amino-8-oxononanoic acid, 2-amino-8-oxooctanoic acid, p-acetyl-L-phenylalanine, p-azidomethyl-L-phenylalanine (pAMF), p-iodo-L-phenylalanine, m-acetylphenylalanine, 2-amino-8-oxononanoic acid, p-propargyloxyphenylalanine, p-propargyl-phenylalanine, 3-methyl-phenylalanine, L-Dopa, fluorinated phenylalanine, isopropyl-L-phenylalanine, p-azido-L-phenylalanine, p-acyl-L-phenylalanine, p-benzoyl-L-phenylalanine, p-bromophenylalanine, p-amino-L-phenylalanine, isopropyl-L-phenylalanine, O-allyltyrosine, O-methyl-L-tyrosine, O-4-allyl-L-tyrosine, 4-propyl-L-tyrosine, phosphonotyrosine, tri-O-acetyl-GlcNAcp-serine, L-phosphoserine, phosphonoserine, L-3-(2-naphthyl)alanine, 2-amino-3-((2-((3-(benzyloxy)-3-oxopropyl)amino)ethyl)selanyl)propanoic acid, 2-amino-3-(phenylselanyl)propanoic, selenocysteine, N6-(((2-azidobenzyl)oxy)carbonyl)-L-lysine, N6-(((3-azidobenzyl)oxy)carbonyl)-L-lysine, or N6-(((4-azidobenzyl)oxy)carbonyl)-L-lysine. In some embodiments, the unnatural amino acid is N6-((azidoethoxy)-carbonyl)-L-lysine (AzK). In some embodiments, the at least one unnatural amino acid is N6-(((2-azidobenzyl)oxy)carbonyl)-L-lysine. In some embodiments, the at least one unnatural amino acid is N6-(((3-azidobenzyl)oxy)carbonyl)-L-lysine. In some embodiments, the at least one unnatural amino acid is N6-(((4-azidobenzyl)oxy)carbonyl)-L-lysine. In some embodiments, the mRNA and the tRNA are stabilized to degradation in the eukaryotic cell. In some embodiments, the polypeptide is produced by translation of the mRNA using the tRNA by a ribosome that is endogenous to the eukaryotic cell.

Aspects disclosed herein provide systems for expression of an unnatural polypeptide comprising: (a) at least one unnatural amino acid; (b) an mRNA encoding the unnatural polypeptide, said mRNA comprising at least one codon comprising one or more first unnatural bases; (c) a tRNA comprising at least one anti-codon comprising one or more second unnatural bases wherein the one or more first unnatural bases and the one or more second unnatural bases form one or more complementary base pairs; and (d) a eukaryotic ribosome capable of translating the mRNA into a polypeptide comprising the unnatural amino acid using the tRNA and tRNA synthetase. The tRNA may be charged with the unnatural amino acid, and/or the system may further comprise a tRNA synthetase and/or one or more nucleic acid constructs comprising a nucleic acid sequence encoding a tRNA synthetase, wherein the tRNA synthetase preferentially aminoacylates the tRNA with the at least one unnatural amino acid. The system may be in vitro (e.g., cell-free, such as a cell lysate or a reconstituted system of purified components) or in a eukaryotic cell. In some embodiments, the at least one codon of the mRNA comprises three contiguous nucleobases (N—N—N); and wherein the one or more first unnatural bases (X) is located at the first position (X—N—N) in the at least one codon of the mRNA. In some embodiments, the at least one codon of the mRNA comprises three contiguous nucleobases (N—N—N); and wherein the one or more first unnatural bases (X) is located at the middle position (N—X—N) in the codon of the mRNA. In some embodiments, the at least one codon of the mRNA comprises three contiguous nucleobases (N—N—N); and wherein the one or more first unnatural bases (X) is located at the last position (N—N—X) in the at least one codon of the mRNA. In some embodiments, the one or more unnatural bases is of the formula

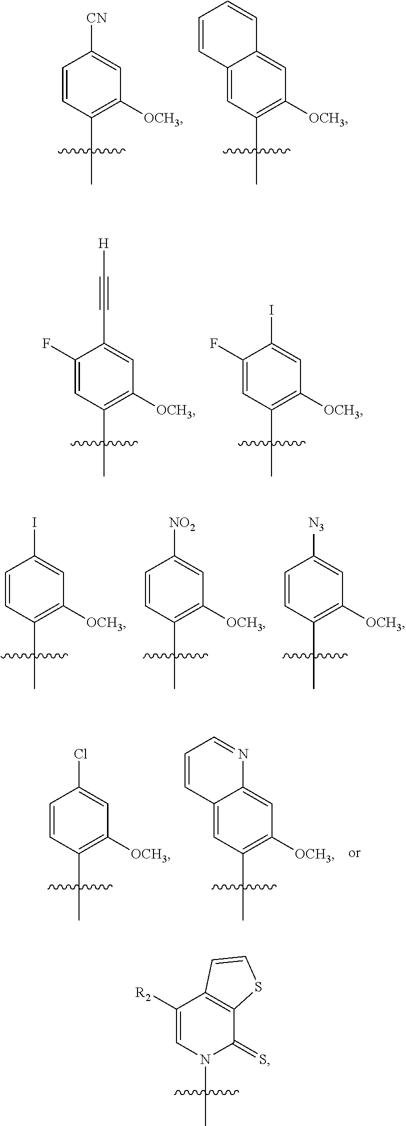

wherein R₂ is selected from the group consisting of hydrogen, alkyl, alkenyl, alkynyl, methoxy, methanethiol, methaneseleno, halogen, cyano, and azido, and the wavy line indicates a bond to a ribosyl moiety. In some embodiments, the one or more first unnatural bases or the one or more second unnatural bases is selected from the group consisting

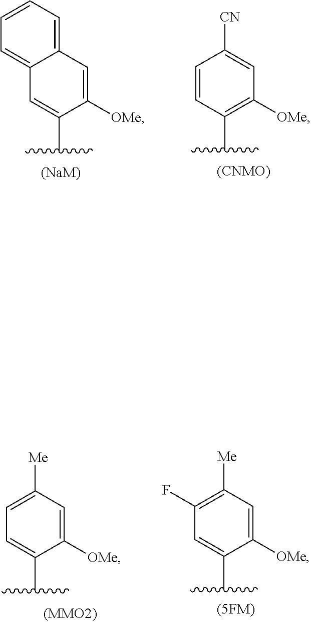

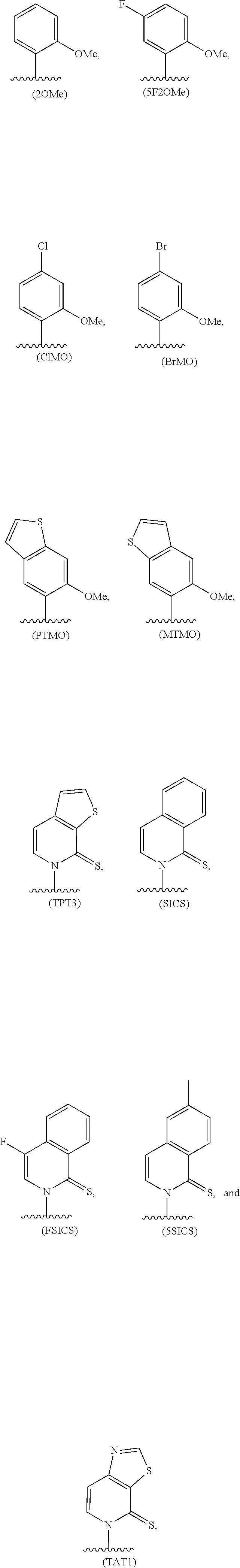

wherein the wavy line indicates a bond to a ribosyl moiety. In some embodiments, when the one or more first unnatural bases is

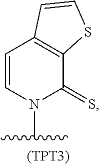

the one or more second unnatural bases is

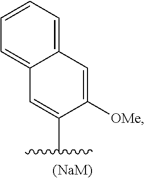

and when the one or more first unnatural bases is

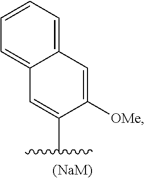

the second unnatural base is

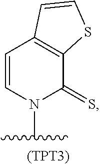

wherein the wavy line indicates a bond to a ribosyl moiety. In some embodiments, when the one or more first unnatural bases is

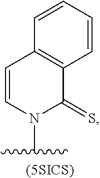

the one or more second unnatural bases is

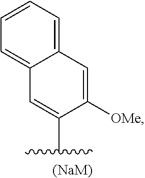

and when the one or more first unnatural bases is

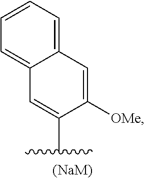

the one or more second unnatural bases is

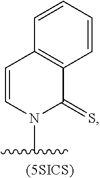

wherein the wavy line indicates a bond to a ribosyl moiety. In some embodiments, when the one or more first unnatural bases is

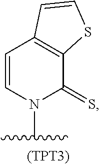

the one or more second unnatural bases is

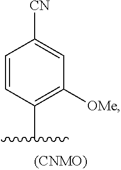 [SMILES: *c1ccc(C#N)cc1OC]

and when the one or more first unnatural bases is

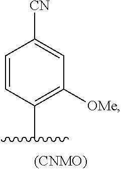 [SMILES: *c1ccc(C#N)cc1OC]

the one or more second unnatural bases is

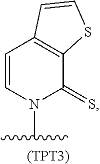

wherein the wavy line indicates a bond to a ribosyl moiety. In some embodiments, when the one or more first unnatural bases is

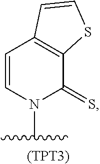

the one or more second unnatural bases is

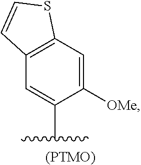

and when the one or more first unnatural bases is

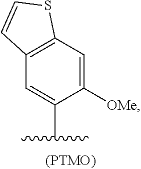 [SMILES: *c1cc2ccsc2cc1OC]

the one or more second unnatural bases is

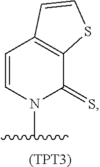

wherein the wavy line indicates a bond to a ribosyl moiety. In some embodiments, when the one or more first unnatural bases is

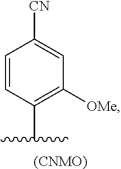

the one or more second unnatural bases is

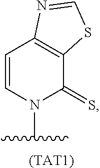

and when the one or more first unnatural bases is

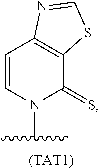

the one or more second unnatural bases is

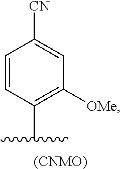

wherein the wavy line indicates a bond to a ribosyl moiety. In some embodiments, when the one or more first unnatural bases is

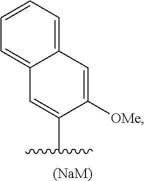

the one or more second unnatural bases is

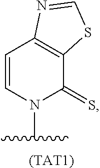

and when the one or more first unnatural bases is

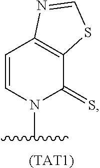

the one or more second unnatural bases is

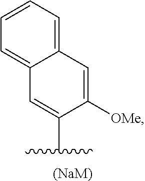

wherein the wavy line indicates a bond to a ribosyl moiety. In some embodiments, when the one or more first unnatural bases is

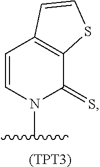

and the one or more second unnatural bases is

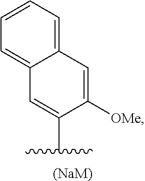

wherein the wavy line indicates a bond to a ribosyl moiety. In some embodiments, the one or more first unnatural bases is selected from

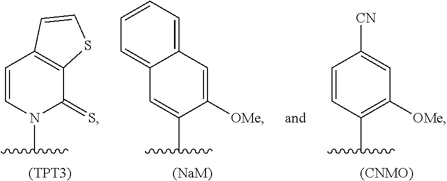

wherein the wavy line indicates a bond to a ribosyl moiety. In some embodiments, the one or more first unnatural bases is

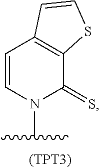

wherein the wavy line indicates a bond to a ribosyl moiety. In some embodiments, the one or more first unnatural bases is

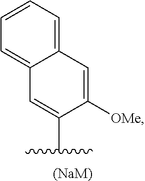

wherein the wavy line indicates a bond to a ribosyl moiety. In some embodiments, the one or more first unnatural bases is

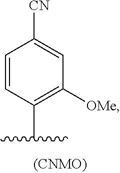 [SMILES: *c1ccc(C#N)cc1OC]

wherein the wavy line indicates a bond to a ribosyl moiety. In some embodiments, the at least one codon of the mRNA comprises three contiguous nucleobases (N—N—N), wherein the one or more first unnatural bases (X) is located at the first position (X—N—N) in the codon of the mRNA, wherein the one or more first unnatural bases is selected from

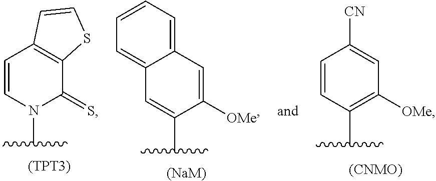

and wherein the wavy line indicates a bond to a ribosyl moiety. In some embodiments, the one or more first unnatural bases is

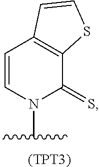

wherein the wavy line indicates a bond to a ribosyl moiety. In some embodiments, the one or more first unnatural bases is

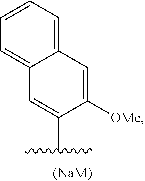

wherein the wavy line indicates a bond to a ribosyl moiety. In some embodiments, the one or more first unnatural base is

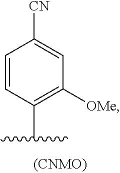 [SMILES: *c1ccc(C#N)cc1OC]

wherein the wavy line indicates a bond to a ribosyl moiety. In some embodiments, the at least one codon of the mRNA comprises three contiguous nucleobases (N—N—N), wherein the one or more first unnatural bases (X) is located at the middle position (N—X—N) in the codon of the mRNA, wherein the one or more first unnatural bases is selected from

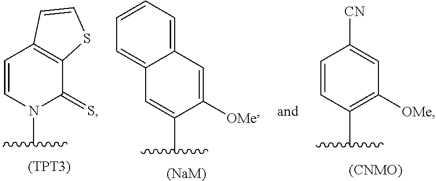

and wherein the wavy line indicates a bond to a ribosyl moiety. In some embodiments, the one or more first unnatural bases is

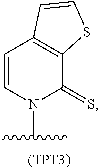

wherein the wavy line indicates a bond to a ribosyl moiety. In some embodiments, the one or more first unnatural bases is

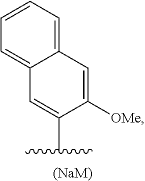

wherein the wavy line indicates a bond to a ribosyl moiety. In some embodiments, the one or more first unnatural base is

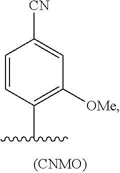 [SMILES: *c1ccc(C#N)cc1OC]

wherein the wavy line indicates a bond to a ribosyl moiety. In some embodiments, the at least one codon of the mRNA comprises three contiguous nucleobases (N—N—N), wherein the one or more first unnatural base (X) is located at the last position (N—N—X) in the codon of the mRNA, wherein the one or more first unnatural base is selected from

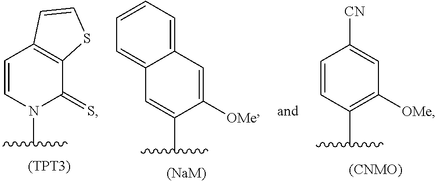

and wherein the wavy line indicates a bond to a ribosyl moiety. In some embodiments, the one or more first unnatural base is

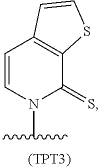

wherein the wavy line indicates a bond to a ribosyl moiety. In some embodiments, the one or more first unnatural bases is

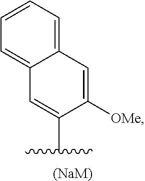

wherein the wavy line indicates a bond to a ribosyl moiety. In some embodiments, the one or more first unnatural bases is

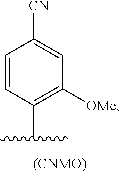 [SMILES: *c1ccc(C#N)cc1OC]

wherein the wavy line indicates a bond to a ribosyl moiety. In some embodiments, the at least one anticodon of the tRNA comprises three contiguous nucleobases (N—N—N); and wherein the one or more second unnatural base (X) is located at the first position (X—N—N) in the anticodon of the tRNA. In some embodiments, the one or more second unnatural bases is selected from

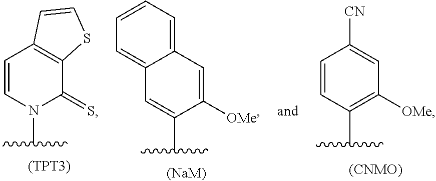

and wherein the wavy line indicates a bond to a ribosyl moiety. In some embodiments, the one or more second unnatural base is

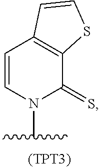

wherein the wavy line indicates a bond to a ribosyl moiety. In some embodiments, the one or more second unnatural bases is

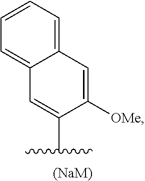

wherein the wavy line indicates a bond to a ribosyl moiety. In some embodiments, the one or more second unnatural bases is

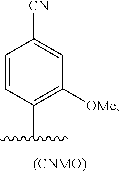 [SMILES: *c1ccc(C#N)cc1OC]

wherein the wavy line indicates a bond to a ribosyl moiety. In some embodiments, the at least one anticodon of the tRNA comprises three contiguous nucleobases (N—N—N); and wherein the one or more second unnatural bases (X) is located at the middle position (N—X—N) in the anticodon of the tRNA. In some embodiments, the one or more second unnatural bases is selected from

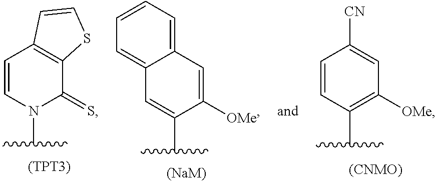

and wherein the wavy line indicates a bond to a ribosyl moiety. In some embodiments, the one or more second unnatural bases is

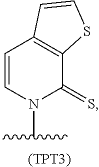

wherein the wavy line indicates a bond to a ribosyl moiety. In some embodiments, the one or more second unnatural bases is

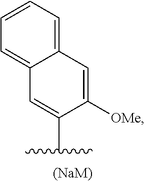

wherein the wavy line indicates a bond to a ribosyl moiety. In some embodiments, the one or more second unnatural bases is

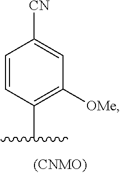 [SMILES: *c1ccc(C#N)cc1OC]

wherein the wavy line indicates a bond to a ribosyl moiety. In some embodiments, the at least one anticodon of the tRNA comprises three contiguous nucleobases (N—N—N); and wherein the one or more second unnatural bases (X) is located at the last position (N—N—X) in the anticodon of the tRNA. In some embodiments, the one or more second unnatural bases is selected from

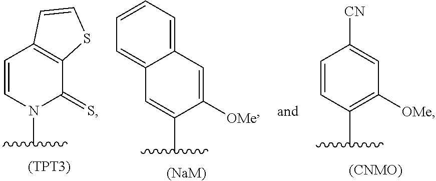

and wherein the wavy line indicates a bond to a ribosyl moiety. In some embodiments, the one or more second unnatural bases is

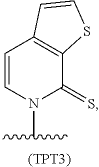

wherein the wavy line indicates a bond to a ribosyl moiety. In some embodiments, the one or more second unnatural bases is

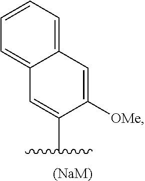

wherein the wavy line indicates a bond to a ribosyl moiety. In some embodiments, the one or more second unnatural bases is

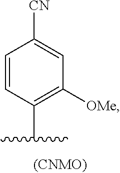 [SMILES: *c1ccc(C#N)cc1OC]

wherein the wavy line indicates a bond to a ribosyl moiety. In some embodiments, the at least one codon and the at least one anticodon each, independently, comprise three contiguous nucleobases (N—N—N), and wherein the at least one codon comprises one or more first unnatural bases (X) located at the first position (X—N—N) of the codon, and the at least one anticodons in the tRNA comprises the one or more second unnatural bases (Y) located at the last position (N—N—Y) of the anticodon. In some embodiments, the one or more first unnatural bases (X) located in the codon of the mRNA and the one or more second unnatural bases (Y) located in the anticodon of the tRNA are the same or are different. In some embodiments, the one or more first unnatural bases (X) located in the codon of the mRNA and the one or more second unnatural bases (Y) located in the anticodon of the tRNA are the same. In some embodiments, the one or more first unnatural bases (X) located in the codon of the mRNA and the one or more second unnatural bases (Y) located in the anticodon of the tRNA are different. In some embodiments, the one or more first unnatural bases (X) located in the codon of the mRNA and the one or more second unnatural bases (Y) located in the anticodon of the tRNA are selected from the group consisting of

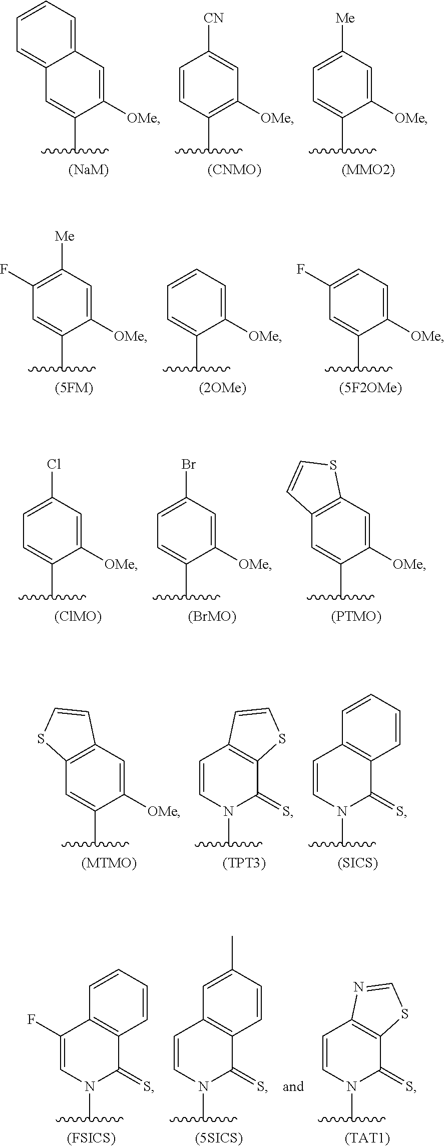

wherein the wavy line indicates a bond to a ribosyl moiety. In some embodiments, the one or more first unnatural bases (X) located in the codon of the mRNA and the one or more second unnatural bases (Y) located in the anticodon of the tRNA are selected from the group consisting of

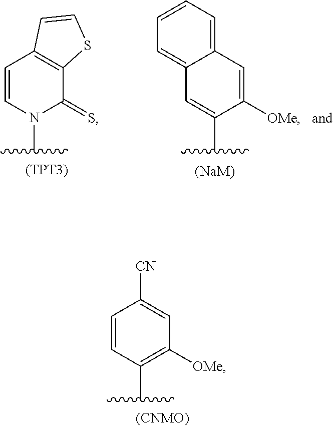

wherein the wavy line indicates a bond to a ribosyl moiety. In some embodiments, the one or more first unnatural bases (X) located in the codon of the mRNA and the one or more second unnatural bases (Y) located in the anticodon of the tRNA are both

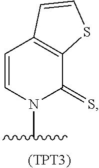

wherein the wavy line indicates a bond to a ribosyl moiety. In some embodiments, the one or more first unnatural bases (X) located in the codon of the mRNA and the one or more second unnatural bases (Y) located in the anticodon of the tRNA are both

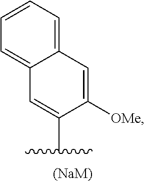

wherein the wavy line indicates a bond to a ribosyl moiety. In some embodiments, the one or more first unnatural bases (X) located in the codon of the mRNA and the one or more second unnatural bases (Y) located in the anticodon of the tRNA are both

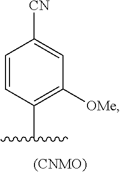 [SMILES: *c1ccc(C#N)cc1OC]

wherein the wavy line indicates a bond to a ribosyl moiety. In some embodiments, the one or more first unnatural base (X) located in the codon of the mRNA is selected from

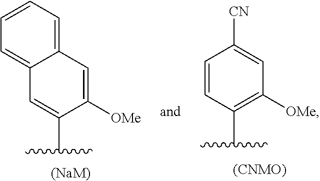

and the one or more second unnatural bases (Y) located in the anticodon of the tRNA is

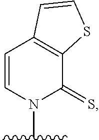 [SMILES: *n1ccc2ccsc2c1=S]

wherein in each case the wavy line indicates a bond to a ribosyl moiety. In some embodiments, the one or more first unnatural bases (X) located in the codon of the mRNA is

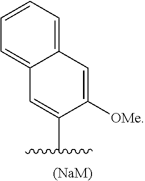

In some embodiments, the one or more first unnatural bases (X) located in the codon of the mRNA is

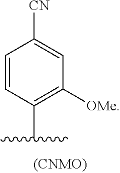 [SMILES: *c1ccc(C#N)cc1OC]

In some embodiments, the at least one codon and the at least one anticodon each, independently, comprise three contiguous nucleobases (N—N—N), and wherein the at least one codon in the mRNA comprises the one or more first unnatural bases (X) located at a middle position (N—X—N) of the at least one codon, and the at least one anticodon in the tRNA comprises the one or more second unnatural bases (Y) located at a middle position (N—Y—N) of the anticodon. In some embodiments, the one or more first unnatural bases (X) located in the codon of the mRNA and the one or more second unnatural bases (Y) located in the anticodon of the tRNA are the same or are different. In some embodiments, the one or more first unnatural bases (X) located in the codon of the mRNA and the one or more second unnatural bases (Y) located in the anticodon of the tRNA are the same. In some embodiments, the one or more first unnatural bases (X) located in the codon of the mRNA and the one or more second unnatural bases (Y) located in the anticodon of the tRNA are different. In some embodiments, the one or more first unnatural bases (X) located in the codon of the mRNA and the one or more second unnatural bases (Y) located in the anticodon of the tRNA are selected from the group consisting of

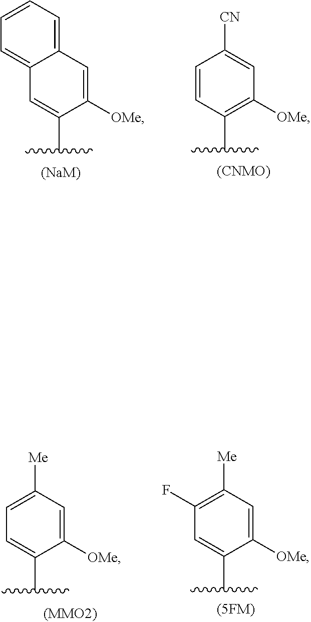

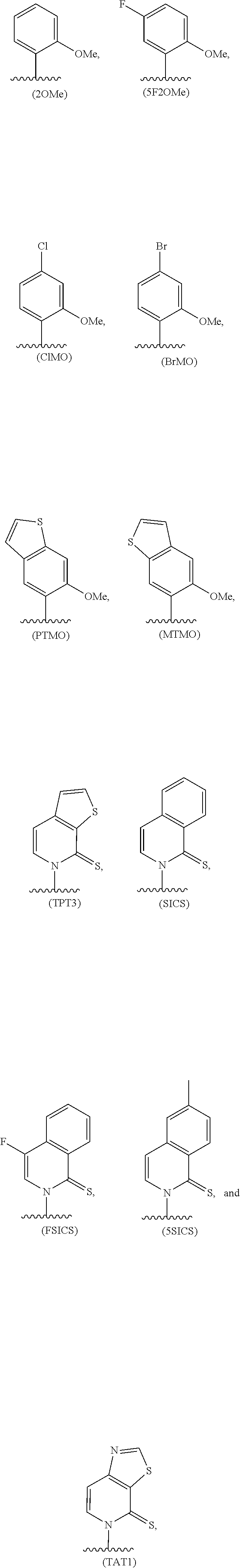

wherein the wavy line indicates a bond to a ribosyl moiety. In some embodiments, the one or more first unnatural bases (X) located in the codon of the mRNA and the one or more second unnatural bases (Y) located in the anticodon of the tRNA are selected from the group consisting of

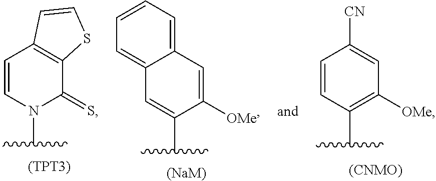

wherein the wavy line indicates a bond to a ribosyl moiety. In some embodiments, the one or more first unnatural bases (X) located in the codon of the mRNA and the one or more second unnatural bases (Y) located in the anticodon of the tRNA are both

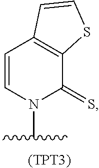

wherein the wavy line indicates a bond to a ribosyl moiety. In some embodiments, the one or more first unnatural bases (X) located in the codon of the mRNA and the one or more second unnatural bases (Y) located in the anticodon of the tRNA are both OMe

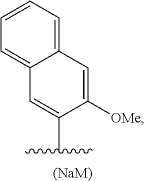

wherein the wavy line indicates a bond to a ribosyl moiety. In some embodiments, the one or more first unnatural bases (X) located in the codon of the mRNA and the one or more second unnatural bases (Y) located in the anticodon of the tRNA are both

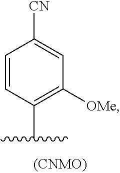 [SMILES: *c1ccc(C#N)cc1OC]

wherein the wavy line indicates a bond to a ribosyl moiety. In some embodiments, the one or more first unnatural bases (X) located in the codon of the mRNA is selected from

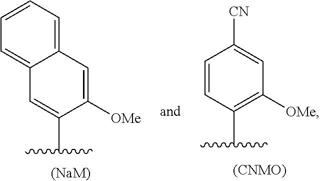

and the one or more second unnatural bases (Y) located in the anticodon of the tRNA is

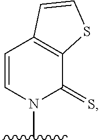 [SMILES: *n1ccc2ccsc2c1=S]

wherein in each case the wavy line indicates a bond to a ribosyl moiety. In some embodiments, the one or more first unnatural bases (X) located in the codon of the mRNA is

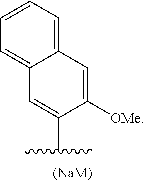

In some embodiments, the one or more first unnatural bases (X) located in the codon of the mRNA is

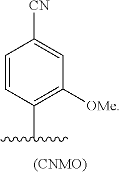 [SMILES: *c1ccc(C#N)cc1OC]

In some embodiments, the at least one codon and the at least one anticodon each, independently, comprise three contiguous nucleobases (N—N—N), and wherein the at least one codon in the mRNA comprises the one or more first unnatural bases (X) located at the last position (N—N—X) of the at least one codon, and the at least one anticodon in the tRNA comprises the one or more second unnatural bases (Y) located at the first position (Y—N—N) of the anticodon. In some embodiments, the one or more first unnatural bases (X) located in the codon of the mRNA and the one or more second unnatural bases (Y) located in the anticodon of the tRNA are the same or are different. In some embodiments, the one or more first unnatural bases (X) located in the codon of the mRNA and the one or more second unnatural bases (Y) located in the anticodon of the tRNA are the same. In some embodiments, the one or more first unnatural bases (X) located in the codon of the mRNA and the one or more second unnatural bases (Y) located in the anticodon of the tRNA are different. In some embodiments, the one or more first unnatural bases (X) located in the codon of the mRNA and the one or more second unnatural bases (Y) located in the anticodon of the tRNA are selected from the group consisting of

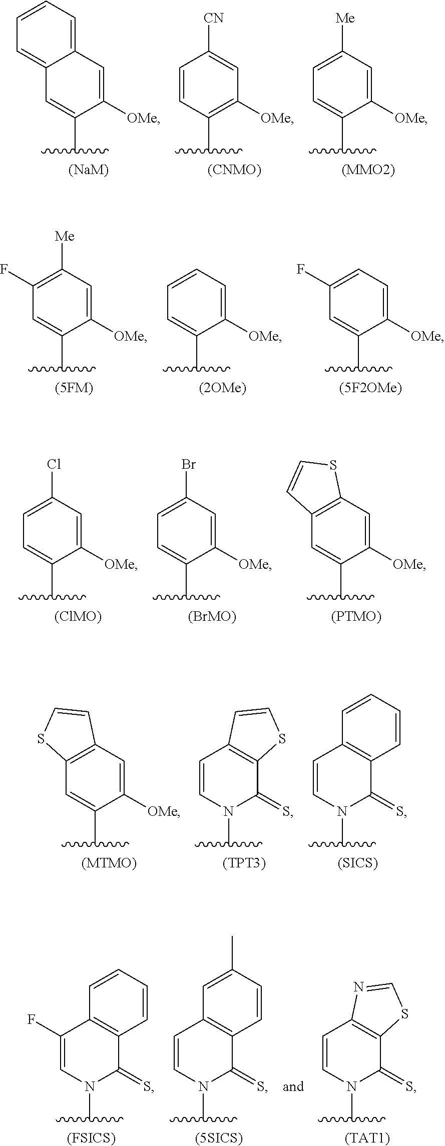

wherein the wavy line indicates a bond to a ribosyl moiety. In some embodiments, the one or more first unnatural bases (X) located in the codon of the mRNA and the one or more second unnatural bases (Y) located in the anticodon of the tRNA are selected from the group consisting of

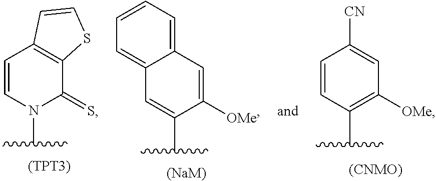

wherein the wavy line indicates a bond to a ribosyl moiety. In some embodiments, the one or more first unnatural bases (X) located in the codon of the mRNA and the one or more second unnatural bases (Y) located in the anticodon of the tRNA are both

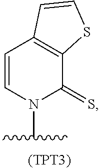

wherein the wavy line indicates a bond to a ribosyl moiety. In some embodiments, the one or more first unnatural bases (X) located in the codon of the mRNA and the one or more second unnatural bases (Y) located in the anticodon of the tRNA are both

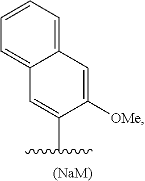

wherein the wavy line indicates a bond to a ribosyl moiety. In some embodiments, the one or more first unnatural bases (X) located in the codon of the mRNA and the one or more second unnatural bases (Y) located in the anticodon of the tRNA are both

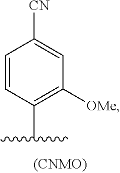 [SMILES: *c1ccc(C#N)cc1OC]

wherein the wavy line indicates a bond to a ribosyl moiety. In some embodiments, the one or more first unnatural bases (X) located in the codon of the mRNA is selected from

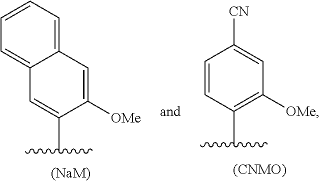

and the one or more second unnatural bases (Y) located in the anticodon of the tRNA is

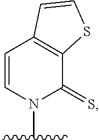 [SMILES: *n1ccc2ccsc2c1=S]

wherein in each case the wavy line indicates a bond to a ribosyl moiety. In some embodiments, the one or more first unnatural bases (X) located in the codon of the mRNA is

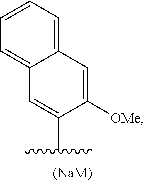

In some embodiments, the one or more first unnatural bases (X) located in the codon of the mRNA is

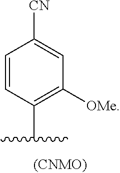 [SMILES: *c1ccc(C#N)cc1OC]

In some embodiments, the at least one codon in the mRNA is selected from AXC, GXC or GXU, wherein X is the unnatural base. In some embodiments, the at least one codon in the mRNA is AXC, wherein X is the unnatural base. In some embodiments, the at least one codon in the mRNA is GXC, wherein X is the unnatural base. In some embodiments, the at least one codon in the mRNA is GXU, wherein X is the unnatural base. In some embodiments, the at least one codon in the mRNA is selected from AXC, GXC or GXU, wherein the at least one anticodon in the tRNA is selected from GYU, GYC, and AYC, wherein X is the one or more first unnatural bases and Y is the one or more second unnatural bases. In some embodiments, X and Y are the same or are different. In some embodiments, X and Y are the same. In some embodiments, X and Y are different. In some embodiments, the at least one codon in the mRNA is AXC and the at least one anticodon in the tRNA is GYU. In some embodiments, X and Y are the same or are different. In some embodiments, X and Y are the same. In some embodiments, X and Y are different. In some embodiments, the at least one codon in the mRNA is GXC and the at least one anticodon in the tRNA is GYC. In some embodiments, X and Y are the same or are different. In some embodiments, X and Y are the same. In some embodiments, X and Y are different. In some embodiments, the at least one codon in the mRNA is GXU and the at least one anticodon is AYC. In some embodiments, X and Y are the same or are different. In some embodiments, X and Y are the same. In some embodiments, X and Y are different. In some embodiments, the tRNA is derived from Methanococcus jannaschii, Methanosarcina barkeri, Methanosarcina mazei, or Methanosarcina acetivorans. In some embodiments, the tRNA synthetase is derived from Methanococcus jannaschii, Methanosarcina barkeri, Methanosarcina mazei, or Methanosarcina acetivorans. In some embodiments, the tRNA and the tRNA synthetase are derived from Methanococcus jannaschii. In some embodiments, the tRNA and the tRNA synthetase are derived from Methanosarcina barkeri. In some embodiments, the tRNA and the tRNA synthetase are derived from Methanosarcina mazei. In some embodiments, the tRNA and the tRNA synthetase are derived from Methanosarcina acetivorans. In some embodiments, the tRNA is derived from Methanococcus jannaschii and tRNA synthetase is derived from Methanosarcina barkeri, Methanosarcina mazei, or Methanosarcina acetivorans. In some embodiments, the tRNA is derived from Methanosarcina barkeri and tRNA synthetase is derived from Methanococcus jannaschii, Methanosarcina mazei, or Methanosarcina acetivorans. In some embodiments, the tRNA is derived from Methanosarcina mazei and tRNA synthetase is derived from Methanococcus jannaschii. Methanosarcina barkeri, or Methanosarcina acetivorans. In some embodiments, the tRNA is derived from Methanosarcina acetivorans and tRNA synthetase is derived from Methanococcus jannaschii, Methanosarcina barkeri, or Methanosarcina mazei. In some embodiments, the tRNA is derived from Methanosarcina mazei and tRNA synthetase is derived from Methanosarcina barkeri. In some embodiments, the cell is a human cell. In some embodiments, the human cell is a HEK293T cell. In some embodiments, the cell is a hamster cell. In some embodiments, the hamster cell is a Chinese hamster ovary (CHO) cell. In some embodiments, the unnatural amino acid: (a) is a lysine analogue; (b) comprises an aromatic side chain; (c) comprises an azido group; (d) comprises an alkyne group; or (e) comprises an aldehyde or ketone group. In some embodiments, the unnatural amino acid is selected from the group consisting of N6-((azidoethoxy)-carbonyl)-L-lysine (AzK), N6-((propargylethoxy)-carbonyl)-L-lysine (PraK), BCN-L-lysine, norbomene lysine, TCO-lysine, methyltetrazine lysine, allyloxycarbonyllysine, 2-amino-8-oxononanoic acid, 2-amino-8-oxooctanoic acid, p-acetyl-L-phenylalanine, p-azidomethyl-L-phenylalanine (pAMF), p-iodo-L-phenylalanine, m-acetylphenylalanine, 2-amino-8-oxononanoic acid, p-propargyloxyphenylalanine, p-propargyl-phenylalanine, 3-methyl-phenylalanine, L-Dopa, fluorinated phenylalanine, isopropyl-L-phenylalanine, p-azido-L-phenylalanine, p-acyl-L-phenylalanine, p-benzoyl-L-phenylalanine, p-bromophenylalanine, p-amino-L-phenylalanine, isopropyl-L-phenylalanine, O-allyltyrosine, O-methyl-L-tyrosine, O-4-allyl-L-tyrosine, 4-propyl-L-tyrosine, phosphonotyrosine, tri-O-acetyl-GlcNAcp-serine, L-phosphoserine, phosphonoserine, L-3-(2-naphthyl)alanine, 2-amino-3-((2-((3-(benzyloxy)-3-oxopropyl)amino)ethyl)selanyl)propanoic acid, 2-amino-3-(phenylselanyl)propanoic, selenocysteine, N6-(((2-azidobenzyl)oxy)carbonyl)-L-lysine, N6-(((3-azidobenzyl)oxy)carbonyl)-L-lysine, or N6-(((4-azidobenzyl)oxy)carbonyl)-L-lysine. In some embodiments, the unnatural amino acid is N6-((azidoethoxy)-carbonyl)-L-lysine (AzK). In some embodiments, the at least one unnatural amino acid is N6-(((2-azidobenzyl)oxy)carbonyl)-L-lysine. In some embodiments, the at least one unnatural amino acid is N6-(((3-azidobenzyl)oxy)carbonyl)-L-lysine. In some embodiments, the at least one unnatural amino acid is N6-(((4-azidobenzyl)oxy)carbonyl)-L-lysine. In some embodiments, the mRNA and the tRNA are stabilized to degradation in the eukaryotic cell. In some embodiments, the polypeptide is produced by translation of the mRNA using the tRNA by a ribosome that is endogenous to the eukaryotic cell.

In one embodiment, the eukaryotic cell comprises an mRNA encoding Enhanced green fluorescent protein (EGFP) with an unnatural codon at position 151 (EGFP151 (NXN); where N refers to one of the natural nucleobases and X refers to NaM), the Methanosarcina mazei tRNAPyl recoded with a cognate unnatural anticodon (tRNAPyl(NYN), where Y refers to TPT3), and the chimeric Methanosarcina barkeri pyrrolysyl-tRNA synthetase (ChPylRS) which can charge the unnatural tRNAPyl with N6-(2-azidoethoxy)-carbonyl-L-lysine (AzK).

### BRIEF DESCRIPTION OF THE DRAWINGS

Various aspects of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A-1C illustrate UBPs and the workflow using the UBPs of the present embodiment. FIG. 1A depicts exemplary unnatural base pairs (UBP) dNaM and dTPT3. FIG. 1B illustrates a workflow using UBPs to site-specifically incorporate non-canonical amino acids (ncAAs) into a protein using an unnatural X-Y base pair. Incorporation of three ncAAs into a protein is shown as an example only; any number of ncAAs may be incorporated. FIG. 1C depicts exemplary UBPs.

FIG. 2 depicts dXTP analogs. Ribose and phosphates have been omitted for clarity.

FIGS. 3A-3B show exemplary unnatural bases.

FIGS. 4A-4G illustrate exemplary unnatural amino acids. These unnatural amino acids (UAAs) have been genetically encoded in proteins (FIG. 4D—UAA #1-42; FIG. 4E—UAA #43—89; FIG. 4F—UAA #90-128; FIG. 4G—UAA #129-167). FIGS. 4D-4G are adopted from Table 1 of Dumas et al., Chemical Science 2015, 6, 50-69.

FIGS. 5A-5B illustrates translation of unnatural codons in HEK293T cells. FIG. 5A shows the average EGFP fluorescence signal of HEK293T cells transfected with unnatural codons with or without cognate tRNAs measured by flow cytometry. FIG. 5B shows the protein shift assay for HEK293T cells transfected with unnatural codon GXC using cell lysate.

FIGS. 6A-6B illustrates translation of unnatural codons in CHO cells. FIG. 6A shows the average EGFP fluorescence signal of CHO cells transfected with unnatural codons (represented by the DNA encoding the unnatural codon) with or without cognate tRNAs (and self-pairing tRNA for codon AGX) measured by flow cytometry. FIG. 6B shows the protein shift assay for CHO cells transfected with unnatural codon AXC, GXC, GXT, GYC and AGX (represented by the DNA encoding the unnatural codon) using purified EGFP.

FIGS. 7A-7B show translation of unnatural codons within CYBA UTRs context in CHO cells. FIG. 7A: Average EGFP fluorescence signal of CHO cells transfected with unnatural codons within CYBA UTRs context, with or without cognate tRNAs (and self-pairing tRNA for codon AGX) measured by flow cytometry. *P<0.05, **P<0.005, ***P<0.0005, ****P<0.00005 (two-tailed paired t test). FIG. 7B: The protein shift assay for CHO cells transfected with unnatural codon GXC and GYC within CYBA UTRs context using purified EGFP.

FIGS. 7C-7D shows protein expression ratio between mRNA with CYBA UTRs and mRNA with CS2 UTRs. FIG. 7C shows the EGFP expression level ratios of different unnatural codons within CYBA UTRs and CS2 UTRs. Expression level was measured by flow cytometry. FIG. 7D shows, using RT-qPCR, mRNA abundancy measured at 4 h post-transcription and 8 h post-transcription. The ratio of the mRNA remaining after 8 h versus the mRNA remaining after 4 h is compared across different mRNA constructs. Note the unnatural codons in FIGS. 7A and 7B are represented by the coding sequence of the DNA encoding the mRNA.

### DETAILED DESCRIPTION OF THE INVENTION

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

Phrases such as “under conditions suitable to provide” or “under conditions sufficient to yield” or the like, in the context of methods of synthesis, as used herein refers to reaction conditions, such as time, temperature, solvent, reactant concentrations, and the like, that are within ordinary skill for an experimenter to vary, that provide a useful quantity or yield of a reaction product. It is not necessary that the desired reaction product be the only reaction product or that the starting materials be entirely consumed, provided the desired reaction product can be isolated or otherwise further used.

By “chemically feasible” is meant a bonding arrangement or a compound where the generally understood rules of organic structure are not violated; for example, a structure within a definition of a claim that would contain in certain situations a pentavalent carbon atom that would not exist in nature would be understood to not be within the claim. The structures disclosed herein, in all of their embodiments are intended to include only “chemically feasible” structures, and any recited structures that are not chemically feasible, for example in a structure shown with variable atoms or groups, are not intended to be disclosed or claimed herein.

An “analog” of a chemical structure, as the term is used herein, refers to a chemical structure that preserves substantial similarity with the parent structure, although it may not be readily derived synthetically from the parent structure. In some embodiments, a nucleotide analog is an unnatural nucleotide. In some embodiments, a nucleoside analog is an unnatural nucleoside. A related chemical structure that is readily derived synthetically from a parent chemical structure is referred to as a “derivative.”

Accordingly, a polynucleotide, as the terms are used herein, refer to DNA, RNA, DNA- or RNA-like polymers such as peptide nucleic acids (PNA), locked nucleic acids (LNA), phosphorothioates, unnatural bases, and the like, which are well-known in the art. Polynucleotides can be synthesized in automated synthesizers, e.g., using phosphoroamidite chemistry or other chemical approaches adapted for synthesizer use.

DNA includes, but is not limited to, cDNA and genomic DNA. DNA may be attached, by covalent or non-covalent means, to another biomolecule, including, but not limited to, RNA and peptide. RNA includes coding RNA, e.g. messenger RNA (mRNA). In some embodiments, RNA is rRNA, RNAi, snoRNA, microRNA, siRNA, snRNA, exRNA, piRNA, long ncRNA, or any combination or hybrid thereof. In some instances, RNA is a component of a ribozyme. DNA and RNA can be in any form, including, but not limited to, linear, circular, supercoiled, single-stranded, and double-stranded.

A peptide nucleic acid (PNA) is a synthetic DNA/RNA analog wherein a peptide-like backbone replaces the sugar-phosphate backbone of DNA or RNA. PNA oligomers show higher binding strength and greater specificity in binding to complementary DNAs, with a PNA/DNA base mismatch being more destabilizing than a similar mismatch in a DNA/DNA duplex. This binding strength and specificity also applies to PNA/RNA duplexes. PNAs are not easily recognized by either nucleases or proteases, making them resistant to enzyme degradation. PNAs are also stable over a wide pH range. See also Nielsen P E, Egholm M, Berg R H, Buchardt O (December 1991). “Sequence-selective recognition of DNA by strand displacement with a thymine-substituted polyamide”, Science 254 (5037): 1497-500. doi:10.1126/science.1962210. PMID 1962210; and, Egholm M, Buchardt O, Christensen L, Behrens C, Freier S M, Driver D A, Berg R H, Kim S K, Nordén B, and Nielsen P E (1993), “PNA Hybridizes to Complementary Oligonucleotides Obeying the Watson-Crick Hydrogen Bonding Rules”. Nature 365 (6446): 566-8. doi:10.1038/365566a0. PMID 7692304

A locked nucleic acid (LNA) is a modified RNA nucleotide, wherein the ribose moiety of an LNA nucleotide is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired. Such oligomers can be synthesized chemically and are commercially available. The locked ribose conformation enhances base stacking and backbone pre-organization. See, for example, Kaur, H; Arora, A; Wengel, J; Maiti, S (2006), “Thermodynamic, Counterion, and Hydration Effects for the Incorporation of Locked Nucleic Acid Nucleotides into DNA Duplexes”, Biochemistry 45 (23): 7347-55. doi:10.1021/bi060307w. PMID 16752924; Owczarzy R.; You Y., Groth C. L., Tataurov A. V. (2011), “Stability and mismatch discrimination of locked nucleic acid-DNA duplexes.”, Biochem. 50 (43): 9352-9367. doi:10.1021/bi200904e. PMC 3201676. PMID 21928795; Alexei A. Koshkin; Sanjay K. Singh, Poul Nielsen, Vivek K. Rajwanshi, Ravindra Kumar, Michael Meldgaard, Carl Erik Olsen, Jesper Wengel (1998), “LNA (Locked Nucleic Acids): Synthesis of the adenine, cytosine, guanine, 5-methylcytosine, thymine and uracil bicyclonucleoside monomers, oligomerisation, and unprecedented nucleic acid recognition”, Tetrahedron 54 (14): 3607-30. doi:10.1016/S0040-4020(98)00094-5; and, Satoshi Obika; Daishu Nanbu, Yoshiyuki Hari, Ken-ichiro Morio, Yasuko In, Toshimasa Ishida, Takeshi Imanishi (1997), “Synthesis of 2′-0,4′-C-methyleneuridine and -cytidine. Novel bicyclic nucleosides having a fixed C₃′-endo sugar puckering”, Tetrahedron Lett. 38 (50): 8735-8. doi:10.1016/S0040-4039(97)10322-7.

A molecular beacon or molecular beacon probe is an oligonucleotide hybridization probe that can detect the presence of a specific nucleic acid sequence in a homogenous solution. Molecular beacons are hairpin shaped molecules with an internally quenched fluorophore whose fluorescence is restored when they bind to a target nucleic acid sequence. See, for example, Tyagi S, Kramer F R (1996), “Molecular beacons: probes that fluoresce upon hybridization”, Nat Biotechnol. 14 (3): 303-8. PMID 9630890; Täpp I, Malmberg L, Rennel E, Wik M, Syvänen A C (2000 April), “Homogeneous scoring of single-nucleotide polymorphisms: comparison of the 5′-nuclease TaqMan assay and Molecular Beacon probes”, Biotechniques 28 (4): 732-8. PMID 10769752; and, Akimitsu Okamoto (2011), “ECHO probes: a concept of fluorescence control for practical nucleic acid sensing”, Chem. Soc. Rev. 40: 5815-5828.

In some embodiments, a nucleobase is generally the heterocyclic base portion of a nucleoside. Nucleobases may be naturally occurring, may be modified, may bear no similarity to natural bases, and may be synthesized, e.g., by organic synthesis. In certain embodiments, a nucleobase comprises any atom or group of atoms capable of interacting with a base of another nucleic acid with or without the use of hydrogen bonds. In certain embodiments, an unnatural nucleobase is not derived from a natural nucleobase. It should be noted that unnatural nucleobases do not necessarily possess basic properties, however, are referred to as nucleobases for simplicity. In some embodiments, when referring to a nucleobase, a “(d)” indicates that the nucleobase can be attached to a deoxyribose or a ribose.

In some embodiments, a nucleoside is a compound comprising a nucleobase moiety and a sugar moiety. Nucleosides include, but are not limited to, naturally occurring nucleosides (as found in DNA and RNA), abasic nucleosides, modified nucleosides, and nucleosides having mimetic bases and/or sugar groups. Nucleosides include nucleosides comprising any variety of substituents. A nucleoside can be a glycoside compound formed through glycosidic linking between a nucleic acid base and a reducing group of a sugar.

The section headings used herein are for organizational purposes only and are not to be construed as limiting the subject matter described.

### Methods, Systems and Compositions Comprising Unnatural Base Pairs in Eukaryotic Cells

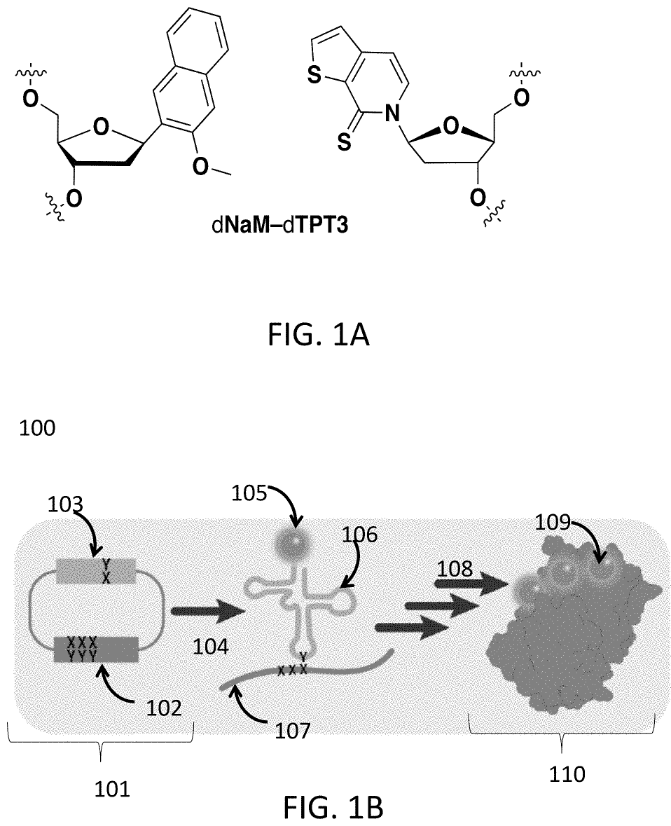

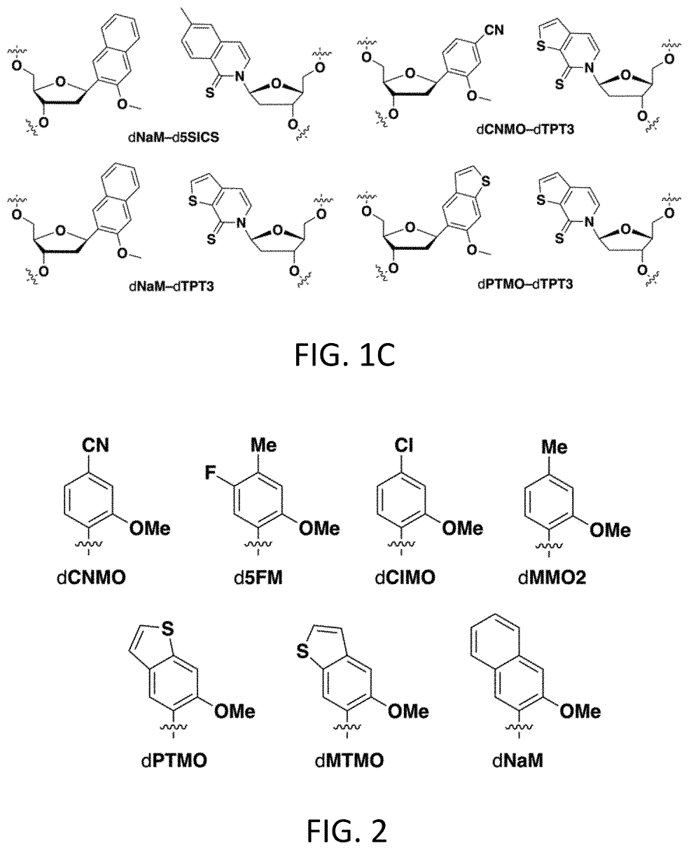

Disclosed herein in certain embodiments are in vivo methods and compositions for producing a nucleic acid with an expanded genetic alphabet (FIG. 1A-3B) in a eukaryotic cell. In some instances, the nucleic acid encodes for an unnatural protein, wherein the unnatural protein comprises at least one an unnatural amino acid. In some cases, an in vivo method or composition described herein utilizes or comprises a semi-synthetic organism. In some instances, the method comprises incorporating at least one unnatural base pair (UBP) into one or more nucleic acids. Such base pairs are formed by pairing between the nucleobases of two nucleosides. In an exemplary workflow provided in in FIG. 1B, DNA 101 coding for a protein 102 and a tRNA 103, each comprising complementary unnatural nucleobases (X, Y) is transcribed 104 to generate a tRNA 106 and mRNA 107. After charging the tRNA with an unnatural amino acid 105, the mRNA 107 is translated 108 to generate a protein 110 comprising one or more unnatural amino acids 109. Methods and compositions described herein in some instances allow for site-specific incorporation of unnatural amino acids with high fidelity and yield. Also described herein are semi-synthetic organisms comprising an expanded genetic alphabet, methods for using the semi-synthetic organisms to produce protein products, including those comprising at least one unnatural amino acid residue.

Selection of unnatural nucleobases allows for optimization of one or more steps in the methods described herein. For example, nucleobases are selected for high efficiency replication, transcription, and/or translation. In some instances, more than one unnatural nucleobase pair is utilized for the methods described herein. For example, a first set of nucleobases comprising a deoxyribo moiety are used for DNA replication (such as a first nucleobase and a second nucleobase, configure to form a first base pair), and a second set of nucleobases (such a third nucleobase and a fourth nucleobase, wherein the third and fourth nucleobases are attached to ribose, configured to form a second base pair) are used for transcription/translation. Complementary pairing between a nucleobase of the first set and a nucleobase of the second set in some instances allow for transcription of genes to generate tRNA or proteins from a DNA template comprising nucleobases from the first set. Complementary pairing between nucleobases of the second set (second base pair) in some instances allows for translation by matching tRNAs comprising unnatural nucleic acids and mRNA. In some cases, nucleobases in the first set are attached to a deoxyribose moiety. In some cases, nucleobases in the first set are attached to ribose moiety. In some instances, nucleobases of both sets are unique. In some instances, at least one nucleobase is the same in both sets. In some instances, a first nucleobase and a third nucleobase are the same. In some embodiments, the first base pair and the second base pair are not the same. In some cases, the first base pair, the second base pair, and the third base pair are not the same.

### Eukaryotic Engineered Organisms

In some embodiments, methods and plasmids disclosed herein are further used to generate eukaryotic engineered organisms, e.g. an organism that incorporates and replicates an unnatural nucleotide or an unnatural nucleic acid base pair (UBP) and may also use the nucleic acid containing the unnatural nucleotide to transcribe mRNA and tRNA which are used to translate proteins containing an unnatural amino acid residue. In some instances, the organism is a semi-synthetic organism (SSO). In some instances, the SSO is not prokaryotic. In some instances, the SSO is mammalian. In some instances, the mammalian SSO is human. In some instances, the mammalian SSO is hamster. In some instances, the human SSO is derived from a HEK293T cell. In some instances, the human SSO is derived from a Chinese hamster ovary (CHO) cell.

In some instances, the cell employed is genetically transformed with an expression cassette encoding a heterologous protein, e.g., a tRNA synthetase. In some embodiments, the tRNA synthetase preferentially aminoacylates the tRNA comprising an anticodon containing an unnatural base with the unnatural amino acid. In some embodiments, the cell comprises a tRNA synthetase that preferentially aminoacylates the tRNA comprising an anticodon containing an unnatural base with the unnatural amino acid.

The cell can be a eukaryotic cell, and the pair of unnatural mutually base-pairing nucleotides can be TPT3 and NaM or CNMO.

Described herein are compositions and methods comprising the use of two or more unnatural base-pairing nucleotides. Such base pairing nucleotides in some cases enter a cell through standard nucleic acid transformation methods known in the art (e.g., electroporation, chemical transformation, or other method in which nucleic acids comprising the unnatural nucleotides can be introduced into the cell). In some cases, three or more unnatural base-pairing nucleotides are used. In some cases, a base pairing unnatural nucleotide enters a cell as part of a polynucleotide, such as an mRNA and/or tRNA. One or more base pairing unnatural nucleotide which enter a cell as part of a polynucleotide (RNA) need not themselves be replicated in-vivo.

In some cases, genetically engineered cells are generated by introduction of nucleic acids, e.g., heterologous nucleic acids, into cells. Any cell described herein can be a host cell and can comprise an expression vector. In some embodiments, the cell is a mammalian cell. In some embodiments, the mammalian cell is a human cell (e.g., HEK293T cell). In some embodiments, the mammalian cell is a hamster cell (e.g., CHO cell). In some embodiments, a cell comprises one or more heterologous polynucleotides. Nucleic acid reagents can be introduced into microorganisms using various techniques. Non-limiting examples of methods used to introduce heterologous nucleic acids into various organisms include; transformation, transfection, transduction, electroporation, ultrasound-mediated transformation, conjugation, particle bombardment and the like. In some instances, the addition of carrier molecules (e.g., bis-benzoimidazolyl compounds, for example, see U.S. Pat. No. 5,595,899) can increase the uptake of DNA in cells typically though to be difficult to transform by conventional methods. Conventional methods of transformation are readily available to the artisan and can be found in Maniatis, T., E. F. Fritsch and J. Sambrook (1982) Molecular Cloning: a Laboratory Manual; Cold Spring Harbor Laboratory, Cold Spring Harbor, N.Y.

In some instances, genetic transformation is obtained using direct transfer of an expression cassette, in but not limited to, plasmids, viral vectors, viral nucleic acids, phage nucleic acids, phages, cosmids, and artificial chromosomes, or via transfer of genetic material in cells or carriers such as cationic liposomes. Such methods are available in the art and readily adaptable for use in the method described herein. Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)). Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991).

### Nucleic Acid Molecules

In some embodiments, a nucleic acid (e.g., also referred to herein as nucleic acid molecule of interest) is from any source or composition, such as RNA, siRNA (short inhibitory RNA), RNAi, tRNA, mRNA or rRNA (ribosomal RNA), for example, and is in any form (e.g., linear, circular, supercoiled, single-stranded, double-stranded, and the like). In some embodiments, nucleic acids comprise nucleotides, nucleosides, or polynucleotides. In some cases, nucleic acids comprise natural and unnatural nucleic acids. In some cases, a nucleic acid also comprises unnatural nucleic acids, such as RNA analogs (e.g., containing base analogs, sugar analogs and/or a non-native backbone and the like). It is understood that the term “nucleic acid” does not refer to or infer a specific length of the polynucleotide chain, thus polynucleotides and oligonucleotides are also included in the definition. Exemplary natural nucleotides include, without limitation, ATP, UTP, CTP, GTP, ADP, UDP, CDP, GDP, AMP, UMP, CMP, GMP, dATP, dTTP, dCTP, dGTP, dADP, dTDP, dCDP, dGDP, dAMP, dTMP, dCMP, and dGMP. Exemplary natural deoxyribonucleotides include dATP, dTTP, dCTP, dGTP, dADP, dTDP, dCDP, dGDP, dAMP, dTMP, dCMP, and dGMP. Exemplary natural ribonucleotides include ATP, UTP, CTP, GTP, ADP, UDP, CDP, GDP, AMP, UMP, CMP, and GMP. For natural RNA, the uracil base is uridine. A nucleic acid sometimes is a vector, plasmid, phagemid, autonomously replicating sequence (ARS), centromere, artificial chromosome, yeast artificial chromosome (e.g., YAC) or other nucleic acid able to replicate or be replicated in a host cell. In some cases, an unnatural nucleic acid is a nucleic acid analogue. In additional cases, an unnatural nucleic acid is from an extracellular source. In other cases, an unnatural nucleic acid is available to the intracellular space of an organism provided herein, e.g., a genetically modified organism. In some embodiments, an unnatural nucleotide is not a natural nucleotide. In some embodiments, a nucleotide that does not comprise a natural base comprises an unnatural nucleobase.

### Unnatural Nucleic Acids

A nucleotide analog, or unnatural nucleotide, comprises a nucleotide which contains some type of modification to either the base, sugar, or phosphate moieties. In some embodiments, a modification comprises a chemical modification. In some cases, modifications occur at the 3′OH or 5′OH group, at the backbone, at the sugar component, or at the nucleotide base. Modifications, in some instances, optionally include non-naturally occurring linker molecules and/or of interstrand or intrastrand cross links. In one aspect, the modified nucleic acid comprises modification of one or more of the 3′OH or 5′OH group, the backbone, the sugar component, or the nucleotide base, and/or addition of non-naturally occurring linker molecules. In one aspect, a modified backbone comprises a backbone other than a phosphodiester backbone. In one aspect, a modified sugar comprises a sugar other than deoxyribose (in modified DNA) or other than ribose (modified RNA). In one aspect, a modified base comprises a base other than adenine, guanine, cytosine or thymine (in modified DNA) or a base other than adenine, guanine, cytosine or uracil (in modified RNA).

In some embodiments, the nucleic acid comprises at least one modified base. In some instances, the nucleic acid comprises 2, 3, 4, 5, 6, 7, 8, 9, 10, 15, 20, or more modified bases. In some cases, modifications to the base moiety include natural and synthetic modifications of A, C, G, and T/U as well as different purine or pyrimidine bases. In some embodiments, a modification is to a modified form of adenine, guanine cytosine or thymine (in modified DNA) or a modified form of adenine, guanine cytosine or uracil (modified RNA).

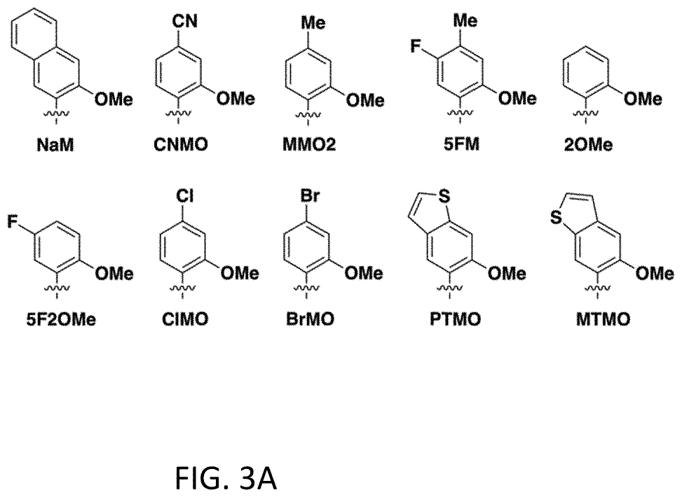

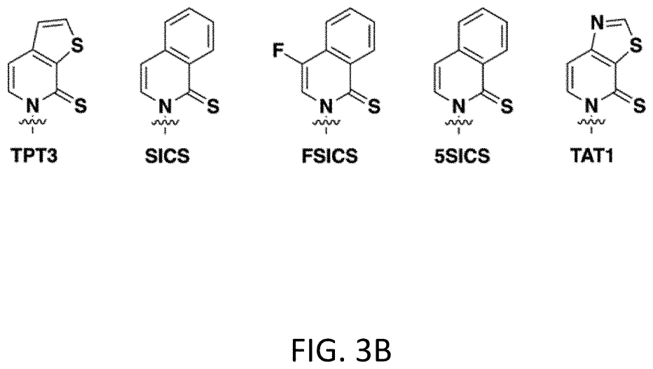

A modified base of a unnatural nucleic acid includes, but is not limited to, uracil-5-yl, hypoxanthin-9-yl (I), 2-aminoadenin-9-yl, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Certain unnatural nucleic acids, such as 5-substituted pyrimidines, 6-azapyrimidines and N-2 substituted purines, N-6 substituted purines, O-6 substituted purines, 2-aminopropyladenine, 5-propynyluracil, 5-propynylcytosine, 5-methylcytosine, those that increase the stability of duplex formation, universal nucleic acids, hydrophobic nucleic acids, promiscuous nucleic acids, size-expanded nucleic acids, fluorinated nucleic acids, 5-substituted pyrimidines, 6-azapyrimidines and N-2, N-6 and 0-6 substituted purines, including 2-aminopropyladenine, 5-propynyluracil and 5-propynylcytosine. 5-methylcytosine (5-me-C), 5-hydroxymethyl cytosine, xanthine, hypoxanthine, 2-aminoadenine, 6-methyl, other alkyl derivatives of adenine and guanine, 2-propyl and other alkyl derivatives of adenine and guanine, 2-thiouracil, 2-thiothymine and 2-thiocytosine, 5-halouracil, 5-halocytosine, 5-propynyl (—C≡C—CH₃) uracil, 5-propynyl cytosine, other alkynyl derivatives of pyrimidine nucleic acids, 6-azo uracil, 6-azo cytosine, 6-azo thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl, other 5-substituted uracils and cytosines, 7-methylguanine, 7-methyladenine, 2-F-adenine, 2-amino-adenine, 8-azaguanine, 8-azaadenine, 7-deazaguanine, 7-deazaadenine, 3-deazaguanine, 3-deazaadenine, tricyclic pyrimidines, phenoxazine cytidine([5,4-b][1,4]benzoxazin-2(3H)-one), phenothiazine cytidine (1H-pyrimido[5,4-b][1,4]benzothiazin-2(3H)-one), G-clamps, phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one), those in which the purine or pyrimidine base is replaced with other heterocycles, 7-deaza-adenine, 7-deazaguanosine, 2-aminopyridine, 2-pyridone, azacytosine, 5-bromocytosine, bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, and 5-iodouracil, 2-amino-adenine, 6-thio-guanine, 2-thio-thymine, 4-thio-thymine, 5-propynyl-uracil, 4-thio-uracil, N4-ethylcytosine, 7-deazaguanine, 7-deaza-8-azaguanine, 5-hydroxycytosine, 2′-deoxyuridine, 2-amino-2′-deoxyadenosine, and those described in U.S. Pat. Nos. 3,687,808; 4,845,205; 4,910,300; 4,948,882; 5,093,232; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121; 5,596,091; 5,614,617; 5,645,985; 5,681,941; 5,750,692; 5,763,588; 5,830,653 and 6,005,096; WO 99/62923; Kandimalla et al., (2001) Bioorg. Med. Chem. 9:807-813; The Concise Encyclopedia of Polymer Science and Engineering, Kroschwitz, J. I., Ed., John Wiley & Sons, 1990, 858-859; Englisch et al., Angewandte Chemie, International Edition, 1991, 30, 613; and Sanghvi, Chapter 15, Antisense Research and Applications, Crooke and Lebleu Eds., CRC Press, 1993, 273-288. Additional base modifications can be found, for example, in U.S. Pat. No. 3,687,808; Englisch et al., Angewandte Chemie, International Edition, 1991, 30, 613. In some instances, an unnatural nucleic acid comprises a nucleobase of FIG. 2. In some instances, an unnatural nucleic acid comprises a nucleobase of FIG. 3A. In some instances, an unnatural nucleic acid comprises a nucleobase of FIG. 3B.

Unnatural nucleic acids comprising various heterocyclic bases and various sugar moieties (and sugar analogs) are available in the art, and the nucleic acid in some cases include one or several heterocyclic bases other than the principal five base components of naturally-occurring nucleic acids. For example, the heterocyclic base includes, in some cases, uracil-5-yl, cytosin-5-yl, adenin-7-yl, adenin-8-yl, guanin-7-yl, guanin-8-yl, 4-aminopyrrolo [2.3-d]pyrimidin-5-yl, 2-amino-4-oxopyrolo [2,3-d]pyrimidin-5-yl, 2-amino-4-oxopyrrolo [2.3-d]pyrimidin-3-yl groups, where the purines are attached to the sugar moiety of the nucleic acid via the 9-position, the pyrimidines via the 1-position, the pyrrolopyrimidines via the 7-position and the pyrazolopyrimidines via the 1-position.

In some embodiments, a modified base of an unnatural nucleic acid is depicted below, wherein the wavy line identifies a point of attachment to the deoxyribose or ribose.

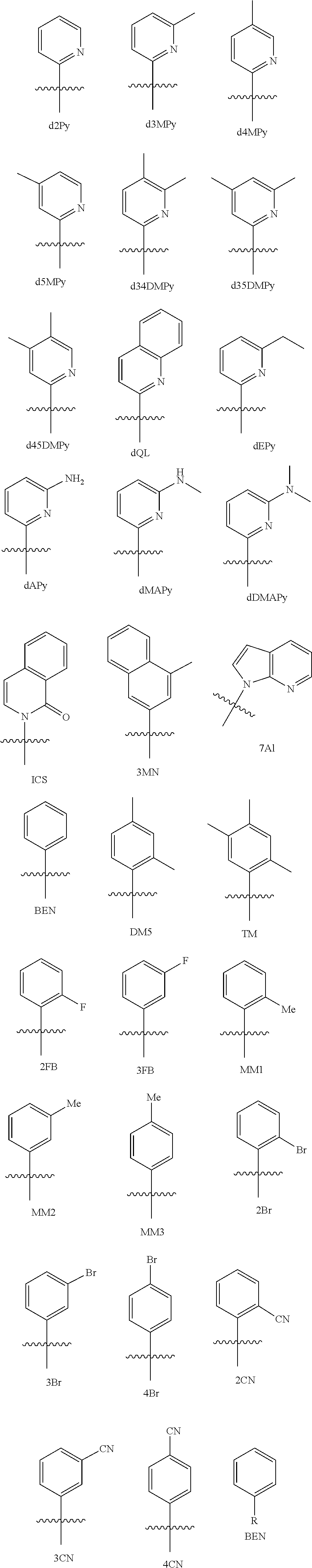

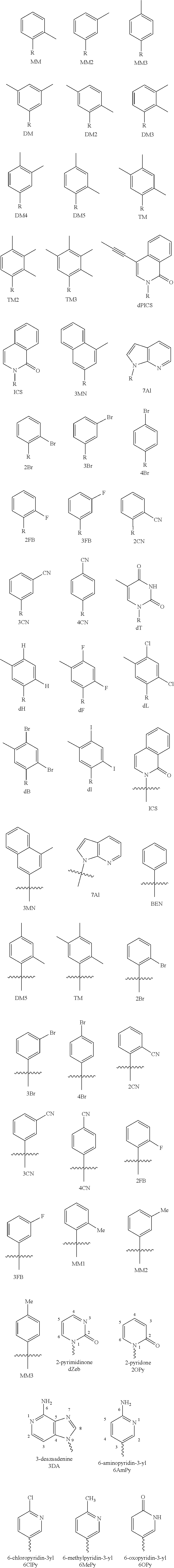

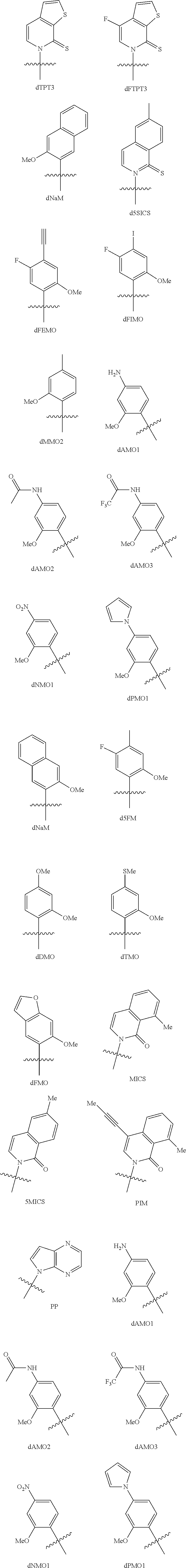

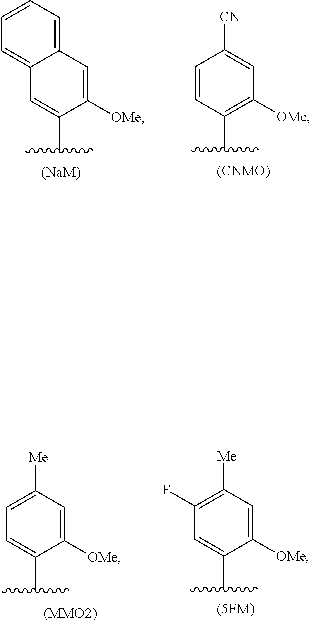

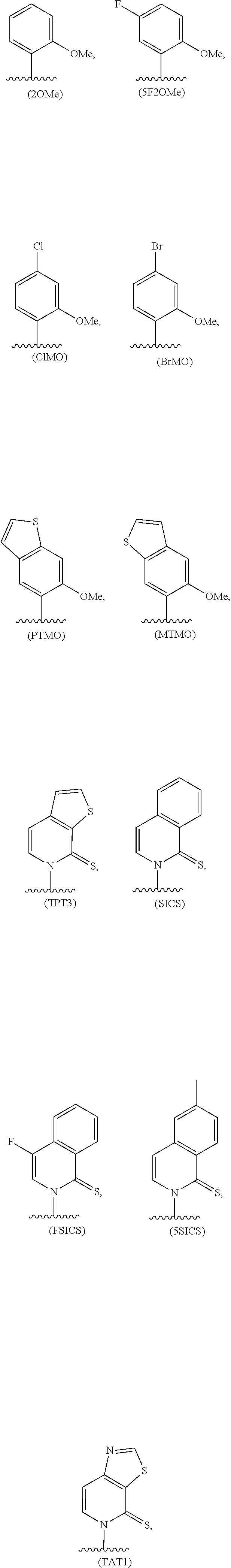

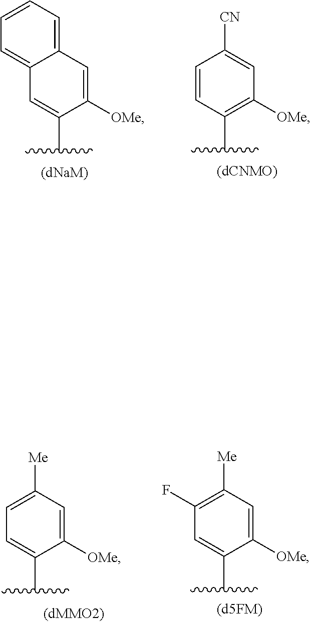

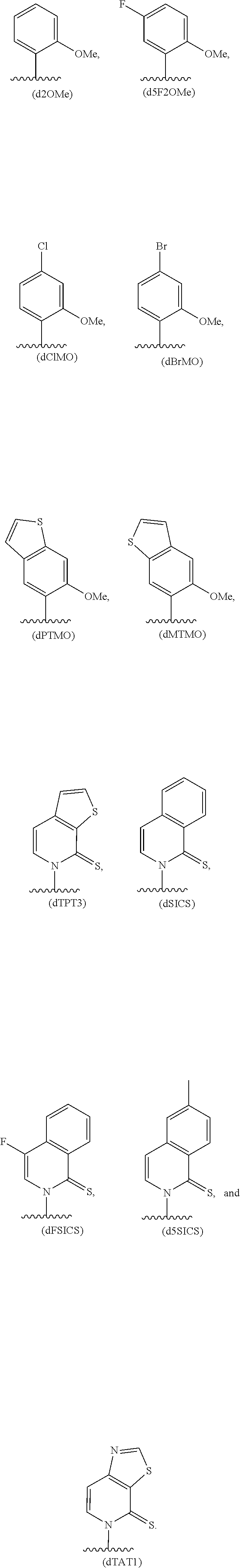

In some embodiments, nucleotide analogs are also modified at the phosphate moiety. Modified phosphate moieties include, but are not limited to, those with modification at the linkage between two nucleotides and contains, for example, a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkage between two nucleotides are through a 3′-5′ linkage or a 2′-5′ linkage, and the linkage contains inverted polarity such as 3′-5′ to 5′-3′ or 2′-5′ to 5′-2′. Various salts, mixed salts and free acid forms are also included. Numerous United States patents teach how to make and use nucleotides containing modified phosphates and include but are not limited to U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

In some embodiments, unnatural nucleic acids include 2′,3′-dideoxy-2′,3′-didehydro-nucleosides (PCT/US2002/006460), 5′-substituted DNA and RNA derivatives (PCT/US2011/033%1; Saha et al., J. Org Chem., 1995, 60, 788-789; Wang et al., Bioorganic & Medicinal Chemistry Letters, 1999, 9, 885-890; and Mikhailov et al., Nucleosides & Nucleotides, 1991, 10 (1-3), 339-343; Leonid et al., 1995, 14 (3-5), 901-905; and Eppacher et al., Helvetica Chimica Acta, 2004, 87, 3004-3020; PCT/JP2000/004720; PCT/JP2003/002342; PCT/JP2004/013216; PCT/JP2005/020435; PCT/JP2006/315479; PCT/JP2006/324484; PCT/JP2009/056718; PCT/JP2010/067560), or 5′-substituted monomers made as the monophosphate with modified bases (Wang et al., Nucleosides Nucleotides & Nucleic Acids, 2004, 23 (1 & 2), 317-337).

In some embodiments, unnatural nucleic acids include modifications at the 5′-position and the 2′-position of the sugar ring (PCT/US94/02993), such as 5′-CH₂-substituted 2′-O-protected nucleosides (Wu et al., Helvetica Chimica Acta, 2000, 83, 1127-1143 and Wu et al., Bioconjugate Chem. 1999, 10, 921-924). In some cases, unnatural nucleic acids include amide linked nucleoside dimers have been prepared for incorporation into oligonucleotides wherein the 3′ linked nucleoside in the dimer (5′ to 3′) comprises a 2′—OCH₃ and a 5′-(S)—CH₃ (Mesmaeker et al., Synlett, 1997, 1287-1290). Unnatural nucleic acids can include 2′-substituted 5′-CH₂ (or O) modified nucleosides (PCT/US92/01020). Unnatural nucleic acids can include 5′-methylenephosphonate DNA and RNA monomers, and dimers (Bohringer et al., Tet. Lett., 1993, 34, 2723-2726; Collingwood et al., Synlett, 1995, 7, 703-705; and Hutter et al., Helvetica Chimica Acta, 2002, 85, 2777-2806). Unnatural nucleic acids can include 5′-phosphonate monomers having a 2′-substitution (US2006/0074035) and other modified 5′-phosphonate monomers (WO1997/35869). Unnatural nucleic acids can include 5′-modified methylenephosphonate monomers (EP614907 and EP629633). Unnatural nucleic acids can include analogs of 5′ or 6′-phosphonate ribonucleosides comprising a hydroxyl group at the 5′ and/or 6′-position (Chen et al., Phosphorus, Sulfur and Silicon, 2002, 777, 1783-1786; Jung et al., Bioorg. Med. Chem., 2000, 8, 2501-2509; Gallier et al., Eur. J. Org. Chem., 2007, 925-933; and Hampton et al., J. Med. Chem., 1976, 19(8), 1029-1033). Unnatural nucleic acids can include 5′-phosphonate deoxyribonucleoside monomers and dimers having a 5′-phosphate group (Nawrot et al., Oligonucleotides, 2006, 16(1), 68-82). Unnatural nucleic acids can include nucleosides having a 6′-phosphonate group wherein the 5′ or/and 6′-position is unsubstituted or substituted with a thio-tert-butyl group (SC(CH₃)₃) (and analogs thereof); a methyleneamino group (CH₂NH₂) (and analogs thereof) or a cyano group (CN) (and analogs thereof) (Fairhurst et al., Synlett, 2001, 4, 467-472; Kappler et al., J. Med. Chem., 1986, 29, 1030-1038; Kappler et al., J. Med. Chem., 1982, 25, 1179-1184; Vrudhula et al., J. Med. Chem., 1987, 30, 888-894; Hampton et al., J. Med. Chem., 1976, 19, 1371-1377; Geze et al., J. Am. Chem. Soc, 1983, 105(26), 7638-7640; and Hampton et al., J. Am. Chem. Soc, 1973, 95(13), 4404-4414).

In some embodiments, unnatural nucleic acids also include modifications of the sugar moiety. In some cases, nucleic acids contain one or more nucleosides wherein the sugar group has been modified. Such sugar modified nucleosides may impart enhanced nuclease stability, increased binding affinity, or some other beneficial biological property. In certain embodiments, nucleic acids comprise a chemically modified ribofuranose ring moiety. Examples of chemically modified ribofuranose rings include, without limitation, addition of substituent groups (including 5′ and/or 2′ substituent groups; bridging of two ring atoms to form bicyclic nucleic acids (BNA); replacement of the ribosyl ring oxygen atom with S, N(R), or C(R₁)(R₂) (R═H, C₁-C₁₂ alkyl or a protecting group); and combinations thereof. Examples of chemically modified sugars can be found in WO2008/101157, US2005/0130923, and WO2007/134181.

In some instances, a modified nucleic acid comprises modified sugars or sugar analogs. Thus, in addition to ribose and deoxyribose, the sugar moiety can be pentose, deoxypentose, hexose, deoxyhexose, glucose, arabinose, xylose, lyxose, or a sugar “analog” cyclopentyl group. The sugar can be in a pyranosyl or furanosyl form. The sugar moiety may be the furanoside of ribose, deoxyribose, arabinose or 2′-O-alkylribose, and the sugar can be attached to the respective heterocyclic bases either in [alpha] or [beta]anomeric configuration. Sugar modifications include, but are not limited to, 2′-alkoxy-RNA analogs, 2′-amino-RNA analogs, 2′-fluoro-DNA, and 2′-alkoxy- or amino-RNA/DNA chimeras. For example, a sugar modification may include 2′-O-methyl-uridine or 2′-O-methyl-cytidine. Sugar modifications include 2′-O-alkyl-substituted deoxyribonucleosides and 2′-O-ethyleneglycol like ribonucleosides. The preparation of these sugars or sugar analogs and the respective “nucleosides” wherein such sugars or analogs are attached to a heterocyclic base (nucleic acid base) is known. Sugar modifications may also be made and combined with other modifications.

Modifications to the sugar moiety include natural modifications of the ribose and deoxy ribose as well as unnatural modifications. Sugar modifications include, but are not limited to, the following modifications at the 2′ position: OH; F; O—, S-, or N-alkyl; O—, S-, or N-alkenyl; O—, S- or N-alkynyl; or O-alkyl-O-alkyl, wherein the alkyl, alkenyl and alkynyl may be substituted or unsubstituted C₁ to C₁₀, alkyl or C₂ to C₁₀ alkenyl and alkynyl. 2′ sugar modifications also include but are not limited to —O[(CH₂)ₙO]m CH₃, —O(CH₂)ₙOCH₃, —O(CH₂)ₙNH₂, —O(CH₂)ₙCH₃, —O(CH₂)ₙONH₂, and —O(CH₂)ₙON[(CH₂)ₙ CH₃)]₂, where n and m are from 1 to about 10.

Other modifications at the 2′ position include but are not limited to: C₁ to C₁₀ lower alkyl, substituted lower alkyl, alkaryl, aralkyl, O-alkaryl, O-aralkyl, SH, SCH₃, OCN, Cl, Br, CN, CF₃, OCF₃, SOCH₃, SO₂ CH₃, ONO₂, NO₂, N3, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of the 5′ terminal nucleotide. Modified sugars also include those that contain modifications at the bridging ring oxygen, such as CH₂ and S. Nucleotide sugar analogs may also have sugar mimetics such as cyclobutyl moieties in place of the pentofuranosyl sugar. There are numerous United States patents that teach the preparation of such modified sugar structures and which detail and describe a range of base modifications, such as U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; and 5,700,920, each of which is herein incorporated by reference in its entirety.

Examples of nucleic acids having modified sugar moieties include, without limitation, nucleic acids comprising 5′-vinyl, 5′-methyl (R or S), 4′-S, 2′-F, 2′—OCH₃, and 2′-O(CH₂)₂OCH₃ substituent groups. The substituent at the 2′ position can also be selected from allyl, amino, azido, thio, O-allyl, O—(C₁-C₁₀ alkyl), OCF₃, O(CH₂)₂SCH₃, O(CH₂)₂—O—N(Rₘ)(Rₙ), and O—CH₂—C(═O)—N(Rₘ)(Rₙ), where each Rₘ and Rₙ is, independently, H or substituted or unsubstituted C₁-C₁₀ alkyl.

In certain embodiments, nucleic acids described herein include one or more bicyclic nucleic acids. In certain such embodiments, the bicyclic nucleic acid comprises a bridge between the 4′ and the 2′ ribosyl ring atoms. In certain embodiments, nucleic acids provided herein include one or more bicyclic nucleic acids wherein the bridge comprises a 4′ to 2′ bicyclic nucleic acid. Examples of such 4′ to 2′ bicyclic nucleic acids include, but are not limited to, one of the formulae: 4′-(CH₂)—O-2′ (LNA); 4′-(CH₂)—S-2′; 4′—(CH₂)₂—O-2′ (ENA); 4′-CH(CH₃)—O-2′ and 4′-CH(CH₂OCH₃)—O-2′, and analogs thereof (see, U.S. Pat. No. 7,399,845); 4′-C(CH₃)(CH₃)—O-2′ and analogs thereof, (see WO2009/006478, WO2008/150729, US2004/0171570, U.S. Pat. No. 7,427,672, Chattopadhyaya et al., J. Org. Chem., 209, 74, 118-134, and WO2008/154401). Also see, for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A, 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129(26) 8362-8379; Elayadi et al., Curr. Opinion Invens. Drugs, 2001, 2, 558-561; Braasch et al., Chem. Biol, 2001, 8, 1-7; Oram et al., Curr. Opinion Mol. Ther., 2001, 3, 239-243; U.S. Pat. Nos. 4,849,513; 5,015,733; 5,118,800; 5,118,802; 7,053,207; 6,268,490; 6,770,748; 6,794,499; 7,034,133; 6,525,191; 6,670,461; and 7,399,845; International Publication Nos. WO2004/106356, WO1994/14226, WO2005/021570, WO2007/090071, and WO2007/134181; U.S. Patent Publication Nos. US2004/0171570, US2007/0287831, and US2008/0039618; U.S. Provisional Application Nos. 60/989,574, 61/026,995, 61/026,998, 61/056,564, 61/086,231, 61/097,787, and 61/099,844; and International Applications Nos. PCT/US2008/064591, PCT US2008/066154, PCT US2008/068922, and PCT/DK98/00393.

In certain embodiments, nucleic acids comprise linked nucleic acids. Nucleic acids can be linked together using any inter nucleic acid linkage. The two main classes of inter nucleic acid linking groups are defined by the presence or absence of a phosphorus atom. Representative phosphorus containing inter nucleic acid linkages include, but are not limited to, phosphodiesters, phosphotriesters, methylphosphonates, phosphoramidate, and phosphorothioates (P═S). Representative non-phosphorus containing inter nucleic acid linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N*-dimethylhydrazine (—CH₂—N(CH₃)—N(CH₃)). In certain embodiments, inter nucleic acids linkages having a chiral atom can be prepared as a racemic mixture, as separate enantiomers, e.g., alkylphosphonates and phosphorothioates. Unnatural nucleic acids can contain a single modification. Unnatural nucleic acids can contain multiple modifications within one of the moieties or between different moieties.

Backbone phosphate modifications to nucleic acid include, but are not limited to, methyl phosphonate, phosphorothioate, phosphoramidate (bridging or non-bridging), phosphotriester, phosphorodithioate, phosphodithioate, and boranophosphate, and may be used in any combination. Other non-phosphate linkages may also be used.

In some embodiments, backbone modifications (e.g., methylphosphonate, phosphorothioate, phosphoroamidate and phosphorodithioate intemucleotide linkages) can confer immunomodulatory activity on the modified nucleic acid and/or enhance their stability in vivo.

In some instances, a phosphorous derivative (or modified phosphate group) is attached to the sugar or sugar analog moiety in and can be a monophosphate, diphosphate, triphosphate, alkylphosphonate, phosphorothioate, phosphorodithioate, phosphoramidate or the like. Exemplary polynucleotides containing modified phosphate linkages or non-phosphate linkages can be found in Peyrottes et al., 1996, Nucleic Acids Res. 24: 1841-1848; Chaturvedi et al., 1996, Nucleic Acids Res. 24:2318-2323; and Schultz et al., (1996) Nucleic Acids Res. 24:2966-2973; Matteucci, 1997, “Oligonucleotide Analogs: an Overview” in Oligonucleotides as Therapeutic Agents, (Chadwick and Cardew, ed.) John Wiley and Sons, New York, N.Y.; Zon, 1993, “Oligonucleoside Phosphorothioates” in Protocols for Oligonucleotides and Analogs, Synthesis and Properties, Humana Press, pp. 165-190; Miller et al., 1971, JACS 93:6657-6665; Jager et al., 1988, Biochem. 27:7247-7246; Nelson et al., 1997, JOC 62:7278-7287; U.S. Pat. No. 5,453,496; and Micklefield, 2001, Curr. Med. Chem. 8: 1157-1179.

In some cases, backbone modification comprises replacing the phosphodiester linkage with an alternative moiety such as an anionic, neutral or cationic group. Examples of such modifications include: anionic intemucleoside linkage; N3′ to P5′ phosphoramidate modification; boranophosphate DNA; prooligonucleotides; neutral intemucleoside linkages such as methylphosphonates; amide linked DNA; methylene(methylimino) linkages; formacetal and thioformacetal linkages; backbones containing sulfonyl groups; morpholino oligos; peptide nucleic acids (PNA); and positively charged deoxyribonucleic guanidine (DNG) oligos (Micklefield, 2001, Current Medicinal Chemistry 8: 1157-1179). A modified nucleic acid may comprise a chimeric or mixed backbone comprising one or more modifications, e.g. a combination of phosphate linkages such as a combination of phosphodiester and phosphorothioate linkages.

Substitutes for the phosphate include, for example, short chain alkyl or cycloalkyl intemucleoside linkages, mixed heteroatom and alkyl or cycloalkyl intemucleoside linkages, or one or more short chain heteroatomic or heterocyclic intemucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Numerous United States patents disclose how to make and use these types of phosphate replacements and include but are not limited to U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439. It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331; and 5,719,262 teach how to make and use PNA molecules, each of which is herein incorporated by reference. See also Nielsen et al., Science, 1991, 254, 1497-1500. It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. K Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EM5OJ, 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1-di-O-hexadecyl-rac-glycero-S—H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochem. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937). Numerous United States patents teach the preparation of such conjugates and include, but are not limited to U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

Described herein are nucleobases used in the compositions and methods for replication, transcription, translation, and incorporation of unnatural amino acids into proteins. In some embodiments, a nucleobase described herein comprises the structure:

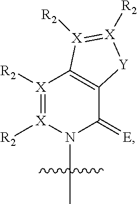

wherein
each X is independently carbon or nitrogen;
R₂ is optional and when present is independently hydrogen, alkyl, alkenyl, alkynyl; methoxy, methanethiol, methaneseleno, halogen, cyano, or azide group;
wherein each Y is independently sulfur, oxygen, selenium, or secondary amine;
wherein each E is independently oxygen, sulfur or selenium; and
wherein the wavy line indicates a point of bonding to a ribosyl, deoxyribosyl, or dideoxyribosyl moiety or an analog thereof, wherein the ribosyl, deoxyribosyl, or dideoxyribosyl moiety or analog thereof is in free form, connected to a mono-phosphate, diphosphate, or triphosphate group, optionally comprising an α-thiotriphosphate, β-thiotriphosphate, or γ-thiotriphosphate group, or is included in an RNA or a DNA or in an RNA analog or a DNA analog. In some embodiments, R₂ is lower alkyl (e.g., C₁-C₆), hydrogen, or halogen. In some embodiments of a nucleobase described herein, R₂ is fluoro. In some embodiments of a nucleobase described herein, X is carbon. In some embodiments of a nucleobase described herein, E is sulfur. In some embodiments of a nucleobase described herein, Y is sulfur. In some embodiments of a nucleobase described herein, a nucleobase has the structure:

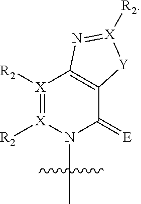

In some embodiments of a nucleobase described herein, E is sulfur and Y is sulfur. In some embodiments of a nucleobase described herein, the wavy line indicates a point of bonding to a ribosyl or deoxyribosyl moiety. In some embodiments of a nucleobase described herein, the wavy line indicates a point of bonding to a ribosyl or deoxyribosyl moiety, connected to a triphosphate group. In some embodiments of a nucleobase described herein is a component of a nucleic acid polymer. In some embodiments of a nucleobase described herein, the nucleobase is a component of a tRNA. In some embodiments of a nucleobase described herein, the nucleobase is a component of an anticodon in a tRNA. In some embodiments of a nucleobase described herein, the nucleobase is a component of an mRNA. In some embodiments of a nucleobase described herein, the nucleobase is a component of a codon of an mRNA. In some embodiments of a nucleobase described herein, the nucleobase is a component of RNA or DNA. In some embodiments of a nucleobase described herein, the nucleobase is a component of a codon in DNA. In some embodiments of a nucleobase described herein, the nucleobase forms a nucleobase pair with another complementary nucleobase.

An unnatural deoxyribonucleic acid (DNA), in some cases, is transcribed into messenger RNA (mRNA) comprising the unnatural bases described herein (e.g., d5SICS, dNaM, dTPT3, dMTMO, dCNMO, dTATI). Exemplary mRNA codons are coded by exemplary regions of the unnatural DNA comprising three contiguous deoxyribonucleotides (NNN) comprising TTX, TGX, CGX, AGX, GAX, CAX, GXT, CXT, GXG, AXG, GXC, AXC, GXA, CXC, TXC, ATX, CTX, TTX, GTX, TAX, or GGX, where X is the unnatural base attached to a 2′ deoxyribosyl moiety. The exemplary mRNA codons resulting from transcription of the exemplary unnatural DNA comprise three contiguous ribonucleotides (NNN) comprising UUX, UGX, CGX, AGX, GAX, CAX, GXU, CXU, GXG, AXG, GXC, AXC, GXA, CXC, UXC, AUX, CUX, UUX, GUX, UAX, or GGX, respectively, wherein X is the unnatural base attached to a ribosyl moiety. In some embodiments, the unnatural base is in a first position in the codon sequence (X—N—N). In some embodiments, the unnatural base is in a second (or middle) position in the codon sequence (N—X—N). In some embodiments, the unnatural base is in a third (last) position in the codon sequence (N—N—X).

The mRNA comprising the codons described herein, in some cases, is translated in vivo in a cell (e.g., eukaryotic cell). Translation of the mRNA comprising the unnatural base described herein is mediated by a transfer RNA (tRNA), comprising an anticodon sequence that is the reverse complement of the mRNA codon sequence described herein. In some embodiments, the tRNA anticodon comprises an unnatural base comprising YAA, XAA, YCA, XCA, YCG, XCG, YCU, XCU, YUC, XUC, YUG, XUG, AYC, AYG, CYC, CYU, GYC, GYU, UYC, GYG, GYA, YAU, XAU, XAG, YAG, XAC, YAC, XUA, YUA, XCC, or YCC, wherein X and Y, each represent an unnatural base, wherein X and Y are not the same. In some embodiments, the unnatural base is in a first position in the anticodon sequence (X/Y—N—N). In some embodiments, the unnatural base is in a second (or middle) position in the anticodon sequence (N—X/Y—N). In some embodiments, the unnatural base is in a third (last) position in the anticodon sequence (N—N—X/Y).

Nucleic Acid Base Pairing Properties

In some embodiments, an unnatural nucleotide forms a base pair (an unnatural base pair; UBP) with another unnatural nucleotide, e.g., during translation. For example, a first unnatural nucleic acid can form a base pair with a second unnatural nucleic acid. For example, one pair of unnatural nucleoside triphosphates that can base pair, e.g., during translation, include a nucleotide comprising (d)₅SICS and a nucleotide comprising (d)NaM. Other examples include but are not limited to: a nucleotide comprising (d) CNMO and a nucleotide comprising (d)TPT3. Such unnatural nucleotides can have a ribose or deoxyribose sugar moiety (indicated by the “(d)”). For example, one pair of unnatural nucleoside triphosphates that can base pair when incorporated into nucleic acids includes a nucleotide comprising TAT1 and a nucleotide comprising NaM. In some embodiments, one pair of unnatural nucleoside triphosphates that can base pair when incorporated into nucleic acids includes a nucleotide comprising dCNMO and a nucleotide comprising TAT1. In some embodiments, one pair of unnatural nucleoside triphosphates that can base pair when incorporated into nucleic acids includes a nucleotide comprising dTPT3 and a nucleotide comprising NaM. In some embodiments, an unnatural nucleic acid does not substantially form a base pair with a natural nucleic acid (A, T, G, C). In some embodiments, an unnatural nucleic acid can form a base pair with a natural nucleic acid.

In some embodiments, an unnatural (deoxy) ribonucleotide is an unnatural (deoxy) ribonucleotide that can form a UBP, but does not substantially form a base pair with each any of the natural (deoxy) ribonucleotides. In some embodiments, an unnatural (deoxy) ribonucleotide is an unnatural (deoxy) ribonucleotide that can form a UBP, but does not substantially form a base pair with one or more natural nucleic acids. For example, an unnatural nucleic acid may not substantially form a base pair with A, T, and, C, but can form a base pair with G. For example, an unnatural nucleic acid may not substantially form a base pair with A, T, and, G, but can form a base pair with C. For example, an unnatural nucleic acid may not substantially form a base pair with C, G, and, A, but can form a base pair with T. For example, an unnatural nucleic acid may not substantially form a base pair with C, G, and, T, but can form a base pair with A. For example, an unnatural nucleic acid may not substantially form a base pair with A and T, but can form a base pair with C and G. For example, an unnatural nucleic acid may not substantially form a base pair with A and C, but can form a base pair with T and G. For example, an unnatural nucleic acid may not substantially form a base pair with A and G, but can form a base pair with C and T. For example, an unnatural nucleic acid may not substantially form a base pair with C and T, but can form a base pair with A and G. For example, an unnatural nucleic acid may not substantially form a base pair with C and G, but can form a base pair with T and G. For example, an unnatural nucleic acid may not substantially form a base pair with T and G, but can form a base pair with A and G. For example, an unnatural nucleic acid may not substantially form a base pair with, G, but can form a base pair with A, T, and, C. For example, an unnatural nucleic acid may not substantially form a base pair with, A, but can form a base pair with G, T, and, C. For example, an unnatural nucleic acid may not substantially form a base pair with, T, but can form a base pair with G, A, and, C. For example, an unnatural nucleic acid may not substantially form a base pair with, C, but can form a base pair with G, T, and, A.

Exemplary, unnatural nucleotides capable of forming an unnatural base pair (UBP) (e.g., in RNA, such as between a tRNA and an mRNA) under conditions in vivo include, but are not limited to, 5SICS, d5SICS, NaM, dNaM, dTPT3, dMTMO, dCNMO, TAT1, and combinations thereof. In some embodiments, unnatural nucleotide base pairs include but are not limited to:

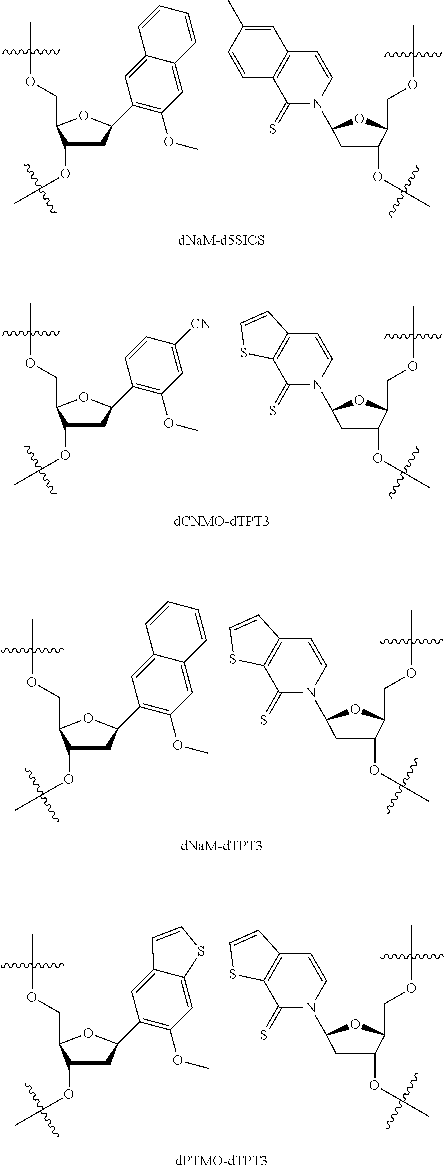

and corresponding ribo (RNA) forms thereof.

Unnatural base pairs (UBP) are formed between the codon sequence of the mRNA and the anticodon sequence of the tRNA to facilitate translation of the mRNA into an unnatural polypeptide. Codon-anticodon UBPs comprise, in some instances, a codon sequence comprising three contiguous nucleic acids read 5′ to 3′ of the mRNA (e.g., UUX), and an anticodon sequence comprising three contiguous nucleic acids ready 5′ to 3′ of the tRNA (e.g., YAA or XAA). In some embodiments, when the mRNA codon is UUX, the tRNA anticodon is YAA or XAA. In some embodiments, when the mRNA codon is UGX, the tRNA anticodon is YCA or XCA. In some embodiments, when the mRNA codon is CGX, the tRNA anticodon is YCG or XCG. In some embodiments, when the mRNA codon is AGX, the tRNA anticodon is YCU or XCU. In some embodiments, when the mRNA codon is GAX, the tRNA anticodon is YUC or XUC. In some embodiments, when the mRNA codon is CAX, the tRNA anticodon is YUG or XUG. In some embodiments, when the mRNA codon is GXU, the tRNA anticodon is AYC. In some embodiments, when the mRNA codon is CXU, the tRNA anticodon is AYG. In some embodiments, when the mRNA codon is GXG, the tRNA anticodon is CYC. In some embodiments, when the mRNA codon is AXG, the tRNA anticodon is CYU. In some embodiments, when the mRNA codon is GXC, the tRNA anticodon is GYC. In some embodiments, when the mRNA codon is AXC, the tRNA anticodon is GYU. In some embodiments, when the mRNA codon is GXA, the tRNA anticodon is UYC. In some embodiments, when the mRNA codon is CXC, the tRNA anticodon is GYG. In some embodiments, when the mRNA codon is UXC, the tRNA anticodon is GYA. In some embodiments, when the mRNA codon is AUX, the tRNA anticodon is YAU or XAU. In some embodiments, when the mRNA codon is CUX, the tRNA anticodon is XAG or YAG. In some embodiments, when the mRNA codon is UUX, the tRNA anticodon is XAA or YAA. In some embodiments, when the mRNA codon is GUX, the tRNA anticodon is XAC or YAC. In some embodiments, when the mRNA codon is UAX, the tRNA anticodon is XUA or YUA. In some embodiments, when the mRNA codon is GGX, the tRNA anticodon is XCC or YCC.

Natural and Unnatural Amino Acids

As used herein, an amino acid residue can refer to a molecule containing both an amino group and a carboxyl group. Suitable amino acids include, without limitation, both the D- and L-isomers of the naturally-occurring amino acids, as well as non-naturally occurring amino acids prepared by organic synthesis or any other method. The term amino acid, as used herein, includes, without limitation, α-amino acids, natural amino acids, non-natural amino acids, and amino acid analogs.

The term “α-amino acid” can refer to a molecule containing both an amino group and a carboxyl group bound to a carbon which is designated the α-carbon. For example:

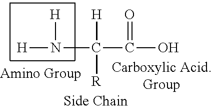

The term “β-amino acid” can refer to a molecule containing both an amino group and a carboxyl group in a β configuration.

“Naturally occurring amino acid” can refer to any one of the twenty amino acids commonly found in peptides synthesized in nature, and known by the one letter abbreviations A, R, N, C, D, Q, E, G, H, I, L, K, M, F, P, S, T, W, Y and V.

The following table shows a summary of the properties of natural amino acids:

| | 3- | 1- | Side- | Side-chain | |
|---|---|---|---|---|---|
| | Letter | Letter | chain | charge (pH | Hydropathy |
| Ammo Acid | Code | Code | Polarity | 7.4) | Index |
| Alanine | Ala | A | nonpolar | neutral | 1.8 |
| Arginine | Arg | R | polar | positive | −4.5 |
| Asparagine | Asn | N | polar | neutral | −3.5 |
| Aspartic acid | Asp | D | polar | negative | −3.5 |
| Cysteine | Cys | C | polar | neutral | 2.5 |
| Glutamic acid | Glu | E | polar | negative | −3.5 |
| Glutamine | Gln | Q | polar | neutral | −3.5 |
| Glycine | Gly | G | nonpolar | neutral | −0.4 |
| Histidine | His | H | polar | positive(10%) | −3.2 |
| | | | | neutral(90%) | |
| Isoleucine | Ile | I | nonpolar | neutral | 4.5 |
| Leucine | Leu | L | nonpolar | neutral | 3.8 |
| Lysine | Lys | K | polar | positive | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyrosine | Tyr | Y | polar | neutral | −1.3 |
| Valine | Val | V | nonpolar | neutral | 4.2 |

“Hydrophobic amino acids” include small hydrophobic amino acids and large hydrophobic amino acids. “Small hydrophobic amino acid” can be glycine, alanine, proline, and analogs thereof. “Large hydrophobic amino acids” can be valine, leucine, isoleucine, phenylalanine, methionine, tryptophan, and analogs thereof. “Polar amino acids” can be serine, threonine, asparagine, glutamine, cysteine, tyrosine, and analogs thereof. “Charged amino acids” can be lysine, arginine, histidine, aspartate, glutamate, and analogs thereof.

An “amino acid analog” can be a molecule which is structurally similar to an amino acid and which can be substituted for an amino acid in the formation of a peptidomimetic macrocycle Amino acid analogs include, without limitation, O-amino acids and amino acids where the amino or carboxy group is substituted by a similarly reactive group (e.g., substitution of the primary amine with a secondary or tertiary amine, or substitution of the carboxy group with an ester).

A non-canonical amino acid (ncAA) or “non-natural amino acid” can be an amino acid which is not one of the twenty amino acids commonly found in peptides synthesized in nature, and known by the one letter abbreviations A, R, N, C, D, Q, E, G, H, I, L, K, M, F, P, S, T, W, Y and V. In some instances, non-natural amino acids are a subset of non-canonical amino acids.

Amino acid analogs can include β-amino acid analogs. Examples of β-amino acid analogs include, but are not limited to, the following: cyclic β-amino acid analogs; β-alanine; (R)-β-phenylalanine; (R)-1,2,3,4-tetrahydro-isoquinoline-3-acetic acid; (R)-3-amino-4-(1-naphthyl)-butyric acid; (R)-3-amino-4-(2,4-dichlorophenyl)butyric acid; (R)-3-amino-4-(2-chlorophenyl)-butyric acid; (R)-3-amino-4-(2-cyanophenyl)-butyric acid; (R)-3-amino-4-(2-fluorophenyl)-butyric acid; (R)-3-amino-4-(2-furyl)-butyric acid; (R)-3-amino-4-(2-methylphenyl)-butyric acid; (R)-3-amino-4-(2-naphthyl)-butyric acid; (R)-3-amino-4-(2-thienyl)-butyric acid; (R)-3-amino-4-(2-trifluoromethylphenyl)-butyric acid; (R)-3-amino-4-(3,4-dichlorophenyl)butyric acid; (R)-3-amino-4-(3,4-difluorophenyl)butyric acid; (R)-3-amino-4-(3-benzothienyl)-butyric acid; (R)-3-amino-4-(3-chlorophenyl)-butyric acid; (R)-3-amino-4-(3-cyanophenyl)-butyric acid; (R)-3-amino-4-(3-fluorophenyl)-butyric acid; (R)-3-amino-4-(3-methylphenyl)-butyric acid; (R)-3-amino-4-(3-pyridyl)-butyric acid; (R)-3-amino-4-(3-thienyl)-butyric acid; (R)-3-amino-4-(3-trifluoromethylphenyl)-butyric acid; (R)-3-amino-4-(4-bromophenyl)-butyric acid; (R)-3-amino-4-(4-chlorophenyl)-butyric acid; (R)-3-amino-4-(4-cyanophenyl)-butyric acid; (R)-3-amino-4-(4-fluorophenyl)-butyric acid; (R)-3-amino-4-(4-iodophenyl)-butyric acid; (R)-3-amino-4-(4-methylphenyl)-butyric acid; (R)-3-amino-4-(4-nitrophenyl)-butyric acid; (R)-3-amino-4-(4-pyridyl)-butyric acid; (R)-3-amino-4-(4-trifluoromethylphenyl)-butyric acid; (R)-3-amino-4-pentafluoro-phenylbutyric acid; (R)-3-amino-5-hexenoic acid; (R)-3-amino-5-hexynoic acid; (R)-3-amino-5-phenylpentanoic acid; (R)-3-amino-6-phenyl-5-hexenoic acid; (S)-1,2,3,4-tetrahydro-isoquinoline-3-acetic acid; (S)-3-amino-4-(1-naphthyl)-butyric acid; (S)-3-amino-4-(2,4-dichlorophenyl)butyric acid; (S)-3-amino-4-(2-chlorophenyl)-butyric acid; (S)-3-amino-4-(2-cyanophenyl)-butyric acid; (S)-3-amino-4-(2-fluorophenyl)-butyric acid; (S)-3-amino-4-(2-furyl)-butyric acid; (S)-3-amino-4-(2-methylphenyl)-butyric acid; (S)-3-amino-4-(2-naphthyl)-butyric acid; (S)-3-amino-4-(2-thienyl)-butyric acid; (S)-3-amino-4-(2-trifluoromethylphenyl)-butyric acid; (S)-3-amino-4-(3,4-dichlorophenyl)butyric acid; (S)-3-amino-4-(3,4-difluorophenyl)butyric acid; (S)-3-amino-4-(3-benzothienyl)-butyric acid; (S)-3-amino-4-(3-chlorophenyl)-butyric acid; (S)-3-amino-4-(3-cyanophenyl)-butyric acid; (S)-3-amino-4-(3-fluorophenyl)-butyric acid; (S)-3-amino-4-(3-methylphenyl)-butyric acid; (S)-3-amino-4-(3-pyridyl)-butyric acid; (S)-3-amino-4-(3-thienyl)-butyric acid; (S)-3-amino-4-(3-trifluoromethylphenyl)-butyric acid; (S)-3-amino-4-(4-bromophenyl)-butyric acid; (S)-3-amino-4-(4-chlorophenyl) butyric acid; (S)-3-amino-4-(4-cyanophenyl)-butyric acid; (S)-3-amino-4-(4-fluorophenyl) butyric acid; (S)-3-amino-4-(4-iodophenyl)-butyric acid; (S)-3-amino-4-(4-methylphenyl)-butyric acid; (S)-3-amino-4-(4-nitrophenyl)-butyric acid; (S)-3-amino-4-(4-pyridyl)-butyric acid; (S)-3-amino-4-(4-trifluoromethylphenyl)-butyric acid; (S)-3-amino-4 pentafluoro-phenylbutyric acid; (S)-3-amino-5-hexenoic acid; (S)-3-amino-5-hexynoic acid; (S)-3-amino-5-phenylpentanoic acid; (S)-3-amino-6-phenyl-5-hexenoic acid; 1,2,5,6-tetrahydropyridine-3-carboxylic acid; 1,2,5,6-tetrahydropyridine-4-carboxylic acid; 3-amino-3-(2-chlorophenyl)-propionic acid; 3-amino-3-(2-thienyl)-propionic acid; 3-amino-3-(3-bromophenyl)-propionic acid; 3-amino-3-(4-chlorophenyl)-propionic acid; 3-amino-3-(4-methoxyphenyl)-propionic acid; 3-amino-4,4,4-trifluoro-butyric acid; 3-aminoadipic acid; D-β-phenylalanine; β-leucine; L-β-homoalanine; L-β-homoaspartic acid γ-benzyl ester; L-β-homoglutamic acid δ-benzyl ester; L-β-homoisoleucine; L-β-homoleucine; L-β-homomethionine; L-β-homophenylalanine; L-β-homoproline; L-β-homotryptophan; L-β-homovaline; L-Nω-benzyloxycarbonyl-β-homolysine; Nω-L-β-homoarginine; O-benzyl-L-β-homohydroxyproline; O-benzyl-L-β-homoserine; O-benzyl-L-β-homothreonine; O-benzyl-L-β-homotyrosine; γ-trityl-L-β-homoasparagine; (R)-β-phenylalanine; L-β-homoaspartic acid γ-t-butyl ester; L-β-homoglutamic acid δ-t-butyl ester; L-Nω-β-homolysine; Nδ-trityl-L-β-homoglutamine; Nω-2,2,4,6,7-pentamethyl-dihydrobenzofuran-5-sulfonyl-L-β-homoarginine; O-t-butyl-L-β-homohydroxy-proline; O-t-butyl-L-β-homoserine; O-t-butyl-L-β-homothreonine; O-t-butyl-L-β-homotyrosine; 2-aminocyclopentane carboxylic acid; and 2-aminocyclohexane carboxylic acid.

Amino acid analogs can include analogs of alanine, valine, glycine or leucine. Examples of amino acid analogs of alanine, valine, glycine, and leucine include, but are not limited to, the following: α-methoxyglycine; α-allyl-L-alanine; α-aminoisobutyric acid; α-methyl-leucine; β-(1-naphthyl)-D-alanine; β-(1-naphthyl)-L-alanine; β-(2-naphthyl)-D-alanine; β-(2-naphthyl)-L-alanine; β-(2-pyridyl)-D-alanine; β-(2-pyridyl)-L-alanine; β-(2-thienyl)-D-alanine; 1-(2-thienyl)-L-alanine; β-(3-benzothienyl)-D-alanine; 1-(3-benzothienyl)-L-alanine; β-(3-pyridyl)-D-alanine; β-(3-pyridyl)-L-alanine; β-(4-pyridyl)-D-alanine; β-(4-pyridyl)-L-alanine; β-chloro-L-alanine; β-cyano-L-alanine; β-cyclohexyl-D-alanine; β-cyclohexyl-L-alanine; β-cyclopenten-1-yl-alanine; β-cyclopentyl-alanine; β-cyclopropyl-L-Ala-OH.dicyclohexylammonium salt; β-t-butyl-D-alanine; β-t-butyl-L-alanine; γ-aminobutyric acid; L-α,β-diaminopropionic acid; 2,4-dinitro-phenylglycine; 2,5-dihydro-D-phenylglycine; 2-amino-4,4,4-trifluorobutyric acid; 2-fluoro-phenylglycine; 3-amino-4,4,4-trifluoro-butyric acid; 3-fluoro-valine; 4,4,4-trifluoro-valine; 4,5-dehydro-L-leu-OH.dicyclohexylammonium salt; 4-fluoro-D-phenylglycine; 4-fluoro-L-phenylglycine; 4-hydroxy-D-phenylglycine; 5,5,5-trifluoro-leucine; 6-aminohexanoic acid; cyclopentyl-D-Gly-OH.dicyclohexylammonium salt; cyclopentyl-Gly-OH.dicyclohexylammonium salt; D-α,β-diaminopropionic acid; D-α-aminobutyric acid; D-α-t-butylglycine; D-(2-thienyl)glycine; D-(3-thienyl)glycine; D-2-aminocaproic acid; D-2-indanylglycine; D-allylglycine-dicyclohexylammonium salt; D-cyclohexylglycine; D-norvaline; D-phenylglycine; β-aminobutyric acid; β-aminoisobutyric acid; (2-bromophenyl)glycine; (2-methoxyphenyl)glycine; (2-methylphenyl)glycine; (2-thiazoyl)glycine; (2-thienyl)glycine; 2-amino-3-(dimethylamino)-propionic acid; L-α,β-diaminopropionic acid; L-α-aminobutyric acid; L-α-t-butylglycine; L-(3-thienyl)glycine; L-2-amino-3-(dimethylamino)-propionic acid; L-2-aminocaproic acid dicyclohexyl-ammonium salt; L-2-indanylglycine; L-allylglycine dicyclohexyl ammonium salt; L-cyclohexylglycine; L-phenylglycine; L-propargylglycine; L-norvaline; N-α-aminomethyl-L-alanine; D-α,γ-diaminobutyric acid; L-α,γ-diaminobutyric acid; β-cyclopropyl-L-alanine; (N-D-(2,4-dinitrophenyl))-L-α,β-diaminopropionic acid; (N-β-1-(4,4-dimethyl-2,6-dioxocyclohex-1-ylidene)ethyl)-D-α,β-diaminopropionic acid; (N-γ-1-(4,4-dimethyl-2,6-dioxocyclohex-1-ylidene)ethyl)-L-α,β-diaminopropionic acid; (N-γ-4-methyltrityl)-L-α,β-diaminopropionic acid; (N-β-allyloxycarbonyl)-L-α,β-diaminopropionic acid; (N—)-1-(4,4-dimethyl-2,6-dioxocyclohex-1-ylidene)ethyl)-D-α,γ-diaminobutyric acid; (N-γ-1-(4,4-dimethyl-2,6-dioxocyclohex-1-ylidene)ethyl)-L-α,γ-diaminobutyric acid; (N-γ-4-methyltrityl)-D-α,γ-diaminobutyric acid; (N-γ-4-methyltrityl)-L-α,γ-diaminobutyric acid; (N-γ-allyloxycarbonyl)-L-α,γ-diaminobutyric acid; D-α,γ-diaminobutyric acid; 4,5-dehydro-L-leucine; cyclopentyl-D-Gly-OH; cyclopentyl-Gly-OH; D-allylglycine; D-homocyclohexylalanine; L-1-pyrenylalanine; L-2-aminocaproic acid; L-allylglycine; L-homocyclohexylalanine; and N-(2-hydroxy-4-methoxy-Bzl)-Gly-OH.

Amino acid analogs can include analogs of arginine or lysine. Examples of amino acid analogs of arginine and lysine include, but are not limited to, the following: citrulline; L-2-amino-3-guanidinopropionic acid; L-2-amino-3-ureidopropionic acid; L-citrulline; Lys(Me)₂—OH; Lys(N₃)—OH; Nδ-benzyloxycarbonyl-L-ornithine; Nω-nitro-D-arginine; Nω-nitro-L-arginine; α-methyl-omithine; 2,6-diaminoheptanedioic acid; L-ornithine; (Nδ-1-(4,4-dimethyl-2,6-dioxo-cyclohex-1-ylidene)ethyl)-D-omithine; (Nδ-1-(4,4-dimethyl-2,6-dioxo-cyclohex-1-ylidene)ethyl)-L-ornithine; (Nδ-4-methyltrityl)-D-omithine; (Nδ-4-methyltrityl)-L-ornithine; D-omithine; L-ornithine; Arg(Me)(Pbf)—OH; Arg(Me)₂—OH (asymmetrical); Arg(Me)₂—OH (symmetrical); Lys(ivDde)—OH; Lys(Me)₂—OH.HCl; Lys(Me3)—OH chloride; Nω-nitro-D-arginine; and Nω-nitro-L-arginine.

Amino acid analogs can include analogs of aspartic or glutamic acids. Examples of amino acid analogs of aspartic and glutamic acids include, but are not limited to, the following: α-methyl-D-aspartic acid; α-methyl-glutamic acid; α-methyl-L-aspartic acid; γ-methylene-glutamic acid; (N-γ-ethyl)-L-glutamine; [N-α-(4-aminobenzoyl)]-L-glutamic acid; 2,6-diaminopimelic acid; L-α-aminosuberic acid; D-2-aminoadipic acid; D-α-aminosuberic acid; α-aminopimelic acid; iminodiacetic acid; L-2-aminoadipic acid; threo-β-methyl-aspartic acid; γ-carboxy-D-glutamic acid γ,γ-di-t-butyl ester; γ-carboxy-L-glutamic acid γ,γ-di-t-butyl ester; Glu(OAll)—OH; L-Asu(OtBu)—OH; and pyroglutamic acid.

Amino acid analogs can include analogs of cysteine and methionine. Examples of amino acid analogs of cysteine and methionine include, but are not limited to, Cys(farnesyl)-OH, Cys(farnesyl)-OMe, α-methyl-methionine, Cys(2-hydroxyethyl)-OH, Cys(3-aminopropyl)-OH, 2-amino-4-(ethylthio)butyric acid, buthionine, buthioninesulfoximine, ethionine, methionine methylsulfonium chloride, selenomethionine, cysteic acid, [2-(4-pyridyl)ethyl]-DL-penicillamine, [2-(4-pyridyl)ethyl]-L-cysteine, 4-methoxybenzyl-D-penicillamine, 4-methoxybenzyl-L-penicillamine, 4-methylbenzyl-D-penicillamine, 4-methylbenzyl-L-penicillamine, benzyl-D-cysteine, benzyl-L-cysteine, benzyl-DL-homocysteine, carbamoyl-L-cysteine, carboxyethyl-L-cysteine, carboxymethyl-L-cysteine, diphenylmethyl-L-cysteine, ethyl-L-cysteine, methyl-L-cysteine, t-butyl-D-cysteine, trityl-L-homocysteine, trityl-D-penicillamine, cystathionine, homocystine, L-homocystine, (2-aminoethyl)-L-cysteine, seleno-L-cystine, cystathionine, Cys(StBu)—OH, and acetamidomethyl-D-penicillamine.

Amino acid analogs can include analogs of phenylalanine and tyrosine. Examples of amino acid analogs of phenylalanine and tyrosine include β-methyl-phenylalanine, β-hydroxyphenylalanine, α-methyl-3-methoxy-DL-phenylalanine, α-methyl-D-phenylalanine, α-methyl-L-phenylalanine, 1,2,3,4-tetrahydroisoquinoline-3-carboxylic acid, 2,4-dichloro-phenylalanine, 2-(trifluoromethyl)-D-phenylalanine, 2-(trifluoromethyl)-L-phenylalanine, 2-bromo-D-phenylalanine, 2-bromo-L-phenylalanine, 2-chloro-D-phenylalanine, 2-chloro-L-phenylalanine, 2-cyano-D-phenylalanine, 2-cyano-L-phenylalanine, 2-fluoro-D-phenylalanine, 2-fluoro-L-phenylalanine, 2-methyl-D-phenylalanine, 2-methyl-L-phenylalanine, 2-nitro-D-phenylalanine, 2-nitro-L-phenylalanine, 2; 4; 5-trihydroxy-phenylalanine, 3,4,5-trifluoro-D-phenylalanine, 3,4,5-trifluoro-L-phenylalanine, 3,4-dichloro-D-phenylalanine, 3,4-dichloro-L-phenylalanine, 3,4-difluoro-D-phenylalanine, 3,4-difluoro-L-phenylalanine, 3,4-dihydroxy-L-phenylalanine, 3,4-dimethoxy-L-phenylalanine, 3,5,3′-triiodo-L-thyronine, 3,5-diiodo-D-tyrosine, 3,5-diiodo-L-tyrosine, 3,5-diiodo-L-thyronine, 3-(trifluoromethyl)-D-phenylalanine, 3-(trifluoromethyl)-L-phenylalanine, 3-amino-L-tyrosine, 3-bromo-D-phenylalanine, 3-bromo-L-phenylalanine, 3-chloro-D-phenylalanine, 3-chloro-L-phenylalanine, 3-chloro-L-tyrosine, 3-cyano-D-phenylalanine, 3-cyano-L-phenylalanine, 3-fluoro-D-phenylalanine, 3-fluoro-L-phenylalanine, 3-fluoro-tyrosine, 3-iodo-D-phenylalanine, 3-iodo-L-phenylalanine, 3-iodo-L-tyrosine, 3-methoxy-L-tyrosine, 3-methyl-D-phenylalanine, 3-methyl-L-phenylalanine, 3-nitro-D-phenylalanine, 3-nitro-L-phenylalanine, 3-nitro-L-tyrosine, 4-(trifluoromethyl)-D-phenylalanine, 4-(trifluoromethyl)-L-phenylalanine, 4-amino-D-phenylalanine, 4-amino-L-phenylalanine, 4-benzoyl-D-phenylalanine, 4-benzoyl-L-phenylalanine, 4-bis(2-chloroethyl)amino-L-phenylalanine, 4-bromo-D-phenylalanine, 4-bromo-L-phenylalanine, 4-chloro-D-phenylalanine, 4-chloro-L-phenylalanine, 4-cyano-D-phenylalanine, 4-cyano-L-phenylalanine, 4-fluoro-D-phenylalanine, 4-fluoro-L-phenylalanine, 4-iodo-D-phenylalanine, 4-iodo-L-phenylalanine, homophenylalanine, thyroxine, 3,3-diphenylalanine, thyronine, ethyl-tyrosine, and methyl-tyrosine.

Amino acid analogs can include analogs of proline. Examples of amino acid analogs of proline include, but are not limited to, 3,4-dehydro-proline, 4-fluoro-proline, cis-4-hydroxy-proline, thiazolidine-2-carboxylic acid, and trans-4-fluoro-proline.

Amino acid analogs can include analogs of serine and threonine. Examples of amino acid analogs of serine and threonine include, but are not limited to, 3-amino-2-hydroxy-5-methylhexanoic acid, 2-amino-3-hydroxy-4-methylpentanoic acid, 2-amino-3-ethoxybutanoic acid, 2-amino-3-methoxybutanoic acid, 4-amino-3-hydroxy-6-methylheptanoic acid, 2-amino-3-benzyloxypropionic acid, 2-amino-3-benzyloxypropionic acid, 2-amino-3-ethoxypropionic acid, 4-amino-3-hydroxybutanoic acid, and α-methylserine.

Amino acid analogs can include analogs of tryptophan. Examples of amino acid analogs of tryptophan include, but are not limited to, the following: α-methyl-tryptophan; β-(3-benzothienyl)-D-alanine; β-(3-benzothienyl)-L-alanine; 1-methyl-tryptophan; 4-methyl-tryptophan; 5-benzyloxy-tryptophan; 5-bromo-tryptophan; 5-chloro-tryptophan; 5-fluoro-tryptophan; 5-hydroxy-tryptophan; 5-hydroxy-L-tryptophan; 5-methoxy-tryptophan; 5-methoxy-L-tryptophan; 5-methyl-tryptophan; 6-bromo-tryptophan; 6-chloro-D-tryptophan; 6-chloro-tryptophan; 6-fluoro-tryptophan; 6-methyl-tryptophan; 7-benzyloxy-tryptophan; 7-bromo-tryptophan; 7-methyl-tryptophan; D-1,2,3,4-tetrahydro-norharman-3-carboxylic acid; 6-methoxy-1,2,3,4-tetrahydronorharman-1-carboxylic acid; 7-azatryptophan; L-1,2,3,4-tetrahydro-norharman-3-carboxylic acid; 5-methoxy-2-methyl-tryptophan; and 6-chloro-L-tryptophan.

Amino acid analogs can be racemic. In some instances, the D isomer of the amino acid analog is used. In some cases, the L isomer of the amino acid analog is used. In some instances, the amino acid analog comprises chiral centers that are in the R or S configuration. Sometimes, the amino group(s) of a β-amino acid analog is substituted with a protecting group, e.g., tert-butyloxycarbonyl (BOC group), 9-fluorenylmethyloxycarbonyl (FMOC), tosyl, and the like. Sometimes, the carboxylic acid functional group of a β-amino acid analog is protected, e.g., as its ester derivative. In some cases, the salt of the amino acid analog is used.

In some embodiments, an unnatural amino acid is an unnatural amino acid described in Liu C. C., Schultz, P. G. Annu. Rev. Biochem. 2010, 79, 413. In some embodiments, an unnatural amino acid comprises N6((2-azidoethoxy)-carbonyl)-L-lysine.

In some embodiments, an amino acid residue described herein (e.g., within a protein) is mutated to an unnatural amino acid prior to binding to a conjugating moiety. In some cases, the mutation to an unnatural amino acid prevents or minimizes a self-antigen response of the immune system. As used herein, the term “unnatural amino acid” refers to an amino acid other than the 20 amino acids that occur naturally in protein. Non-limiting examples of unnatural amino acids include: p-acetyl-L-phenylalanine, p-iodo-L-phenylalanine, p-methoxyphenylalanine, O-methyl-L-tyrosine, p-propargyloxyphenylalanine, p-propargyl-phenylalanine, L-3-(2-naphthyl)alanine, 3-methyl-phenylalanine, O-4-allyl-L-tyrosine, 4-propyl-L-tyrosine, tri-O-acetyl-GlcNAcp-serine, L-Dopa, fluorinated phenylalanine, isopropyl-L-phenylalanine, p-azido-L-phenylalanine, p-acyl-L-phenylalanine, p-benzoyl-L-phenylalanine, p-Boronophenylalanine, O-propargyltyrosine, L-phosphoserine, phosphonoserine, phosphonotyrosine, p-bromophenylalanine, selenocysteine, p-amino-L-phenylalanine, isopropyl-L-phenylalanine, N6-((azidoethoxy)-carbonyl)-L-lysine, AzK), N6-(((2-azidobenzyl)oxy)carbonyl)-L-lysine, N6-(((3-azidobenzyl)oxy)carbonyl)-L-lysine, or N6-(((4-azidobenzyl)oxy)carbonyl)-L-lysine, an unnatural analogue of a tyrosine amino acid; an unnatural analogue of a glutamine amino acid; an unnatural analogue of a phenylalanine amino acid; an unnatural analogue of a serine amino acid; an unnatural analogue of a threonine amino acid; an alkyl, aryl, acyl, azido, cyano, halo, hydrazine, hydrazide, hydroxyl, alkenyl, alkynyl, ether, thiol, sulfonyl, seleno, ester, thioacid, borate, boronate, phospho, phosphono, phosphine, heterocyclic, enone, imine, aldehyde, hydroxylamine, keto, or amino substituted amino acid, or a combination thereof; an amino acid with a photoactivatable cross-linker; a spin-labeled amino acid; a fluorescent amino acid; a metal binding amino acid; a metal-containing amino acid; a radioactive amino acid; a photocaged and/or photoisomerizable amino acid; a biotin or biotin-analogue containing amino acid; a keto containing amino acid; an amino acid comprising polyethylene glycol or polyether; a heavy atom substituted amino acid; a chemically cleavable or photocleavable amino acid; an amino acid with an elongated side chain; an amino acid containing a toxic group; a sugar substituted amino acid; a carbon-linked sugar-containing amino acid; a redox-active amino acid; an α-hydroxy containing acid; an amino thio acid; an α, α disubstituted amino acid; a β-amino acid; a cyclic amino acid other than proline or histidine, and an aromatic amino acid other than phenylalanine, tyrosine or tryptophan.

In some embodiments, the unnatural amino acid comprises a selective reactive group, or a reactive group for site-selective labeling of a target protein or polypeptide. In some instances, the chemistry is a biorthogonal reaction (e.g., biocompatible and selective reactions). In some cases, the chemistry is a Cu(I)-catalyzed or “copper-free” alkyne-azide triazole-forming reaction, the Staudinger ligation, inverse-electron-demand Diels-Alder (IEDDA) reaction, “photo-click” chemistry, or a metal-mediated process such as olefin metathesis and Suzuki-Miyaura or Sonogashira cross-coupling. In some embodiments, the unnatural amino acid comprises a photoreactive group, which crosslinks, upon irradiation with, e.g., UV. In some embodiments, the unnatural amino acid comprises a photo-caged amino acid. In some instances, the unnatural amino acid is a para-substituted, meta-substituted, or an ortho-substituted amino acid derivative.

In some instances, the unnatural amino acid comprises p-acetyl-L-phenylalanine, p-azidomethyl-L-phenylalanine (pAMF), p-iodo-L-phenylalanine, O-methyl-L-tyrosine, p-methoxyphenylalanine, p-propargyloxyphenylalanine, p-propargyl-phenylalanine, L-3-(2-naphthyl)alanine, 3-methyl-phenylalanine, O-4-allyl-L-tyrosine, 4-propyl-L-tyrosine, tri-O-acetyl-GlcNAcp-serine, L-Dopa, fluorinated phenylalanine, isopropyl-L-phenylalanine, p-azido-L-phenylalanine, p-acyl-L-phenylalanine, p-benzoyl-L-phenylalanine, L-phosphoserine, phosphonoserine, phosphonotyrosine, p-bromophenylalanine, p-amino-L-phenylalanine, or isopropyl-L-phenylalanine.

In some cases, the unnatural amino acid is 3-aminotyrosine, 3-nitrotyrosine, 3,4-dihydroxy-phenylalanine, or 3-iodotyrosine. In some cases, the unnatural amino acid is phenylselenocysteine. In some instances, the unnatural amino acid is a benzophenone, ketone, iodide, methoxy, acetyl, benzoyl, or azide containing phenylalanine derivative. In some instances, the unnatural amino acid is a benzophenone, ketone, iodide, methoxy, acetyl, benzoyl, or azide containing lysine derivative. In some instances, the unnatural amino acid comprises an aromatic side chain. In some instances, the unnatural amino acid does not comprise an aromatic side chain. In some instances, the unnatural amino acid comprises an azido group. In some instances, the unnatural amino acid comprises a Michael-acceptor group. In some instances, Michael-acceptor groups comprise an unsaturated moiety capable of forming a covalent bond through a 1,2-addition reaction. In some instances, Michael-acceptor groups comprise electron-deficient alkenes or alkynes. In some instances, Michael-acceptor groups include but are not limited to alpha,beta unsaturated: ketones, aldehydes, sulfoxides, sulfones, nitriles, imines, or aromatics. In some instances, the unnatural amino acid is dehydroalanine. In some instances, the unnatural amino acid comprises an aldehyde or ketone group. In some instances, the unnatural amino acid is a lysine derivative comprising an aldehyde or ketone group. In some instances, the unnatural amino acid is a lysine derivative comprising one or more O, N, Se, or S atoms at the beta, gamma, or delta position. In some instances, the unnatural amino acid is a lysine derivative comprising O, N, Se, or S atoms at the gamma position. In some instances, the unnatural amino acid is a lysine derivative wherein the epsilon N atom is replaced with an oxygen atom. In some instances, the unnatural amino acid is a lysine derivative that is not naturally-occurring post-translationally modified lysine.

In some instances, the unnatural amino acid is an amino acid comprising a side chain, wherein the sixth atom from the alpha position comprises a carbonyl group. In some instances, the unnatural amino acid is an amino acid comprising a side chain, wherein the sixth atom from the alpha position comprises a carbonyl group, and the fifth atom from the alpha position is nitrogen. In some instances, the unnatural amino acid is an amino acid comprising a side chain, wherein the seventh atom from the alpha position is an oxygen atom.

In some instances, the unnatural amino acid is a serine derivative comprising selenium. In some instances, the unnatural amino acid is selenoserine (2-amino-3-hydroselenopropanoic acid). In some instances, the unnatural amino acid is 2-amino-3-((2-((3-(benzyloxy)-3-oxopropyl)amino)ethyl)selanyl)propanoic acid. In some instances, the unnatural amino acid is 2-amino-3-(phenylselanyl)propanoic acid. In some instances, the unnatural amino acid comprises selenium, wherein oxidation of the selenium results in the formation of an unnatural amino acid comprising an alkene.

In some instances, the unnatural amino acid comprises a cyclooctynyl group. In some instances, the unnatural amino acid comprises a transcycloctenyl group. In some instances, the unnatural amino acid comprises a norbornenyl group. In some instances, the unnatural amino acid comprises a cyclopropenyl group. In some instances, the unnatural amino acid comprises a diazirine group. In some instances, the unnatural amino acid comprises a tetrazine group.

In some instances, the unnatural amino acid is a lysine derivative, wherein the side-chain nitrogen is carbamoylated. In some instances, the unnatural amino acid is a lysine derivative, wherein the side-chain nitrogen is acylated. In some instances, the unnatural amino acid is 2-amino-6-{[(tert-butoxy)carbonyl]amino}hexanoic acid. In some instances, the unnatural amino acid is 2-amino-6-{[(tert-butoxy)carbonyl]amino}hexanoic acid. In some instances, the unnatural amino acid is N6-Boc-N6-methyllysine. In some instances, the unnatural amino acid is N6-acetyllysine. In some instances, the unnatural amino acid is pyrrolysine. In some instances, the unnatural amino acid is N6-trifluoroacetyllysine. In some instances, the unnatural amino acid is 2-amino-6-{[(benzyloxy)carbonyl]amino}hexanoic acid. In some instances, the unnatural amino acid is 2-amino-6-{[(p-iodobenzyloxy)carbonyl]amino}hexanoic acid. In some instances, the unnatural amino acid is 2-amino-6-{[(p-nitrobenzyloxy)carbonyl]amino}hexanoic acid. In some instances, the unnatural amino acid is N6-prolyllysine. In some instances, the unnatural amino acid is 2-amino-6-{[(cyclopentyloxy)carbonyl]amino}hexanoic acid. In some instances, the unnatural amino acid is N6-(cyclopentanecarbonyl) lysine. In some instances, the unnatural amino acid is N6-(tetrahydrofuran-2-carbonyl) lysine. In some instances, the unnatural amino acid is N6-(3-ethynyltetrahydrofuran-2-carbonyl) lysine. In some instances, the unnatural amino acid is N6-((prop-2-yn-1-yloxy)carbonyl) lysine. In some instances, the unnatural amino acid is 2-amino-6-{[(2-azidocyclopentyloxy)carbonyl]amino}hexanoic acid. In some instances, the unnatural amino acid is N6-((2-azidoethoxy)carbonyl) lysine. In some instances, the unnatural amino acid is 2-amino-6-{[(2-nitrobenzyloxy)carbonyl]amino}hexanoic acid. In some instances, the unnatural amino acid is 2-amino-6-{[(2-cyclooctynyloxy)carbonyl]amino}hexanoic acid. In some instances, the unnatural amino acid is N6-(2-aminobut-3-ynoyl) lysine. In some instances, the unnatural amino acid is 2-amino-6-((2-aminobut-3-ynoyl)oxy)hexanoic acid. In some instances, the unnatural amino acid is N6-(allyloxycarbonyl) lysine. In some instances, the unnatural amino acid is N6-(butenyl-4-oxycarbonyl) lysine. In some instances, the unnatural amino acid is N6-(pentenyl-5-oxycarbonyl) lysine. In some instances, the unnatural amino acid is N6-((but-3-yn-1-yloxy)carbonyl)-lysine. In some instances, the unnatural amino acid is N6-((pent4-yn-1-yloxy)carbonyl)-lysine. In some instances, the unnatural amino acid is N6-(thiazolidine-4-carbonyl) lysine. In some instances, the unnatural amino acid is 2-amino-8-oxononanoic acid. In some instances, the unnatural amino acid is 2-amino-8-oxooctanoic acid. In some instances, the unnatural amino acid is N6-(2-oxoacetyl) lysine.

In some instances, the unnatural amino acid is N6-propionyllysine. In some instances, the unnatural amino acid is N6-butyryllysine, In some instances, the unnatural amino acid is N6-(but-2-enoyl) lysine, In some instances, the unnatural amino acid is N6-((bicyclo[2.2.1]hept-5-en-2-yloxy)carbonyl) lysine. In some instances, the unnatural amino acid is N6-((spiro[2.3]hex-1-en-5-ylmethoxy)carbonyl) lysine. In some instances, the unnatural amino acid is N6-(((4-(1-(trifluoromethyl)cycloprop-2-en-1-yl)benzyl)oxy)carbonyl) lysine. In some instances, the unnatural amino acid is N6-((bicyclo[2.2.1]hept-5-en-2-ylmethoxy)carbonyl) lysine. In some instances, the unnatural amino acid is cysteinyllysine. In some instances, the unnatural amino acid is N6-((1-(6-nitrobenzo[d][1,3]dioxol-5-yl)ethoxy)carbonyl) lysine. In some instances, the unnatural amino acid is N6-((2-(3-methyl-3H-diazirin-3-yl)ethoxy)carbonyl) lysine. In some instances, the unnatural amino acid is N6-((3-(3-methyl-3H-diazirin-3-yl)propoxy)carbonyl) lysine. In some instances, the unnatural amino acid is N6-((meta nitrobenyloxy)N6-methylcarbonyl) lysine. In some instances, the unnatural amino acid is N6-((bicyclo[6.1.0]non-4-yn-9-ylmethoxy)carbonyl)-lysine. In some instances, the unnatural amino acid is N6-((cyclohept-3-en-1-yloxy)carbonyl)-L-lysine.

In some embodiments, the unnatural amino acid is incorporated into a protein by an unnatural codon comprising an unnatural nucleotide.

In some instances, incorporation of the unnatural amino acid into a protein is mediated by an orthogonal, modified synthetase/tRNA pair. Such orthogonal pairs comprise a natural or mutated synthetase that is capable of charging the unnatural tRNA with a specific unnatural amino acid, often while minimizing charging of a) other endogenous amino acids or alternate unnatural amino acids onto the unnatural tRNA and b) any other (including endogenous) tRNAs. Such orthogonal pairs comprise tRNAs that are capable of being charged by the synthetase, while avoiding being charged with other endogenous amino acids by endogenous synthetases. In some embodiments, such pairs are identified from various organisms, such as bacteria, yeast, Archaea, or human sources. In some embodiments, an orthogonal synthetase/tRNA pair comprises components from a single organism. In some embodiments, an orthogonal synthetase/tRNA pair comprises components from two different organisms. In some embodiments, an orthogonal synthetase/tRNA pair comprising components that prior to modification, promote translation of different amino acids. In some embodiments, an orthogonal synthetase is a modified alanine synthetase. In some embodiments, an orthogonal synthetase is a modified arginine synthetase. In some embodiments, an orthogonal synthetase is a modified asparagine synthetase. In some embodiments, an orthogonal synthetase is a modified aspartic acid synthetase. In some embodiments, an orthogonal synthetase is a modified cysteine synthetase. In some embodiments, an orthogonal synthetase is a modified glutamine synthetase. In some embodiments, an orthogonal synthetase is a modified glutamic acid synthetase. In some embodiments, an orthogonal synthetase is a modified alanine glycine. In some embodiments, an orthogonal synthetase is a modified histidine synthetase. In some embodiments, an orthogonal synthetase is a modified leucine synthetase. In some embodiments, an orthogonal synthetase is a modified isoleucine synthetase. In some embodiments, an orthogonal synthetase is a modified lysine synthetase. In some embodiments, an orthogonal synthetase is a modified methionine synthetase. In some embodiments, an orthogonal synthetase is a modified phenylalanine synthetase. In some embodiments, an orthogonal synthetase is a modified proline synthetase. In some embodiments, an orthogonal synthetase is a modified serine synthetase. In some embodiments, an orthogonal synthetase is a modified threonine synthetase. In some embodiments, an orthogonal synthetase is a modified tryptophan synthetase. In some embodiments, an orthogonal synthetase is a modified tyrosine synthetase. In some embodiments, an orthogonal synthetase is a modified valine synthetase. In some embodiments, an orthogonal synthetase is a modified phosphoserine synthetase. In some embodiments, an orthogonal tRNA is a modified alanine tRNA. In some embodiments, an orthogonal tRNA is a modified arginine tRNA. In some embodiments, an orthogonal tRNA is a modified asparagine tRNA. In some embodiments, an orthogonal tRNA is a modified aspartic acid tRNA. In some embodiments, an orthogonal tRNA is a modified cysteine tRNA. In some embodiments, an orthogonal tRNA is a modified glutamine tRNA. In some embodiments, an orthogonal tRNA is a modified glutamic acid tRNA. In some embodiments, an orthogonal tRNA is a modified alanine glycine. In some embodiments, an orthogonal tRNA is a modified histidine tRNA. In some embodiments, an orthogonal tRNA is a modified leucine tRNA. In some embodiments, an orthogonal tRNA is a modified isoleucine tRNA. In some embodiments, an orthogonal tRNA is a modified lysine tRNA. In some embodiments, an orthogonal tRNA is a modified methionine tRNA. In some embodiments, an orthogonal tRNA is a modified phenylalanine tRNA. In some embodiments, an orthogonal tRNA is a modified proline tRNA. In some embodiments, an orthogonal tRNA is a modified serine tRNA. In some embodiments, an orthogonal tRNA is a modified threonine tRNA. In some embodiments, an orthogonal tRNA is a modified tryptophan tRNA. In some embodiments, an orthogonal tRNA is a modified tyrosine tRNA. In some embodiments, an orthogonal tRNA is a modified valine tRNA. In some embodiments, an orthogonal tRNA is a modified phosphoserine tRNA.

In some embodiments, the unnatural amino acid is incorporated into a protein by an aminoacyl (aaRS or RS)-tRNA synthetase-tRNA pair. Exemplary aaRS-tRNA pairs include, but are not limited to, Methanococcus jannaschii (Mj-Tyr) aaRS/tRNA pairs, E. coli TyrRS (Ec-Tyr)/B. stearothermophilus tRNA_{CUA} pairs, E. coli LeuRS (Ec-Leu)/B. stearothermophilus tRNA_{CUA} pairs, and pyrrolysyl-tRNA pairs. In some instances, the unnatural amino acid is incorporated into a protein by a Mj-TyrRS/tRNA pair. Exemplary unnatural amino acids (UAAs) that can be incorporated by a Mj-TyrRS/tRNA pair include, but are not limited to, para-substituted phenylalanine derivatives such as p-aminophenylalanine and p-methoyphenylalanine; meta-substituted tyrosine derivatives such as 3-aminotyrosine, 3-nitrotyrosine, 3,4-dihydroxyphenylalanine, and 3-iodotyrosine; phenylselenocysteine; p-boronopheylalanine; and o-nitrobenzyltyrosine.

In some instances, the unnatural amino acid is incorporated into a protein by a Ec-Tyr/tRNA_{CUA} or a Ec-Leu/tRNA_{CUA} pair. Exemplary UAAs that can be incorporated by a Ec-Tyr/tRNA_{CUA} or a Ec-Leu/tRNA_{CUA} pair include, but are not limited to, phenylalanine derivatives containing benzophenone, ketone, iodide, or azide substituents; O-propargyltyrosine; α-aminocaprylic acid, O-methyl tyrosine, O-nitrobenzyl cysteine; and 3-(naphthalene-2-ylamino)-2-amino-propanoic acid.

In some instances, the unnatural amino acid is incorporated into a protein by a pyrrolysyl-tRNA pair. In some cases, the PylRS is obtained from an archaebacterial species, e.g., from a methanogenic archaebacterium. In some cases, the PylRS is obtained from Methanosarcina barkeri, Methanosarcina mazei, or Methanosarcina acetivorans. Exemplary UAAs that can be incorporated by a pyrrolysyl-tRNA pair include, but are not limited to, amide and carbamate substituted lysines such as 2-amino-6-((R)-tetrahydrofuran-2-carboxamido)hexanoic acid, N-ε-D-prolyl-L-lysine, and N-ε-cyclopentyloxycarbonyl-L-lysine; N-ε-Acryloyl-L-lysine; N-ε-[(1-(6-nitrobenzo[d][1,3]dioxol-5-yl)ethoxy)carbonyl]-L-lysine; and N-ε-(1-methylcyclopro-2-enecarboxamido) lysine.

In some instances, an unnatural amino acid is incorporated into a protein described herein by a synthetase disclosed in U.S. Pat. Nos. 9,988,619 and 9,938,516. Exemplary UAAs that can be incorporated by such synthetases include para-methylazido-L-phenylalanine, aralkyl, heterocyclyl, heteroaralkyl unnatural amino acids, and others. In some embodiments, such UAAs comprise pyridyl, pyrazinyl, pyrazolyl, triazolyl, oxazolyl, thiazolyl, thiophenyl, or other heterocycle. Such amino acids in some embodiments comprise azides, tetrazines, or other chemical group capable of conjugation to a coupling partner, such as a water soluble moiety. In some embodiments, such synthetases are expressed and used to incorporate UAAs into proteins in-vivo. In some embodiments, such synthetases are used to incorporate UAAs into proteins using a cell-free translation system, such as a cell lysate or a reconstituted system of purified components. The tRNA can be charged with the unnatural amino acid in the cell free system, or in a separate reaction beforehand (such that the charged tRNA would be added directly to the system comprising the ribosomes, mRNA, and other components, without needing to add the synthetase or a construct encoding the synthetase to the system).

Systems for in vitro translation are described, e.g., in Zeenko et al., RNA 14:593-602 (2008); Spirin, Trends Biotechnol. 2004:538-545 (2004); and Endo et al., Curr. Opin. Biotechnol. 17:373-380 (2006). The systems may be prepared from cell lysates (e.g., extracts) or reconstituted from purified components. The systems may comprise, in addition to ribosomes, tRNAs, and other components described herein, one or more translation initiation factors; ATP; and one or more translation termination factors. In some embodiments, the system further comprises one or more molecular chaperones, which may assist with folding of the nascent polypeptide during and/or following translation.

In some instances, an unnatural amino acid is incorporated into a protein described herein by a naturally occurring synthetase. In some embodiments, an unnatural amino acid is incorporated into a protein by an organism that is auxotrophic for one or more amino acids. In some embodiments, synthetases corresponding to the auxotrophic amino acid are capable of charging the corresponding tRNA with an unnatural amino acid. In some embodiments, the unnatural amino acid is selenocysteine, or a derivative thereof. In some embodiments, the unnatural amino acid is selenomethionine, or a derivative thereof. In some embodiments, the unnatural amino acid is an aromatic amino acid, wherein the aromatic amino acid comprises an aryl halide, such as an iodide. In embodiments, the unnatural amino acid is structurally similar to the auxotrophic amino acid.

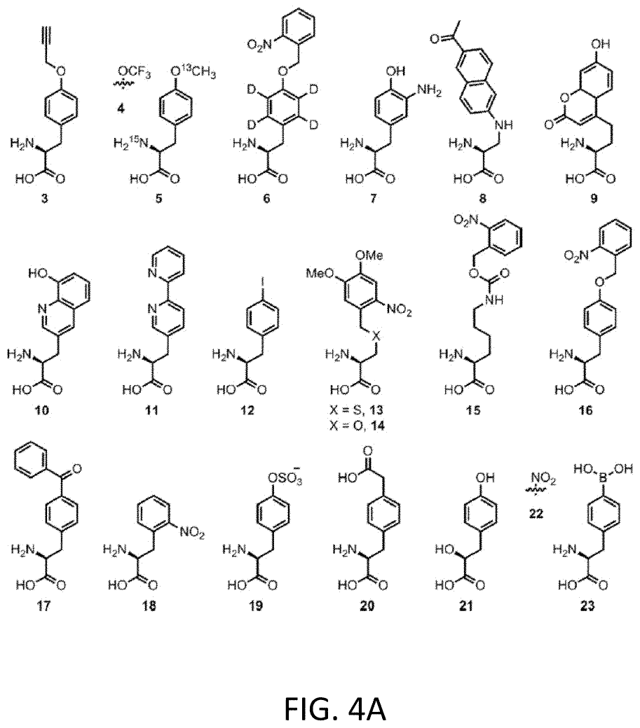

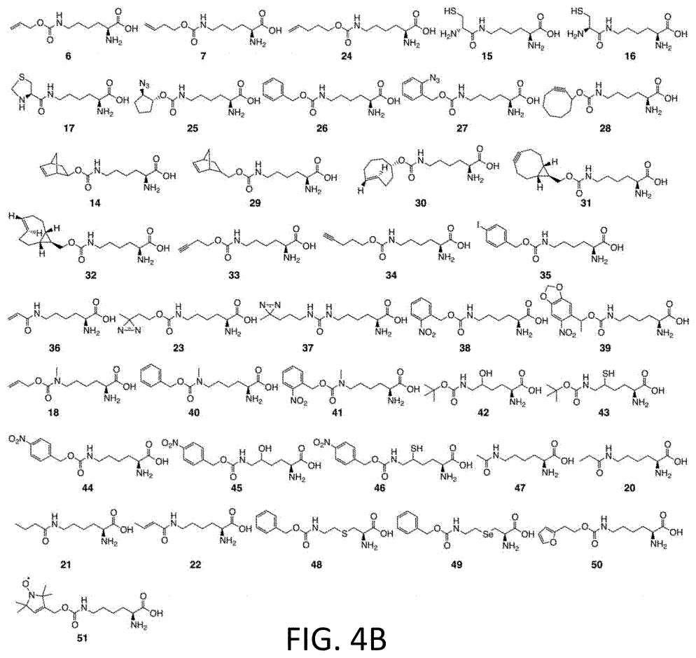

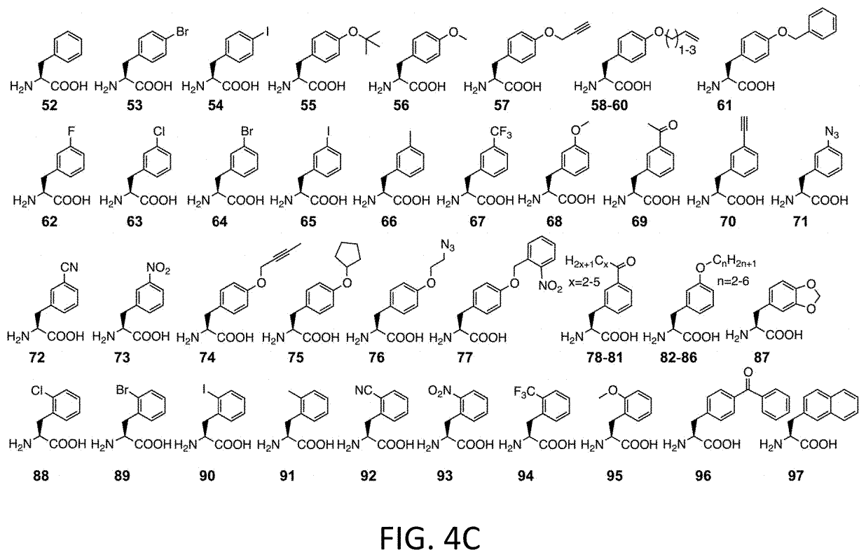

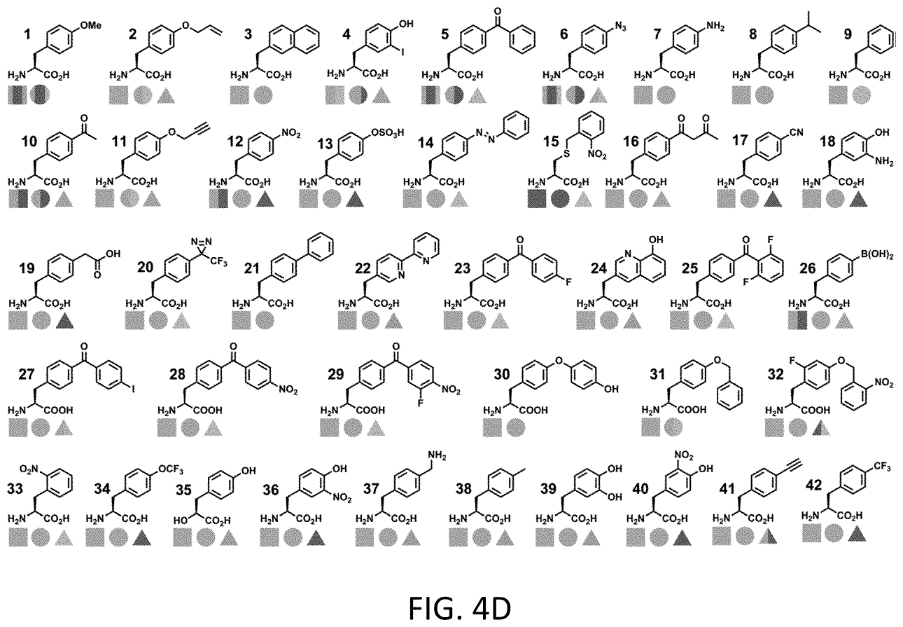

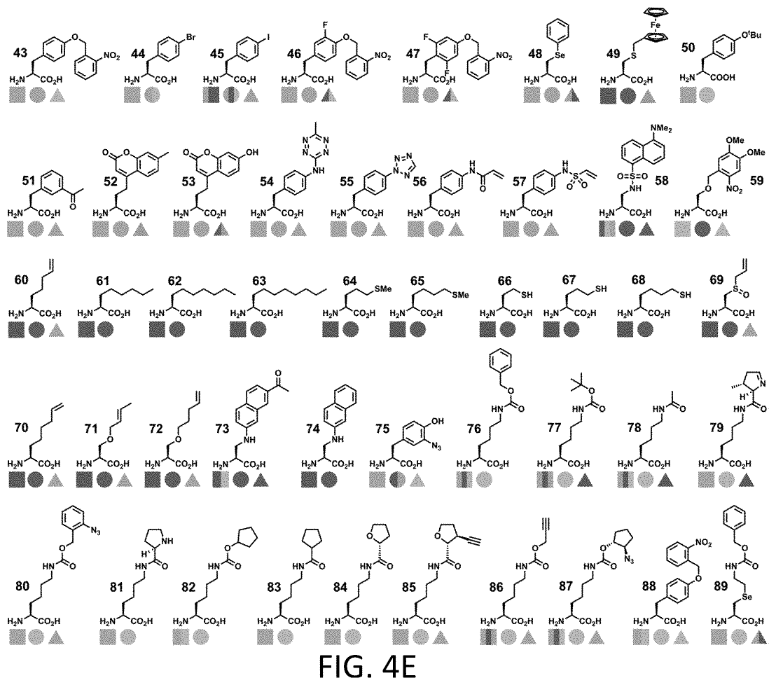

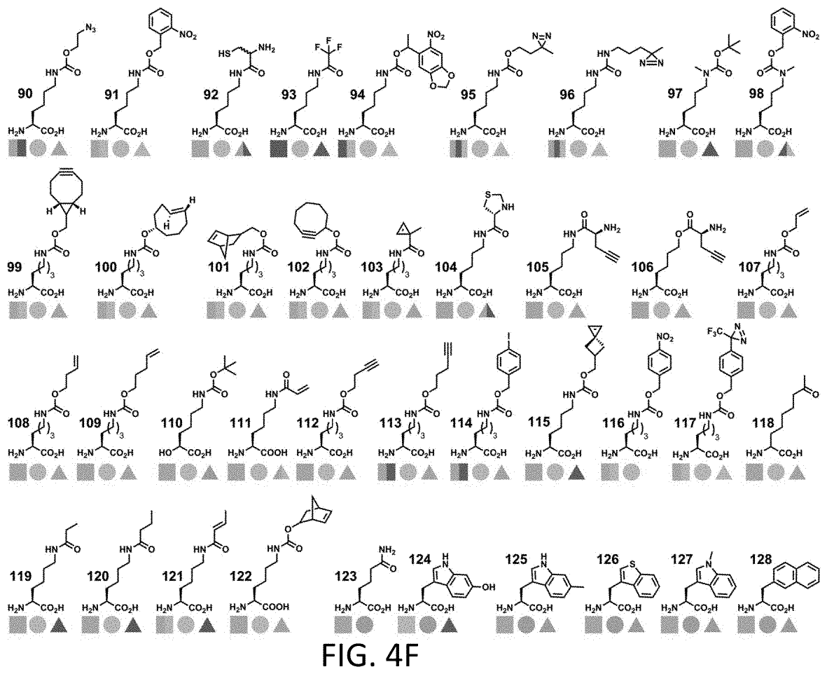

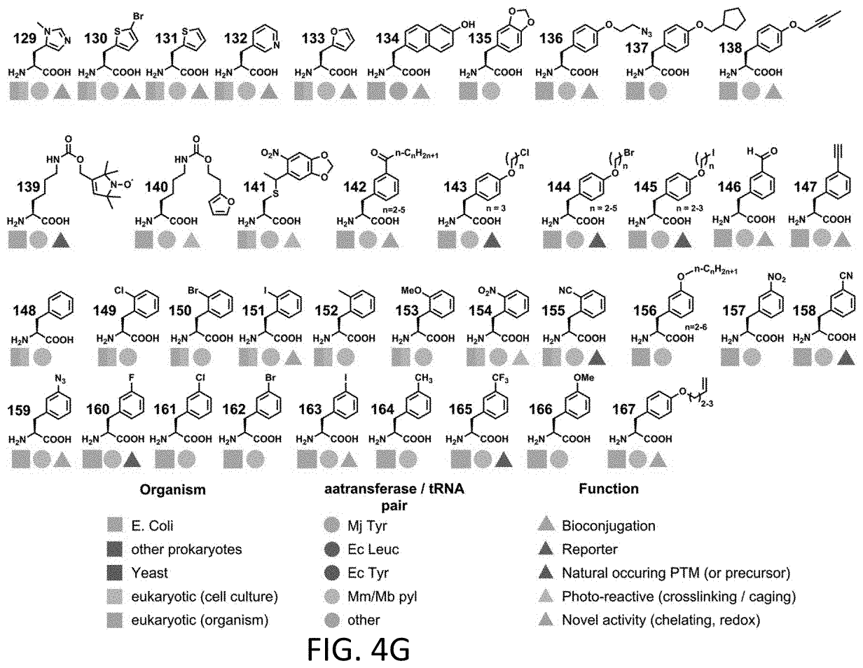

In some instances, the unnatural amino acid comprises an unnatural amino acid illustrated in FIG. 4A.

In some instances, the unnatural amino acid comprises a lysine or phenylalanine derivative or analogue. In some instances, the unnatural amino acid comprises a lysine derivative or a lysine analogue. In some instances, the unnatural amino acid comprises a pyrrolysine (Pyl). In some instances, the unnatural amino acid comprises a phenylalanine derivative or a phenylalanine analogue. In some instances, the unnatural amino acid is an unnatural amino acid described in Wan, et al., “Pyrrolysyl-tRNA synthetase: an ordinary enzyme but an outstanding genetic code expansion tool,” Biochem Biophys Aceta 1844(6): 1059-4070 (2014). In some instances, the unnatural amino acid comprises an unnatural amino acid illustrated in FIG. 4B and FIG. 4C.

In some embodiments, the unnatural amino acid comprises an unnatural amino acid illustrated in FIG. 4D-FIG. 4G (adopted from Table 1 of Dumas et al., Chemical Science 2015, 6, 50-69).

In some embodiments, an unnatural amino acid incorporated into a protein described herein is disclosed in U.S. Pat. Nos. 9,840,493; 9,682,934; US 2017/0260137; U.S. Pat. No. 9,938,516; or US 2018/0086734. Exemplary UAAs that can be incorporated by such synthetases include para-methylazido-L-phenylalanine, aralkyl, heterocyclyl, and heteroaralkyl, and lysine derivative unnatural amino acids. In some embodiments, such UAAs comprise pyridyl, pyrazinyl, pyrazolyl, triazolyl, oxazolyl, thiazolyl, thiophenyl, or other heterocycle. Such amino acids in some embodiments comprise azides, tetrazines, or other chemical group capable of conjugation to a coupling partner, such as a water soluble moiety. In some embodiments, a UAA comprises an azide attached to an aromatic moiety via an alkyl linker. In some embodiments, an alkyl linker is a C₁-C₁₀ linker. In some embodiments, a UAA comprises a tetrazine attached to an aromatic moiety via an alkyl linker. In some embodiments, a UAA comprises a tetrazine attached to an aromatic moiety via an amino group. In some embodiments, a UAA comprises a tetrazine attached to an aromatic moiety via an alkylamino group. In some embodiments, a UAA comprises an azide attached to the terminal nitrogen (e.g., N6 of a lysine derivative, or N5, N4, or N3 of a derivative comprising a shorter alkyl side chain) of an amino acid side chain via an alkyl chain. In some embodiments, a UAA comprises a tetrazine attached to the terminal nitrogen of an amino acid side chain via an alkyl chain. In some embodiments, a UAA comprises an azide or tetrazine attached to an amide via an alkyl linker. In some embodiments, the UAA is an azide or tetrazine-containing carbamate or amide of 3-aminoalanine, serine, lysine, or derivative thereof. In some embodiments, such UAAs are incorporated into proteins in-vivo. In some embodiments, such UAAs are incorporated into proteins in a cell-free system.

### Cell Types

In some embodiments, many types of cells/microorganisms are used, e.g., for transforming or genetically engineering. In some embodiments, a cell is eukaryotic cell. In some cases, the cell is a eukaryotic cell, such as a cultured animal, plant, or human cell. In additional cases, the cell is present in an organism such as a plant or animal.

In some embodiments, an engineered microorganism is a single cell organism, often capable of dividing and proliferating. A microorganism can include one or more of the following features: aerobe, anaerobe, filamentous, non-filamentous, monoploid, dipoid, auxotrophic and/or non-auxotrophic. In certain embodiments, an engineered microorganism is a non-prokaryotic microorganism. In some embodiments, an engineered microorganism is a eukaryotic microorganism (e.g., yeast, fungi, amoeba). In some embodiments, an engineered microorganism is a fungus. In some embodiments, an engineered organism is a yeast.

Any suitable yeast may be selected as a host microorganism, engineered microorganism, genetically modified organism or source for a heterologous or modified polynucleotide. Yeast include, but are not limited to, Yarrowia yeast (e.g., Y. lipolytica (formerly classified as Candida lipolytica)), Candida yeast (e.g., C. revkaufi, C. viswanathii, C. pulcherrima, C. tropicalis, C. utilis), Rhodotorula yeast (e.g., R. glutinus, R. graminis), Rhodosporidium yeast (e.g., R. toruloides), Saccharomyces yeast (e.g., S. cerevisiae, S. bayanus, S. pastorianus, S. carlsbergensis), Cryptococcus yeast, Trichosporon yeast (e.g., T. pullans, T. cutaneum), Pichia yeast (e.g., P. pastoris) and Lipomyces yeast (e.g., L. starkeyii, L. lipoferus). In some embodiments, a suitable yeast is of the genus Arachniotus, Aspergillus, Aureobasidium, Auxarthron, Blastomyces, Candida, Chrysosporium, Chrysosporium Debaryomyces, Coccidioides, Cryptococcus, Gymnoascus, Hansenula, Histoplasma, Issatchenkia, Kluyveromyces, Lipomyces, Issatchenkia, Microsporum, Myxotrichum, Myxozyma, Oidiodendron, Pachysolen, Penicillium, Pichia, Rhodosporidium, Rhodotorula, Rhodotorula, Saccharomyces, Schizosaccharomyces, Scopulariopsis, Sepedonium, Trichosporon, or Yarrowia. In some embodiments, a suitable yeast is of the species Arachniotus flavoluteus, Aspergillus flavus, Aspergillus fumigatus, Aspergillus niger, Aureobasidium pullulans, Auxarthron thaxteri, Blastomyces dermatitidis, Candida albicans, Candida dubliniensis, Candida famata, Candida glabrata, Candida guilliermondii, Candida kefyr, Candida krusei, Candida lambica, Candida lipolytica, Candida lustitaniae, Candida parapsilosis, Candida pulcherrima, Candida revkaufi, Candida rugosa, Candida tropicalis, Candida utilis, Candida viswanathii, Candida xestobii, Chrysosporium keratinophilum, Coccidiodes immitis, Cryptococcus albidus var. diffluens, Cryptococcus laurentii, Cryptococcus neoformans, Debaryomyces hansenii, Gymnoascus dugwayensis, Hansenula anomala, Histoplasma capsulatum, Issatchenkia occidentalis, Isstachenkia orientalis, Kluyveromyces lactis, Kluyveromyces marxianus, Kluyveromyces thermotolerans, Kluyveromyces waltii, Lipomyces lipoferus, Lipomyces starkeyii, Microsporum gypseum, Myxotrichum deflexum, Oidiodendron echinulatum, Pachysolen tannophilis, Penicillium notatum, Pichia anomala, Pichia pastoris, Pichia stipitis, Rhodosporidium toruloides, Rhodotorula glutinus, Rhodotorula graminis, Saccharomyces cerevisiae, Saccharomyces kluyveri, Schizosaccharomyces pombe, Scopulariopsis acremonium, Sepedonium chrysospermum, Trichosporon cutaneum, Trichosporon pullans, Yarrowia lipolytica, or Yarrowia lipolytica (formerly classified as Candida lipolytica). In some embodiments, a yeast is a Y. lipolytica strain that includes, but is not limited to, ATCC20362, ATCC8862, ATCC18944, ATCC20228, ATCC76982 and LGAM S (7)1 strains (Papanikolaou S., and Aggelis G., Bioresour. Technol. 82(1):43-9 (2002)). In certain embodiments, a yeast is a Candida species (i.e., Candida spp.) yeast. Any suitable Candida species can be used and/or genetically modified for production of a fatty dicarboxylic acid (e.g., octanedioic acid, decanedioic acid, dodecanedioic acid, tetradecanedioic acid, hexadecanedioic acid, octadecanedioic acid, eicosanedioic acid). In some embodiments, suitable Candida species include, but are not limited to Candida albicans, Candida dubliniensis, Candida famata, Candida glabrata, Candida guilliermondii, Candida kefyr, Candida krusei, Candida lambica, Candida lipolytica, Candida lustitaniae, Candida parapsilosis, Candida pulcherrima, Candida revkaufi, Candida rugosa, Candida tropicalis, Candida utilis, Candida viswanathii, Candida xestobii and any other Candida spp. yeast described herein. Non-limiting examples of Candida spp. strains include, but are not limited to, sAA001 (ATCC20336), sAA002 (ATCC20913), sAA003 (ATCC20962), sAA496 (US2012/0077252), sAA106 (US2012/0077252), SU-2 (ura3-/ura3-), H5343 (beta oxidation blocked; U.S. Pat. No. 5,648,247) strains. Any suitable strains from Candida spp. yeast may be utilized as parental strains for genetic modification.

Yeast genera, species and strains are often so closely related in genetic content that they can be difficult to distinguish, classify and/or name. In some cases strains of C. lipolytica and Y. lipolytica can be difficult to distinguish, classify and/or name and can be, in some cases, considered the same organism. In some cases, various strains of C. tropicalis and C. viswanathii can be difficult to distinguish, classify and/or name (for example see Arie et. al., J. Gen. Appl. Microbiol., 46, 257-262 (2000). Some C. tropicalis and C. viswanathii strains obtained from ATCC as well as from other commercial or academic sources can be considered equivalent and equally suitable for the embodiments described herein. In some embodiments, some parental strains of C. tropicalis and C. viswanathii are considered to differ in name only.

Any suitable fungus may be selected as a host microorganism, engineered microorganism or source for a heterologous polynucleotide. Non-limiting examples of fungi include, but are not limited to, Aspergillus fungi (e.g., A. parasiticus, A. nidulans), Thraustochytrium fungi, Schizochytrium fungi and Rhizopus fungi (e.g., R. arrhizus, R. oryzae, R. nigricans). In some embodiments, a fungus is an A. parasiticus strain that includes, but is not limited to, strain ATCC24690, and in certain embodiments, a fungus is an A. nidulans strain that includes, but is not limited to, strain ATCC38163.

Cells from non-microbial organisms can be utilized as a host microorganism, engineered microorganism or source for a heterologous polynucleotide. Examples of such cells, include, but are not limited to, insect cells (e.g., Drosophila (e.g., D. melanogaster), Spodoptera (e.g., S. frugiperda Sf9 or Sf21 cells) and Trichoplusia (e.g., High-Five cells); nematode cells (e.g., C. elegans cells); avian cells; amphibian cells (e.g., Xenopus laevis cells); reptilian cells; mammalian cells (e.g., NIH3T3, 293, CHO, COS, VERO, C127, BHK, Per-C6, Bowes melanoma and HeLa cells); and plant cells (e.g., Arabidopsis thaliana, Nicotania tabacum, Cuphea acinifolia, Cuphea aequipetala, Cuphea angustifolia, Cuphea appendiculata, Cuphea avigera, Cuphea avigera var. pulcherrima, Cuphea axilliflora, Cuphea bahiensis, Cuphea baillonis, Cuphea brachypoda, Cuphea bustamanta, Cuphea calcarata, Cuphea calophylla, Cuphea calophylla subsp. mesostemon, Cuphea carthagenensis, Cuphea circaeoides, Cuphea confertiflora, Cuphea cordata, Cuphea crassiflora, Cuphea cyanea, Cuphea decandra, Cuphea denticulata, Cuphea disperma, Cuphea epilobiifolia, Cuphea ericoides, Cuphea flava, Cuphea flavisetula, Cuphea fuchsiifolia, Cuphea gaumeri, Cuphea glutinosa, Cuphea heterophylla, Cuphea hookeriana, Cuphea hyssopifolia (Mexican-heather), Cuphea hyssopoides, Cuphea ignea, Cuphea ingrata, Cuphea jorullensis, Cuphea lanceolata, Cuphea linarioides, Cuphea llavea, Cuphea lophostoma, Cuphea lutea, Cuphea lutescens, Cuphea melanium, Cuphea melvilla, Cuphea micrantha, Cuphea micropetala, Cuphea mimuloides, Cuphea nitidula, Cuphea palustris, Cuphea parsonsia, Cuphea pascuorum, Cuphea paucipetala, Cuphea procumbens, Cuphea pseudosilene, Cuphea pseudovaccinium, Cuphea pulchra, Cuphea racemosa, Cuphea repens, Cuphea salicifolia, Cuphea salvadorensis, Cuphea schumannii, Cuphea sessiliflora, Cuphea sessilifolia, Cuphea setosa, Cuphea spectabilis, Cuphea spermacoce, Cuphea splendida, Cuphea splendida var. viridiflava, Cuphea strigulosa, Cuphea subuligera, Cuphea teleandra, Cuphea thymoides, Cuphea tolucana, Cuphea urens, Cuphea utriculosa, Cuphea viscosissima, Cuphea watsoniana, Cuphea wrightii, Cuphea lanceolata).

Microorganisms or cells used as host organisms or source for a heterologous polynucleotide are commercially available. Microorganisms and cells described herein, and other suitable microorganisms and cells are available, for example, from Invitrogen Corporation, (Carlsbad, Calif.), American Type Culture Collection (Manassas, Va.), and Agricultural Research Culture Collection (NRRL; Peoria, Ill.). Host microorganisms and engineered microorganisms may be provided in any suitable form. For example, such microorganisms may be provided in liquid culture or solid culture (e.g., agar-based medium), which may be a primary culture or may have been passaged (e.g., diluted and cultured) one or more times. Microorganisms also may be provided in frozen form or dry form (e.g., lyophilized). Microorganisms may be provided at any suitable concentration.

### Nucleic Acid Reagents & Tools

A nucleotide and/or nucleic acid reagent (or polynucleotide) for use with a method, cell, or engineered microorganism described herein comprises one or more ORFs with or without an unnatural nucleotide. An ORF may be from any suitable source, sometimes from genomic DNA, mRNA, reverse transcribed RNA or complementary DNA (cDNA) or a nucleic acid library comprising one or more of the foregoing, and is from any organism species that contains a nucleic acid sequence of interest, protein of interest, or activity of interest. Non-limiting examples of organisms from which an ORF can be obtained include bacteria, yeast, fungi, human, insect, nematode, bovine, equine, canine, feline, rat or mouse, for example. In some embodiments, a nucleotide and/or nucleic acid reagent or other reagent described herein is isolated or purified. ORFs may be created that include unnatural nucleotides via published in vitro methods. In some cases, a nucleotide or nucleic acid reagent comprises an unnatural nucleobase.

A nucleic acid reagent sometimes comprises a nucleotide sequence adjacent to an ORF that is translated in conjunction with the ORF and encodes an amino acid tag. The tag-encoding nucleotide sequence is located 3′ and/or 5′ of an ORF in the nucleic acid reagent, thereby encoding a tag at the C-terminus or N-terminus of the protein or peptide encoded by the ORF. Any tag that does not abrogate in vitro transcription and/or translation may be utilized and may be appropriately selected by the artisan. Tags may facilitate isolation and/or purification of the desired ORF product from culture or fermentation media. In some instances, libraries of nucleic acid reagents are used with the methods and compositions described herein. For example, a library of at least 100, 1000, 2000, 5000, 10,000, or more than 50,000 unique polynucleotides are present in a library, wherein each polynucleotide comprises at least one unnatural nucleobase.

A nucleic acid or nucleic acid reagent, with or without an unnatural nucleotide, can comprise certain elements, e.g., regulatory elements, often selected according to the intended use of the nucleic acid. Any of the following elements can be included in or excluded from a nucleic acid reagent. A nucleic acid reagent, for example, may include one or more or all of the following nucleotide elements: one or more promoter elements, one or more 5′ untranslated regions (5′UTRs), one or more regions into which a target nucleotide sequence may be inserted (an “insertion element”), one or more target nucleotide sequences, one or more 3′ untranslated regions (3′UTRs), and one or more selection elements. A nucleic acid reagent can be provided with one or more of such elements and other elements may be inserted into the nucleic acid before the nucleic acid is introduced into the desired organism. In some embodiments, a provided nucleic acid reagent comprises a promoter, 5′UTR, optional 3′UTR and insertion element(s) by which a target nucleotide sequence is inserted (i.e., cloned) into the nucleotide acid reagent. In certain embodiments, a provided nucleic acid reagent comprises a promoter, insertion element(s) and optional 3′UTR, and a 5′ UTR/target nucleotide sequence is inserted with an optional 3′UTR. The elements can be arranged in any order suitable for expression in the chosen expression system (e.g., expression in a chosen organism, or expression in a cell free system, for example), and in some embodiments a nucleic acid reagent comprises the following elements in the 5′ to 3′ direction: (1) promoter element, 5′UTR, and insertion element(s); (2) promoter element, 5′UTR, and target nucleotide sequence; (3) promoter element, 5′UTR, insertion element(s) and 3′UTR; and (4) promoter element, 5′UTR, target nucleotide sequence and 3′UTR. In some embodiments, the UTR can be optimized to alter or increase transcription or translation of the ORF that are either fully natural or that contain unnatural nucleotides.

The nucleic acid (e.g., mRNA) comprising the nucleobase described herein, in some cases, comprises a 5′ UTR and/or 3′ UTR that enhances mRNA stability in vivo (e.g., in the eukaryotic cell, or eukaryotic SSO. In some instances, the 5′ or 3′ UTR, or both, are engineered to reduce mRNA degradation or decay in vivo. A non-limiting example of a 5′ and 3′ UTR that enhances mRNA stability in the eukaryotic systems disclosed herein is the CS2 3′ and 5′ UTRs. In some embodiments, the mRNA is modified to reduce removal rates of the poly(A) tail of the mRNA, as compared to mRNA comprising the nucleobases described herein that is not otherwise modified. In some embodiments, cis-acting AU-rich elements (AREs) are blocked from intra- and extra-cellular signaling that promotes mRNA decay. In some embodiments, premature stop codons in the mRNA are removed from the mRNA to reduce non-sense mediated decay (NMD) of the mRNA.

In some cases, the 5′ and/or 3′ UTR increases translation of the mRNA into a polypeptide directly or indirectly. Non-limiting examples of how a 5′ UTR or a 3′ UTR influences the translation of the mRNA into the polypeptide directly includes recruitment of RNA-binding proteins that bind to 5′ or 3′ cis-elements and effect the recruitment of the ribosome or effector proteins (e.g., mRNA deadenylases, decapping enzymes). Non-limiting examples of how a 5′UTR or 3′ UTR influences the translation of the mRNA into the polypeptide indirectly includes the formation of 5′ and 3′ UTR secondary structures that block or enhance binding of RNA-binding proteins to the 5′ or 3′ UTR regions, and mRNA subcellular localization.

In some embodiments, the 5′UTR and/or 3′ UTR increases the translation efficiency of the mRNA in vitro or in vivo, relative to the translation efficiency of an mRNA containing the nucleobase that is not engineered. In some embodiments, the translation efficiency is increased by engineering the mRNA to reduce skipping of select AUG (start codons) by the ribosome during scanning. In some embodiments, the mRNA comprise sequence elements that improve start codon recognition such as Kozak sequences, or variations thereof. In some embodiments, the 5′ UTR of the mRNA is engineered to reduce overall guanine-cytosine (GC) content.

In some embodiments, the formation of secondary structures in the mRNA (e.g. RNA G-quadruplex structures, RG4s) involving the AUG start codon within the 5′ UTR is reduced, thereby increasing the efficiency of translation from that AUG. In some embodiments, the 5′ UTR is engineered to have a negative folding free energy (ΔG), relative to an mRNA that is not engineered. In some embodiments, the ΔG is at most −40, −41, −42, −43, −44, −45, −46, −47, −48, −49, −50, −51, −52, −53, −54, −55, −56, −57, −58, −59, or −60. In some embodiments, the mRNA is chemically modified at the 5′ UTR or 3′ UTR to promote translation efficiency. In some embodiments, the chemical modification is a N⁶-methyladenosine. In an in vitro system (e.g., an engineered eukaryotic cell or semi-synthetic organism), overexpression of eIF4A, the subunit of the eIF4F complex that promotes the unwinding of RNA secondary structures in cooperation with eIF3B and eIF4H, increases translation efficiency of the mRNA. In some embodiments, knock out or knockdown of stabilizing proteins (e.g. fragile X mental retardation protein (FMRP)) that promote secondary structure formation of the mRNA, reduces formation of secondary structures, thereby increasing translation efficiency of the mRNA. In some embodiments, the trans-acting agents (e.g., RNA's, small molecules, proteins) are introduced into the cell (e.g., eukaryotic cell) to promote translation of the mRNA.

In some instances, the 5′ UTR and/or 3′ UTR promote subcellular localization of mRNA, thereby promoting translation of the mRNA in vivo. In some embodiments, the 3′ or 5′ UTR cis-acting elements such as mRNA zip codes are modified such that binding of the mRNA zip codes by zip-code-binding proteins (e.g., Staufen) is repressed or enhanced, thereby increasing translation efficiency of the mRNA.

Nucleic acid reagents, e.g., expression cassettes and/or expression vectors (e.g., for expressing a heterologous tRNA synthetase), can include a variety of regulatory elements, including promoters, enhancers, translational initiation sequences, transcription termination sequences and other elements. A “promoter” is generally a sequence or sequences of DNA that function when in a relatively fixed location in regard to the transcription start site. For example, the promoter can be upstream of the nucleoside triphosphate transporter nucleic acid segment. A “promoter” contains core elements required for basic interaction of RNA polymerase and transcription factors and can contain upstream elements and response elements. “Enhancer” generally refers to a sequence of DNA that functions at no fixed distance from the transcription start site and can be either 5′ or 3″ to the transcription unit. Furthermore, enhancers can be within an intron as well as within the coding sequence itself. They are usually between 10 and 300 by in length, and they function in cis. Enhancers function to increase transcription from nearby promoters. Enhancers, like promoters, also often contain response elements that mediate the regulation of transcription. Enhancers often determine the regulation of expression and can be used to alter or optimize ORF expression, including ORFs that are fully natural or that contain unnatural nucleotides.

As noted above, nucleic acid reagents may also comprise one or more 5′ UTR's, and one or more 3′UTR's. For example, expression vectors used in eukaryotic host cells (e.g., yeast, fungi, insect, plant, animal, human or nucleated cells) and prokaryotic host cells (e.g., virus, bacterium) can contain sequences that signal for the termination of transcription which can affect mRNA expression. These regions can be transcribed as polyadenylated segments in the untranslated portion of the mRNA encoding tissue factor protein. The 3′ untranslated regions also include transcription termination sites. In some preferred embodiments, a transcription unit comprises a polyadenylation region. One benefit of this region is that it increases the likelihood that the transcribed unit will be processed and transported like mRNA. The identification and use of polyadenylation signals in expression constructs is well established. In some preferred embodiments, homologous polyadenylation signals can be used in the transgene constructs.

A 5′ UTR may comprise one or more elements endogenous to the nucleotide sequence from which it originates, and sometimes includes one or more exogenous elements. A 5′ UTR can originate from any suitable nucleic acid, such as genomic DNA, plasmid DNA, RNA or mRNA, for example, from any suitable organism (e.g., virus, bacterium, yeast, fungi, plant, insect or mammal). The artisan may select appropriate elements for the 5′ UTR based upon the chosen expression system (e.g., expression in a chosen organism, or expression in a cell free system, for example). A 5′ UTR sometimes comprises one or more of the following elements known to the artisan: enhancer sequences (e.g., transcriptional or translational), transcription initiation site, transcription factor binding site, translation regulation site, translation initiation site, translation factor binding site, accessory protein binding site, feedback regulation agent binding sites, Pribnow box, TATA box, −35 element, E-box (helix-loop-helix binding element), ribosome binding site, replicon, internal ribosome entry site (IRES), silencer element and the like. In some embodiments, a promoter element may be isolated such that all 5′ UTR elements necessary for proper conditional regulation are contained in the promoter element fragment, or within a functional subsequence of a promoter element fragment.

A 5′ UTR in the nucleic acid reagent can comprise a translational enhancer nucleotide sequence. A translational enhancer nucleotide sequence often is located between the promoter and the target nucleotide sequence in a nucleic acid reagent. A translational enhancer sequence often binds to a ribosome, sometimes is an 18 S rRNA-binding ribonucleotide sequence (i.e., a 40 S ribosome binding sequence) and sometimes is an internal ribosome entry sequence (IRES). An IRES generally forms an RNA scaffold with precisely placed RNA tertiary structures that contact a 40 S ribosomal subunit via a number of specific intermolecular interactions. Examples of ribosomal enhancer sequences are known and can be identified by the artisan (e.g., Mignone et al., Nucleic Acids Research 33: D141-D146 (2005); Paulous et al., Nucleic Acids Research 31: 722-733 (2003); Akbergenov et al., Nucleic Acids Research 32: 239-247 (2004); Mignone et al., Genome Biology 3(3): reviews0004.1-0001.10 (2002); Gallie, Nucleic Acids Research 30: 3401-3411 (2002); Shaloiko et al., DOI: 10.1002/bit.20267; and Gallie et al., Nucleic Acids Research 15: 3257-3273 (1987)).

A translational enhancer sequence sometimes is a eukaryotic sequence, such as a Kozak consensus sequence or other sequence (e.g., hydroid polyp sequence, GenBank accession no. U07128). A translational enhancer sequence sometimes is a prokaryotic sequence, such as a Shine-Dalgarno consensus sequence. In certain embodiments, the translational enhancer sequence is a viral nucleotide sequence. A translational enhancer sequence sometimes is from a 5′ UTR of a plant virus, such as Tobacco Mosaic Virus (TMV), Alfalfa Mosaic Virus (AMV); Tobacco Etch Virus (ETV); Potato Virus Y (PVY); Turnip Mosaic (poty) Virus and Pea Seed Borne Mosaic Virus, for example. In certain embodiments, an omega sequence about 67 bases in length from TMV is included in the nucleic acid reagent as a translational enhancer sequence (e.g., devoid of guanosine nucleotides and includes a 25 nucleotide long poly (CAA) central region).

A 3′ UTR may comprise one or more elements endogenous to the nucleotide sequence from which it originates and sometimes includes one or more exogenous elements. A 3′ UTR may originate from any suitable nucleic acid, such as genomic DNA, plasmid DNA, RNA or mRNA, for example, from any suitable organism (e.g., a virus, bacterium, yeast, fungi, plant, insect or mammal). The artisan can select appropriate elements for the 3′ UTR based upon the chosen expression system (e.g., expression in a chosen organism, for example). A 3′ UTR sometimes comprises one or more of the following elements known to the artisan: transcription regulation site, transcription initiation site, transcription termination site, transcription factor binding site, translation regulation site, translation termination site, translation initiation site, translation factor binding site, ribosome binding site, replicon, enhancer element, silencer element and polyadenosine tail. A 3′ UTR often includes a polyadenosine tail and sometimes does not, and if a polyadenosine tail is present, one or more adenosine moieties may be added or deleted from it (e.g., about 5, about 10, about 15, about 20, about 25, about 30, about 35, about 40, about 45 or about 50 adenosine moieties may be added or subtracted).

In some embodiments, modification of a 5′ UTR and/or a 3′ UTR is used to alter (e.g., increase, add, decrease or substantially eliminate) the activity of a promoter. Alteration of the promoter activity can in turn alter the activity of a peptide, polypeptide or protein (e.g., enzyme activity for example), by a change in transcription of the nucleotide sequence(s) of interest from an operably linked promoter element comprising the modified 5′ or 3′ UTR. For example, a microorganism can be engineered by genetic modification to express a nucleic acid reagent comprising a modified 5′ or 3′ UTR that can add a novel activity (e.g., an activity not normally found in the host organism) or increase the expression of an existing activity by increasing transcription from a homologous or heterologous promoter operably linked to a nucleotide sequence of interest (e.g., homologous or heterologous nucleotide sequence of interest), in certain embodiments. In some embodiments, a microorganism can be engineered by genetic modification to express a nucleic acid reagent comprising a modified 5′ or 3′ UTR that can decrease the expression of an activity by decreasing or substantially eliminating transcription from a homologous or heterologous promoter operably linked to a nucleotide sequence of interest, in certain embodiments.

Expression of a heterologous polypeptide such as a tRNA synthetase from an expression cassette or expression vector can be controlled by any promoter capable of expression in prokaryotic cells or eukaryotic cells. A promoter element typically is required for DNA synthesis and/or RNA synthesis. A promoter element often comprises a region of DNA that can facilitate the transcription of a particular gene, by providing a start site for the synthesis of RNA corresponding to a gene. Promoters generally are located near the genes they regulate, are located upstream of the gene (e.g., 5′ of the gene), and are on the same strand of DNA as the sense strand of the gene, in some embodiments. In some embodiments, a promoter element can be isolated from a gene or organism and inserted in functional connection with a polynucleotide sequence to allow altered and/or regulated expression. A non-native promoter (e.g., promoter not normally associated with a given nucleic acid sequence) used for expression of a nucleic acid often is referred to as a heterologous promoter. In certain embodiments, a heterologous promoter and/or a 5′UTR can be inserted in functional connection with a polynucleotide that encodes a polypeptide having a desired activity as described herein. The terms “operably linked” and “in functional connection with” as used herein with respect to promoters, refer to a relationship between a coding sequence and a promoter element. The promoter is operably linked or in functional connection with the coding sequence when expression from the coding sequence via transcription is regulated, or controlled by, the promoter element. The terms “operably linked” and “in functional connection with” are utilized interchangeably herein with respect to promoter elements.

A promoter often interacts with a RNA polymerase. A polymerase is an enzyme that catalyzes synthesis of nucleic acids using a preexisting nucleic acid reagent. When the template is a DNA template, an RNA molecule is transcribed before protein is synthesized. Enzymes having polymerase activity suitable for use in the present methods include any polymerase that is active in the chosen system with the chosen template to synthesize protein. In some embodiments, a promoter (e.g., a heterologous promoter) also referred to herein as a promoter element, can be operably linked to a nucleotide sequence or an open reading frame (ORF). Transcription from the promoter element can catalyze the synthesis of an RNA corresponding to the nucleotide sequence or ORF sequence operably linked to the promoter, which in turn leads to synthesis of a desired peptide, polypeptide or protein.

Promoter elements sometimes exhibit responsiveness to regulatory control. Promoter elements also sometimes can be regulated by a selective agent. That is, transcription from promoter elements sometimes can be turned on, turned off, up-regulated or down-regulated, in response to a change in environmental, nutritional or internal conditions or signals (e.g., heat inducible promoters, light regulated promoters, feedback regulated promoters, hormone influenced promoters, tissue specific promoters, oxygen and pH influenced promoters, promoters that are responsive to selective agents (e.g., kanamycin) and the like, for example). Promoters influenced by environmental, nutritional or internal signals frequently are influenced by a signal (direct or indirect) that binds at or near the promoter and increases or decreases expression of the target sequence under certain conditions. As with all methods disclosed herein, the inclusion of natural or modified promoters can be used to alter or optimize expression of a fully natural ORF (e.g. a aaRS) or an ORF containing an unnatural nucleotide (e.g. an mRNA or a tRNA).

Non-limiting examples of selective or regulatory agents that influence transcription from a promoter element used in embodiments described herein include, without limitation, (1) nucleic acid segments that encode products that provide resistance against otherwise toxic compounds (e.g., antibiotics); (2) nucleic acid segments that encode products that are otherwise lacking in the recipient cell (e.g., essential products, tRNA genes, auxotrophic markers); (3) nucleic acid segments that encode products that suppress the activity of a gene product; (4) nucleic acid segments that encode products that can be readily identified (e.g., phenotypic markers such as antibiotics (e.g., β-lactamase), β-galactosidase, green fluorescent protein (GFP), yellow fluorescent protein (YFP), red fluorescent protein (RFP), cyan fluorescent protein (CFP), and cell surface proteins); (5) nucleic acid segments that bind products that are otherwise detrimental to cell survival and/or function; (6) nucleic acid segments that otherwise inhibit the activity of any of the nucleic acid segments described in Nos. 1-5 above (e.g., antisense oligonucleotides); (7) nucleic acid segments that bind products that modify a substrate (e.g., restriction endonucleases); (8) nucleic acid segments that can be used to isolate or identify a desired molecule (e.g., specific protein binding sites); (9) nucleic acid segments that encode a specific nucleotide sequence that can be otherwise non-functional (e.g., for PCR amplification of subpopulations of molecules); (10) nucleic acid segments that, when absent, directly or indirectly confer resistance or sensitivity to particular compounds; (11) nucleic acid segments that encode products that either are toxic or convert a relatively non-toxic compound to a toxic compound (e.g., Herpes simplex thymidine kinase, cytosine deaminase) in recipient cells; (12) nucleic acid segments that inhibit replication, partition or heritability of nucleic acid molecules that contain them; (13) nucleic acid segments that encode conditional replication functions, e.g., replication in certain hosts or host cell strains or under certain environmental conditions (e.g., temperature, nutritional conditions, and the like); and/or (14) nucleic acids that encode one or more mRNAs or tRNA that comprise unnatural nucleotides. In some embodiments, the regulatory or selective agent can be added to change the existing growth conditions to which the organism is subjected (e.g., growth in liquid culture, growth in a fermenter, growth on solid nutrient plates and the like for example).

In some embodiments, regulation of a promoter element can be used to alter (e.g., increase, add, decrease or substantially eliminate) the activity of a peptide, polypeptide or protein (e.g., enzyme activity for example). For example, a microorganism can be engineered by genetic modification to express a nucleic acid reagent that can add a novel activity (e.g., an activity not normally found in the host organism) or increase the expression of an existing activity by increasing transcription from a homologous or heterologous promoter operably linked to a nucleotide sequence of interest (e.g., homologous or heterologous nucleotide sequence of interest), in certain embodiments. In some embodiments, a microorganism can be engineered by genetic modification to express a nucleic acid reagent that can decrease expression of an activity by decreasing or substantially eliminating transcription from a homologous or heterologous promoter operably linked to a nucleotide sequence of interest, in certain embodiments.

Nucleic acids encoding heterologous proteins, e.g., tRNA synthetases, can be inserted into or employed with any suitable expression system. In some embodiments, a nucleic acid reagent sometimes is stably integrated into the chromosome of the host organism, or a nucleic acid reagent can be a deletion of a portion of the host chromosome, in certain embodiments (e.g., genetically modified organisms, where alteration of the host genome confers the ability to selectively or preferentially maintain the desired organism carrying the genetic modification). Such nucleic acid reagents (e.g., nucleic acids or genetically modified organisms whose altered genome confers a selectable trait to the organism) can be selected for their ability to guide production of a desired protein or nucleic acid molecule. When desired, the nucleic acid reagent can be altered such that codons encode for (i) the same amino acid, using a different tRNA than that specified in the native sequence, or (ii) a different amino acid than is normal, including unconventional or unnatural amino acids (including detectably labeled amino acids).

Recombinant expression is usefully accomplished using an expression cassette that can be part of a vector, such as a plasmid. A vector can include a promoter operably linked to nucleic acid. A vector can also include other elements required for transcription and translation as described herein. An expression cassette, expression vector, and sequences in a cassette or vector can be heterologous to the cell to which the unnatural nucleotides are contacted.

A variety of prokaryotic and eukaryotic expression vectors suitable for carrying, encoding and/or expressing heterologous protein such as a tRNA synthetase can be produced. Such expression vectors include, for example, pET, pET3d, pCR2.1, pBAD, pUC, and yeast vectors. The vectors can be used, for example, in a variety of in vivo and in vitro situations. Non-limiting examples of prokaryotic promoters that can be used include SP6, T7, T5, tac, bla, trp, gal, lac, or maltose promoters. Non-limiting examples of eukaryotic promoters that can be used include constitutive promoters, e.g., viral promoters such as CMV, SV40 and RSV promoters, as well as regulatable promoters, e.g., an inducible or repressible promoter such as a tet promoter, a hsp70 promoter, and a synthetic promoter regulated by CRE. Vectors for bacterial expression include pGEX-5X-3, and for eukaryotic expression include pCIneo-CMV. Viral vectors that can be employed include those relating to lentivirus, adenovirus, adeno-associated virus, herpes virus, vaccinia virus, polio virus, AIDS virus, neuronal trophic virus, Sindbis and other viruses. Also useful are any viral families which share the properties of these viruses which make them suitable for use as vectors. Retroviral vectors that can be employed include those described in Verma, American Society for Microbiology, pp. 229-232, Washington, (1985). For example, such retroviral vectors can include Murine Maloney Leukemia virus, MMLV, and other retroviruses that express desirable properties. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral nucleic acid.

### Cloning

Any convenient cloning strategy known in the art may be utilized to incorporate an element, such as an ORF, into a nucleic acid reagent. Known methods can be utilized to insert an element into the template independent of an insertion element, such as (1) cleaving the template at one or more existing restriction enzyme sites and ligating an element of interest and (2) adding restriction enzyme sites to the template by hybridizing oligonucleotide primers that include one or more suitable restriction enzyme sites and amplifying by polymerase chain reaction (described in greater detail herein). Other cloning strategies take advantage of one or more insertion sites present or inserted into the nucleic acid reagent, such as an oligonucleotide primer hybridization site for PCR, for example, and others described herein. In some embodiments, a cloning strategy can be combined with genetic manipulation such as recombination (e.g., recombination of a nucleic acid reagent with a nucleic acid sequence of interest into the genome of the organism to be modified, as described further herein). In some embodiments, the cloned ORF(s) can produce (directly or indirectly) modified or wild type polymerases), by engineering a microorganism with one or more ORFs of interest, which microorganism comprises altered activities of polymerase activity.

A nucleic acid may be specifically cleaved by contacting the nucleic acid with one or more specific cleavage agents. Specific cleavage agents often will cleave specifically according to a particular nucleotide sequence at a particular site. Examples of enzyme specific cleavage agents include without limitation endonucleases (e.g., DNase (e.g., DNase I, II); RNase (e.g., RNase E, F, H, P); Cleavase™ enzyme; Taq DNA polymerase; E. coli DNA polymerase I and eukaryotic structure-specific endonucleases; murine FEN-1 endonucleases; type I, II or III restriction endonucleases such as Acc I, Afl III, Alu I, Alw44 I, Apa I, Asn I, Ava I, Ava II, BamH I, Ban II, Bcl I, Bgl I. Bgl II, Bln I, BsaI, Bsm I, BsmBI, BssH II, BstE II, Cfo I, CIa I, Dde I, Dpn I, Dra I, EcIX I, EcoR I, EcoR I, EcoR II, EcoR V, Hae II, Hae II, Hind II, Hind III, Hpa I, Hpa II, Kpn I, Ksp I, Mlu I, MIuN I, Msp I, Nci I, Nco I, Nde I, Nde II, Nhe I, Not I, Nru I, Nsi I, Pst I, Pvu I, Pvu II, Rsa I, Sac I, Sal I, Sau3A I, Sca I, ScrF I, Sfi I, Sma I, Spe I, Sph I, Ssp I, Stu I, Sty I, Swa I, Taq I, Xba I, Xho I); glycosylases (e.g., uracil-DNA glycolsylase (UDG), 3-methyladenine DNA glycosylase, 3-methyladenine DNA glycosylase II, pyrimidine hydrate-DNA glycosylase, FaPy-DNA glycosylase, thymine mismatch-DNA glycosylase, hypoxanthine-DNA glycosylase, 5-Hydroxymethyluracil DNA glycosylase (HmUDG), 5-Hydroxymethylcytosine DNA glycosylase, or 1,N6-etheno-adenine DNA glycosylase); exonucleases (e.g., exonuclease III); ribozymes, and DNAzymes. Sample nucleic acid may be treated with a chemical agent, or synthesized using modified nucleotides, and the modified nucleic acid may be cleaved. In non-limiting examples, sample nucleic acid may be treated with (i) alkylating agents such as methylnitrosourea that generate several alkylated bases, including N3-methyladenine and N3-methylguanine, which are recognized and cleaved by alkyl purine DNA-glycosylase; (ii) sodium bisulfite, which causes deamination of cytosine residues in DNA to form uracil residues that can be cleaved by uracil N-glycosylase; and (iii) a chemical agent that converts guanine to its oxidized form, 8-hydroxyguanine, which can be cleaved by formamidopyrimidine DNA N-glycosylase. Examples of chemical cleavage processes include without limitation alkylation, (e.g., alkylation of phosphorothioate-modified nucleic acid); cleavage of acid lability of P3′—N5′-phosphoroamidate-containing nucleic acid; and osmium tetroxide and piperidine treatment of nucleic acid.

In some embodiments, the nucleic acid reagent includes one or more recombinase insertion sites. A recombinase insertion site is a recognition sequence on a nucleic acid molecule that participates in an integration/recombination reaction by recombination proteins. For example, the recombination site for Cre recombinase is loxP, which is a 34 base pair sequence comprised of two 13 base pair inverted repeats (serving as the recombinase binding sites) flanking an 8 base pair core sequence (e.g., Sauer, Curr. Opin. Biotech. 5:521-527 (1994)). Other examples of recombination sites include attB, attP, attL, and attR sequences, and mutants, fragments, variants and derivatives thereof, which are recognized by the recombination protein λ Int and by the auxiliary proteins integration host factor (IHF), FIS and excisionase (Xis) (e.g., U.S. Pat. Nos. 5,888,732; 6,143,557; 6,171,861; 6,270,969; 6,277,608; and 6,720,140; U.S. patent Appln. Ser. Nos. 09/517,466, and 09/732,914; U.S. Patent Publication No. US2002/0007051; and Landy, Curr. Opin. Biotech. 3:699-707 (1993)).

Examples of recombinase cloning nucleic acids are in Gateway® systems (Invitrogen, California), which include at least one recombination site for cloning desired nucleic acid molecules in vivo or in vitro. In some embodiments, the system utilizes vectors that contain at least two different site-specific recombination sites, often based on the bacteriophage lambda system (e.g., att1 and att2), and are mutated from the wild-type (att0) sites. Each mutated site has a unique specificity for its cognate partner att site (i.e., its binding partner recombination site) of the same type (for example attB1 with attP1, or attL1 with attR1) and will not cross-react with recombination sites of the other mutant type or with the wild-type att0 site. Different site specificities allow directional cloning or linkage of desired molecules thus providing desired orientation of the cloned molecules. Nucleic acid fragments flanked by recombination sites are cloned and subcloned using the Gateway® system by replacing a selectable marker (for example, ccdB) flanked by att sites on the recipient plasmid molecule, sometimes termed the Destination Vector. Desired clones are then selected by transformation of a ccdB sensitive host strain and positive selection for a marker on the recipient molecule. Similar strategies for negative selection (e.g., use of toxic genes) can be used in other organisms such as thymidine kinase (TK) in mammals and insects.

A nucleic acid reagent sometimes contains one or more origin of replication (ORI) elements. In some embodiments, a template comprises two or more ORIs, where one functions efficiently in one organism (e.g., a bacterium) and another function efficiently in another organism (e.g., a eukaryote, like yeast for example). In some embodiments, an ORI may function efficiently in one species (e.g., S. cerevisiae, for example) and another ORI may function efficiently in a different species (e.g., S. pombe, for example). A nucleic acid reagent also sometimes includes one or more transcription regulation sites.

A nucleic acid reagent, e.g., an expression cassette or vector, can include nucleic acid sequence encoding a marker product. A marker product is used to determine if a gene has been delivered to the cell and once delivered is being expressed. Example marker genes include the E. coli lacZ gene which encodes β-galactosidase and green fluorescent protein. In some embodiments the marker can be a selectable marker. When such selectable markers are successfully transferred into a host cell, the transformed host cell can survive if placed under selective pressure. There are two widely used distinct categories of selective regimes. The first category is based on a cell's metabolism and the use of a mutant cell line which lacks the ability to grow independent of a supplemented media. The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin (Southern et al., J. Molec. Appl. Genet. 1: 327 (1982)), mycophenolic acid, (Mulligan et al., Science 209: 1422 (1980)) or hygromycin, (Sugden, et al., Mol. Cell. Biol. 5: 410-413 (1985)).

A nucleic acid reagent can include one or more selection elements (e.g., elements for selection of the presence of the nucleic acid reagent, and not for activation of a promoter element which can be selectively regulated). Selection elements often are utilized using known processes to determine whether a nucleic acid reagent is included in a cell. In some embodiments, a nucleic acid reagent includes two or more selection elements, where one functions efficiently in one organism, and another functions efficiently in another organism. Examples of selection elements include, but are not limited to, (1) nucleic acid segments that encode products that provide resistance against otherwise toxic compounds (e.g., antibiotics); (2) nucleic acid segments that encode products that are otherwise lacking in the recipient cell (e.g., essential products, tRNA genes, auxotrophic markers); (3) nucleic acid segments that encode products that suppress the activity of a gene product; (4) nucleic acid segments that encode products that can be readily identified (e.g., phenotypic markers such as antibiotics (e.g., β-lactamase), β-galactosidase, green fluorescent protein (GFP), yellow fluorescent protein (YFP), red fluorescent protein (RFP), cyan fluorescent protein (CFP), and cell surface proteins); (5) nucleic acid segments that bind products that are otherwise detrimental to cell survival and/or function; (6) nucleic acid segments that otherwise inhibit the activity of any of the nucleic acid segments described in Nos. 1-5 above (e.g., antisense oligonucleotides); (7) nucleic acid segments that bind products that modify a substrate (e.g., restriction endonucleases); (8) nucleic acid segments that can be used to isolate or identify a desired molecule (e.g., specific protein binding sites); (9) nucleic acid segments that encode a specific nucleotide sequence that can be otherwise non-functional (e.g., for PCR amplification of subpopulations of molecules); (10) nucleic acid segments that, when absent, directly or indirectly confer resistance or sensitivity to particular compounds; (11) nucleic acid segments that encode products that either are toxic or convert a relatively non-toxic compound to a toxic compound (e.g., Herpes simplex thymidine kinase, cytosine deaminase) in recipient cells; (12) nucleic acid segments that inhibit replication, partition or heritability of nucleic acid molecules that contain them; and/or (13) nucleic acid segments that encode conditional replication functions, e.g., replication in certain hosts or host cell strains or under certain environmental conditions (e.g., temperature, nutritional conditions, and the like).

A nucleic acid reagent can be of any form useful for in vivo transcription and/or translation. A nucleic acid sometimes is a plasmid, such as a supercoiled plasmid, sometimes is a yeast artificial chromosome (e.g., YAC), sometimes is a linear nucleic acid (e.g., a linear nucleic acid produced by PCR or by restriction digest), sometimes is single-stranded and sometimes is double-stranded. A nucleic acid reagent sometimes is prepared by an amplification process, such as a polymerase chain reaction (PCR) process or transcription-mediated amplification process (TMA). In TMA, two enzymes are used in an isothermal reaction to produce amplification products detected by light emission (e.g., Biochemistry Jun. 25, 1996; 35(25):8429-38). Standard PCR processes are known (e.g., U.S. Pat. Nos. 4,683,202; 4,683,195; 4,965,188; and 5,656,493), and generally are performed in cycles. Each cycle includes heat denaturation, in which hybrid nucleic acids dissociate; cooling, in which primer oligonucleotides hybridize; and extension of the oligonucleotides by a polymerase (i.e., Taq polymerase). An example of a PCR cyclical process is treating the sample at 95° C. for 5 minutes; repeating forty-five cycles of 95° C. for 1 minute, 59° C. for 1 minute, 10 seconds, and 72° C. for 1 minute 30 seconds; and then treating the sample at 72° C. for 5 minutes. Multiple cycles frequently are performed using a commercially available thermal cycler. PCR amplification products sometimes are stored for a time at a lower temperature (e.g., at 4° C.) and sometimes are frozen (e.g., at −20° C.) before analysis.

Cloning strategies analogous to those described above may be employed to produce DNA containing unnatural nucleotides. For example, oligonucleotides containing the unnatural nucleotides at desired positions are synthesized using standard solid-phase synthesis and purified by HPLC. The oligonucleotides are then inserted into the plasmid containing required sequence context (i.e. UTRs and coding sequence) using a cloning method (such as Golden Gate Assembly) with cloning sites, such as BsaI sites (although others discussed above may be used).

### Kits/Article of Manufacture

Disclosed herein, in certain embodiments, are kits and articles of manufacture for use with one or more methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

In some embodiments, a kit includes a suitable packaging material to house the contents of the kit. In some cases, the packaging material is constructed by well-known methods, preferably to provide a sterile, contaminant-free environment. The packaging materials employed herein can include, for example, those customarily utilized in commercial kits sold for use with nucleic acid sequencing systems. Exemplary packaging materials include, without limitation, glass, plastic, paper, foil, and the like, capable of holding within fixed limits a component set forth herein.

The packaging material can include a label which indicates a particular use for the components. The use for the kit that is indicated by the label can be one or more of the methods set forth herein as appropriate for the particular combination of components present in the kit. For example, a label can indicate that the kit is useful for a method of synthesizing a polynucleotide or for a method of determining the sequence of a nucleic acid.

Instructions for use of the packaged reagents or components can also be included in a kit. The instructions will typically include a tangible expression describing reaction parameters, such as the relative amounts of kit components and sample to be admixed, maintenance time periods for reagent/sample admixtures, temperature, buffer conditions, and the like.

It will be understood that not all components necessary for a particular reaction need be present in a particular kit. Rather one or more additional components can be provided from other sources. The instructions provided with a kit can identify the additional component(s) that are to be provided and where they can be obtained.

In some embodiments, a kit is provided that is useful for stably incorporating an unnatural nucleic acid into a cellular nucleic acid, e.g., using the methods provided by the present invention for preparing genetically engineered mammalian cells (e.g., CHO or HEK293T cells). In one embodiment, a kit described herein includes a genetically engineered cell and one or more unnatural nucleic acids.

In additional embodiments, the kit described herein provides a cell and a nucleic acid molecule containing a heterologous gene for introduction into the cell to thereby provide a genetically engineered cell, such as expression vectors comprising the nucleic acid of any of the embodiments hereinabove described in this paragraph.

In some embodiments, a cell described herein is delivered to an organism, which may be a multicellular organism, such as a mammal, e.g., a human. As such, eukaryotic cells comprising a polypeptide having an unnatural amino acid can be introduced to an organism.

### NUMBERED EMBODIMENTS

The present disclosure includes the following non-limiting numbered embodiments:

### Embodiment 1

A method of producing a polypeptide comprising one or more unnatural amino acids in a eukaryotic cell, comprising:
(a) providing a eukaryotic cell comprising:
   (i) a transfer RNA (tRNA) with an anticodon comprising a first unnatural base;
   (ii) a messenger RNA (mRNA) with a codon comprising a second unnatural base, wherein the first and second unnatural bases form an unnatural base pair (UBP) in the eukaryotic cell;
(b) translating the polypeptide comprising the one or more unnatural amino acids from the mRNA using the tRNA by a ribosome that is endogenous to the eukaryotic cell.

### Embodiment 2

The method of embodiment 1, wherein the codon of the mRNA comprises three contiguous nucleobases (N—N—N); and wherein the first unnatural base (X) is located at the first position (X—N—N) in the codon of the mRNA.

### Embodiment 3

The method of embodiment 1, wherein the codon of the mRNA comprises three contiguous nucleobases (N—N—N); and wherein the first unnatural base (X) is located at the middle position (N—X—N) in the codon of the mRNA.

### Embodiment 4

The method of embodiment 1, wherein the codon of the mRNA comprises three contiguous nucleobases (N—N—N); and wherein the first unnatural base (X) is located at the last position (N—N—X) in the codon of the mRNA.

### Embodiment 5

The method of any one of embodiments 1 to 4, wherein the first unnatural base or the second unnatural base is selected from the group consisting of:
(i) 2-thiouracil, 2-thio-thymine, 2′-deoxyuridine, 4-thio-uracil, 4-thio-thymine, uracil-5-yl, hypoxanthin-9-yl (I), 5-halouracil; 5-propynyl-uracil, 6-azo-thymine, 6-azo-uracil, 5-methylaminomethyluracil, 5-methoxyaminomethyl-2-thiouracil, pseudouracil, uracil-5-oxacetic acid methylester, uracil-5-oxacetic acid, 5-methyl-2-thiouracil, 3-(3-amino-3-N-2-carboxypropyl) uracil, 5-methyl-2-thiouracil, 4-thiouracil, 5-methyluracil, 5′-methoxycarboxymethyluracil, 5-methoxyuracil, uracil-5-oxyacetic acid, 5-(carboxyhydroxylmethyl) uracil, 5-carboxymethylaminomethyl-2-thiouridine, 5-carboxymethylaminomethyluracil, or dihydrouracil;
(ii) 5-hydroxymethyl cytosine, 5-trifluoromethyl cytosine, 5-halocytosine, 5-propynyl cytosine, 5-hydroxycytosine, cyclocytosine, cytosine arabinoside, 5,6-dihydrocytosine, 5-nitrocytosine, 6-azo cytosine, azacytosine, N4-ethylcytosine, 3-methylcytosine, 5-methylcytosine, 4-acetylcytosine, 2-thiocytosine, phenoxazine cytidine ([5,4-b][1,4]benzoxazin-2(3H)-one), phenothiazine cytidine (1H-pyrimido[5,4-b][1, 4]benzothiazin-2(3H)-one), phenoxazine cytidine (9-(2-aminoethoxy)-H-pyrimido[5,4-b][1,4]benzoxazin-2(3H)-one), carbazole cytidine (2H-pyrimido[4,5-b]indol-2-one), or pyridoindole cytidine (H-pyrido [3′,2′:4,5]pyrrolo [2,3-d]pyrimidin-2-one);
(iii)₂-aminoadenine, 2-propyl adenine, 2-amino-adenine, 2-F-adenine, 2-amino-propyl-adenine, 2-amino-2′-deoxyadenosine, 3-deazaadenine, 7-methyladenine, 7-deaza-adenine, 8-azaadenine, 8-halo, 8-amino, 8-thiol, 8-thioalkyl, and 8-hydroxyl substituted adenines, N6-isopentenyladenine, 2-methyladenine, 2,6-diaminopurine, 2-methythio-N6-isopentenyladenine, or 6-aza-adenine;
(iv) 2-methylguanine, 2-propyl and alkyl derivatives of guanine, 3-deazaguanine, 6-thio-guanine, 7-methylguanine, 7-deazaguanine, 7-deazaguanosine, 7-deaza-8-azaguanine, 8-azaguanine, 8-halo, 8-amino, 8-thiol, 8-thioalkyl, and 8-hydroxyl substituted guanines, 1-methylguanine, 2,2-dimethylguanine, 7-methylguanine, or 6-aza-guanine; and
(v) hypoxanthine, xanthine, 1-methylinosine, queosine, beta-D-galactosylqueosine, inosine, beta-D-mannosylqueosine, wybutoxosine, hydroxyurea, (acp3)w, 2-aminopyridine, or 2-pyridone.

### Embodiment 6

The method of any one of embodiments 1 to 4, wherein the first unnatural base or the second unnatural base is selected from the group consisting of

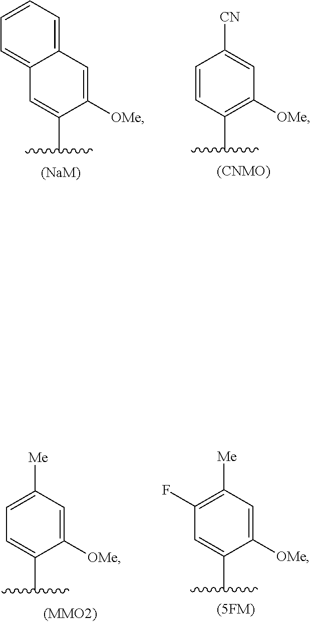

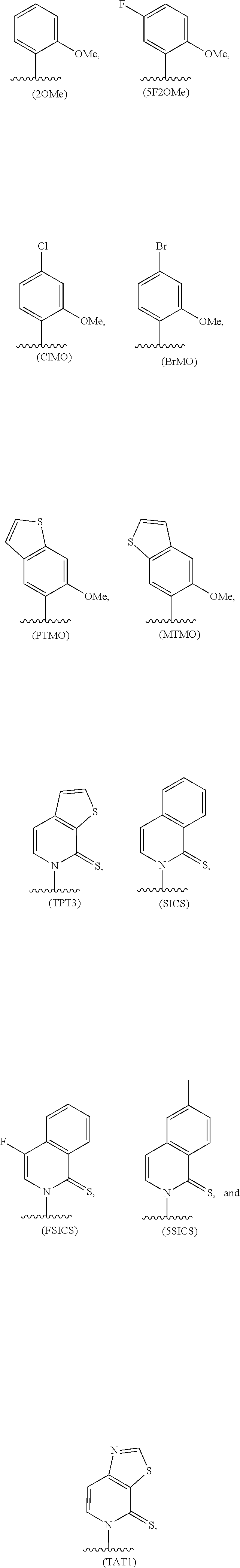

wherein the wavy line indicates a bond to a ribosyl moiety.

### Embodiment 7

The method of embodiment 6, when the first unnatural base is

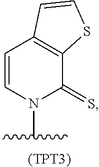

the second unnatural base is

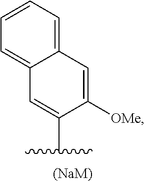

and when the first unnatural base is

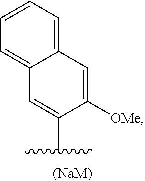

the second unnatural base is

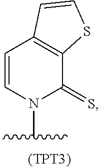

wherein the wavy line indicates a bond to a ribosyl moiety.

### Embodiment 8

The method of embodiment 6, when the first unnatural base is

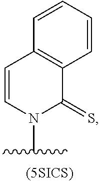

the second unnatural base is

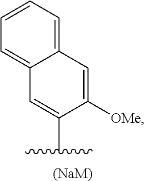

and when the first unnatural base is

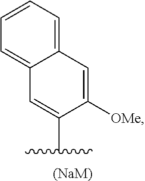

the second unnatural base is

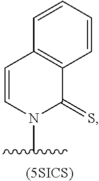

wherein the wavy line indicates a bond to a ribosyl moiety.

### Embodiment 9

The method of embodiment 6, when the first unnatural base is

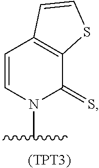

the second unnatural base is

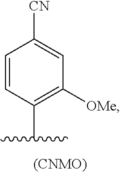 [SMILES: *c1ccc(C#N)cc1OC]

and when the first unnatural base is

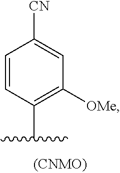 [SMILES: *c1ccc(C#N)cc1OC]

the second unnatural base is

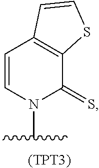

wherein the wavy line indicates a bond to a ribosyl moiety.

### Embodiment 10

The method of embodiment 6, when the first unnatural base is

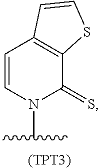

the second unnatural base is

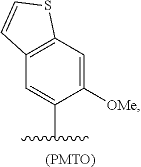

and when the first unnatural base is

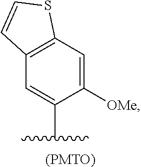

the second unnatural base is

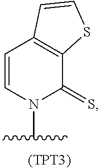

wherein the wavy line indicates a bond to a ribosyl moiety.

### Embodiment 11

The method of embodiment 6, when the first unnatural base is

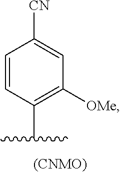 [SMILES: *c1ccc(C#N)cc1OC]

the second unnatural base is

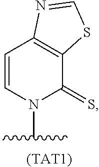

and when the first unnatural base is

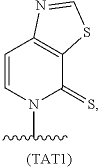

the second unnatural base is

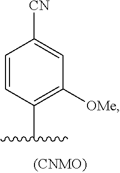 [SMILES: *c1ccc(C#N)cc1OC]

wherein the wavy line indicates a bond to a ribosyl moiety.

### Embodiment 12

The method of embodiment 6, when the first unnatural base is

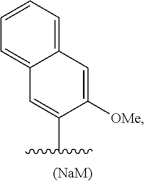

the second unnatural base is

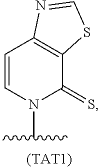

and when the first unnatural base is

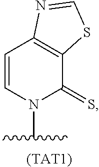

the second unnatural base is

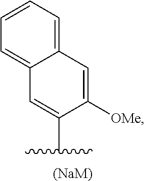

wherein the wavy line indicates a bond to a ribosyl moiety.

### Embodiment 13

The method of any one of embodiments 1 to 12, wherein the first unnatural base or the second unnatural base comprise a modified sugar moiety selected from the group consisting of:

a modification at the 2′ position:
OH, substituted lower alkyl, alkaryl, aralkyl, O-alkaryl or O-aralkyl, SH, SCH₃, OCN, Cl,
Br, CN, CF₃, OCF₃, SOCH₃, SO₂CH₃, ONO₂, NO₂, N₃, NH₂F;
O-alkyl, S-alkyl, N-alkyl;
O-alkenyl, S-alkenyl, N-alkenyl;
O-alkynyl, S-alkynyl, N-alkynyl;
O-alkyl-O-alkyl, 2′-F, 2′—OCH₃, 2′—O(CH₂)₂OCH₃ wherein the alkyl, alkenyl and alkynyl may be substituted or unsubstituted C₁-C₁₀, alkyl, C₂-C₁₀ alkenyl, C₂-C₁₀ alkynyl, —
O[(CH₂)ₙO]ₘCH₃, —O(CH₂)ₙOCH₃, —O(CH₂)ₙNH₂, —O(CH₂)ₙCH₃, —O(CH₂)ₙ—NH₂, and —
O(CH₂)ₙON[(CH₂)ₙCH₃)]₂, wherein n and m are from 1 to about 10;
and/or a modification at the 5′ position:
5′-vinyl, 5′-methyl (R or S);
a modification at the 4′ position:
4′-S, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and any combination thereof.

### Embodiment 14

The method of any one of embodiments 1 to 13, wherein the method is a human cell.

### Embodiment 15

The method of embodiment 14, wherein the human cell is a HEK293T cell.

### Embodiment 16

The method of any one of embodiments 1 to 13, wherein the cell is a hamster cell.

### Embodiment 17

The method of embodiment 16, wherein the hamster cell is a Chinese hamster ovary (CHO) cell.

### Embodiment 18

The method of any one of embodiments 1 to 17, wherein the unnatural amino acid:
is a lysine analogue;
comprises an aromatic side chain;
comprises an azido group;
comprises an alkyne group; or
comprises an aldehyde or ketone group.

### Embodiment 19

The method of any one of embodiments 1 to 17, wherein the unnatural amino acid is selected from the group consisting of N6-((azidoethoxy)-carbonyl)-L-lysine (AzK), N6-((propargylethoxy)-carbonyl)-L-lysine (PraK), BCN-L-lysine, norbomene lysine, TCO-lysine, methyltetrazine lysine, allyloxycarbonyllysine, 2-amino-8-oxononanoic acid, 2-amino-8-oxooctanoic acid, p-acetyl-L-phenylalanine, p-azidomethyl-L-phenylalanine (pAMF), p-iodo-L-phenylalanine, m-acetylphenylalanine, 2-amino-8-oxononanoic acid, p-propargyloxyphenylalanine, p-propargyl-phenylalanine, 3-methyl-phenylalanine, L-Dopa, fluorinated phenylalanine, isopropyl-L-phenylalanine, p-azido-L-phenylalanine, p-acyl-L-phenylalanine, p-benzoyl-L-phenylalanine, p-bromophenylalanine, p-amino-L-phenylalanine, isopropyl-L-phenylalanine, O-allyltyrosine, O-methyl-L-tyrosine, O-4-allyl-L-tyrosine, 4-propyl-L-tyrosine, phosphonotyrosine, tri-O-acetyl-GlcNAcp-serine, L-phosphoserine, phosphonoserine, L-3-(2-naphthyl)alanine, 2-amino-3-((2-((3-(benzyloxy)-3-oxopropyl)amino)ethyl)selanyl)propanoic acid, 2-amino-3-(phenylselanyl)propanoic, selenocysteine, N6-(((2-azidobenzyl)oxy)carbonyl)-L-lysine, N6-(((3-azidobenzyl)oxy)carbonyl)-L-lysine, or N6-(((4-azidobenzyl)oxy)carbonyl)-L-lysine.

### Embodiment 20

The method of embodiment 19, wherein the unnatural amino acid is N6-((azidoethoxy)-carbonyl)-L-lysine (AzK).

### Embodiment 21

A method of producing a polypeptide in a eukaryotic cell, wherein the polypeptide comprises one or more unnatural amino acids, the method comprising:

(a) providing a eukaryotic cell, the eukaryotic cell comprising:

(i) an mRNA comprising a codon, wherein the codon comprises or more unnatural bases;

(ii) a tRNA comprising an anti-codon, wherein the anti-codon comprises one or more unnatural bases, and wherein the one or more unnatural bases comprising the codon in the mRNA and the one or more unnatural bases comprising the anti-codon in the tRNA form a complimentary base pair; and

(iii) a tRNA synthetase, wherein the tRNA synthetase preferentially aminoacylates the tRNA with the one or more unnatural amino acids compared to a natural amino acid; and

(b) providing the one more unnatural amino acids to the eukaryotic cell, wherein the eukaryotic cell produces the polypeptide comprising the one or more unnatural amino acids.

### Embodiment 22

The method of embodiment 21, wherein the codon of the mRNA comprises three contiguous nucleobases (N—N—N); and wherein the first unnatural base (X) is located at the first position (X—N—N) in the codon of the mRNA.

### Embodiment 23

The method of embodiment 21, wherein the codon of the mRNA comprises three contiguous nucleobases (N—N—N); and wherein the first unnatural base (X) is located at the middle position (N—X—N) in the codon of the mRNA.

### Embodiment 24

The method of embodiment 21, wherein the codon of the mRNA comprises three contiguous nucleobases (N—N—N); and wherein the first unnatural base (X) is located at the last position (N—N—X) in the codon of the mRNA.

### Embodiment 25

The method of any one of embodiments 21 to 24, wherein the one or more unnatural bases comprising the codon in the mRNA is of the formula

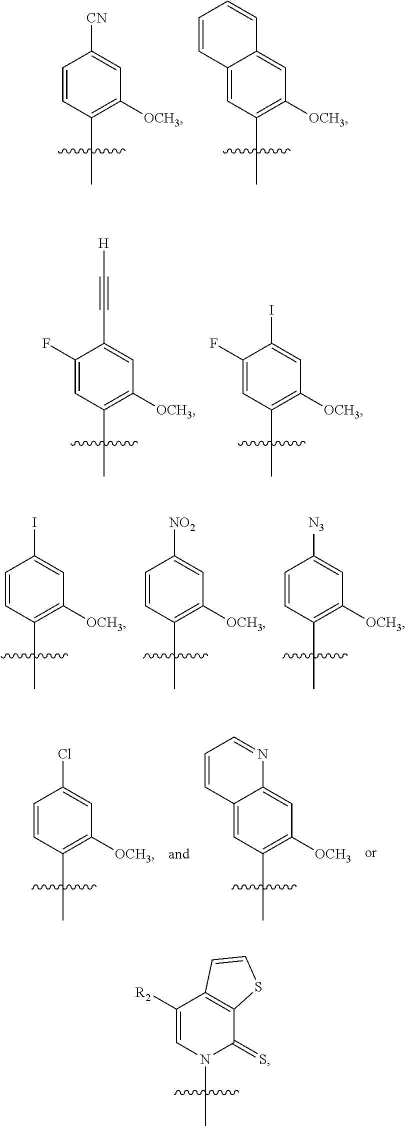

wherein R₂ is selected from the group consisting of hydrogen, alkyl, alkenyl, alkynyl, methoxy, methanethiol, methaneseleno, halogen, cyano, and azido, and the wavy line indicates a bond to a ribosyl moiety.

### Embodiment 26

The method of any one of embodiments 21 to 24, wherein the first unnatural base or the second unnatural base is selected from the group consisting of

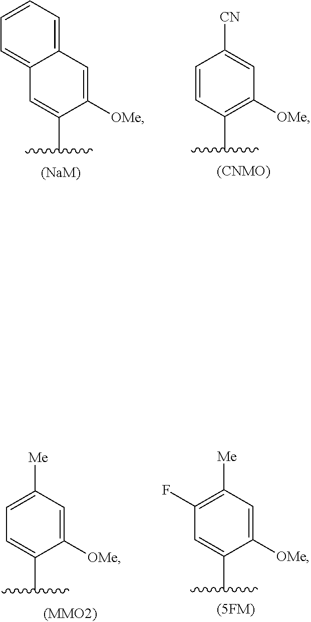

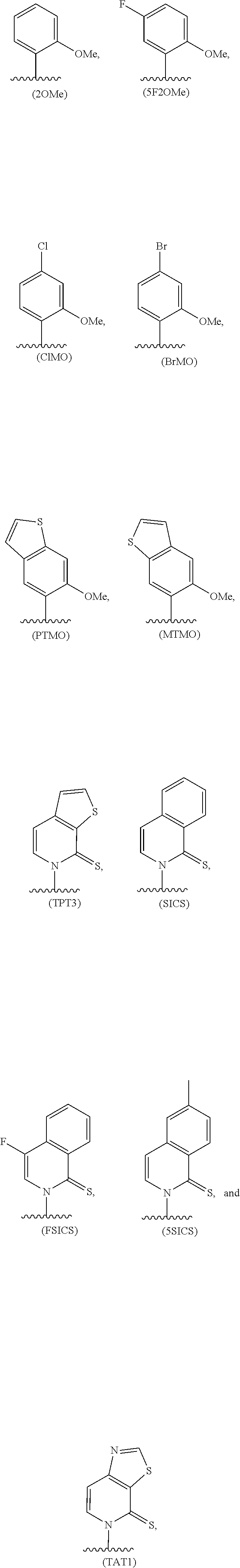

wherein the wavy line indicates a bond to a ribosyl moiety.

### Embodiment 27

The method of embodiment 26, when the first unnatural base is

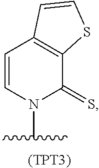

the second unnatural base is

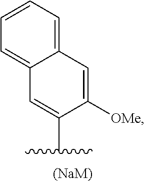

and when the first unnatural base is

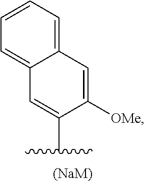

the second unnatural base is

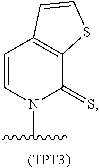

wherein the wavy line indicates a bond to a ribosyl moiety.

### Embodiment 28

The method of embodiment 26, when the first unnatural base is

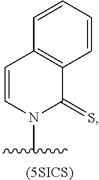

the second unnatural base is

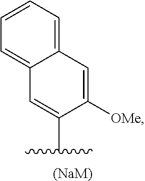

and when the first unnatural base is

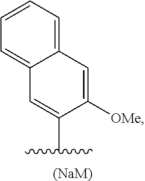

the second unnatural base is

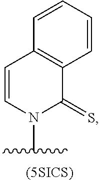

wherein the wavy line indicates a bond to a ribosyl moiety.

### Embodiment 29

The method of embodiment 26, when the first unnatural base is

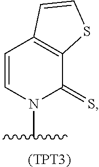

the second unnatural base is

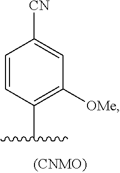 [SMILES: *c1ccc(C#N)cc1OC]

and when the first unnatural base is

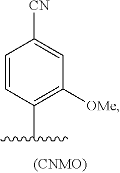 [SMILES: *c1ccc(C#N)cc1OC]

the second unnatural base is

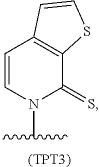

wherein the wavy line indicates a bond to a ribosyl moiety.

### Embodiment 30

The method of embodiment 26, when the first unnatural base is

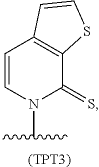

the second unnatural base is

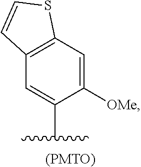

and when the first unnatural base is

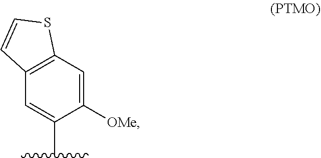 [SMILES: *c1cc2ccsc2cc1OC]

the second unnatural base is

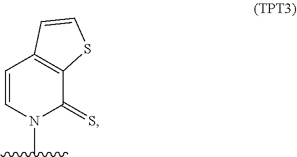 [SMILES: *n1ccc2ccsc2c1=S]

wherein the wavy line indicates a bond to a ribosyl moiety.

### Embodiment 31

The method of embodiment 26, when the first unnatural base is

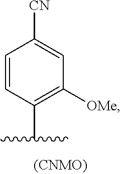 [SMILES: *c1ccc(C#N)cc1OC]

the second unnatural base is

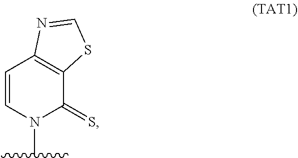 [SMILES: *n1ccc2ncsc2c1=S]

and when the first unnatural base is

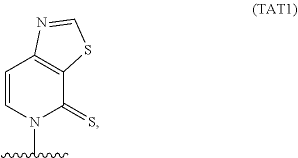 [SMILES: *n1ccc2ncsc2c1=S]

the second unnatural base is

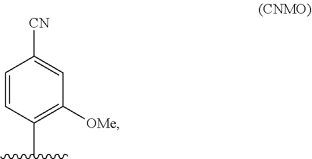 [SMILES: *c1ccc(C#N)cc1OC]

wherein the wavy line indicates a bond to a ribosyl moiety.

### Embodiment 32

The method of embodiment 26, when the first unnatural base is

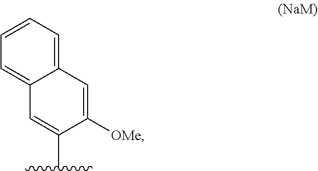 [SMILES: *c1cc2ccccc2cc1OC]

the second unnatural base is

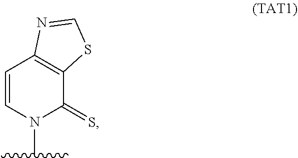 [SMILES: *n1ccc2ncsc2c1=S]

and when the first unnatural base is

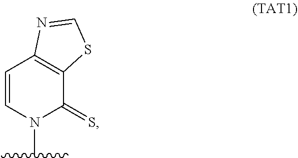 [SMILES: *n1ccc2ncsc2c1=S]

the second unnatural base is

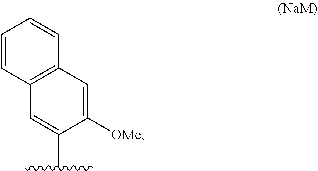 [SMILES: *c1cc2ccccc2cc1OC]

wherein the wavy line indicates a bond to a ribosyl moiety.

### Embodiment 33

The method of embodiment 26, when the first unnatural base is

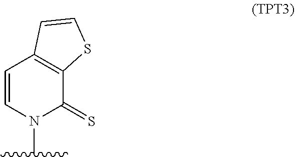 [SMILES: *n1ccc2ccsc2c1=S]

the second unnatural base is

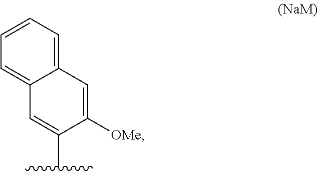 [SMILES: *c1cc2ccccc2cc1OC]

wherein the wavy line indicates a bond to a ribosyl moiety.

### Embodiment 34

The method of any one of embodiments 21 to 24, wherein the unnatural nucleotide comprising the codon in the mRNA is selected from

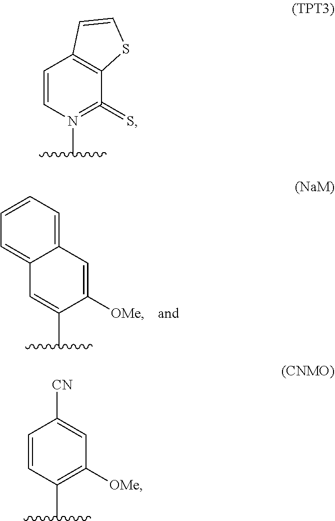

wherein the wavy line indicates a bond to a ribosyl moiety.

### Embodiment 35

The method of embodiment 34, wherein the unnatural nucleotide comprising the codon in the mRNA is

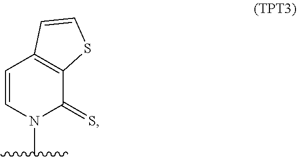 [SMILES: *n1ccc2ccsc2c1=S]

wherein the wavy line indicates a bond to a ribosyl moiety.

### Embodiment 36

The method of embodiment 34, wherein the unnatural nucleotide comprising the codon in the mRNA is

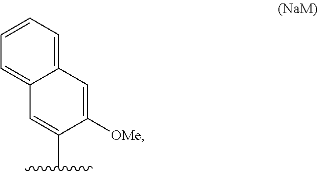 [SMILES: *c1cc2ccccc2cc1OC]

wherein the wavy line indicates a bond to a ribosyl moiety.

### Embodiment 37

The method of embodiment 34, wherein the unnatural nucleotide comprising the codon in the mRNA is

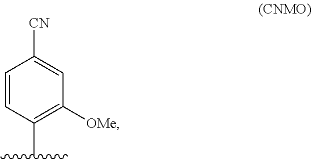 [SMILES: *c1ccc(C#N)cc1OC]

wherein the wavy line indicates a bond to a ribosyl moiety.

### Embodiment 38

The method of embodiment 21, wherein the codon of the mRNA comprises three contiguous nucleobases (N—N—N), wherein the unnatural base (X) is located at the first position (X—N—N) in e codon of the mRNA, wherein the unnatural base is selected from

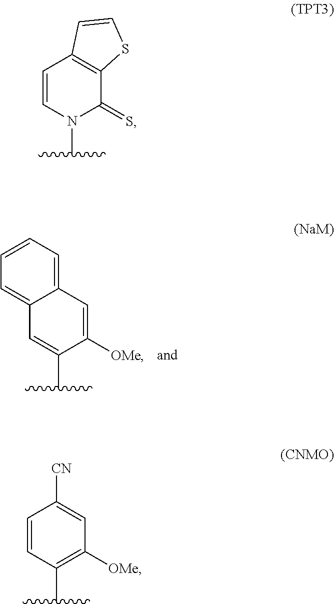 [SMILES: *n1ccc2ccsc2c1=S ; *c1cc2ccccc2cc1OC ; *c1ccc(C#N)cc1OC]

and wherein the wavy line indicates a bond to a ribosyl moiety.

### Embodiment 39

The method of embodiment 38, wherein the unnatural base is

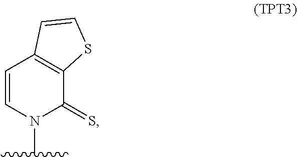 [SMILES: *n1ccc2ccsc2c1=S]

wherein the wavy line indicates a bond to a ribosyl moiety.

### Embodiment 40

The method of embodiment 38, wherein the unnatural base is

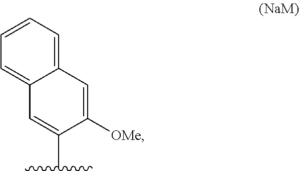 [SMILES: *c1cc2ccccc2cc1OC]

wherein the wavy line indicates a bond to a ribosyl moiety.

### Embodiment 41

The method of embodiment 38, wherein the unnatural base is

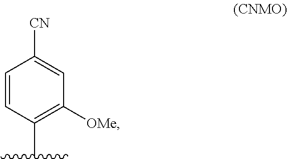 [SMILES: *c1ccc(C#N)cc1OC]

wherein the wavy line indicates a bond to a ribosyl moiety.

### Embodiment 42

The method of embodiment 21, wherein the codon of the mRNA comprises three contiguous nucleobases (N—N—N), wherein the unnatural base (X) is located at the middle position (N—X—N) in the codon of the mRNA, wherein the unnatural base is selected from

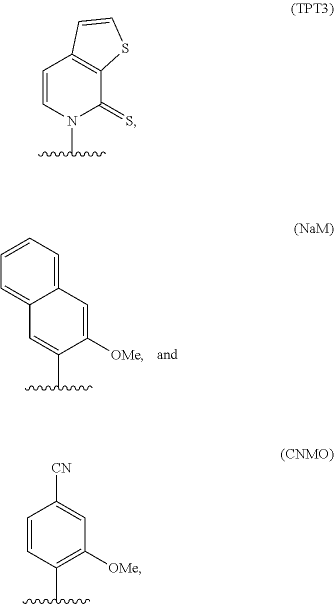 [SMILES: *n1ccc2ccsc2c1=S ; *c1cc2ccccc2cc1OC ; *c1ccc(C#N)cc1OC]

and wherein the wavy line indicates a bond to a ribosyl moiety.

### Embodiment 43

The method of embodiment 42, wherein the unnatural base is

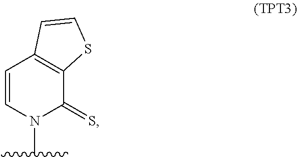 [SMILES: *n1ccc2ccsc2c1=S]

wherein the wavy line indicates a bond to a ribosyl moiety.

### Embodiment 44

The method of embodiment 42, wherein the unnatural base is

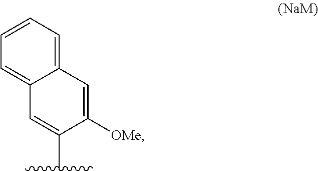 [SMILES: *c1cc2ccccc2cc1OC]

wherein the wavy line indicates a bond to a ribosyl moiety.

### Embodiment 45

The method of embodiment 42, wherein the unnatural base is

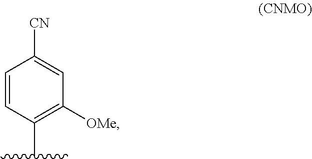 [SMILES: *c1ccc(C#N)cc1OC]

wherein the wavy line indicates a bond to a ribosyl moiety.

### Embodiment 46

The method of embodiment 21, wherein the codon of the mRNA comprises three contiguous nucleobases (N—N—N), wherein the unnatural base (X) is located at the last position (N—N—X) in the codon of the mRNA, wherein the unnatural base is selected from

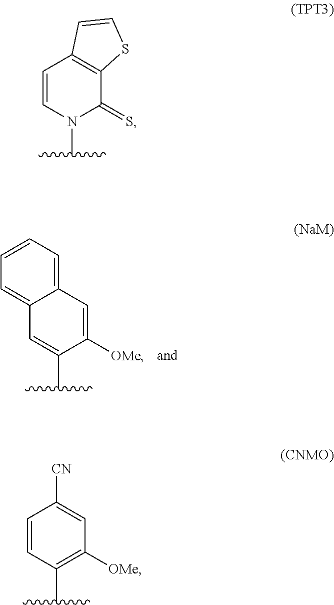 [SMILES: *n1ccc2ccsc2c1=S ; *c1cc2ccccc2cc1OC ; *c1ccc(C#N)cc1OC]

and wherein the wavy line indicates a bond to a ribosyl moiety.

### Embodiment 47

The method of embodiment 46, wherein the unnatural base is

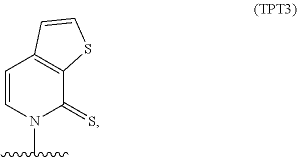 [SMILES: *n1ccc2ccsc2c1=S]

wherein the wavy line indicates a bond to a ribosyl moiety.

### Embodiment 48

The method of embodiment 46, wherein the unnatural base is

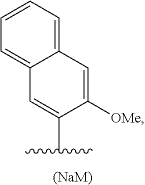

wherein the wavy line indicates a bond to a ribosyl moiety.

### Embodiment 49

The method of embodiment 46, wherein the unnatural base is

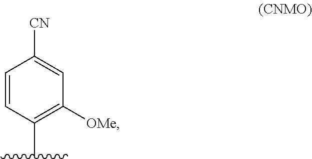 [SMILES: *c1ccc(C#N)cc1OC]

wherein the wavy line indicates a bond to a ribosyl moiety.

### Embodiment 50

The method of embodiment 21, wherein the anticodon of the tRNA comprises three contiguous nucleobases (N—N—N); and wherein the first unnatural base (X) is located at the first position (X—N—N) in the anticodon of the tRNA.

### Embodiment 51

The method of embodiment 50, wherein the unnatural base is selected from

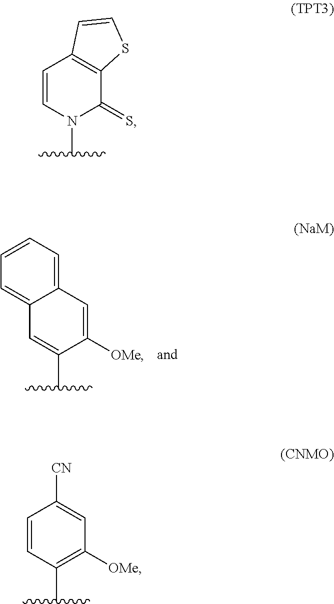 [SMILES: *n1ccc2ccsc2c1=S ; *c1cc2ccccc2cc1OC ; *c1ccc(C#N)cc1OC]

and wherein the wavy line indicates a bond to a ribosyl moiety.

### Embodiment 52

The method of embodiment 51, wherein the unnatural base is

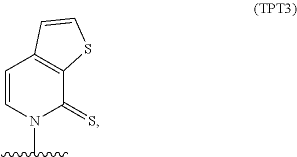 [SMILES: *n1ccc2ccsc2c1=S]

wherein the wavy line indicates a bond to a ribosyl moiety.

### Embodiment 53

The method of embodiment 51, wherein the unnatural base is

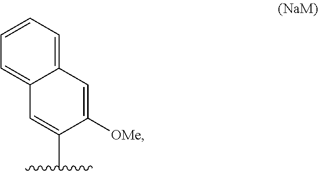 [SMILES: *c1cc2ccccc2cc1OC]

wherein the wavy line indicates a bond to a ribosyl moiety.

### Embodiment 54

The method of embodiment 51, wherein the unnatural base is

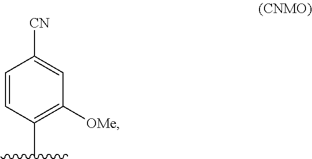 [SMILES: *c1ccc(C#N)cc1OC]

wherein the wavy line indicates a bond to a ribosyl moiety.

### Embodiment 55

The method of embodiment 21, wherein the anticodon of the tRNA comprises three contiguous nucleobases (N—N—N); and wherein the first unnatural base (X) is located at the middle position (N—X—N) in the anticodon of the tRNA.

### Embodiment 56

The method of embodiment 55, wherein the unnatural base is selected from

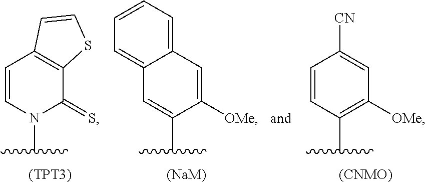

and wherein the wavy line indicates a bond to a ribosyl moiety.

### Embodiment 57

The method of embodiment 55, wherein the unnatural base is

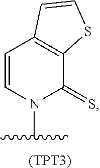

wherein the wavy line indicates a bond to a ribosyl moiety.

### Embodiment 58

The method of embodiment 55, wherein the unnatural base is

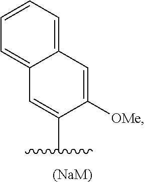

wherein the wavy line indicates a bond to a ribosyl moiety.

### Embodiment 59

The method of embodiment 55, wherein the unnatural base is

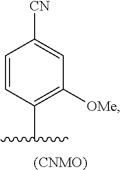

wherein the wavy line indicates a bond to a ribosyl moiety.

### Embodiment 60

The method of embodiment 21, wherein the anticodon of the tRNA comprises three contiguous nucleobases (N—N—N); and wherein the first unnatural base (X) is located at the last position (N—N—X) in the anticodon of the tRNA.

### Embodiment 61

The method of embodiment 60, wherein the unnatural base is selected from

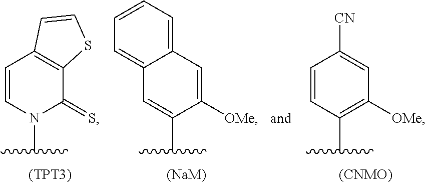

and wherein the wavy line indicates a bond to a ribosyl moiety.

### Embodiment 62

The method of embodiment 61, wherein the unnatural base is

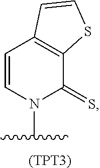

wherein the wavy line indicates a bond to a ribosyl moiety.

### Embodiment 63

The method of embodiment 61, wherein the unnatural base is

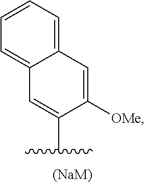

wherein the wavy line indicates a bond to a ribosyl moiety.

### Embodiment 64

The method of embodiment 61, wherein the unnatural base is

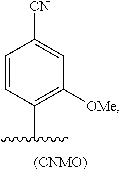

wherein the wavy line indicates a bond to a ribosyl moiety.

### Embodiment 65

The method of embodiment 21, wherein the codon and the anticodon each comprise three contiguous nucleobases (N—N—N), wherein the codon in the mRNA comprises the first unnatural base (X) located at a first position (X—N—N) of the codon, and the anticodon in the tRNA comprises the second unnatural base (Y) located at the last position (N—N—Y) of the anticodon.

### Embodiment 66

The method of embodiment 65, wherein the first unnatural base (X) located in the codon of the mRNA and the second unnatural base (Y) located in the anticodon of the tRNA are the same or are different.

### Embodiment 67

The method of embodiment 66, wherein the first unnatural base (X) located in the codon of the mRNA and the second unnatural base (Y) located in the anticodon of the tRNA are the same.

### Embodiment 68

The method of embodiment 66, wherein the first unnatural base (X) located in the codon of the mRNA and the second unnatural base (Y) located in the anticodon of the tRNA are different.

### Embodiment 69

The method of any one of embodiments 65 to 68, wherein the first unnatural base (X) located in the codon of the mRNA and the second unnatural base (Y) located in the anticodon of the tRNA are selected from the group consisting of

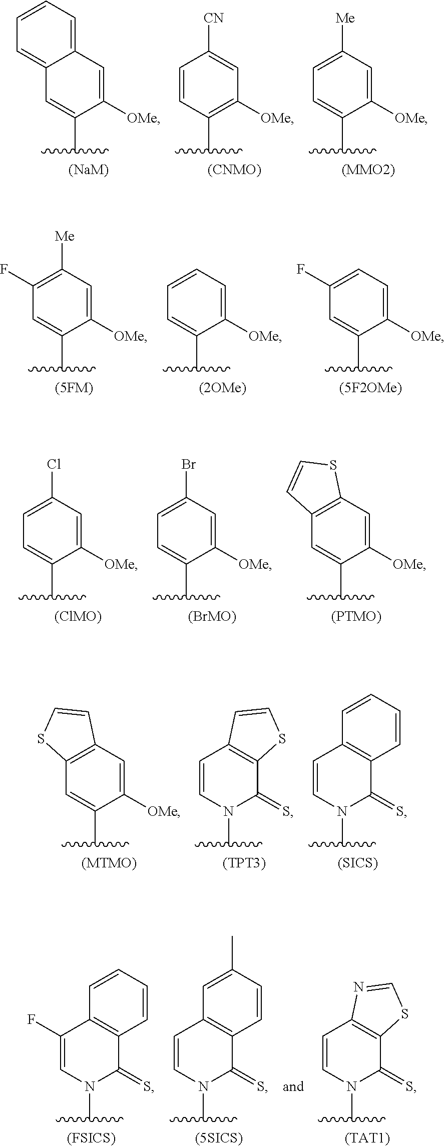

wherein the wavy line indicates a bond to a ribosyl moiety.

### Embodiment 70

The method of embodiment 69, wherein the first unnatural base (X) located in the codon of the mRNA and the second unnatural base (Y) located in the anticodon of the tRNA are selected from the group consisting of

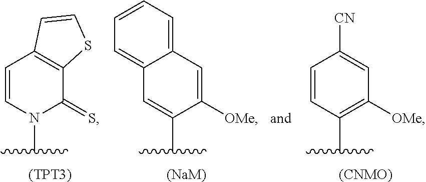

wherein the wavy line indicates a bond to a ribosyl moiety.

### Embodiment 71

The method of embodiment 70, wherein the first unnatural base (X) located in the codon of the mRNA and the second unnatural base (Y) located in the anticodon of the tRNA are both

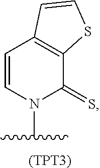

wherein the wavy line indicates a bond to a ribosyl moiety.

### Embodiment 72

The method of embodiment 70, wherein the first unnatural base (X) located in the cod second unnatural base (Y) located in the anticodon of the tRNA are both

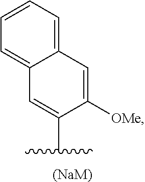

wherein the wavy line indicates a bond to a ribosyl moiety.

### Embodiment 73

The method of embodiment 70, wherein the first unnatural base (X) located in the codon of the mRNA and the second unnatural base (Y) located in the anticodon of the tRNA are both

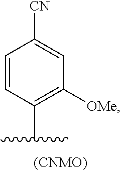

wherein the wavy line indicates a bond to a ribosyl moiety.

### Embodiment 74

The method of embodiment 70, wherein the first unnatural base (X) located in the codon of the mRNA is selected from

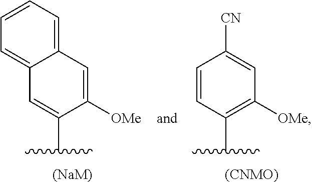

and the second unnatural base (Y) located in the anticodon of the tRNA is

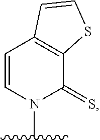 [SMILES: *n1ccc2ccsc2c1=S]

wherein in each case the wavy line indicates a bond to a ribosyl moiety.

### Embodiment 75

The method of embodiment 74, wherein the first unnatural base (X) located in the codon of the mRNA is

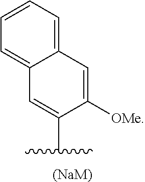

### Embodiment 76

The method of embodiment 74, wherein the first unnatural base (X) located in the codon of the mRNA is (CNMO).

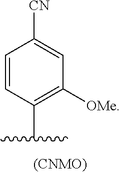 [SMILES: *c1ccc(C#N)cc1OC]

### Embodiment 77

The method of embodiment 21, wherein the codon and the anticodon each comprise three contiguous nucleobases (N—N—N), wherein the codon in the mRNA comprises a first unnatural base (X) located at the middle position (N—X—N) of the codon, and the anticodon in the tRNA comprises a second unnatural base (Y) located at the middle position (N—Y—N) of the anticodon.

### Embodiment 78

The method of embodiment 77, wherein the first unnatural base (X) located in the codon of the mRNA and the second unnatural base (Y) located in the anticodon of the tRNA are the same or are different.

### Embodiment 79

The method of embodiment 78, wherein the first unnatural base (X) located in the codon of the mRNA and the second unnatural base (Y) located in the anticodon of the tRNA are the same.

### Embodiment 80

The method of embodiment 78, wherein the first unnatural base (X) located in the codon of the mRNA and the second unnatural base (Y) located in the anticodon of the tRNA are different.

### Embodiment 81

The method of any one of embodiments 77 to 79, wherein the first unnatural base (X) located in the codon of the mRNA and the second unnatural base (Y) located in the anticodon of the tRNA are selected from the group consisting of

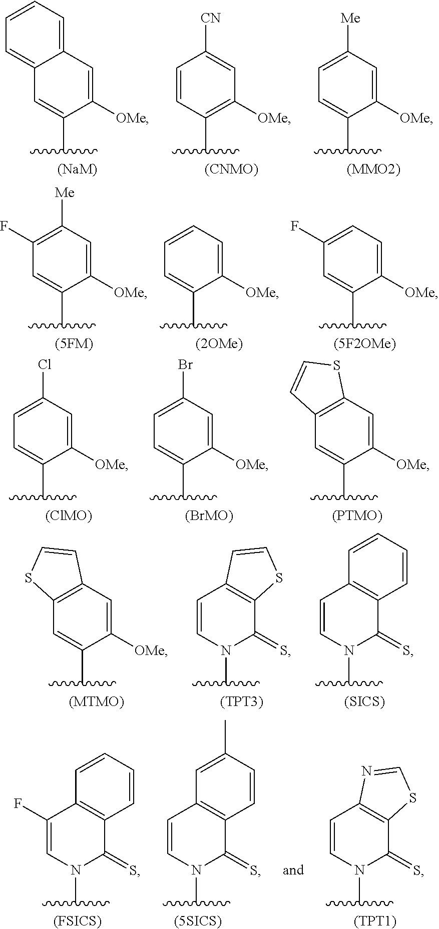

wherein the wavy line indicates a bond to a ribosyl moiety.

### Embodiment 82

The method of embodiment 81, wherein the first unnatural base (X) located in the codon of the mRNA and the second unnatural base (Y) located in the anticodon of the tRNA are selected from the group consisting of

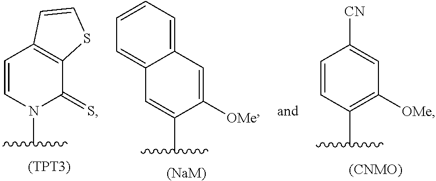

wherein the wavy line indicates a bond to a ribosyl moiety.

### Embodiment 83

The method of embodiment 82, wherein the first unnatural base (X) located in the codon of the mRNA ad the second unnatural base (Y) located in the anticodon of the tRNA are both

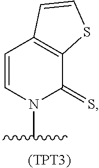

wherein the wavy line indicates a bond to a ribosyl moiety.

### Embodiment 84

The method of embodiment 82, wherein the first unnatural base (X) located in the codon of the mRNA and the second unnatural base (Y) located in the anticodon of the tRNA are both

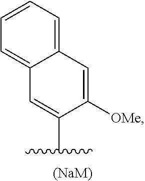

wherein the wavy line indicates a bond to a ribosyl moiety.

### Embodiment 85

The method of embodiment 82, wherein the first unnatural base (X) located in the codon of the mRNA and the second unnatural base (Y) located in the anticodon of the tRNA are both

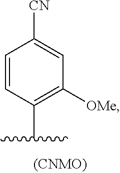 [SMILES: *c1ccc(C#N)cc1OC]

wherein the wavy line indicates a bond to a ribosyl moiety.

### Embodiment 86

The method of embodiment 82, wherein the first unnatural base (X) located in the codon of the mRNA is selected from

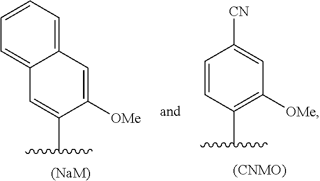

and the second unnatural base (Y) located in the anticodon of the tRNA is

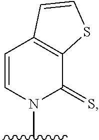 [SMILES: *n1ccc2ccsc2c1=S]

wherein in each case the wavy line indicates a bond to a ribosyl moiety.

### Embodiment 87

The method of embodiment 86, wherein the first unnatural base (X) located in OMe the codon of the mRNA is

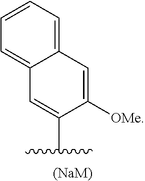

### Embodiment 88

The method of embodiment 86, wherein the first unnatural base (X) located in the codon of the mRNA is

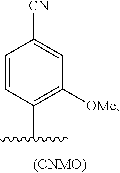 [SMILES: *c1ccc(C#N)cc1OC]

### Embodiment 89

The method of embodiment 21, wherein the codon and the anticodon each comprise three contiguous nucleobases (N—N—N), wherein the codon in the mRNA comprises a first unnatural base (X) located at the last position (N—N—X) of the codon, and the anticodon in the tRNA comprises a second unnatural base (Y) located at the first position (Y—N—N) of the anticodon.

### Embodiment 90

The method of embodiment 89, wherein the first unnatural base (X) located in the codon of the mRNA and the second unnatural base (Y) located in the anticodon of the tRNA are the same or are different.

### Embodiment 91

The method of embodiment 89, wherein the first unnatural base (X) located in the codon of the mRNA and the second unnatural base (Y) located in the anticodon of the tRNA are the same.

### Embodiment 92

The method of embodiment 89, wherein the first unnatural base (X) located in the codon of the mRNA and the second unnatural base (Y) located in the anticodon of the tRNA are different.

### Embodiment 93

The method of any one of embodiments 89 to 92, wherein the first unnatural base (X) located in the codon of the mRNA and the second unnatural base (Y) located in the anticodon of the tRNA are selected from the group consisting of

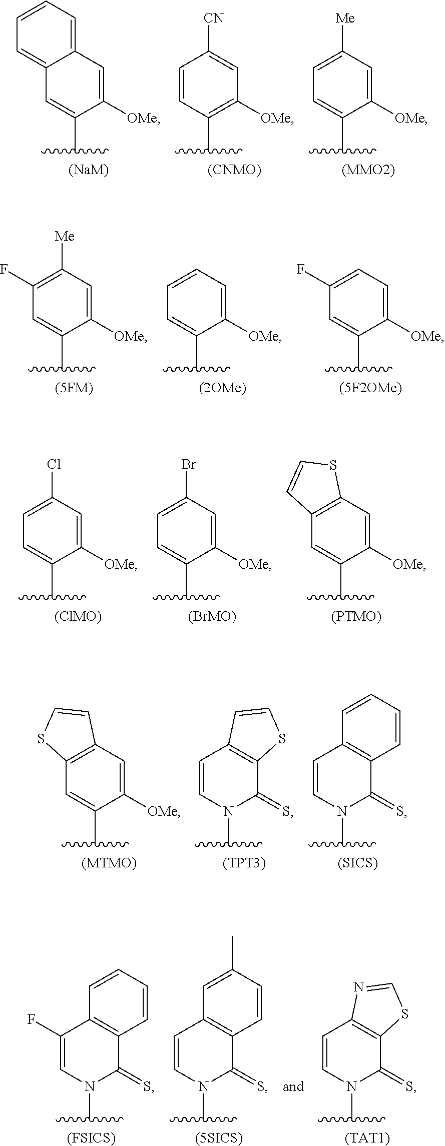

wherein the wavy line indicates a bond to a ribosyl moiety.

### Embodiment 94

The method of embodiment 93, wherein the first unnatural base (X) located in the codon of the mRNA and the second unnatural base (Y) located in the anticodon of the tRNA are selected from the group consisting of

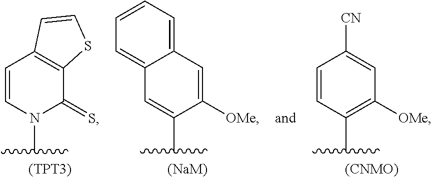

wherein the wavy line indicates a bond to a ribosyl moiety.

### Embodiment 95

The method of embodiment 94, wherein the first unnatural base (X) located in the codon of the mRNA and the second unnatural base (Y) located in the anticodon of the tRNA are both

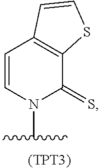

wherein the wavy line indicates a bond to a ribosyl moiety.

### Embodiment 96

The method of embodiment 94, wherein the first unnatural base (X) located in the codon of the mRNA and the second unnatural base (Y) located in the anticodon of the tRNA are both

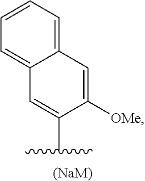

wherein the wavy line indicates a bond to a ribosyl moiety.

### Embodiment 97

The method of embodiment 94, wherein the first unnatural base (X) located in the codon of the mRNA and the second unnatural base (Y) located in the anticodon of the tRNA are both

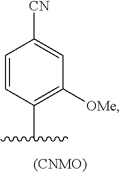 [SMILES: *c1ccc(C#N)cc1OC]

wherein the wavy line indicates a bond to a ribosyl moiety.

### Embodiment 98

The method of embodiment 94, wherein the first unnatural base (X) located in the codon of the mRNA is selected from

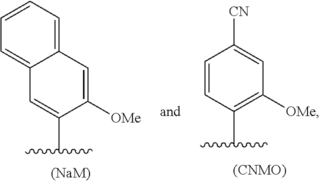

and the second unnatural base (Y) located in the anticodon of the tRNA is

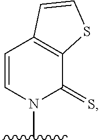 [SMILES: *n1ccc2ccsc2c1=S]

wherein in each case the wavy line indicates a bond to a ribosyl moiety.

### Embodiment 99

The method of embodiment 98, wherein the first unnatural base (X) located in the codon of the mRNA is

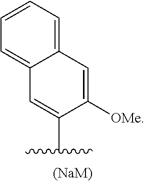

### Embodiment 100

The method of embodiment 98, wherein the first unnatural base (X) located in the codon of the mRNA is

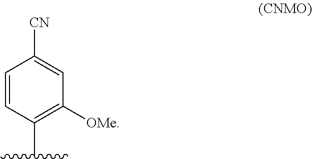 [SMILES: *c1ccc(C#N)cc1OC]

### Embodiment 101

The method of any one of embodiments 21, 23, 25 to 37, 42 to 45, 55 to 59, and 77 to 88, wherein the codon in the mRNA is selected from AXC, GXC or GXU, wherein X is the unnatural base.

### Embodiment 102

The method of embodiment 101, wherein the codon in the mRNA is AXC, wherein X is the unnatural base.

### Embodiment 103

The method of embodiment 101, wherein the codon in the mRNA is GXC, wherein X is the unnatural base.

### Embodiment 104

The method of embodiment 101, wherein the codon in the mRNA is GXU, wherein X is the unnatural base.

### Embodiment 105

The method of any one of embodiments 21, 23, 25 to 37, 42 to 45, 55 to 59, and 77 to 88, wherein the codon in the mRNA is selected from AXC, GXC or GXU, wherein the anticodon in the tRNA is selected from GYU, GYC, and AYC, wherein X is a first unnatural base and Y is a second unnatural base.

### Embodiment 106

The method of embodiment 105, wherein X and Y are the same or are different.

### Embodiment 107

The method of embodiment 106, wherein X and Y are the same.

### Embodiment 108

The method of embodiment 106, wherein X and Y are different.

### Embodiment 109

The method of embodiment 105, wherein the codon in the mRNA is AXC and the anticodon in the tRNA is GYU.

### Embodiment 110

The method of embodiment 109, wherein X and Y are the same or are different.

### Embodiment 111

The method of embodiment 109, wherein X and Y are the same.

### Embodiment 112

The method of embodiment 109, wherein X and Y are different.

### Embodiment 113

The method of embodiment 106, wherein the codon in the mRNA is GXC and the anticodon in the tRNA is GYC.

### Embodiment 114

The method of embodiment 113, wherein X and Y are the same or are different.

### Embodiment 115

The method of embodiment 113, wherein X and Y are the same.

### Embodiment 116

The method of embodiment 113, wherein X and Y are different.

### Embodiment 117

The method of embodiment 106, wherein the codon in the mRNA is GXU and the anticodon is AYC.

### Embodiment 118

The method of embodiment 117, wherein X and Y are the same or are different.

### Embodiment 119

The method of embodiment 117, wherein X and Y are the same.

### Embodiment 120

The method of embodiment 117, wherein X and Y are different.

### Embodiment 121

The method of any one of embodiments 21 to 120, wherein the tRNA is derived from Methanococcus jannaschii, Methanosarcina barkeri, Methanosarcina mazei, or Methanosarcina acetivorans.

### Embodiment 122

The method of any one of embodiments 21 to 120, wherein the tRNA synthetase is derived from Methanococcus jannaschii, Methanosarcina barkeri, Methanosarcina mazei, or Methanosarcina acetivorans.

### Embodiment 123

The method of embodiment 122, wherein tRNA and tRNA synthetase are derived from Methanococcus jannaschii.

### Embodiment 124

The method of embodiment 122, wherein tRNA and tRNA synthetase are derived from Methanosarcina barkeri.

### Embodiment 125

The method of embodiment 122, wherein tRNA and tRNA synthetase are derived from Methanosarcina mazei.

### Embodiment 126

The method of embodiment 122, wherein tRNA and tRNA synthetase are derived from Methanosarcina acetivorans.

### Embodiment 127

The method of any one of embodiments 21 to 120, wherein the tRNA is derived from Methanococcus jannaschii and tRNA synthetase is derived from Methanosarcina barkeri, Methanosarcina mazei, or Methanosarcina acetivorans.

### Embodiment 128

The method of any one of embodiments 21 to 120, wherein the tRNA is derived from Methanosarcina barkeri and tRNA synthetase is derived from Methanococcus jannaschii, Methanosarcina mazei, or Methanosarcina acetivorans.

### Embodiment 129

The method of any one of embodiments 21 to 120, wherein the tRNA is derived from Methanosarcina mazei and tRNA synthetase is derived from Methanococcus jannaschii. Methanosarcina barkeri, or Methanosarcina acetivorans.

### Embodiment 130

The method of any one of embodiments 21 to 120, wherein the tRNA is derived from Methanosarcina acetivorans and tRNA synthetase is derived from Methanococcus jannaschii, Methanosarcina barkeri, or Methanosarcina mazei.

### Embodiment 131

The method of any one of embodiments 21 to 120, wherein the tRNA is derived from Methanosarcina mazei and tRNA synthetase is derived from Methanosarcina barkeri.

### Embodiment 132

The method of any one of embodiments 21 to 120, wherein the cell is a human cell.

### Embodiment 133

The method of embodiment 132, wherein the human cell is a HEK293T cell.

### Embodiment 134

The method of any one of embodiments 21 to 120, wherein the cell is a hamster cell.

### Embodiment 135

The method of embodiment 134, wherein the hamster cell is a Chinese hamster ovary (CHO) cell.

### Embodiment 136

The method of any one of embodiments 21 to 135, wherein the unnatural amino acid:
is a lysine analogue;
comprises an aromatic side chain;
comprises an azido group;
comprises an alkyne group; or
comprises an aldehyde or ketone group.

### Embodiment 137

The method of any one of embodiments 21 to 135, wherein the unnatural amino acid is selected from the group consisting of N6-((azidoethoxy)-carbonyl)-L-lysine (AzK), N6-((propargylethoxy)-carbonyl)-L-lysine (PraK), BCN-L-lysine, norbomene lysine, TCO-lysine, methyltetrazine lysine, allyloxycarbonyllysine, 2-amino-8-oxononanoic acid, 2-amino-8-oxooctanoic acid, p-acetyl-L-phenylalanine, p-azidomethyl-L-phenylalanine (pAMF), p-iodo-L-phenylalanine, m-acetylphenylalanine, 2-amino-8-oxononanoic acid, p-propargyloxyphenylalanine, p-propargyl-phenylalanine, 3-methyl-phenylalanine, L-Dopa, fluorinated phenylalanine, isopropyl-L-phenylalanine, p-azido-L-phenylalanine, p-acyl-L-phenylalanine, p-benzoyl-L-phenylalanine, p-bromophenylalanine, p-amino-L-phenylalanine, isopropyl-L-phenylalanine, O-allyltyrosine, O-methyl-L-tyrosine, O-4-allyl-L-tyrosine, 4-propyl-L-tyrosine, phosphonotyrosine, tri-O-acetyl-GlcNAcp-serine, L-phosphoserine, phosphonoserine, L-3-(2-naphthyl)alanine, 2-amino-3-((2-((3-(benzyloxy)-3-oxopropyl)amino)ethyl)selanyl)propanoic acid, 2-amino-3-(phenylselanyl)propanoic, selenocysteine, N6-(((2-azidobenzyl)oxy)carbonyl)-L-lysine, N6-(((3-azidobenzyl)oxy)carbonyl)-L-lysine, or N6-(((4-azidobenzyl)oxy)carbonyl)-L-lysine.

### Embodiment 138

The method of embodiment 137, wherein the unnatural amino acid is N6-((azidoethoxy)-carbonyl)-L-lysine (AzK).

### Embodiment 139

A system for expression of an unnatural polypeptide in a eukaryotic cell comprising:
(a) at least one unnatural amino acid;
(b) an mRNA encoding the unnatural polypeptide, said mRNA comprising at least one codon comprising one or more first unnatural bases;
(c) a tRNA comprising at least one anti-codon comprising one or more second unnatural bases wherein the one or more first unnatural bases and the one or more second unnatural bases form one or more complementary base pairs;
(d) one or more nucleic acid constructs comprising a nucleic acid sequence encoding a tRNA synthetase, wherein the tRNA synthetase preferentially aminoacylates the tRNA with the at least one unnatural amino acid; and
(e) a eukaryotic cell capable of translating the mRNA into a polypeptide comprising the unnatural amino acid using the tRNA and tRNA synthetase.

### Embodiment 140

The system of embodiment 139, wherein the at least one codon of the mRNA comprises three contiguous nucleobases (N—N—N); and wherein the one or more first unnatural bases (X) is located at the first position (X—N—N) in the at least one codon of the mRNA.

### Embodiment 141

The system of embodiment 139, wherein the at least one codon of the mRNA comprises three contiguous nucleobases (N—N—N); and wherein the one or more first unnatural bases (X) is located at the middle position (N—X—N) in the codon of the mRNA.

### Embodiment 142

The system of embodiment 139, wherein the at least one codon of the mRNA comprises three contiguous nucleobases (N—N—N); and wherein the one or more first unnatural bases (X) is located at the last position (N—N—X) in the at least one codon of the mRNA.

### Embodiment 143

The system of any one of embodiments 139 to 142, wherein the one or more unnatural bases is of the formula

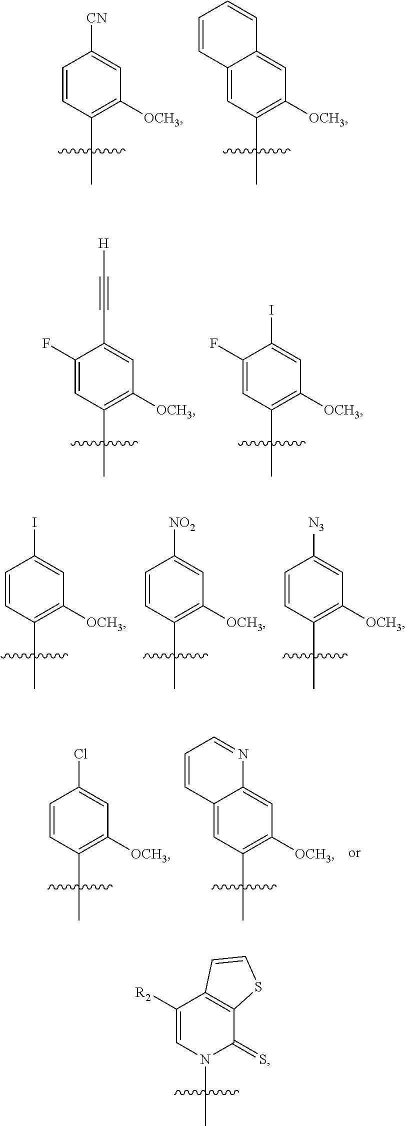

wherein R₂ is selected from the group consisting of hydrogen, alkyl, alkenyl, alkynyl, methoxy, methanethiol, methaneseleno, halogen, cyano, and azido, and the wavy line indicates a bond to a ribosyl moiety.

### Embodiment 144

The system of any one of embodiments 139 to 142, wherein the one or more first unnatural bases or the one or more second unnatural bases is selected from the group consisting of

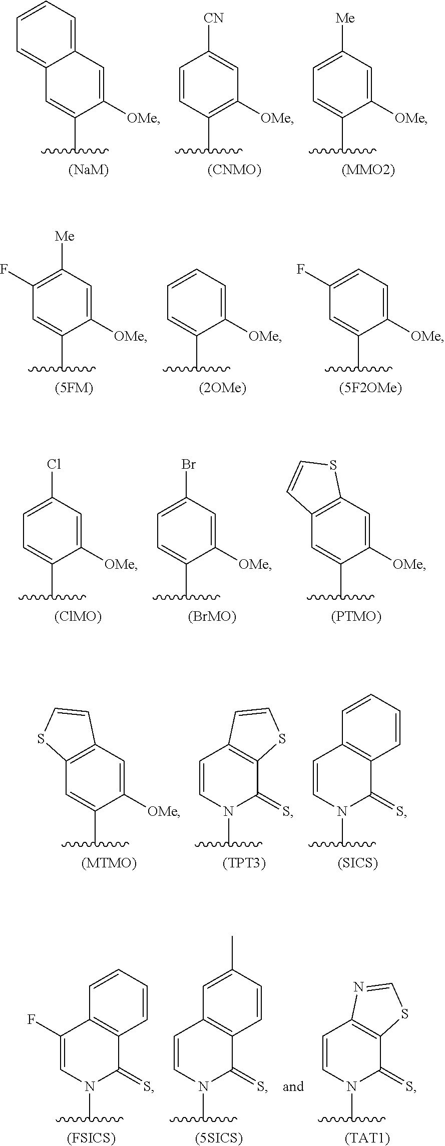

wherein the wavy line indicates a bond to a ribosyl moiety.

### Embodiment 145

The system of embodiment 144, when the one or more first unnatural bases is

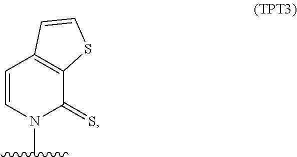 [SMILES: *n1ccc2ccsc2c1=S]

The one or more second unnatural bases is

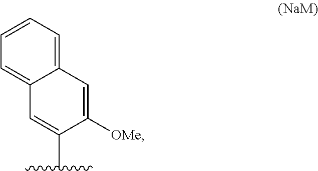 [SMILES: *c1cc2ccccc2cc1OC]

and when the one or more first unnatural bases is

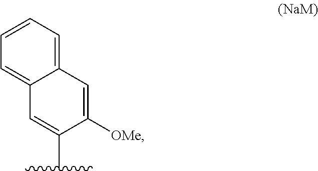 [SMILES: *c1cc2ccccc2cc1OC]

the second unnatural base is

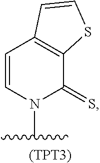

wherein the wavy line indicates a bond to a ribosyl moiety.

### Embodiment 146

The system of embodiment 144, when the one or more first unnatural bases is

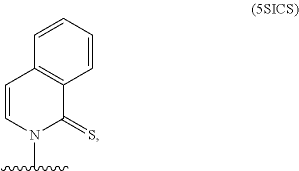 [SMILES: *n1ccc2ccccc2c1=S]

the one or more second unnatural bases is

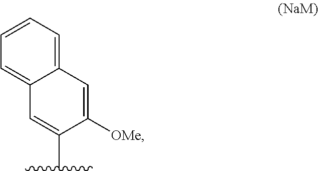 [SMILES: *c1cc2ccccc2cc1OC]

and when the one or more first unnatural base

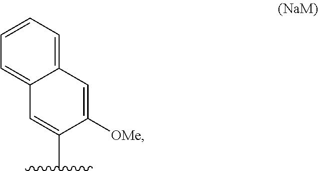 [SMILES: *c1cc2ccccc2cc1OC]

the one or more second unnatural bases is

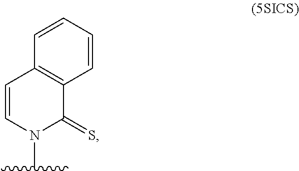 [SMILES: *n1ccc2ccccc2c1=S]

wherein the wavy line indicates a bond to a ribosyl moiety.

### Embodiment 147

The system of embodiment 144, when the one or more first unnatural bases is

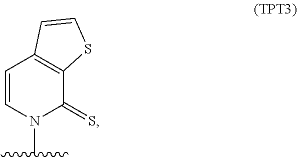 [SMILES: *n1ccc2ccsc2c1=S]

the one or more second unnatural bases is

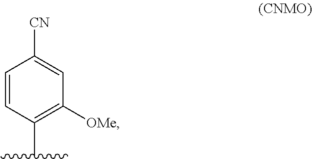 [SMILES: *c1ccc(C#N)cc1OC]

and when the one or more first unnatural bases is

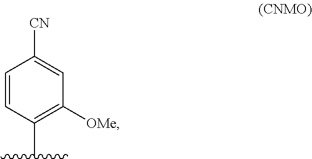 [SMILES: *c1ccc(C#N)cc1OC]

the one or more second unnatural bases is

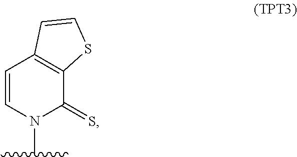 [SMILES: *n1ccc2ccsc2c1=S]

wherein the wavy line indicates a bond to a ribosyl moiety.

### Embodiment 148

The system of embodiment 144, when the one or more first unnatural bases is

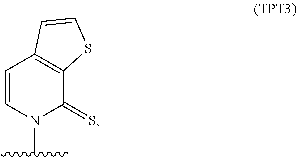 [SMILES: *n1ccc2ccsc2c1=S]

the one or more second unnatural bases is

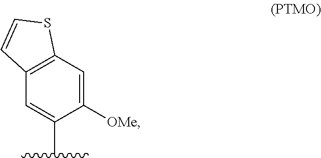 [SMILES: *c1cc2ccsc2cc1OC]

and when the one or more first unnatural bases is

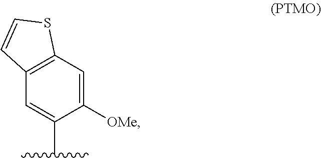 [SMILES: *c1cc2ccsc2cc1OC]

the one or more second unnatural bases is

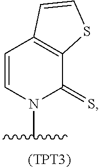

wherein the wavy line indicates a bond to a ribosyl moiety.

### Embodiment 149

The system of embodiment 144, when the one or more first unnatural bases is

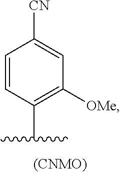 [SMILES: *c1ccc(C#N)cc1OC]

the one or more second unnatural bases is

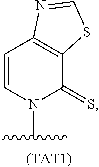

and when the one or more first unnatural bases is

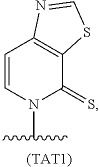

the one or more second unnatural bases is

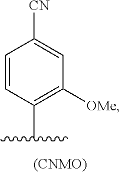 [SMILES: *c1ccc(C#N)cc1OC]

wherein the wavy line indicates a bond to a ribosyl moiety.

### Embodiment 150

The system of embodiment 144, when the one or more first unnatural bases is

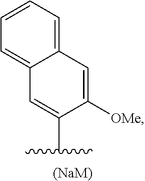

the one or more second unnatural bases is

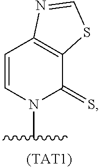

and when the one or more first unnatural bases is

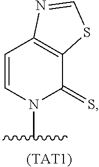

the one or more second unnatural bases is

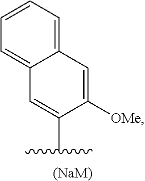

wherein the wavy line indicates a bond to a ribosyl moiety.

### Embodiment 151

The system of embodiment 144, when the one or more first unnatural bases is

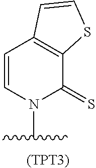

the one or more second unnatural bases is

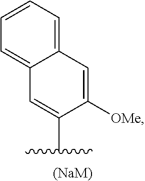

wherein the wavy line indicates a bond to a ribosyl moiety.

### Embodiment 152

The system of any one of embodiments 139 to 142, wherein the one or more first unnatural bases is selected from

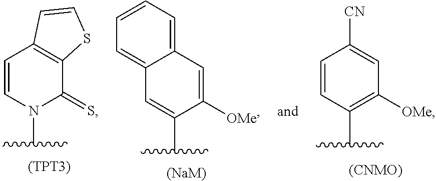

wherein the wavy line indicates a bond to a ribosyl moiety.

### Embodiment 153

The system of embodiment 152, wherein the one or more first unnatural bases

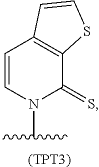

wherein the wavy line indicates a bond to a ribosyl moiety.

### Embodiment 154

The system of embodiment 152, wherein the one or more first unnatural bases is

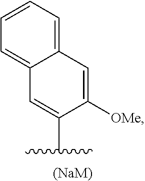

wherein the wavy line indicates a bond to a ribosyl moiety.

### Embodiment 155

The system of embodiment 152, wherein the one or more first unnatural bases

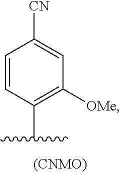 [SMILES: *c1ccc(C#N)cc1OC]

wherein the wavy line indicates a bond to a ribosyl moiety.

### Embodiment 156

The system of embodiment 139, wherein the at least one codon of the mRNA comprises three contiguous nucleobases (N—N—N), wherein the one or more first unnatural bases (X) is located at the first position (X—N—N) in the codon of the mRNA, wherein the one or more first unnatural bases is selected from

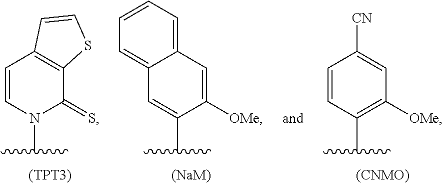

and wherein the wavy line indicates a bond to a ribosyl moiety.

### Embodiment 157

The system of embodiment 156, wherein the one or more first unnatural bases is

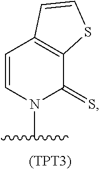

wherein the wavy line indicates a bond to a ribosyl moiety.

### Embodiment 158

The stem of embodiment 156, wherein the one or more first unnatural bases is

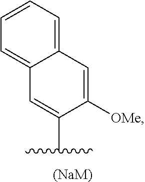

wherein the wavy line indicates a bond to a ribosyl moiety.

### Embodiment 159

The stem of embodiment 156, wherein the one or more first unnatural base is

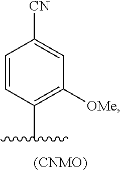

wherein the wavy line indicates a bond to a ribosyl moiety.

### Embodiment 160

The system of embodiment 139, wherein the at least one codon of the mRNA comprises three contiguous nucleobases (N—N—N), wherein the one or more first unnatural bases (X) is located at the middle position (N—X—N) in the codon of the mRNA, wherein the one or more first unnatural bases is selected from

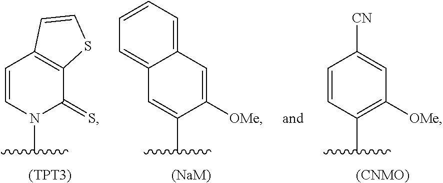

and wherein the wavy line indicates a bond to a ribosyl moiety.

### Embodiment 161

The system of embodiment 160, wherein the one or more first unnatural bases is

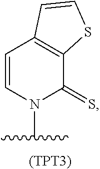

wherein the wavy line indicates a bond to a ribosyl moiety.

### Embodiment 162

The system of embodiment 160, wherein the one or more first unnatural bases is

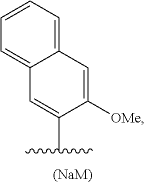

wherein the wavy line indicates a bond to a ribosyl moiety.

### Embodiment 163

The system of embodiment 160, wherein the one or more first unnatural base is

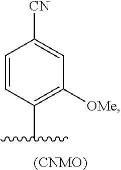

wherein the wavy line indicates a bond to a ribosyl moiety.

### Embodiment 164

The system of embodiment 139, wherein the at least one codon of the mRNA comprises three contiguous nucleobases (N—N—N), wherein the one or more first unnatural base (X) is located at the last position (N—N—X) in the codon of the mRNA, wherein the one or more first unnatural base is selected from

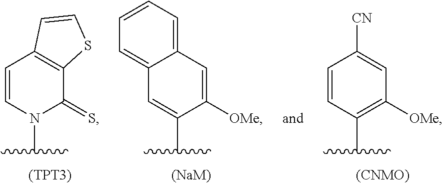

and wherein the wavy line indicates a bond to a ribosyl moiety.

### Embodiment 165

The system of embodiment 164, wherein the one or more first unnatural base is

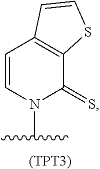

wherein the wavy line indicates a bond to a ribosyl moiety.

### Embodiment 166

The stem of embodiment 164, wherein the one or more first unnatural bases is

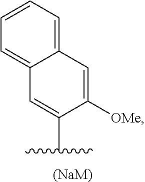

wherein the wavy line indicates a bond to a ribosyl moiety.

### Embodiment 167

The system of embodiment 164, wherein the one or more first unnatural bases is

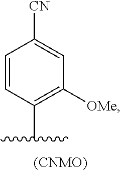

wherein the wavy line indicates a bond to a ribosyl moiety.

### Embodiment 168

The system of embodiment 139, wherein the at least one anticodon of the tRNA comprises three contiguous nucleobases (N—N—N); and wherein the one or more second unnatural base (X) is located at the first position (X—N—N) in the anticodon of the tRNA.

### Embodiment 169

The system of embodiment 168, wherein the one or more second unnatural bases is selected from

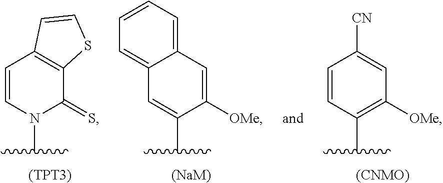

and wherein the wavy line indicates a bond to a ribosyl moiety.

### Embodiment 170

The system of embodiment 168, wherein the one or more second unnatural base is

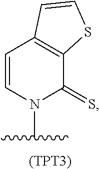

wherein the wavy line indicates a bond to a ribosyl moiety.

### Embodiment 171

The system of embodiment 168, wherein the one or more second unnatural bases is

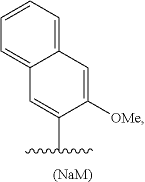

wherein the wavy line indicates a bond to a ribosyl moiety.

### Embodiment 172

The system of embodiment 168, wherein the one or more second unnatural bases is

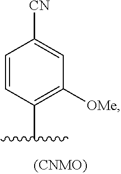 [SMILES: *c1ccc(C#N)cc1OC]

wherein the wavy line indicates a bond to a ribosyl moiety.

### Embodiment 173

The system of embodiment 139, wherein the at least one anticodon of the tRNA comprises three contiguous nucleobases (N—N—N); and wherein the one or more second unnatural bases (X) is located at the middle position (N—X—N) in the anticodon of the tRNA.

### Embodiment 174

The system of embodiment 173, wherein the one or more second unnatural bases is selected from

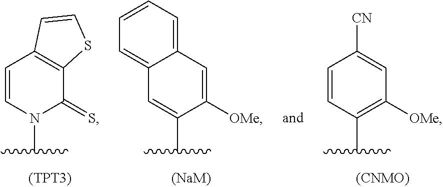

and wherein the wavy line indicates a bond to a ribosyl moiety.

### Embodiment 175

The system of embodiment 173, wherein the one or more second unnatural bases is

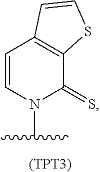

wherein the wavy line indicates a bond to a ribosyl moiety.

### Embodiment 176

The system of embodiment 173, wherein the one or more second unnatural bases is

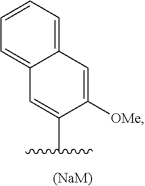

wherein the wavy line indicates a bond to a ribosyl moiety.

### Embodiment 177

The stem of embodiment 173, wherein the one or more second unnatural bases is

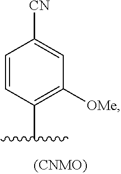 [SMILES: *c1ccc(C#N)cc1OC]

wherein the wavy line indicates a bond to a ribosyl moiety.

### Embodiment 178

The system of embodiment 139, wherein the at least one anticodon of the tRNA comprises three contiguous nucleobases (N—N—N); and wherein the one or more second unnatural bases (X) is located at the last position (N—N—X) in the anticodon of the tRNA.

### Embodiment 179

The system of embodiment 178, wherein the one or more second unnatural base is selected from

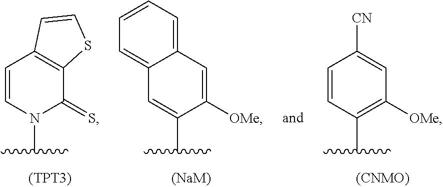

and wherein the wavy line indicates a bond to a ribosyl moiety.

### Embodiment 180

The system of embodiment 178, wherein the one or more second unnatural bases is

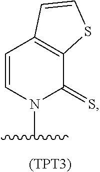

wherein the wavy line indicates a bond to a ribosyl moiety.

### Embodiment 181

The system of embodiment 178, wherein the one or more second unnatural bases is

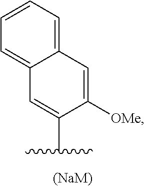

wherein the wavy line indicates a bond to a ribosyl moiety.

### Embodiment 182

The system of embodiment 178, wherein the one or more second unnatural bases is

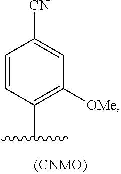 [SMILES: *c1ccc(C#N)cc1OC]

wherein the wavy line indicates a bond to a ribosyl moiety.

### Embodiment 183

The system of embodiment 139, wherein the at least one codon and the at least one anticodon each, independently, comprise three contiguous nucleobases (N—N—N), and wherein the at least one codon comprises one or more first unnatural bases (X) located at the first position (X—N—N) of the codon, and the at least one anticodons in the tRNA comprises the one or more second unnatural bases (Y) located at the last position (N—N—Y) of the anticodon.

### Embodiment 184

The system of embodiment 183, wherein the one or more first unnatural bases (X) located in the codon of the mRNA and the one or more second unnatural bases (Y) located in the anticodon of the tRNA are the same or are different.

### Embodiment 185

The system of embodiment 184, wherein the one or more first unnatural bases (X) located in the codon of the mRNA and the one or more second unnatural bases (Y) located in the anticodon of the tRNA are the same.

### Embodiment 186

The system of embodiment 184, wherein the one or more first unnatural bases (X) located in the codon of the mRNA and the one or more second unnatural bases (Y) located in the anticodon of the tRNA are different.

### Embodiment 187

The system of any one of embodiments 183 to 186, wherein the one or more first unnatural bases (X) located in the codon of the mRNA and the one or more second unnatural bases (Y) located in the anticodon of the tRNA are selected from the group consisting of

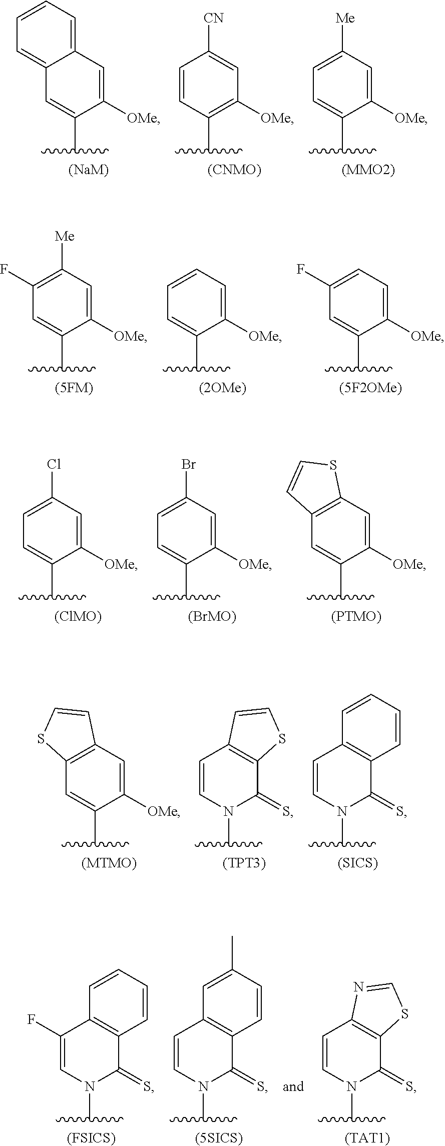

wherein the wavy line indicates a bond to a ribosyl moiety.

### Embodiment 188

The system of embodiment 187, wherein the one or more first unnatural bases (X) located in the codon of the mRNA and the one or more second unnatural bases (Y) located in the anticodon of the tRNA are selected from the group consisting of

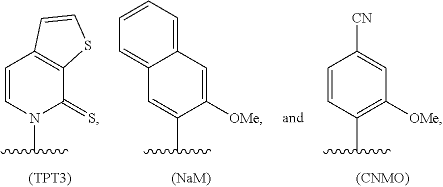

wherein the wavy line indicates a bond to a ribosyl moiety.

### Embodiment 189

The system of embodiment 188, wherein the one or more first unnatural bases (X) located in the codon of the mRNA and the one or more second unnatural bases (Y) located in the anticodon of the tRNA are both

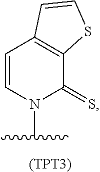

wherein the wavy line indicates a bond to a ribosyl moiety.

### Embodiment 190

The system of embodiment 188, wherein the one or more first unnatural bases (X) located in the codon of the mRNA and the one or more second unnatural bases (Y) located in the anticodon of the tRNA are both

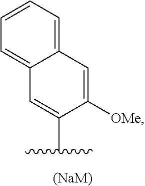

wherein the wavy line indicates a bond to a ribosyl moiety.

### Embodiment 191

The system of embodiment 188, wherein the one or more first unnatural bases (X) located in the codon of the mRNA and the one or more second unnatural bases (Y) located in the anticodon of the tRNA are both

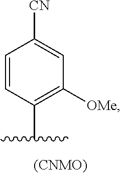 [SMILES: *c1ccc(C#N)cc1OC]

wherein the wavy line indicates a bond to a ribosyl moiety.

### Embodiment 192

The system of embodiment 188, wherein the one or more first unnatural base (X) located in the codon of the mRNA is selected from

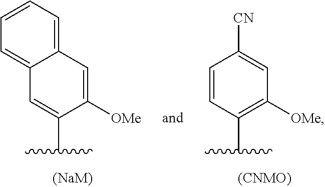

and the one or more second unnatural bases (Y) located in the anticodon of the tRNA is

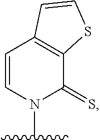 [SMILES: *n1ccc2ccsc2c1=S]

wherein in each case the wavy line indicates a bond to a ribosyl moiety.

### Embodiment 193

The system of embodiment 192, wherein the one or more first unnatural bases (X) located in the codon of the mRNA is

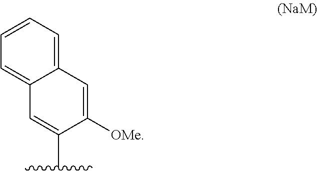 [SMILES: *c1cc2ccccc2cc1OC]

### Embodiment 194

The system of embodiment 192 wherein the ne or more first unnatural bases (X) located in the codon of the mRNA is

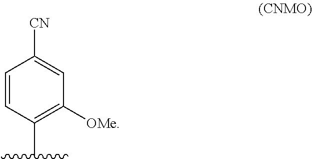 [SMILES: *c1ccc(C#N)cc1OC]

### Embodiment 195

The system of embodiment 139, wherein the at least one codon and the at least one anticodon each, independently, comprise three contiguous nucleobases (N—N—N), and wherein the at least one codon in the mRNA comprises the one or more first unnatural bases (X) located at a middle position (N—X—N) of the at least one codon, and the at least one anticodon in the tRNA comprises the one or more second unnatural bases (Y) located at a middle position (N—Y—N) of the anticodon.

### Embodiment 196

The system of embodiment 195, wherein the one or more first unnatural bases (X) located in the codon of the mRNA and the one or more second unnatural bases (Y) located in the anticodon of the tRNA are the same or are different.

### Embodiment 197

The system of embodiment 195, wherein the one or more first unnatural bases (X) located in the codon of the mRNA and the one or more second unnatural bases (Y) located in the anticodon of the tRNA are the same.

### Embodiment 198

The system of embodiment 195, wherein the one or more first unnatural bases (X) located in the codon of the mRNA and the one or more second unnatural bases (Y) located in the anticodon of the tRNA are different.

### Embodiment 199

The system of any one of embodiments 195 to 198, wherein the one or more first unnatural bases (X) located in the codon of the mRNA and the one or more second unnatural bases (Y) located in the anticodon of the tRNA are selected from the group consisting of

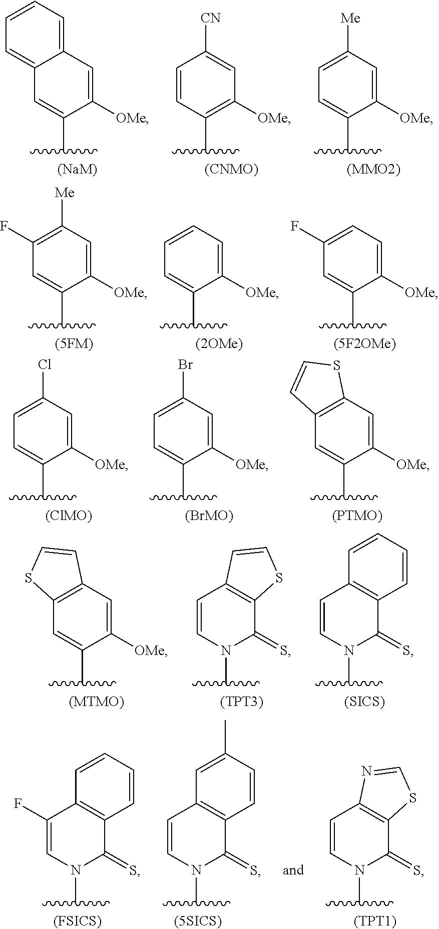

wherein the wavy line indicates a bond to a ribosyl moiety.

### Embodiment 200

The system of embodiment 199, wherein the one or more first unnatural bases (X) located in the codon of the mRNA and the one or more second unnatural bases (Y) located in the anticodon of the tRNA are selected from the group consisting of

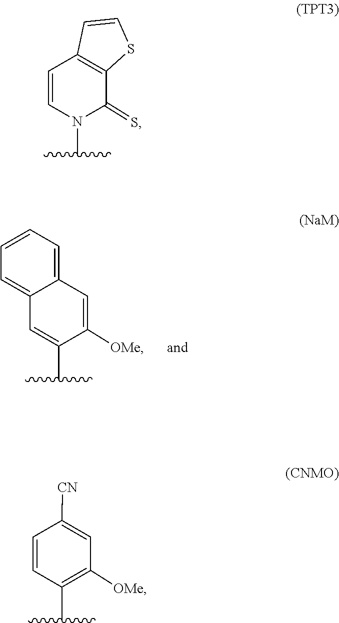 [SMILES: *n1ccc2ccsc2c1=S ; *c1cc2ccccc2cc1OC ; "and" ; *c1ccc(C#N)cc1OC]

wherein the wavy line indicates a bond to a ribosyl moiety.

### Embodiment 201

The system of embodiment 200, wherein the one or more first unnatural bases (X) located in the codon of the mRNA and the one or more second unnatural bases (Y) located in the anticodon of the tRNA are both

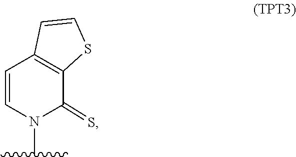 [SMILES: *n1ccc2ccsc2c1=S]

wherein the wavy line indicates a bond to a ribosyl moiety.

### Embodiment 202

The system of embodiment 200, wherein the one or more first unnatural bases (X) located in the codon of the mRNA and the one or more second unnatural bases (Y) located in the anticodon of the tRNA are both

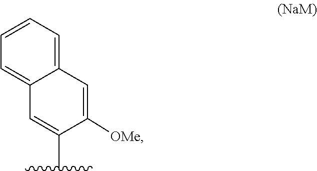 [SMILES: *c1cc2ccccc2cc1OC]

wherein the wavy line indicates a bond to a ribosyl moiety.

### Embodiment 203

The system of embodiment 200, wherein the one or more first unnatural bases (X) located in the codon of the mRNA and the one or more second unnatural bases (Y) located in the anticodon of the tRNA are both

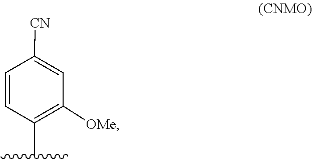 [SMILES: *c1ccc(C#N)cc1OC]

wherein the wavy line indicates a bond to a ribosyl moiety.

### Embodiment 204

The system of embodiment 200, wherein the one or more first unnatural bases located in the codon of the mRNA is selected from

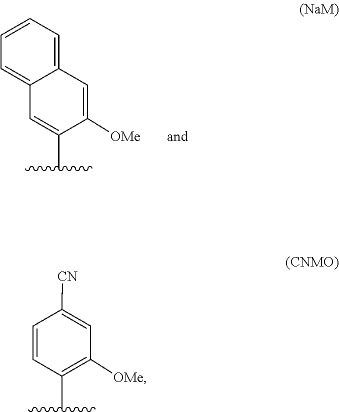 [SMILES: *c1cc2ccccc2cc1OC ; "and" ; *c1ccc(C#N)cc1OC]

(CNMO), and the one or more second unnatural bases (Y) located in the anticodon of the tRNA is

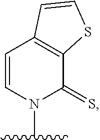 [SMILES: *n1ccc2ccsc2c1=S]

wherein in each case the wavy line indicates a bond to a ribosyl moiety.

### Embodiment 205

The system of embodiment 204, wherein the one or more first unnatural bases (X) located in the codon of the mRNA is

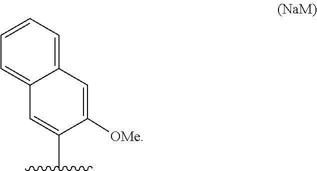 [SMILES: *c1cc2ccccc2cc1OC]

### Embodiment 206

The system of embodiment 204, wherein the one or more first unnatural bases (X) located in the codon of the mRNA is

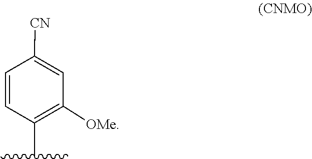 [SMILES: *c1ccc(C#N)cc1OC]

### Embodiment 207

The system of embodiment 139, wherein the at least one codon and the at least one anticodon each, independently, comprise three contiguous nucleobases (N—N—N), and wherein the at least one codon in the mRNA comprises the one or more first unnatural bases (X) located at the last position (N—N—X) of the at least one codon, and the at least one anticodon in the tRNA comprises the one or more second unnatural bases (Y) located at the first position (Y—N—N) of the anticodon.

### Embodiment 208

The system of embodiment 207, wherein the one or more first unnatural bases (X) located in the codon of the mRNA and the one or more second unnatural bases (Y) located in the anticodon of the tRNA are the same or are different.

### Embodiment 209

The system of embodiment 208, wherein the one or more first unnatural bases (X) located in the codon of the mRNA and the one or more second unnatural bases (Y) located in the anticodon of the tRNA are the same.

### Embodiment 210

The system of embodiment 208, wherein the one or more first unnatural bases (X) located in the codon of the mRNA and the one or more second unnatural bases (Y) located in the anticodon of the tRNA are different.

### Embodiment 211

The system of any one of embodiments 207 to 210, wherein the one or more first unnatural bases (X) located in the codon of the miRNA and the one or more second unnatural bases (Y) located in the anticodon of the tRNA are selected from the group consisting of

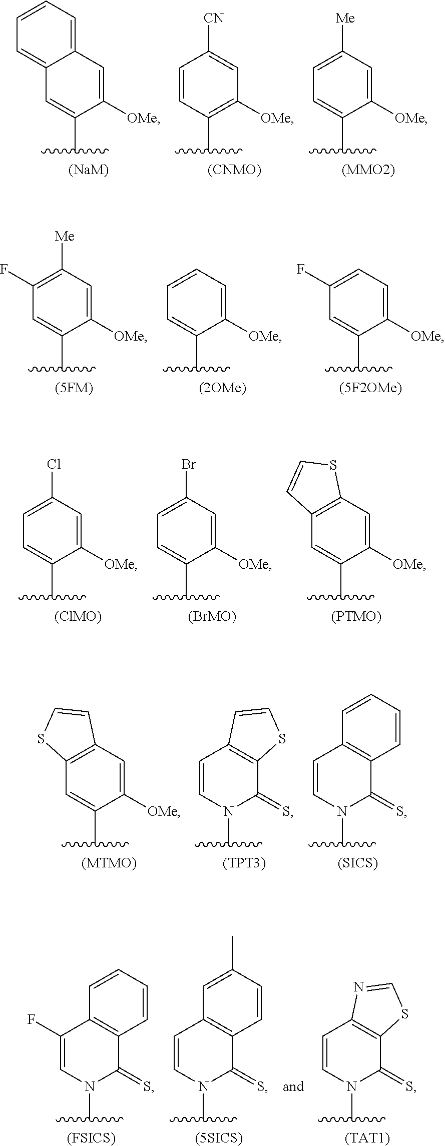

wherein the wavy line indicates a bond to a ribosyl moiety.

### Embodiment 212

The system of embodiment 211, wherein the one or more first unnatural bases (X) located in the codon of the mRNA and the one or more second unnatural bases (Y) located in the anticodon of the tRNA are selected from the group consisting of

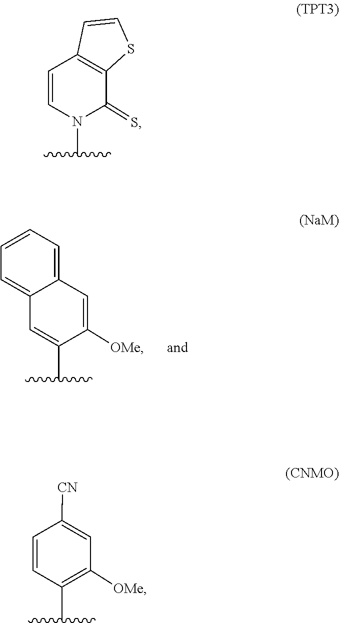 [SMILES: *n1ccc2ccsc2c1=S ; *c1cc2ccccc2cc1OC ; "and" ; *c1ccc(C#N)cc1OC]

wherein the wavy line indicates a bond to a ribosyl moiety.

### Embodiment 213

The system of embodiment 212, wherein the one or more first unnatural bases (X) located in the codon of the mRNA and the one or more second unnatural bases (Y) located in the anticodon of the tRNA are both

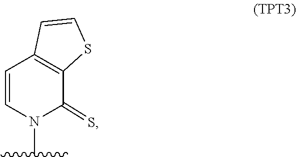 [SMILES: *n1ccc2ccsc2c1=S]

wherein the wavy line indicates a bond to a ribosyl moiety.

### Embodiment 214

The system of embodiment 212, wherein the one or more first unnatural bases (X) located in the codon of the mRNA and the one or more second unnatural bases (Y) located in the anticodon of the tRNA are both

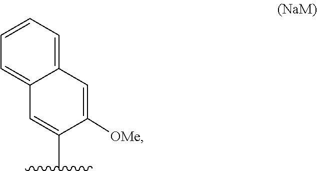 [SMILES: *c1cc2ccccc2cc1OC]

wherein the wavy line indicates a bond to a ribosyl moiety.

### Embodiment 215

The system of embodiment 212, wherein the one or more first unnatural bases (X) located in the codon of the mRNA and the one or more second unnatural bases (Y) located in the anticodon of the tRNA are both

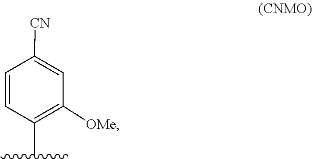 [SMILES: *c1ccc(C#N)cc1OC]

wherein the wavy line indicates a bond to a ribosyl moiety.

### Embodiment 216

The system of embodiment 212, wherein the one or more first unnatural bases located in the codon of the mRNA is selected from

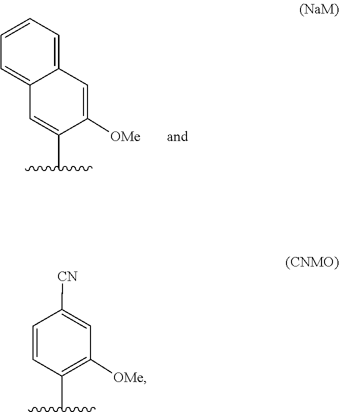 [SMILES: *c1cc2ccccc2cc1OC ; "and" ; *c1ccc(C#N)cc1OC]

(CNMO), and the one or more second unnatural bases (Y) located in the anticodon of the tRNA is

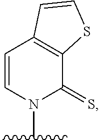 [SMILES: *n1ccc2ccsc2c1=S]

wherein in each case the wavy line indicates a bond to a ribosyl moiety.

### Embodiment 217

The system of embodiment 216, wherein the one or more first unnatural bases (X) located in the codon of the mRNA is

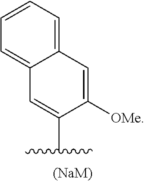

### Embodiment 218

The system of embodiment 216, wherein the one or more first unnatural bases (X) located in the codon of the mRNA is

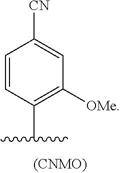 [SMILES: *c1ccc(C#N)cc1OC]

### Embodiment 219

The system of any one of embodiments 139 to 218, wherein the at least one codon in the mRNA is selected from AXC, GXC or GXU, wherein X is the unnatural base.

### Embodiment 220

The system of embodiment 219, wherein the at least one codon in the mRNA is AXC, wherein X is the unnatural base.

### Embodiment 221

The system of embodiment 219, wherein the at least one codon in the mRNA is GXC, wherein X is the unnatural base.

### Embodiment 222

The system of embodiment 219, wherein the at least one codon in the mRNA is GXU, wherein X is the unnatural base.

### Embodiment 223

The system of any one of embodiments 139 to 218, wherein the at least one codon in the mRNA is selected from AXC, GXC or GXU, wherein the at least one anticodon in the tRNA is selected from GYU, GYC, and AYC, wherein X is the one or more first unnatural bases and Y is the one or more second unnatural bases.

### Embodiment 224

The system of embodiment 223, wherein X and Y are the same or are different.

### Embodiment 225

The system of embodiment 224, wherein X and Y are the same.

### Embodiment 226

The system of embodiment 224, wherein X and Y are different.

### Embodiment 227

The system of embodiment 223, wherein the at least one codon in the mRNA is AXC and the at least one anticodon in the tRNA is GYU.

### Embodiment 228

The system of embodiment 227, wherein X and Y are the same or are different.

### Embodiment 229

The system of embodiment 228, wherein X and Y are the same.

### Embodiment 230

The system of embodiment 228, wherein X and Y are different.

### Embodiment 231

The system of embodiment 223, wherein the at least one codon in the mRNA is GXC and the at least one anticodon in the tRNA is GYC.

### Embodiment 232

The system of embodiment 231, wherein X and Y are the same or are different.

### Embodiment 233

The system of embodiment 232, wherein X and Y are the same.

### Embodiment 234

The system of embodiment 232, wherein X and Y are different.

### Embodiment 235

The system of embodiment 223, wherein the at least one codon in the mRNA is GXU and the at least one anticodon is AYC.

### Embodiment 236

The system of embodiment 235, wherein X and Y are the same or are different.

### Embodiment 237

The system of embodiment 236, wherein X and Y are the same.

### Embodiment 238

The system of embodiment 236, wherein X and Y are different.

### Embodiment 239

The system of any one of embodiments 139 to 238, wherein the tRNA is derived from Methanococcus jannaschii, Methanosarcina barkeri, Methanosarcina mazei, or Methanosarcina acetivorans.

### Embodiment 240

The system of any one of embodiments 139 to 238 wherein the tRNA synthetase is derived from Methanococcus jannaschii, Methanosarcina barkeri, Methanosarcina mazei, or Methanosarcina acetivorans.

### Embodiment 241

The system of embodiment 240, wherein tRNA and tRNA synthetase are derived from Methanococcus jannaschii.

### Embodiment 242

The system of embodiment 240, wherein tRNA and tRNA synthetase are derived from Methanosarcina barkeri.

### Embodiment 243

The system of embodiment 240, wherein tRNA and tRNA synthetase are derived from Methanosarcina mazei.

### Embodiment 244

The system of embodiment 240, wherein tRNA and tRNA synthetase are derived from Methanosarcina acetivorans.

### Embodiment 245

The system of any one of embodiments 139 to 239, wherein the tRNA is derived from Methanococcus jannaschii and tRNA synthetase is derived from Methanosarcina barkeri, Methanosarcina mazei, or Methanosarcina acetivorans.

### Embodiment 246

The system of any one of embodiments 139 to 239, wherein the tRNA is derived from Methanosarcina barkeri and tRNA synthetase is derived from Methanococcus jannaschii, Methanosarcina mazei, or Methanosarcina acetivorans.

### Embodiment 247

The system of any one of embodiments 139 to 239, wherein the tRNA is derived from Methanosarcina mazei and tRNA synthetase is derived from Methanococcus jannaschii. Methanosarcina barkeri, or Methanosarcina acetivorans.

### Embodiment 248

The system of any one of embodiments 139 to 239, wherein the tRNA is derived from Methanosarcina acetivorans and tRNA synthetase is derived from Methanococcus jannaschii, Methanosarcina barkeri, or Methanosarcina mazei.

### Embodiment 249

The system of any one of embodiments 139 to 239, wherein the tRNA is derived from Methanosarcina mazei and tRNA synthetase is derived from Methanosarcina barkeri.

### Embodiment 250

The system of any one of embodiments 139 to 249, wherein the cell is a human cell.

### Embodiment 251

The system of embodiment 250, wherein the human cell is a HEK293T cell.

### Embodiment 252

The system of any one of embodiments 139 to 239, wherein the cell is a hamster cell.

### Embodiment 253

The system of embodiment 252, wherein the hamster cell is a Chinese hamster ovary (CHO) cell.

### Embodiment 254

The system of any one of embodiments 139 to 253, wherein the unnatural amino acid:
is a lysine analogue;
comprises an aromatic side chain;
comprises an azido group;
comprises an alkyne group; or
comprises an aldehyde or ketone group.

### Embodiment 255

The system of any one of embodiments 139 to 253, wherein the unnatural amino acid is selected from the group consisting of N6-((azidoethoxy)-carbonyl)-L-lysine (AzK), N6-((propargylethoxy)-carbonyl)-L-lysine (PraK), BCN-L-lysine, norbomene lysine, TCO-lysine, methyltetrazine lysine, allyloxycarbonyllysine, 2-amino-8-oxononanoic acid, 2-amino-8-oxooctanoic acid, p-acetyl-L-phenylalanine, p-azidomethyl-L-phenylalanine (pAMF), p-iodo-L-phenylalanine, m-acetylphenylalanine, 2-amino-8-oxononanoic acid, p-propargyloxyphenylalanine, p-propargyl-phenylalanine, 3-methyl-phenylalanine, L-Dopa, fluorinated phenylalanine, isopropyl-L-phenylalanine, p-azido-L-phenylalanine, p-acyl-L-phenylalanine, p-benzoyl-L-phenylalanine, p-bromophenylalanine, p-amino-L-phenylalanine, isopropyl-L-phenylalanine, O-allyltyrosine, O-methyl-L-tyrosine, O-4-allyl-L-tyrosine, 4-propyl-L-tyrosine, phosphonotyrosine, tri-O-acetyl-GlcNAcp-serine, L-phosphoserine, phosphonoserine, L-3-(2-naphthyl)alanine, 2-amino-3-((2-((3-(benzyloxy)-3-oxopropyl)amino)ethyl)selanyl)propanoic acid, 2-amino-3-(phenylselanyl)propanoic, selenocysteine, N6-(((2-azidobenzyl)oxy)carbonyl)-L-lysine, N6-(((3-azidobenzyl)oxy)carbonyl)-L-lysine, or N6-(((4-azidobenzyl)oxy)carbonyl)-L-lysine.

### Embodiment 256

The system of embodiment 255, wherein the unnatural amino acid is N6-((azidoethoxy)-carbonyl)-L-lysine (AzK).

### Embodiment 257

The method of any one of embodiments 21 to 138, wherein the mRNA and the tRNA are stabilized to degradation in the eukaryotic cell.

### Embodiment 258

The method of any one of embodiments 21 to 138 and 257, wherein the polypeptide is produced by translation of the mRNA using the tRNA by a ribosome that is endogenous to the eukaryotic cell.

### Embodiment 259

The system of any one of embodiments 139 to 256, wherein the mRNA and the tRNA are stabilized to degradation in the eukaryotic cell.

### Embodiment 260

The system of any one of 139 to 256 and 259, wherein polypeptide is produced by translation of the mRNA using the tRNA by a ribosome that is endogenous to the eukaryotic cell.

### Embodiment 261

A eukaryotic cell comprising:
(a) a messenger RNA (mRNA) with a codon comprising a first unnatural base; and
(b) a transfer RNA (tRNA) with an anticodon comprising a second unnatural base, wherein the first and second unnatural bases are capable of forming an unnatural base pair (UBP) in the eukaryotic cell, and wherein the mRNA is capable of being translated in the cell to produce a polypeptide comprising at least one unnatural amino acid.

### Embodiment 262

The eukaryotic cell of embodiment 261, wherein the tRNA is charged with an unnatural amino acid.

### Embodiment 263

The eukaryotic cell of any one of embodiments 261-262, further comprising a polypeptide translated from the mRNA, wherein the polypeptide comprises the unnatural amino acid, optionally wherein the polypeptide comprises a eukaryotic glycosylation pattern.

### Embodiment 264

The eukaryotic cell of any one of embodiments 261-263, further comprising a tRNA synthetase, wherein the tRNA synthetase preferentially aminoacylates the tRNA with the unnatural amino acid.

### Embodiment 265

The eukaryotic cell of any one of embodiments 261-264, wherein the codon of the mRNA comprises three contiguous nucleobases (N—N—N); and wherein the first unnatural base (X) is located at the first position (X—N—N) in the codon of the mRNA.

### Embodiment 266

The eukaryotic cell of any one of embodiments 261-265, wherein the codon of the mRNA comprises three contiguous nucleobases (N—N—N); and wherein the first unnatural base (X) is located at the middle position (N—X—N) in the codon of the mRNA.

### Embodiment 267

The eukaryotic cell of any one of embodiments 261-266, wherein the codon of the mRNA comprises three contiguous nucleobases (N—N—N); and wherein the first unnatural base (X) is located at the last position (N—N—X) in the codon of the mRNA.

### Embodiment 268

The eukaryotic cell of any one of embodiments 261-267, wherein the first unnatural base or the second unnatural base is selected from the group consisting of:
(i) 2-thiouracil, 2-thio-thymine, 2′-deoxyuridine, 4-thio-uracil, 4-thio-thymine, uracil-5-yl, hypoxanthin-9-yl (I), 5-halouracil; 5-propynyl-uracil, 6-azo-thymine, 6-azo-uracil, 5-methylaminomethyluracil, 5-methoxyaminomethyl-2-thiouracil, pseudouracil, uracil-5-oxacetic acid methylester, uracil-5-oxacetic acid, 5-methyl-2-thiouracil, 3-(3-amino-3-N-2-carboxypropyl) uracil, 5-methyl-2-thiouracil, 4-thiouracil, 5-methyluracil, 5′-methoxycarboxymethyluracil, 5-methoxyuracil, uracil-5-oxyacetic acid, 5-(carboxyhydroxylmethyl) uracil, 5-carboxymethylaminomethyl-2-thiouridine, 5-carboxymethylaminomethyluracil, or dihydrouracil;
(ii) 5-hydroxymethyl cytosine, 5-trifluoromethyl cytosine, 5-halocytosine, 5-propynyl cytosine, 5-hydroxycytosine, cyclocytosine, cytosine arabinoside, 5,6-dihydrocytosine, 5-nitrocytosine, 6-azo cytosine, azacytosine, N4-ethylcytosine, 3-methylcytosine, 5-methylcytosine, 4-acetylcytosine, 2-thiocytosine, phenoxazine cytidine([5,4-b][1,4]benzoxazin-2(3H)-one), phenothiazine cytidine (1H-pyrimido[5,4-b][1, 4]benzothiazin-2(3H)-one), phenoxazine cytidine (9-(2-aminoethoxy)-H-pyrimido[5,4-b][1,4]benzoxazin-2(3H)-one), carbazole cytidine (2H-pyrimido[4,5-b]indol-2-one), or pyridoindole cytidine (H-pyrido [3′,2′:4,5]pyrrolo [2,3-d]pyrimidin-2-one);
(iii)₂-aminoadenine, 2-propyl adenine, 2-amino-adenine, 2-F-adenine, 2-amino-propyl-adenine, 2-amino-2′-deoxyadenosine, 3-deazaadenine, 7-methyladenine, 7-deaza-adenine, 8-azaadenine, 8-halo, 8-amino, 8-thiol, 8-thioalkyl, and 8-hydroxyl substituted adenines, N6-isopentenyladenine, 2-methyladenine, 2,6-diaminopurine, 2-methythio-N6-isopentenyladenine, or 6-aza-adenine;
(iv) 2-methylguanine, 2-propyl and alkyl derivatives of guanine, 3-deazaguanine, 6-thio-guanine, 7-methylguanine, 7-deazaguanine, 7-deazaguanosine, 7-deaza-8-azaguanine, 8-azaguanine, 8-halo, 8-amino, 8-thiol, 8-thioalkyl, and 8-hydroxyl substituted guanines, 1-methylguanine, 2,2-dimethylguanine, 7-methylguanine, or 6-aza-guanine; and
(v) hypoxanthine, xanthine, 1-methylinosine, queosine, beta-D-galactosylqueosine, inosine, beta-D-mannosylqueosine, wybutoxosine, hydroxyurea, (acp3)w, 2-aminopyridine, or 2-pyridone.

### Embodiment 269

The eukaryotic cell of any one of embodiments 261-267, wherein the first unnatural base and the second unnatural base are each, independently, selected from the group consisting of

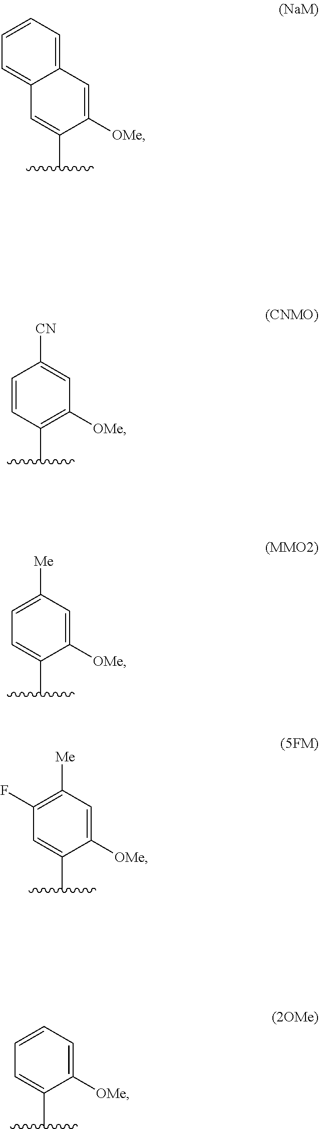 [SMILES: *c1cc2ccccc2cc1OC ; *c1ccc(C#N)cc1OC ; *c1ccc(C)cc1OC ; *c1cc(F)c(C)cc1OC ; *c1ccccc1OC]

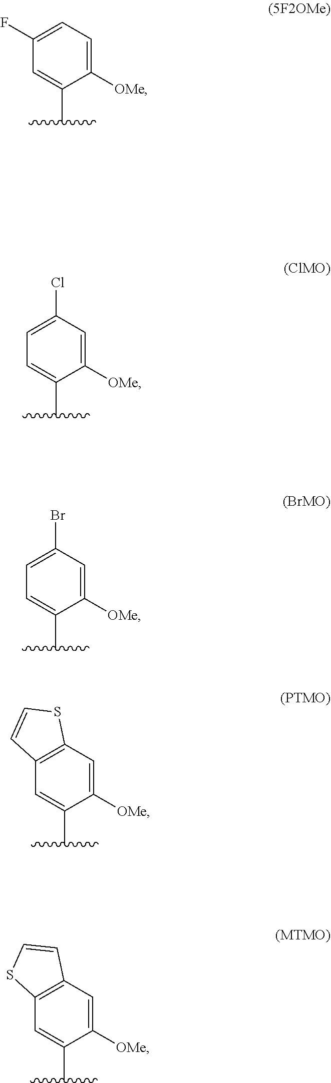 [SMILES: *c1cc(F)ccc1OC ; *c1ccc(Cl)cc1OC ; *c1ccc(Br)cc1OC ; *c1cc2ccsc2cc1OC ; *c1cc2sccc2cc1OC]

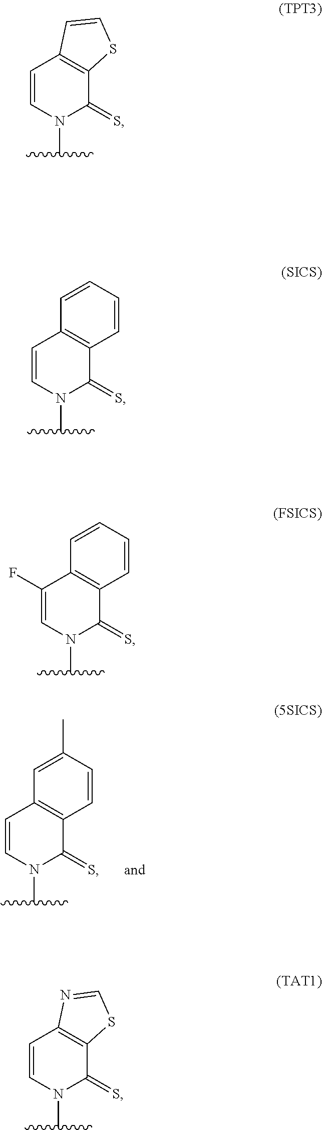 [SMILES: *n1ccc2ccsc2c1=S ; *n1ccc2ccccc2c1=S ; *n1cc(F)c2ccccc2c1=S ; *n1ccc2cc(C)ccc2c1=S ; "and" ; *n1ccc2ncsc2c1=S]

wherein the wavy line indicates a bond to a ribosyl moiety.

### Embodiment 270

The eukaryotic cell of any one of embodiments 261-267, when the first unnatural base is

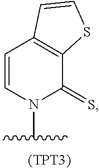

the second unnatural base is

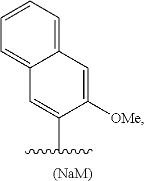

and when the first unnatural base is

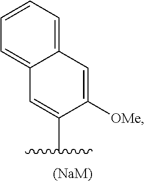

the second unnatural base is

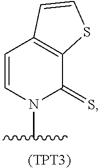

wherein the wavy line indicates a bond to a ribosyl moiety.

### Embodiment 271

The eukaryotic cell of any one of embodiments 261-267, when the first unnatrual base is

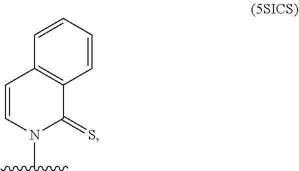 [SMILES: *n1ccc2ccccc2c1=S]

the second unnatural base is

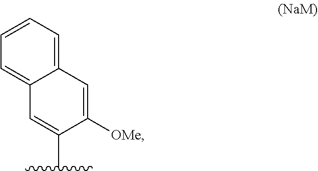 [SMILES: *c1cc2ccccc2cc1OC]

and when the first unnatural base is

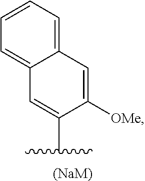

the second unnatural base is

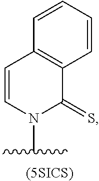

wherein the wavy line indicates a bond to a ribosyl moiety.

### Embodiment 272

The eukaryotic cell ofany one of embodiments 261-267, when the first unnatural base is

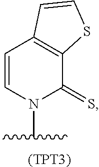

the second unnatural base is

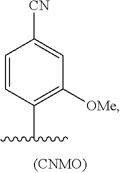 [SMILES: *c1ccc(C#N)cc1OC]

and when the first unnatural base is

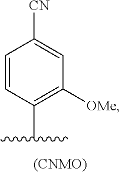 [SMILES: *c1ccc(C#N)cc1OC]

the second unnatural base is

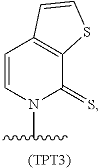

wherein the wavy line indicates a bond to a ribosyl moiety.

### Embodiment 273

The eukaryotic cell of any one of embodiments 261-267, when the first unnatural base is

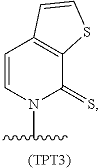

the second unnatural base is

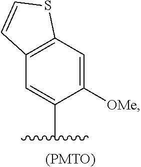

and when the first unnatural base is

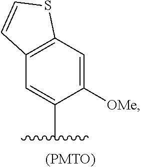

the second unnatural base is

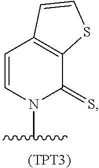

wherein the wavy line indicates a bond to a ribosyl moiety.

### Embodiment 274

The eukaryotic cell of any one of embodiments 261-267, when the first unnatural base is

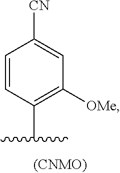 [SMILES: *c1ccc(C#N)cc1OC]

the second unnatural base is

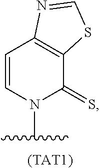

and when the first unnatural base is

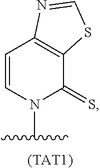

the second unnatural base is

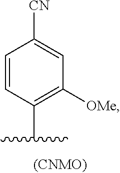 [SMILES: *c1ccc(C#N)cc1OC]

wherein the wavy line indicates a bond to a ribosyl moiety.

### Embodiment 275

The eukaryotic cell of any one of embodiments 261-267, when the first unnatural base is

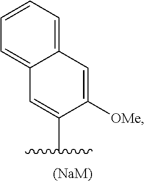

the second unnatural base is

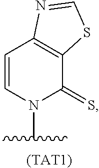

and when the first unnatural base is

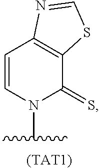

the second unnatural base is

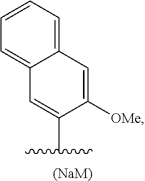

wherein the wavy line indicates a bond to a ribosyl moiety.

### Embodiment 276

The eukaryotic cell of any one of embodiments 261-275, wherein the first unnatural base or the second unnatural base comprise a modified sugar moiety selected from the group consisting of:

a modification at the 2′ position:
OH, substituted lower alkyl, alkaryl, aralkyl, O-alkaryl or O-aralkyl, SH, SCH₃, OCN, Cl,
Br, CN, CF₃, OCF₃, SOCH₃, SO₂CH₃, ONO₂, NO₂, N3, NH₂F;
O-alkyl, S-alkyl, N-alkyl;
O-alkenyl, S-alkenyl, N-alkenyl;
O-alkynyl, S-alkynyl, N-alkynyl;
O-alkyl-O-alkyl, 2′-F, 2′—OCH₃, 2′—O(CH₂)₂OCH₃ wherein the alkyl, alkenyl and alkynyl may be substituted or unsubstituted C₁-C₁₀, alkyl, C₂-C₁₀ alkenyl, C₂-C₁₀ alkynyl, —
O[(CH₂)ₙO]ₘCH₃, —O(CH₂)ₙOCH₃, —O(CH₂)ₙNH₂, —O(CH₂)ₙCH₃, —O(CH₂)ₙ—NH₂, and —
O(CH₂)ₙON[(CH₂)ₙCH₃)]₂, wherein n and m are from 1 to about 10;
and/or a modification at the 5′ position:
5′-vinyl, 5′-methyl (R or S);
a modification at the 4′ position:
4′-S, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and any combination thereof.

### Embodiment 277

The eukaryotic cell of any one of embodiments 263-276, wherein the at least one unnatural amino acid:
is a lysine analogue;
comprises an aromatic side chain;
comprises an azido group;
comprises an alkyne group; or
comprises an aldehyde or ketone group.

### Embodiment 278

The eukaryotic cell of embodiment 277, wherein the at least one unnatural amino acid is selected from the group consisting of N6-((azidoethoxy)-carbonyl)-L-lysine (AzK), N6-((propargylethoxy)-carbonyl)-L-lysine (PraK), BCN-L-lysine, norbomene lysine, TCO-lysine, methyltetrazine lysine, allyloxycarbonyllysine, 2-amino-8-oxononanoic acid, 2-amino-8-oxooctanoic acid, p-acetyl-L-phenylalanine, p-azidomethyl-L-phenylalanine (pAMF), p-iodo-L-phenylalanine, m-acetylphenylalanine, 2-amino-8-oxononanoic acid, p-propargyloxyphenylalanine, p-propargyl-phenylalanine, 3-methyl-phenylalanine, L-Dopa, fluorinated phenylalanine, isopropyl-L-phenylalanine, p-azido-L-phenylalanine, p-acyl-L-phenylalanine, p-benzoyl-L-phenylalanine, p-bromophenylalanine, p-amino-L-phenylalanine, isopropyl-L-phenylalanine, O-allyltyrosine, O-methyl-L-tyrosine, O-4-allyl-L-tyrosine, 4-propyl-L-tyrosine, phosphonotyrosine, tri-O-acetyl-GlcNAcp-serine, L-phosphoserine, phosphonoserine, L-3-(2-naphthyl)alanine, 2-amino-3-((2-((3-(benzyloxy)-3-oxopropyl)amino)ethyl)selanyl)propanoic acid, 2-amino-3-(phenylselanyl)propanoic, selenocysteine, N6-(((2-azidobenzyl)oxy)carbonyl)-L-lysine, N6-(((3-azidobenzyl)oxy)carbonyl)-L-lysine, or N6-(((4-azidobenzyl)oxy)carbonyl)-L-lysine.

### Embodiment 279

The eukaryotic cell of embodiment 278, wherein the at least one unnatural amino acid is N6-((azidoethoxy)-carbonyl)-L-lysine (AzK).

### Embodiment 280

The eukaryotic cell of any one of embodiments 261-279, wherein the eukaryotic cell is a human cell.

### Embodiment 281

The eukaryotic cell of the immediately preceding embodiment, wherein the human cell is a HEK293T cell.

### Embodiment 282

The eukaryotic cell of any one of embodiments 261 to 279, wherein the cell is a mammalian cell, optionally wherein the mammalian cell is a hamster cell.

### Embodiment 283

The eukaryotic cell of the immediately preceding embodiment, wherein the mammalian cell is a Chinese hamster ovary (CHO) cell.

### Embodiment 284

The eukaryotic cell of any one of embodiments 261-283, wherein the cell is isolated, optionally wherein the cell is purified.

### Embodiment 285

The eukaryotic cell of any one of embodiments 261-284, further comprising a polypeptide translated from the mRNA, wherein the polypeptide comprises the unnatural amino acid and a mammalian glycosylation pattern.

### Embodiment 285.1

A semi-synthetic organism comprising the eukaryotic cell of any one of embodiments 261-285.

### Embodiment 286

A eukaryotic cell culture comprising a plurality of eukaryotic cells of any one of embodiments 261-285.

### Embodiment 286.1

A method of delivering a cell to an organism, comprising contacting the organism with the cell of any one of embodiments 261-285.

### Embodiment 286.2

The method of embodiment 286.1, wherein the organism is a mammal, optionally wherein the mammal is a human.

### Embodiment 287

A method of producing a polypeptide comprising at least one unnatural amino acid in a eukaryotic cell, comprising:
(a) introducing into the cell:
   (i) a messenger RNA (mRNA) with a codon comprising a first unnatural base; and
   (ii) a transfer RNA (tRNA) with an anticodon comprising a second unnatural base in the eukaryotic cell, wherein the first and second unnatural bases are capable of forming an unnatural base pair (UBP) in the eukaryotic cell; and
(b) translating the polypeptide comprising the at least one unnatural amino acid from the mRNA using the tRNA.

### Embodiment 288

The method of the preceding embodiment, wherein the tRNA is charged with an unnatural amino acid.

### Embodiment 289

A method of producing a polypeptide comprising at least one unnatural amino acid in a eukaryotic cell, comprising:
(a) providing a eukaryotic cell comprising:
   (i) a messenger RNA (mRNA) with a codon comprising a first unnatural base;
   (ii) a transfer RNA (tRNA) with an anticodon comprising a second unnatural base, wherein the first and second unnatural bases are capable of forming an unnatural base pair (UBP) in the eukaryotic cell;
(b) translating the polypeptide comprising the at least one unnatural amino acid from the mRNA using the tRNA by a ribosome that is endogenous to the eukaryotic cell.

### Embodiment 290

A method of producing a polypeptide in a eukaryotic cell, wherein the polypeptide comprises at least one unnatural amino acid, the method comprising:
(a) providing a eukaryotic cell, the eukaryotic cell comprising:
   (i) an mRNA comprising a codon, wherein the codon comprises a first unnatural base;
   (ii) a tRNA comprising an anti-codon, wherein the anti-codon comprises a second unnatural base, and wherein the first and second unnatural bases are capable of forming a complimentary base pair; and
(b) a tRNA synthetase, wherein the tRNA synthetase preferentially aminoacylates the tRNA with the at least one unnatural amino acid compared to a natural amino acid; and
(c) providing the one more unnatural amino acids to the eukaryotic cell, wherein the eukaryotic cell produces the polypeptide comprising the at least one unnatural amino acid.

### Embodiment 291

The method of any one of embodiments 287 to 290, wherein the codon of the mRNA comprises three contiguous nucleobases (N—N—N); and wherein the first unnatural base (X) is located at the first position (X—N—N) in the codon of the mRNA.

### Embodiment 292

The method of any one of embodiments 287 to 290, wherein the codon of the mRNA comprises three contiguous nucleobases (N—N—N); and wherein the first unnatural base (X) is located at the middle position (N—X—N) in the codon of the mRNA.

### Embodiment 293

The method of any one of embodiments 287 to 290, wherein the codon of the mRNA comprises three contiguous nucleobases (N—N—N); and wherein the first unnatural base (X) is located at the last position (N—N—X) in the codon of the mRNA.

### Embodiment 294

The method of any one of embodiments 287 to 293, wherein the one or more unnatural bases comprising the codon in the mRNA is of the formula

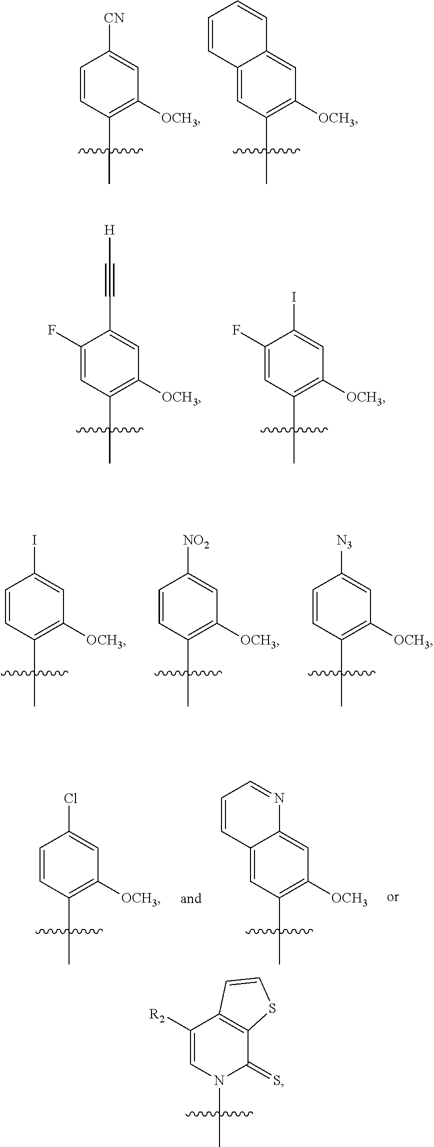

wherein R₂ is selected from the group consisting of hydrogen, alkyl, alkenyl, alkynyl, methoxy, methanethiol, methaneseleno, halogen, cyano, and azido, and the wavy line indicates a bond to a ribosyl moiety.

### Embodiment 295

The method of any one of embodiments 287 to 293, wherein the first unnatural base or the second unnatural base is selected from the group consisting of:
(i) 2-thiouracil, 2-thio-thymine, 2′-deoxyuridine, 4-thio-uracil, 4-thio-thymine, uracil-5-yl, hypoxanthin-9-yl (I), 5-halouracil; 5-propynyl-uracil, 6-azo-thymine, 6-azo-uracil, 5-methylaminomethyluracil, 5-methoxyaminomethyl-2-thiouracil, pseudouracil, uracil-5-oxacetic acid methylester, uracil-5-oxacetic acid, 5-methyl-2-thiouracil, 3-(3-amino-3-N-2-carboxypropyl) uracil, 5-methyl-2-thiouracil, 4-thiouracil, 5-methyluracil, 5′-methoxycarboxymethyluracil, 5-methoxyuracil, uracil-5-oxyacetic acid, 5-(carboxyhydroxylmethyl) uracil, 5-carboxymethylaminomethyl-2-thiouridine, 5-carboxymethylaminomethyluracil, or dihydrouracil;
(ii) 5-hydroxymethyl cytosine, 5-trifluoromethyl cytosine, 5-halocytosine, 5-propynyl cytosine, 5-hydroxycytosine, cyclocytosine, cytosine arabinoside, 5,6-dihydrocytosine, 5-nitrocytosine, 6-azo cytosine, azacytosine, N4-ethylcytosine, 3-methylcytosine, 5-methylcytosine, 4-acetylcytosine, 2-thiocytosine, phenoxazine cytidine([5,4-b][1,4]benzoxazin-2(3H)-one), phenothiazine cytidine (1H-pyrimido[5,4-b][1, 4]benzothiazin-2(3H)-one), phenoxazine cytidine (9-(2-aminoethoxy)-H-pyrimido[5,4-b][1,4]benzoxazin-2(3H)-one), carbazole cytidine (2H-pyrimido[4,5-b]indol-2-one), or pyridoindole cytidine (H-pyrido [3′,2′:4,5]pyrrolo [2,3-d]pyrimidin-2-one);
(iii)₂-aminoadenine, 2-propyl adenine, 2-amino-adenine, 2-F-adenine, 2-amino-propyl-adenine, 2-amino-2′-deoxyadenosine, 3-deazaadenine, 7-methyladenine, 7-deaza-adenine, 8-azaadenine, 8-halo, 8-amino, 8-thiol, 8-thioalkyl, and 8-hydroxyl substituted adenines, N6-isopentenyladenine, 2-methyladenine, 2,6-diaminopurine, 2-methythio-N6-isopentenyladenine, or 6-aza-adenine;
(iv) 2-methylguanine, 2-propyl and alkyl derivatives of guanine, 3-deazaguanine, 6-thio-guanine, 7-methylguanine, 7-deazaguanine, 7-deazaguanosine, 7-deaza-8-azaguanine, 8-azaguanine, 8-halo, 8-amino, 8-thiol, 8-thioalkyl, and 8-hydroxyl substituted guanines, 1-methylguanine, 2,2-dimethylguanine, 7-methylguanine, or 6-aza-guanine; and
(v) hypoxanthine, xanthine, 1-methylinosine, queosine, beta-D-galactosylqueosine, inosine, beta-D-mannosylqueosine, wybutoxosine, hydroxyurea, (acp3)w, 2-aminopyridine, or 2-pyridone.

### Embodiment 296

The method of any one of embodiments 287 to 295, wherein the first unnatural base or the second unnatural base is selected from the group consisting of

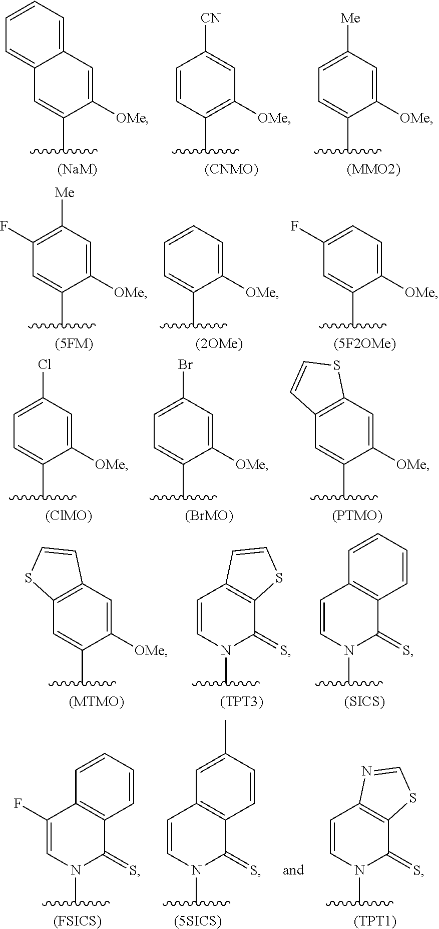

wherein the wavy line indicates a bond to a ribosyl moiety.

### Embodiment 297

The method of embodiment 2%, wherein when the first unnatural base is

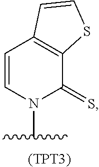

the second unnatural base is

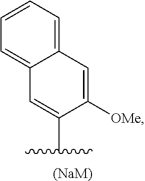

and when the first unnatural base is

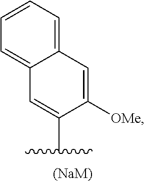

the second unnatural base is

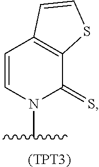

wherein the wavy line indicates a bond to a ribosyl moiety.

### Embodiment 298

The method of embodiment 2%, wherein when the first unnatural base is

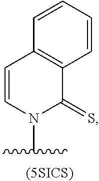

the second unnatural base is

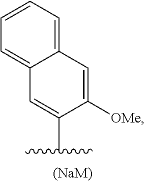

and when the first unnatural base is

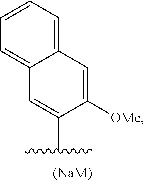

the second unnatural base is

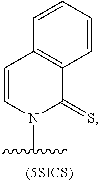

wherein the wavy line indicates a bond to a ribosyl moiety.

### Embodiment 299

The method of embodiment 2%, wherein when the first unnatural base is

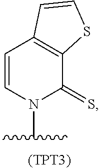

the second unnatural base is

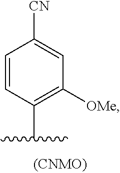 [SMILES: *c1ccc(C#N)cc1OC]

and when the first unnatural base is

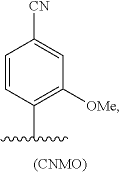 [SMILES: *c1ccc(C#N)cc1OC]

the second unnatural base is

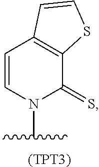

wherein the wavy line indicates a bond to a ribosyl moiety.

### Embodiment 300

The method of embodiment 2%, wherein when the first unnatural base is

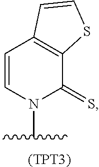

the second unnatural base is

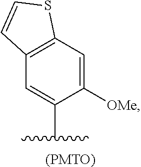

and when the first unnatural base is

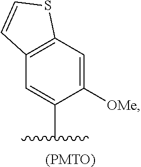

the second unnatural base is

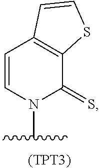

wherein the wavy line indicates a bond to a ribosyl moiety.

### Embodiment 301

The method of embodiment 296, wherein when the first unnatural base is

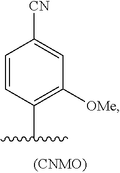 [SMILES: *c1ccc(C#N)cc1OC]

the second unnatural base is

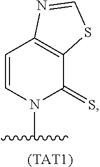

and when the first unnatural base is

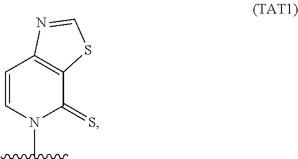 [SMILES: *n1ccc2ncsc2c1=S]

the second unnatural base is

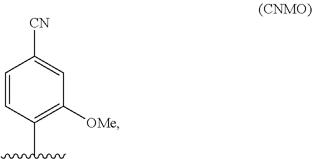 [SMILES: *c1ccc(C#N)cc1OC]

wherein the wavy line indicates a bond to a ribosyl moiety.

### Embodiment 302

The method of embodiment 2%, wherein when the first unnatural base is

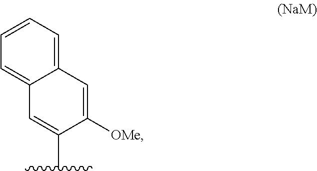 [SMILES: *c1cc2ccccc2cc1OC]

the second unnatural base is

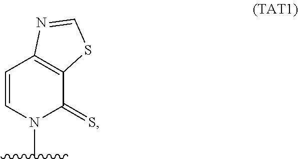 [SMILES: *n1ccc2ncsc2c1=S]

and when the first unnatural base is

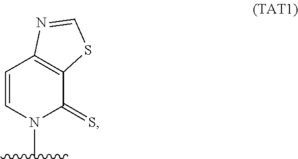 [SMILES: *n1ccc2ncsc2c1=S]

the second unnatural base is

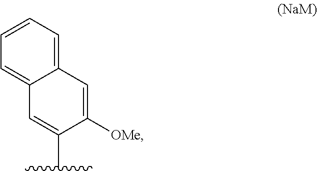 [SMILES: *c1cc2ccccc2cc1OC]

wherein the wavy line indicates a bond to a ribosyl moiety.

### Embodiment 303

The method of any one of embodiments 287 to 2%, wherein the codon of the mRNA comprises three contiguous nucleobases (N—N—N), wherein the first unnatural base (X) is located at the first position X—N— in the codon of the mRNA, wherein the unnatural base is selected from

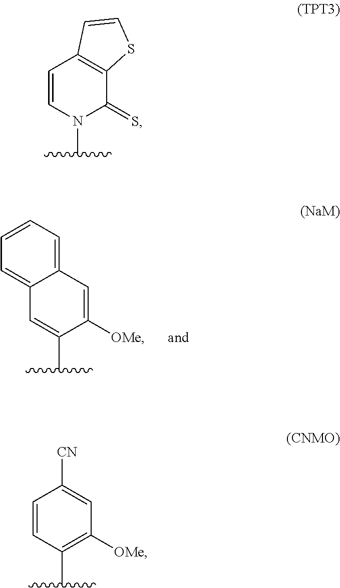 [SMILES: *n1ccc2ccsc2c1=S ; *c1cc2ccccc2cc1OC ; "and" ; *c1ccc(C#N)cc1OC]

and wherein the wavy line indicates a bond to a ribosyl moiety.

### Embodiment 304

The method of any one of embodiments 287 to 2%, wherein the codon of the mRNA comprises three contiguous nucleobases (N—N—N), wherein the first unnatural base (X) is located at the middle position (N—X—N) in the codon of the mRNA, wherein the unnatural base is selected from

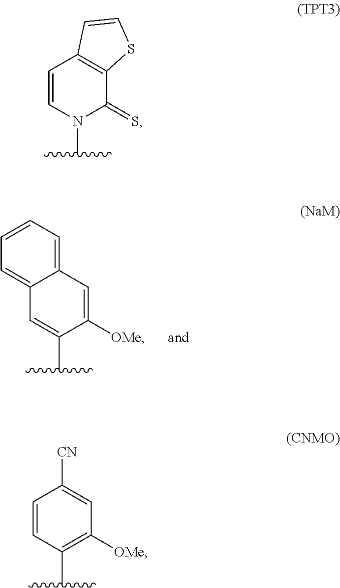 [SMILES: *n1ccc2ccsc2c1=S ; *c1cc2ccccc2cc1OC ; "and" ; *c1ccc(C#N)cc1OC]

and wherein the wavy line indicates a bond to a ribosyl moiety.

### Embodiment 305

The method of any one of embodiments 287 to 2%, wherein the codon of the mRNA comprises three contiguous nucleobases (N—N—N), wherein the first unnatural base (X) is located at the last position (N—N—X) in the codon of the mRNA, wherein the unnatural base is selected from

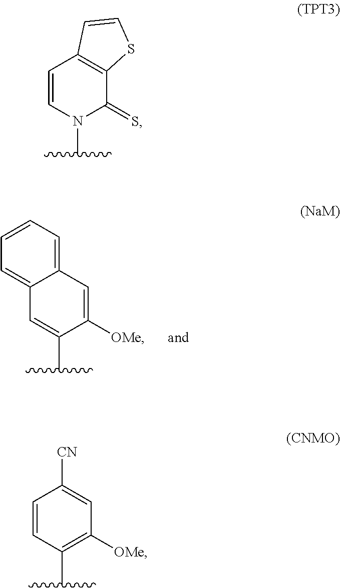 [SMILES: *n1ccc2ccsc2c1=S ; *c1cc2ccccc2cc1OC ; "and" ; *c1ccc(C#N)cc1OC]

and wherein the wavy line indicates a bond to a ribosyl moiety.

### Embodiment 306

The method of any one of embodiments 287 to 2%, wherein the anticodon of the tRNA comprises three contiguous nucleobases (N—N—N); and wherein the second unnatural base (X) is located at the first position (X—N—N) in the anticodon of the tRNA, wherein the unnatural base is selected from

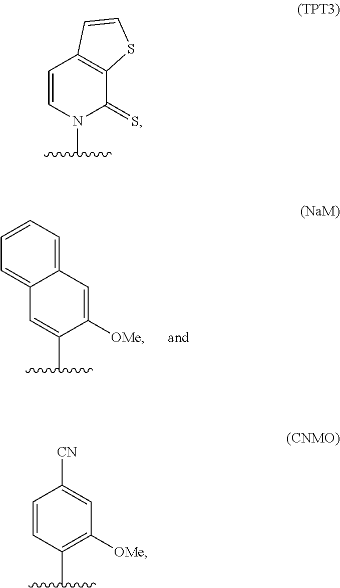 [SMILES: *n1ccc2ccsc2c1=S ; *c1cc2ccccc2cc1OC ; "and" ; *c1ccc(C#N)cc1OC]

and wherein the wavy line indicates a bond to a ribosyl moiety.

### Embodiment 307

The method of any one of embodiments 287 to 2%, wherein the anticodon of the tRNA comprises three contiguous nucleobases (N—N—N); and wherein the second unnatural base (X) is located at the middle position (N—X—N) in the anticodon of the tRNA, wherein the unnatural base is selected from

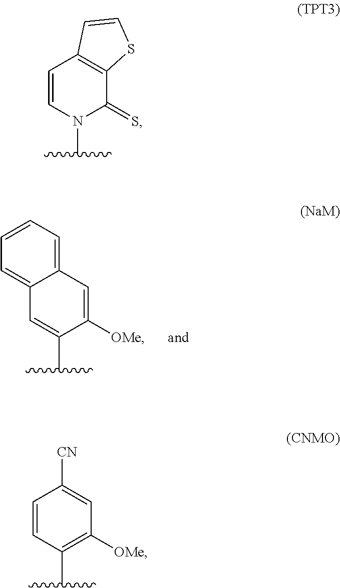 [SMILES: *n1ccc2ccsc2c1=S ; *c1cc2ccccc2cc1OC ; "and" ; *c1ccc(C#N)cc1OC]

and wherein the wavy line indicates a bond to a ribosyl moiety.

### Embodiment 308

The method of any one of embodiments 287 to 2%, wherein the anticodon of the tRNA comprises three contiguous nucleobases (N—N—N); and wherein the second unnatural base (X) is located at the last position (N—N—X) in the anticodon of the tRNA, wherein the unnatural base is selected from

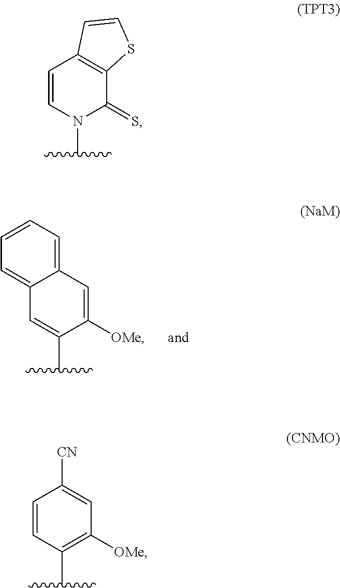 [SMILES: *n1ccc2ccsc2c1=S ; *c1cc2ccccc2cc1OC ; "and" ; *c1ccc(C#N)cc1OC]

and wherein the wavy line indicates a bond to a ribosyl moiety.

### Embodiment 41

The method of any one of embodiments 287 to 2%, wherein the codon and the anticodon each comprise three contiguous nucleobases (N—N—N), wherein the first unnatural base (X) of the codon in the mRNA is located at a first position (X—N—N) of the codon, and the second unnatural base (Y) of the anticodon of the tRNA is located at the last position (N—N—Y) of the anticodon.

### Embodiment 310

The method of any one of embodiments 287 to 2%, wherein the codon and the anticodon each comprise three contiguous nucleobases (N—N—N), wherein the codon in the mRNA comprises a first unnatural base (X) located at the middle position (N—X—N) of the codon, and the anticodon in the tRNA comprises a second unnatural base (Y) located at the middle position (N—Y—N) of the anticodon.

### Embodiment 311

The method of any one of embodiments 287 to 2%, wherein the codon and the anticodon each comprise three contiguous nucleobases (N—N—N), wherein the codon in the mRNA comprises a first unnatural base (X) located at the last position (N—N—X) of the codon, and the anticodon in the tRNA comprises a second unnatural base (Y) located at the first position (Y—N—N) of the anticodon.

### Embodiment 312

The method of any one of embodiments 309 to 311, wherein the first unnatural base (X) located in the codon of the mRNA and the second unnatural base (Y) located in the anticodon of the tRNA are the same or are different.

### Embodiment 313

The method of any one of embodiments 309 to 312, wherein the first unnatural base (X) located in the codon of the mRNA and the second unnatural base (Y) located in the anticodon of the tRNA are selected from the group consisting of

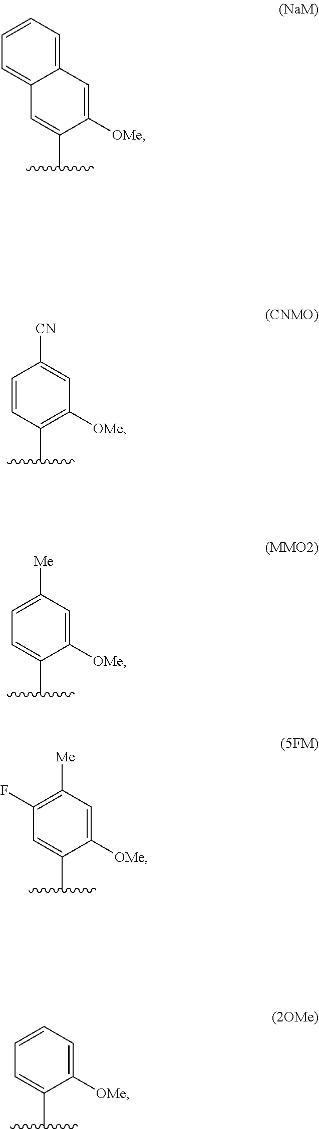 [SMILES: *c1cc2ccccc2cc1OC ; *c1ccc(C#N)cc1OC ; *c1ccc(C)cc1OC ; *c1cc(F)c(C)cc1OC ; *c1ccccc1OC]

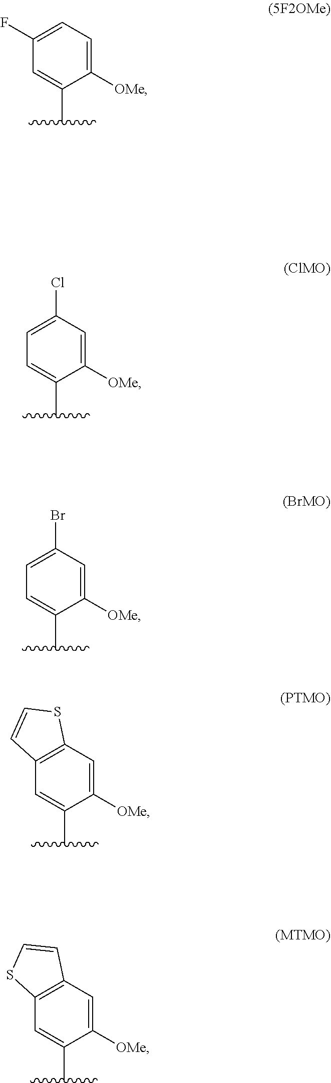 [SMILES: *c1cc(F)ccc1OC ; *c1ccc(Cl)cc1OC ; *c1ccc(Br)cc1OC ; *c1cc2ccsc2cc1OC ; *c1cc2sccc2cc1OC]

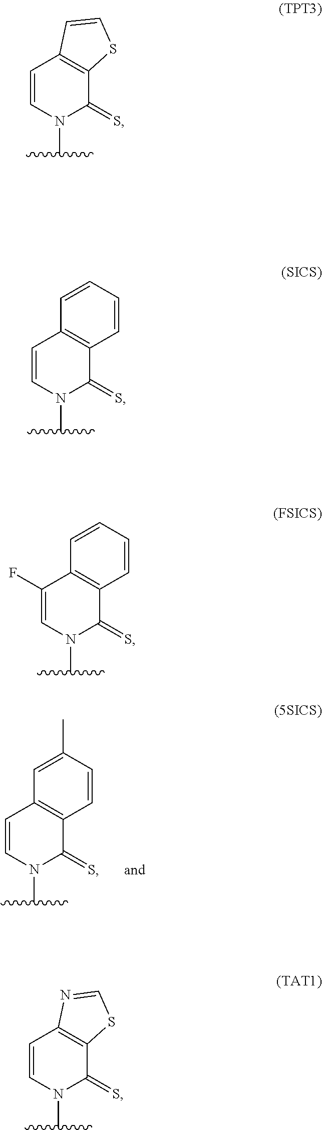 [SMILES: *n1ccc2ccsc2c1=S ; *n1ccc2ccccc2c1=S ; *n1cc(F)c2ccccc2c1=S ; *n1ccc2cc(C)ccc2c1=S ; "and" ; *n1ccc2ncsc2c1=S]

wherein the wavy line indicates a bond to a ribosyl moiety.

### Embodiment 314

The method of embodiment 313, wherein the first unnatural base (X) located in the codon of the mRNA and the second unnatural base (Y) located in the anticodon of the tRNA are selected from the group consisting of

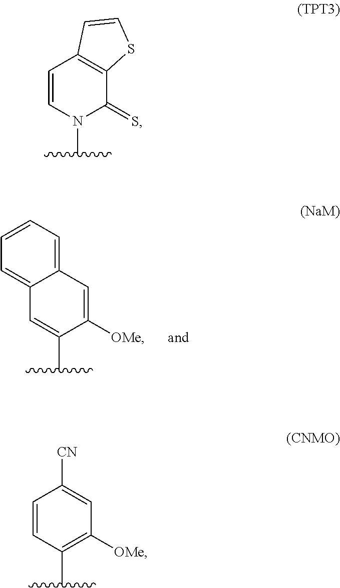 [SMILES: *n1ccc2ccsc2c1=S ; *c1cc2ccccc2cc1OC ; "and" ; *c1ccc(C#N)cc1OC]

wherein the wavy line indicates a bond to a ribosyl moiety.

### Embodiment 315

The method of embodiment 314, wherein the first unnatural base (X) located in the codon of the mRNA and the second unnatural base (Y) located in the anticodon of the tRNA are both

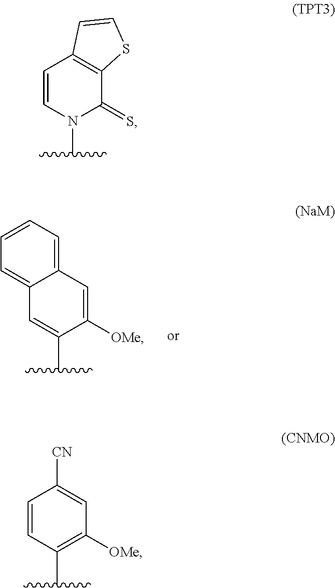 [SMILES: *n1ccc2ccsc2c1=S ; *c1cc2ccccc2cc1OC ; "or" ; *c1ccc(C#N)cc1OC]

wherein the wavy line indicates a bond to a ribosyl moiety.

### Embodiment 316

The method of embodiment 314, wherein the first unnatural base (X) located in the codon of the mRNA is selected from

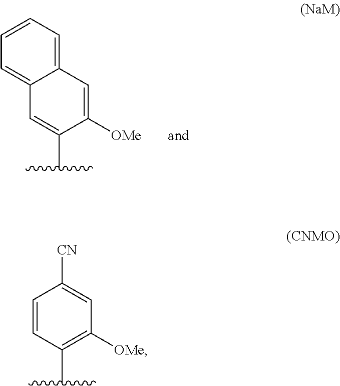 [SMILES: *c1cc2ccccc2cc1OC ; "and" ; *c1ccc(C#N)cc1OC]

and the second unnatural base (Y) located in the anticodon of the tRNA is

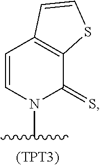

wherein in each case the wavy line indicates a bond to a ribosyl moiety.

### Embodiment 317

The method of any one of embodiments 287 to 290, 292, 294 to 302, 304, 307, and 410, wherein the codon in the mRNA is selected from AXC, GXC or GXU, wherein X is the first unnatural base.

### Embodiment 318

The method of the immediately preceding embodiment, wherein the anticodon in the tRNA is selected from GYU, GYC, and AYC, and Y is a second unnatural base.

### Embodiment 319

The method of embodiment 318, wherein the codon in the mRNA is AXC and the anticodon in the tRNA is GYU.

### Embodiment 320

The method of embodiment 318, wherein the codon in the mRNA is GXC and the anticodon in the tRNA is GYC.

### Embodiment 321

The method of embodiment 318, wherein the codon in the mRNA is GXU and the anticodon is AYC.

### Embodiment 322

The method of any one of embodiments 287 to 321, wherein the first unnatural base or the second unnatural base comprise a modified sugar moiety selected from the group consisting of:

a modification at the 2′ position:
OH, substituted lower alkyl, alkaryl, aralkyl, O-alkaryl or O-aralkyl, SH, SCH₃, OCN, Cl,
Br, CN, CF₃, OCF₃, SOCH₃, SO₂CH₃, ONO₂, NO₂, N3, NH₂F;
O-alkyl, S-alkyl, N-alkyl;
O-alkenyl, S-alkenyl, N-alkenyl;
O-alkynyl, S-alkynyl, N-alkynyl;
O-alkyl-O-alkyl, 2′-F, 2′—OCH₃, 2′—O(CH₂)₂OCH₃ wherein the alkyl, alkenyl and alkynyl may be substituted or unsubstituted C₁-C₁₀, alkyl, C₂-C₁₀ alkenyl, C₂-C₁₀ alkynyl, —
O[(CH₂)ₙO]ₘCH₃, —O(CH₂)ₙOCH₃, —O(CH₂)ₙNH₂, —O(CH₂)ₙCH₃, —O(CH₂)ₙ—NH₂, and —
O(CH₂)ₙON[(CH₂)ₙCH₃)]₂, wherein n and m are from 1 to about 10;
and/or a modification at the 5′ position:
5′-vinyl, 5′-methyl (R or S);
a modification at the 4′ position:
4′-S, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and any combination thereof.

### Embodiment 323

The method of any one of embodiments 287 to 322, wherein the at least one unnatural amino acid:
is a lysine analogue;
comprises an aromatic side chain;
comprises an azido group;
comprises an alkyne group; or
comprises an aldehyde or ketone group.

### Embodiment 324

The method of any one of embodiments 287 to 322, wherein at least one unnatural amino acid is selected from the group consisting of N6-((azidoethoxy)-carbonyl)-L-lysine (AzK), N6-((propargylethoxy)-carbonyl)-L-lysine (PraK), BCN-L-lysine, norbomene lysine, TCO-lysine, methyltetrazine lysine, allyloxycarbonyllysine, 2-amino-8-oxononanoic acid, 2-amino-8-oxooctanoic acid, p-acetyl-L-phenylalanine, p-azidomethyl-L-phenylalanine (pAMF), p-iodo-L-phenylalanine, m-acetylphenylalanine, 2-amino-8-oxononanoic acid, p-propargyloxyphenylalanine, p-propargyl-phenylalanine, 3-methyl-phenylalanine, L-Dopa, fluorinated phenylalanine, isopropyl-L-phenylalanine, p-azido-L-phenylalanine, p-acyl-L-phenylalanine, p-benzoyl-L-phenylalanine, p-bromophenylalanine, p-amino-L-phenylalanine, isopropyl-L-phenylalanine, O-allyltyrosine, O-methyl-L-tyrosine, O-4-allyl-L-tyrosine, 4-propyl-L-tyrosine, phosphonotyrosine, tri-O-acetyl-GlcNAcp-serine, L-phosphoserine, phosphonoserine, L-3-(2-naphthyl)alanine, 2-amino-3-((2-((3-(benzyloxy)-3-oxopropyl)amino)ethyl)selanyl)propanoic acid, 2-amino-3-(phenylselanyl)propanoic, selenocysteine, N6-(((2-azidobenzyl)oxy)carbonyl)-L-lysine, N6-(((3-azidobenzyl)oxy)carbonyl)-L-lysine, or N6-(((4-azidobenzyl)oxy)carbonyl)-L-lysine.

### Embodiment 325

The method of embodiment 324, wherein the unnatural amino acid is N6-((azidoethoxy)-carbonyl)-L-lysine (AzK).

### Embodiment 326

The method of any one of embodiments 287 to 325, wherein the cell is a human cell.

### Embodiment 327

The method of embodiment 326, wherein the human cell is a HEK293T cell.

### Embodiment 328

The method of any one of embodiments 287 to 325, wherein the cell is a hamster cell.

### Embodiment 329

The method of embodiment 328, wherein the hamster cell is a Chinese hamster ovary (CHO) cell.

### Embodiment 330

The method of any one of embodiments 287 to 329, wherein the tRNA is derived from Methanococcus jannaschii, Methanosarcina barkeri, Methanosarcina mazei, or Methanosarcina acetivorans.

### Embodiment 331

The method of any one of embodiments 287 to 330, wherein the cell comprises a tRNA synthetase derived from Methanococcus jannaschii, Methanosarcina barkeri, Methanosarcina mazei, or Methanosarcina acetivorans.

### Embodiment 332

A system for expression of an unnatural polypeptide comprising:
(a) at least one unnatural amino acid;
(b) an mRNA encoding the unnatural polypeptide, said mRNA comprising at least one codon comprising one or more first unnatural bases;
(c) a tRNA comprising at least one anti-codon comprising one or more second unnatural bases wherein the one or more first unnatural bases and the one or more second unnatural bases are capable of forming one or more complementary base pairs;
(d) a eukaryotic ribosome capable of translating the mRNA into a polypeptide comprising the unnatural amino acid using the tRNA and tRNA synthetase, wherein the tRNA is charged with the unnatural amino acid, or the system further comprises a tRNA synthetase or one or more nucleic acid constructs comprising a nucleic acid sequence encoding a tRNA synthetase, wherein the tRNA synthetase preferentially aminoacylates the tRNA with the at least one unnatural amino acid.

### Embodiment 333

The system of embodiment 332, wherein the at least one codon of the mRNA comprises three contiguous nucleobases (N—N—N); and wherein the one or more first unnatural bases (X) is located at the first position (X—N—N) in the at least one codon of the mRNA.

### Embodiment 334

The system of embodiment 332, wherein the at least one codon of the mRNA comprises three contiguous nucleobases (N—N—N); and wherein the one or more first unnatural bases (X) is located at the middle position (N—X—N) in the codon of the mRNA.

### Embodiment 335

The system of embodiment 332, wherein the at least one codon of the mRNA comprises three contiguous nucleobases (N—N—N); and wherein the one or more first unnatural bases (X) is located at the last position (N—N—X) in the at least one codon of the mRNA.

### Embodiment 336

The system of any one of embodiments 332 to 335, wherein the one or more unnatural bases is of the formula

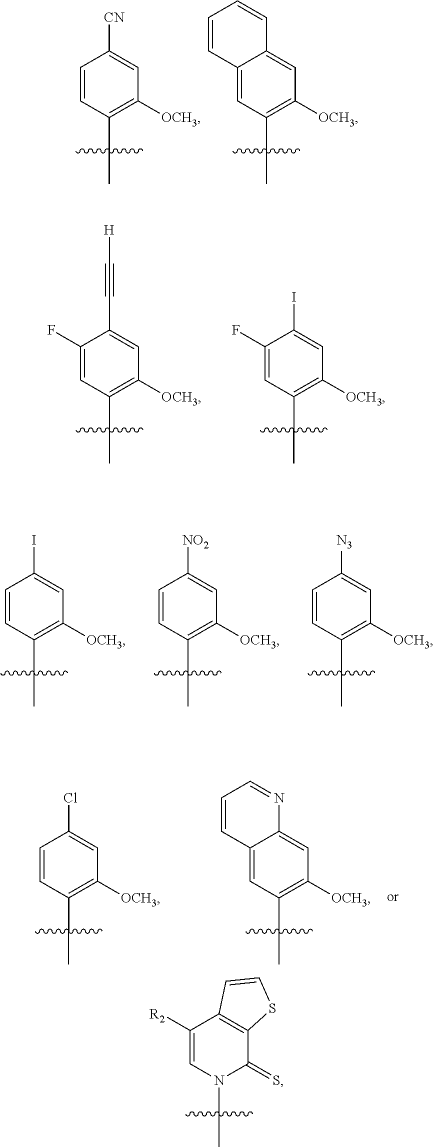

wherein R₂ is selected from the group consisting of hydrogen, alkyl, alkenyl, alkynyl, methoxy, methanethiol, methaneseleno, halogen, cyano, and azido, and the wavy line indicates a bond to a ribosyl moiety.

### Embodiment 337

The system of any one of embodiments 332 to 335, wherein the one or more first unnatural bases or the one or more second unnatural bases is selected from the group consisting of

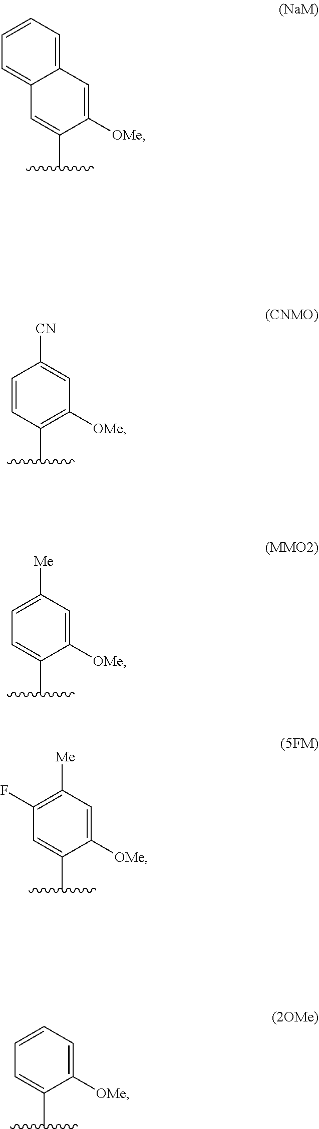 [SMILES: *c1cc2ccccc2cc1OC ; *c1ccc(C#N)cc1OC ; *c1ccc(C)cc1OC ; *c1cc(F)c(C)cc1OC ; *c1ccccc1OC]

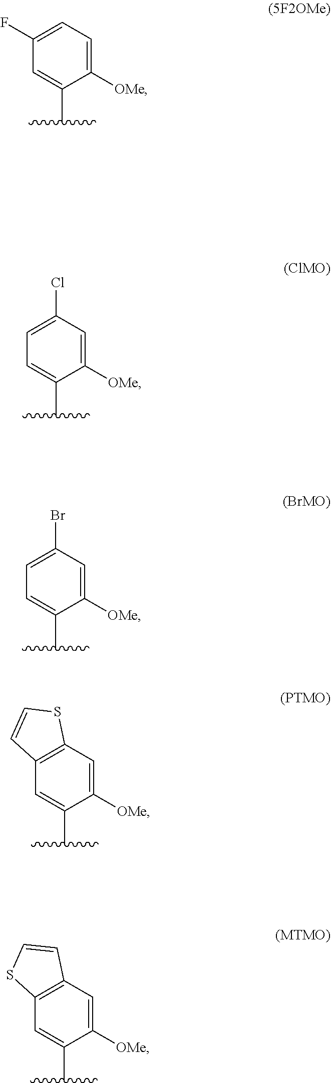 [SMILES: *c1cc(F)ccc1OC ; *c1ccc(Cl)cc1OC ; *c1ccc(Br)cc1OC ; *c1cc2ccsc2cc1OC ; *c1cc2sccc2cc1OC]

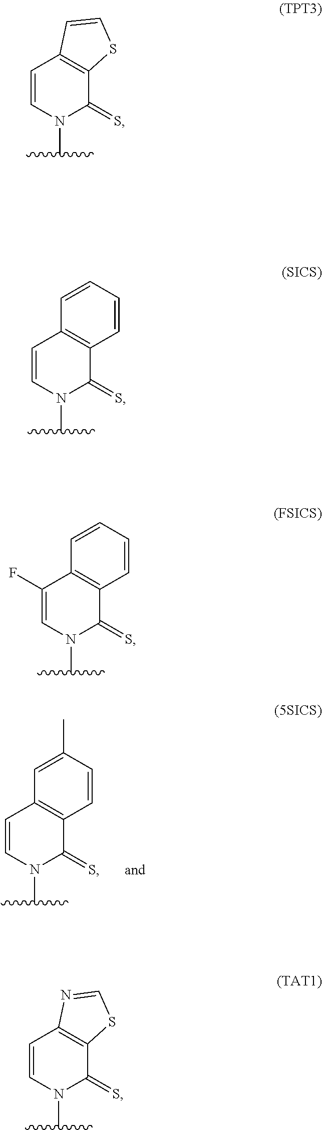 [SMILES: *n1ccc2ccsc2c1=S ; *n1ccc2ccccc2c1=S ; *n1cc(F)c2ccccc2c1=S ; *n1ccc2cc(C)ccc2c1=S ; "and" ; *n1ccc2ncsc2c1=S]

wherein the wavy line indicates a bond to a ribosyl moiety.

### Embodiment 338

The system of embodiment 337, when the one or more first unnatural bases is

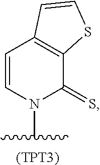

The one or more second unnatural bases is

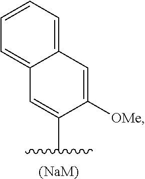

and when the one or more first unnatural bases is

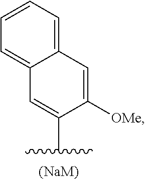

the second unnatural base is

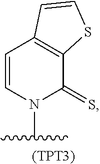

wherein the wavy line indicates a bond to a ribosyl moiety.

### Embodiment 339

The system of embodiment 337, when the one or more first unnatural bases is

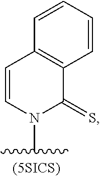 [SMILES: *n1ccc2ccccc2c1=S]

the one or more second unnatural bases is

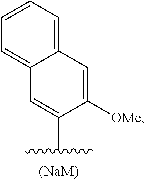

and when the one or more first unnatural base is

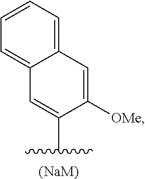

the one or more second unnatural bases is

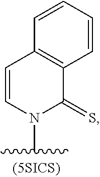 [SMILES: *n1ccc2ccccc2c1=S]

wherein the wavy line indicates a bond to a ribosyl moiety.

### Embodiment 340

The system of embodiment 337, when the one or more first unnatural bases is

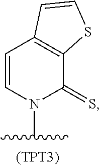

the one or more second unnatural bases is

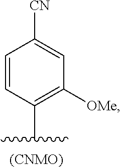

and when the one or more first unnatural bases is

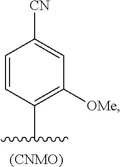

the one or more second unnatural bases

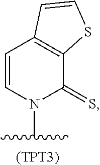

wherein the wavy line indicates a bond to a ribosyl moiety.

### Embodiment 341

The system of embodiment 337, when the one or more first unnatural bases is

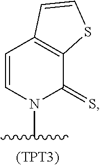

the one or more second unnatural bases is

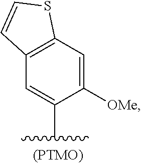 [SMILES: *c1cc2ccsc2cc1OC]

and when the one or more first unnatural bases is

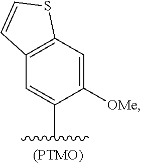 [SMILES: *c1cc2ccsc2cc1OC]

the one or more second unnatural bases is

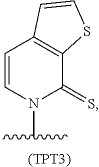

wherein the wavy line indicates a bond to a ribosyl moiety.

### Embodiment 342

The system of embodiment 337, when the one or more first unnatural bases is

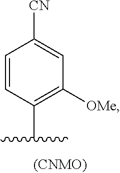 [SMILES: *c1ccc(C#N)cc1OC]

the one or more second unnatural bases is

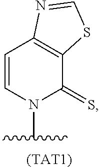

and when the one or more first unnatural bases is

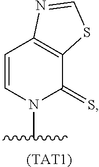

the one or more second unnatural bases is

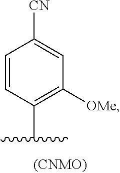 [SMILES: *c1ccc(C#N)cc1OC]

wherein the wavy line indicates a bond to a ribosyl moiety.

### Embodiment 343

The system of embodiment 337, when the one or more first unnatural bases is

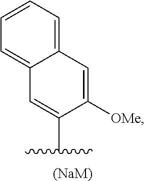

the one or more second unnatural bases is

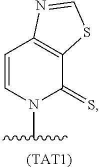

and when the one or more first unnatural bases is

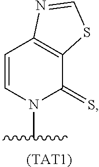

the one or more second unnatural bases is

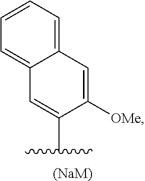

wherein the wavy line indicates a bond to a ribosyl moiety.

### Embodiment 344

The system of embodiment 337, when the one or more first unnatural bases is

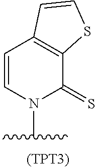

and

the one or more second unnatural bases is

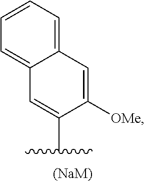

wherein the wavy line indicates a bond to a ribosyl moiety.

### Embodiment 345

The system of any one of embodiments 332 to 335, wherein the one or more first unnatural bases is selected from

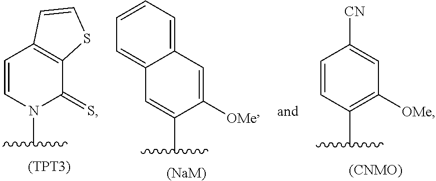

wherein the wavy line indicates a bond to a ribosyl moiety.

### Embodiment 346

The system of embodiment 332, wherein the at least one codon of the mRNA comprises three contiguous nucleobases (N—N—N), wherein the one or more first unnatural bases (X) is located at the first position (X—N—N) in the codon of the mRNA, wherein the one or more first unnatural bases is selected from

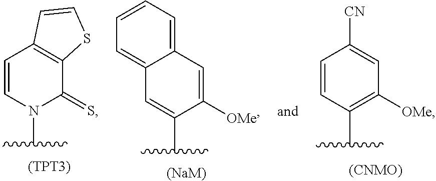

and wherein the wavy line indicates a bond to a ribosyl moiety.

### Embodiment 347

The system of embodiment 332, wherein the at least one codon of the mRNA comprises three contiguous nucleobases (N—N—N), wherein the one or more first unnatural bases (X) is located at the middle position (N—X—N in the codon of the mRNA, wherein the one or more first unnatural bases is selected from

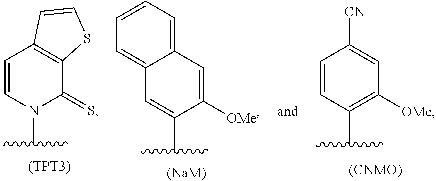

and wherein the wavy line indicates a bond to a ribosyl moiety.

### Embodiment 348

The system of embodiment 332, wherein the at least one codon of the mRNA comprises three contiguous nucleobases (N—N—N), wherein the one or more first unnatural base (X) is located at the last position (N—N—X) in the codon of the mRNA, wherein the one or more first unnatural base is selected from

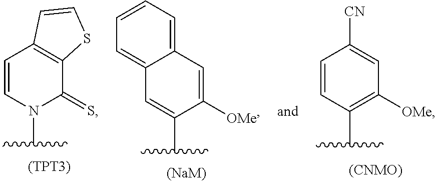

and wherein the wavy line indicates a bond to a ribosyl moiety.

### Embodiment 349

The system of embodiment 332, wherein the at least one anticodon of the tRNA comprises three contiguous nucleobases (N—N—N); and wherein the one or more second unnatural base (X) is located at the first position (X—N—N) in the anticodon of the tRNA, wherein the one or more second unnatural bases is selected from

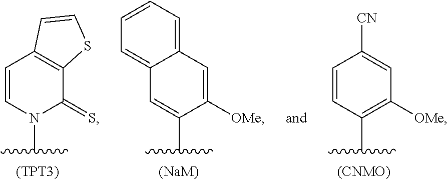

and wherein the wavy line indicates a bond to a ribosyl moiety.

### Embodiment 350

The system of embodiment 332, wherein the at least one anticodon of the tRNA comprises three contiguous nucleobases (N—N—N); and wherein the one or more second unnatural bases (X) is located at the middle position (N—X—N) in the anticodon of the tRNA, wherein the one or more second unnatural bases is selected from

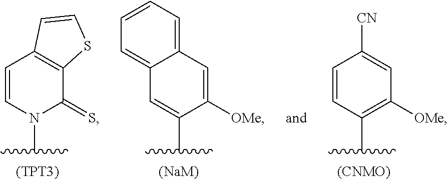

and wherein the wavy line indicates a bond to a ribosyl moiety.

### Embodiment 351

The system of embodiment 332, wherein the at least one anticodon of the tRNA comprises three contiguous nucleobases (N—N—N); and wherein the one or more second unnatural bases (X) is located at the last position (N—N—X) in the anticodon of the tRNA, wherein the one or more second unnatural base is selected from

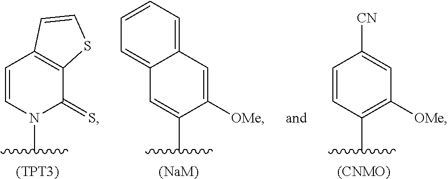

and (CNMO), and wherein the wavy line indicates a bond to a ribosyl moiety.

### Embodiment 352

The system of embodiment 332, wherein the at least one codon and the at least one anticodon each, independently, comprise three contiguous nucleobases (N—N—N), and wherein the at least one codon comprises one or more first unnatural bases (X) located at the first position (X—N—N) of the codon, and the at least one anticodons in the tRNA comprises the one or more second unnatural bases (Y) located at the last position (N—N—Y) of the anticodon.

### Embodiment 353

The system of embodiment 352, wherein the one or more first unnatural bases (X) located in the codon of the mRNA and the one or more second unnatural bases (Y) located in the anticodon of the tRNA are the same or are different.

### Embodiment 354

The system of any one of embodiments 352 to 353, wherein the one or more first unnatural bases (X) located in the codon of the mRNA and the one or more second unnatural bases (Y) located in the anticodon of the tRNA are selected from the group consisting of

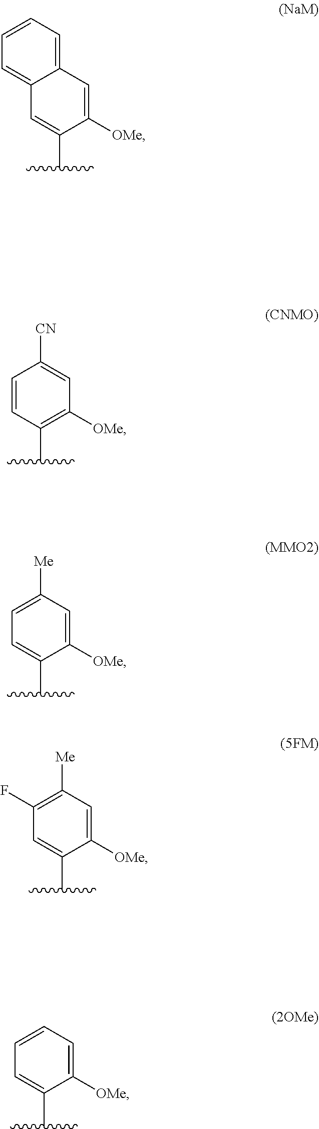 [SMILES: *c1cc2ccccc2cc1OC ; *c1ccc(C#N)cc1OC ; *c1ccc(C)cc1OC ; *c1cc(F)c(C)cc1OC ; *c1ccccc1OC]

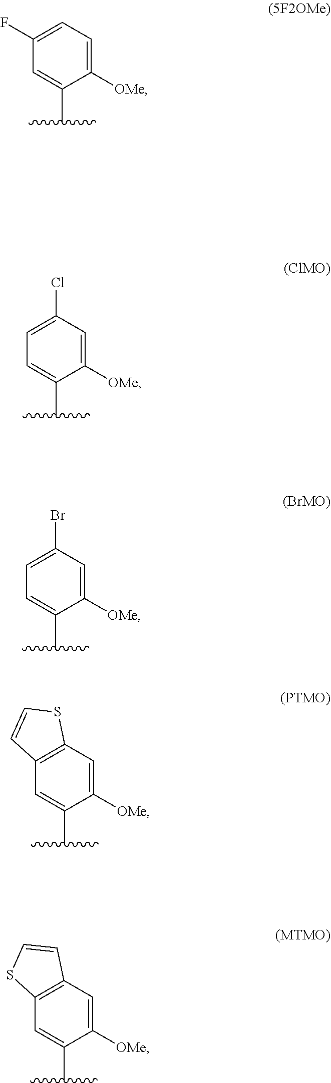 [SMILES: *c1cc(F)ccc1OC ; *c1ccc(Cl)cc1OC ; *c1ccc(Br)cc1OC ; *c1cc2ccsc2cc1OC ; *c1cc2sccc2cc1OC]

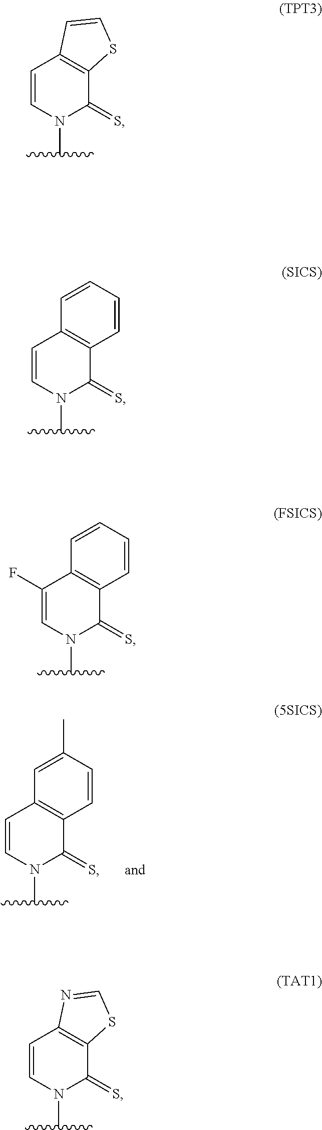 [SMILES: *n1ccc2ccsc2c1=S ; *n1ccc2ccccc2c1=S ; *n1cc(F)c2ccccc2c1=S ; *n1ccc2cc(C)ccc2c1=S ; "and" ; *n1ccc2ncsc2c1=S]

wherein the wavy line indicates a bond to a ribosyl moiety.

### Embodiment 355

The system of embodiment 354, wherein the one or more first unnatural bases (X) located in the codon of the mRNA and the one or more second unnatural bases (Y) located in the anticodon of the tRNA are selected from the group consisting of

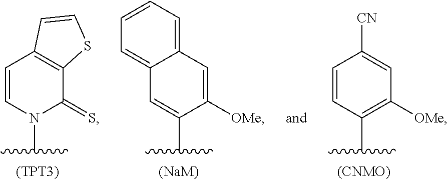

wherein the wavy line indicates a bond to a ribosyl moiety.

### Embodiment 356

The system of embodiment 355, wherein the one or more first unnatural base (X) located in the codon of the mRNA is selected from

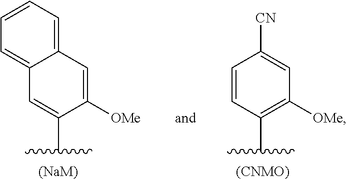

and the one or more second unnatural bases (Y) located in the anticodon of the tRNA is

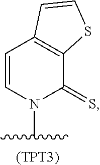

wherein in each case the wavy line indicates a bond to a ribosyl moiety.

### Embodiment 357

The system of embodiment 332, wherein the at least one codon and the at least one anticodon each, independently, comprise three contiguous nucleobases (N—N—N), and wherein the at least one codon in the mRNA comprises the one or more first unnatural bases (X) located at a middle position (N—X—N) of the at least one codon, and the at least one anticodon in the tRNA comprises the one or more second unnatural bases (Y) located at a middle position (N—Y—N) of the anticodon.

### Embodiment 358

The system of embodiment 357, wherein the one or more first unnatural bases (X) located in the codon of the mRNA and the one or more second unnatural bases (Y) located in the anticodon of the tRNA are the same or are different.

### Embodiment 359

The system of any one of embodiments 357 to 358, wherein the one or more first unnatural bases (X) located in the codon of the mRNA and the one or more second unnatural bases (Y) located in the anticodon of the tRNA are selected from the group consisting of

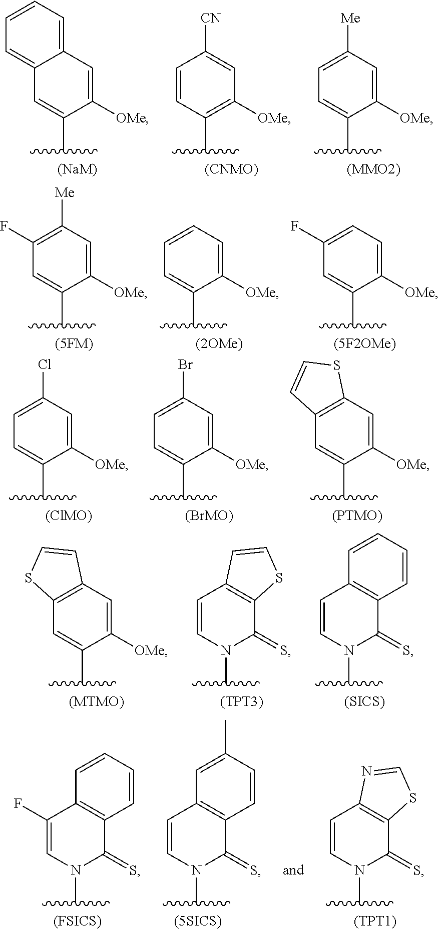

wherein the wavy line indicates a bond to a ribosyl moiety.

### Embodiment 360

The system of embodiment 359, wherein the one or more first unnatural bases (X) located in the codon of the mRNA and the one or more second unnatural bases (Y) located in the anticodon of the tRNA are selected from the group consisting of

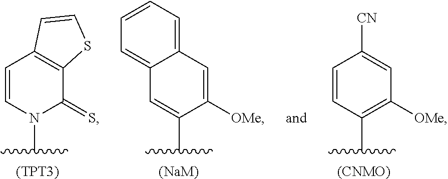

wherein the wavy line indicates a bond to a ribosyl moiety.

### Embodiment 361

The system of embodiment 360, wherein the one or more first unnatural bases (X) located in the codon of the mRNA is selected from

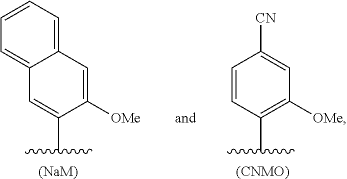

and the one or more second unnatural bases (Y) located in the anticodon of the tRNA is

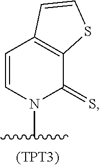

wherein in each case the wavy line indicates a bond to a ribosyl moiety.

### Embodiment 362

The system of embodiment 332, wherein the at least one codon and the at least one anticodon each, independently, comprise three contiguous nucleobases (N—N—N), and wherein the at least one codon in the mRNA comprises the one or more first unnatural bases (X) located at the last position (N—N—X) of the at least one codon, and the at least one anticodon in the tRNA comprises the one or more second unnatural bases (Y) located at the first position (Y—N—N) of the anticodon.

### Embodiment 363

The system of embodiment 362, wherein the one or more first unnatural bases (X) located in the codon of the mRNA and the one or more second unnatural bases (Y) located in the anticodon of the tRNA are the same or are different.

### Embodiment 364

The system of any one of embodiments 362 to 363, wherein the one or more first unnatural bases (X) located in the codon of the mRNA and the one or more second unnatural bases (Y) located in the anticodon of the tRNA are selected from the group consisting of

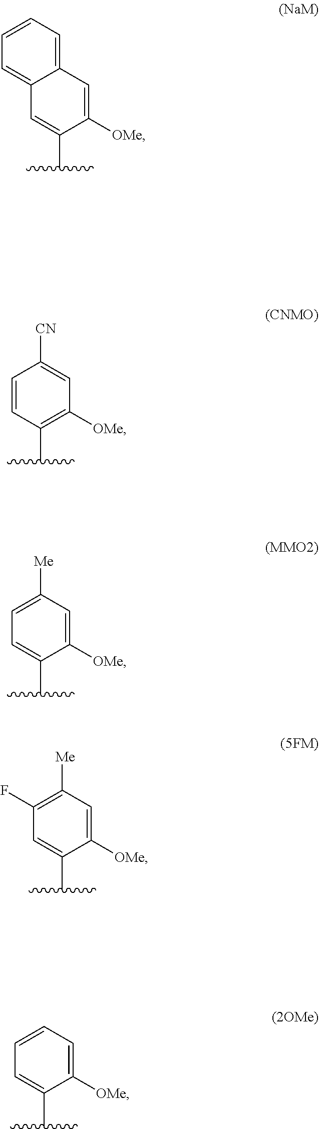 [SMILES: *c1cc2ccccc2cc1OC ; *c1ccc(C#N)cc1OC ; *c1ccc(C)cc1OC ; *c1cc(F)c(C)cc1OC ; *c1ccccc1OC]

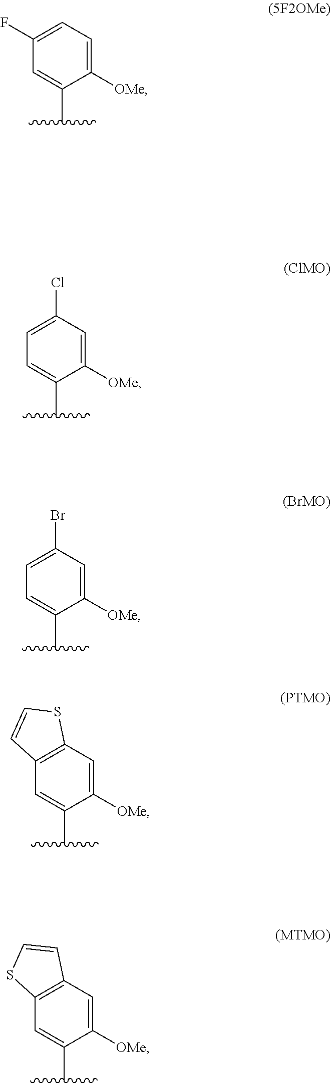 [SMILES: *c1cc(F)ccc1OC ; *c1ccc(Cl)cc1OC ; *c1ccc(Br)cc1OC ; *c1cc2ccsc2cc1OC ; *c1cc2sccc2cc1OC]

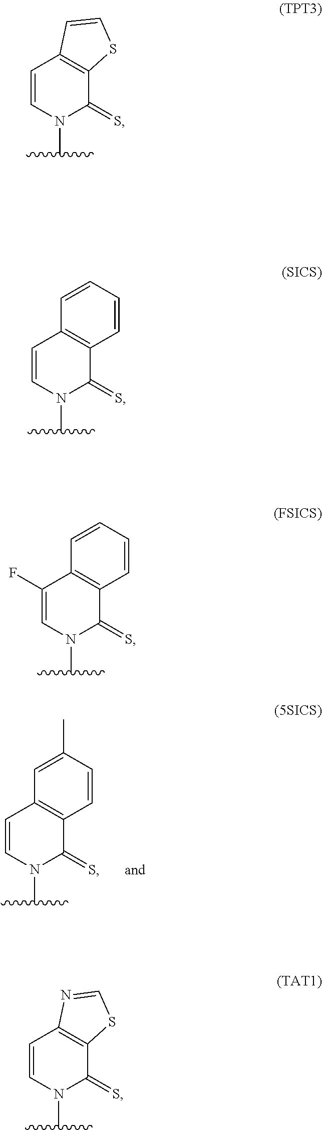 [SMILES: *n1ccc2ccsc2c1=S ; *n1ccc2ccccc2c1=S ; *n1cc(F)c2ccccc2c1=S ; *n1ccc2cc(C)ccc2c1=S ; "and" ; *n1ccc2ncsc2c1=S]

wherein the wavy line indicates a bond to a ribosyl moiety.

### Embodiment 365

The system of embodiment 364, wherein the one or more first unnatural bases (X) located in the codon of the mRNA and the one or more second unnatural bases (Y) located in the anticodon of the tRNA are selected from the group consisting of

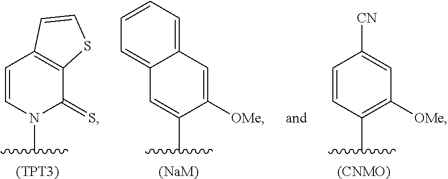

wherein the wavy line indicates a bond to a ribosyl moiety.

### Embodiment 366

The system of embodiment 365, wherein the one or more first unnatural bases (X) located in the codon of the mRNA is selected from

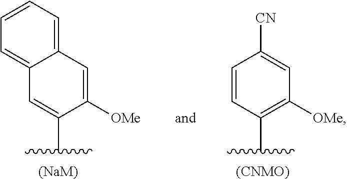

and the one or more second unnatural bases (Y) located in the anticodon of the tRNA is

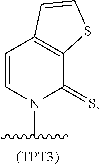

wherein in each case the wavy line indicates a bond to a ribosyl moiety.

### Embodiment 367

The system of any one of embodiments 332 to 366, wherein the at least one codon in the mRNA is selected from AXC, GXC or GXU, wherein X is the one or more first unnatural bases.

### Embodiment 368

The system of the immediately preceding embodiment, wherein the at least one anticodon in the tRNA is selected from GYU, GYC, and AYC, and Y is the one or more second unnatural bases.

### Embodiment 369

The system of embodiment 368, wherein the at least one codon in the mRNA is AXC and the at least one anticodon in the tRNA is GYU.

### Embodiment 370

The system of embodiment 368, wherein the at least one codon in the mRNA is GXC and the at least one anticodon in the tRNA is GYC.

### Embodiment 371

The system of embodiment 368, wherein the at least one codon in the mRNA is GXU and the at least one anticodon is AYC.

### Embodiment 372

The system of any one of embodiments 332 to 371, wherein the tRNA is derived from Methanococcus jannaschii, Methanosarcina barkeri, Methanosarcina mazei, or Methanosarcina acetivorans.

### Embodiment 373

The system of any one of embodiments 332 to 372, wherein the tRNA synthetase is derived from Methanococcus jannaschii, Methanosarcina barkeri, Methanosarcina mazei, or Methanosarcina acetivorans.

### Embodiment 374

The system of any one of claims 332 to 373, which is in a eukaryotic cell.

### Embodiment 374.1

The system of any one of embodiments 332 to 373, which is in a human cell.

### Embodiment 375

The system of embodiment 374.1, wherein the human cell is a HEK293T cell.

### Embodiment 376

The system of any one of embodiments 332 to 373, which is in a mammalian cell.

### Embodiment 376.1

The system of any one of embodiments 332 to 373, which is in a hamster cell.

### Embodiment 377

The system of embodiment 376.1, wherein the hamster cell is a Chinese hamster ovary (CHO) cell.

### Embodiment 377.1

The system of any one of embodiments 332 to 377, wherein the mRNA and the tRNA are stabilized to degradation in the eukaryotic cell.

### Embodiment 377.2

The system of any one of embodiments 332 to 377.1, wherein polypeptide is produced by translation of the mRNA using the tRNA by a ribosome that is endogenous to the eukaryotic cell.

### Embodiment 377.3

The system of any one of claims 332 to 373, which is in vitro or cell-free.

### Embodiment 378

The system of any one of embodiments 332 to 377.3, wherein the unnatural amino acid:
is a lysine analogue;
comprises an aromatic side chain;
comprises an azido group;
comprises an alkyne group; or
comprises an aldehyde or ketone group.

### Embodiment 379

The system of any one of embodiments 332 to 378, wherein the unnatural amino acid is selected from the group consisting of N6-((azidoethoxy)-carbonyl)-L-lysine (AzK), N6-((propargylethoxy)-carbonyl)-L-lysine (PraK), BCN-L-lysine, norbomene lysine, TCO-lysine, methyltetrazine lysine, allyloxycarbonyllysine, 2-amino-8-oxononanoic acid, 2-amino-8-oxooctanoic acid, p-acetyl-L-phenylalanine, p-azidomethyl-L-phenylalanine (pAMF), p-iodo-L-phenylalanine, m-acetylphenylalanine, 2-amino-8-oxononanoic acid, p-propargyloxyphenylalanine, p-propargyl-phenylalanine, 3-methyl-phenylalanine, L-Dopa, fluorinated phenylalanine, isopropyl-L-phenylalanine, p-azido-L-phenylalanine, p-acyl-L-phenylalanine, p-benzoyl-L-phenylalanine, p-bromophenylalanine, p-amino-L-phenylalanine, isopropyl-L-phenylalanine, O-allyltyrosine, O-methyl-L-tyrosine, O-4-allyl-L-tyrosine, 4-propyl-L-tyrosine, phosphonotyrosine, tri-O-acetyl-GlcNAcp-serine, L-phosphoserine, phosphonoserine, L-3-(2-naphthyl)alanine, 2-amino-3-((2-((3-(benzyloxy)-3-oxopropyl)amino)ethyl)selanyl)propanoic acid, 2-amino-3-(phenylselanyl)propanoic, selenocysteine, N6-(((2-azidobenzyl)oxy)carbonyl)-L-lysine, N6-(((3-azidobenzyl)oxy)carbonyl)-L-lysine, or N6-(((4-azidobenzyl)oxy)carbonyl)-L-lysine.

### Embodiment 380

The system of embodiment 379, wherein the unnatural amino acid is N6-((azidoethoxy)-carbonyl)-L-lysine (AzK).

### Embodiment 381

The system of any one of embodiments 332 to 380, wherein the tRNA is charged with the unnatural amino acid.

### Embodiment 382

The method of any one of embodiments 287 to 331, wherein the mRNA and the tRNA are stabilized to degradation in the eukaryotic cell.

### Embodiment 383

The method of any one of embodiments 287 to 331 and 382, wherein the polypeptide is produced by translation of the mRNA using the tRNA by a ribosome that is endogenous to the eukaryotic cell.

### EXAMPLES

These examples are provided for illustrative purposes only and not to limit the scope of the claims provided herein. Detailed methods are provided as the final example herein.

### Example 1: Translation of Unnatural Codons in HEK293T Cells

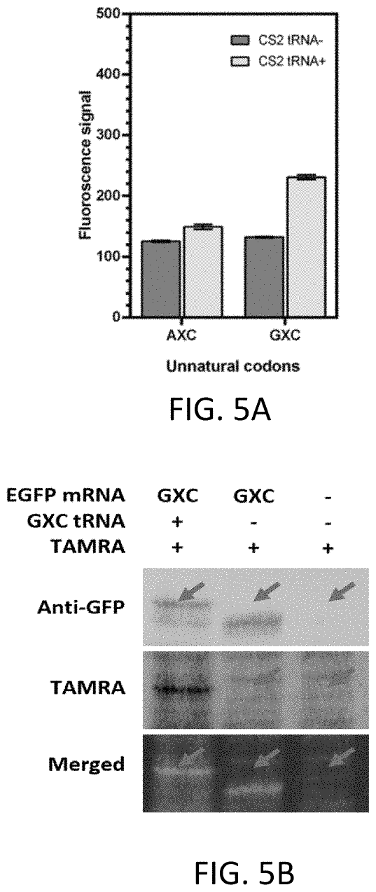

Plasmids encoding EGFP(AXC)¹⁵¹ and EGFP(GXC)¹⁵¹ were constructed with CS2 3′ and 5′ UTR sequences flanking the coding sequence to enhance mRNA stability. The codons AXC and GXC were chosen as they have been shown to be decoded well in the E. coli SSO. The desired mRNAs and cognate tRNAs were produced by in vitro transcription reactions using T7 RNA polymerase. ChPylRS was introduced on a plasmid (pcDNA3.1_C211_IRES_mCherry) harboring a bicistronic sequence encoding both ChPylRS and the mCherry marker connected by an internal ribosome binding site. HEK293T cells were transfected with this plasmid when they reached 50% confluence. Cells were grown for 24 h to allow for the expression of the ChPylRS, and then N6-((azidoethoxy)-carbonyl)-L-lysine (AzK) was added to the medium and cells were transfected with mRNA only, as a control, or mRNA and the corresponding cognate unnatural tRNA. Cells were harvested after an additional 24 h and EGFP production in cells expressing the mCherry marker was quantified via flow cytometry. In controls without tRNA, transfection with EGFP(AXC)¹⁵¹ and EGFP(GXC)¹⁵¹ mRNA resulted in low but detectable levels of EGFP signal, presumably resulting from readthrough of the unnatural codons when their cognate tRNAs were absent. In contrast, cells transfected with both unnatural mRNA and cognate unnatural tRNA exhibited increased fluorescence. While the increase was modest with EGFP(AXC)¹⁵¹, it was more significant with EGFP(GXC)¹⁵¹ (FIG. 5A).

Based on the relatively larger tRNA-dependent increase in fluorescence, the protein produced with the with the EGFP(GXC)¹⁵¹ construct was examined. Total cell lysate was subjected to strain-promoted click chemistry to attach a carboxy-tetramethyl-rhodamine (TAMRA) dye (DBCO-TAMRA), which has been shown to shift the electrophoretic mobility of EGFP as analyzed by SDS-PAGE and thus enables an assessment of the fidelity of N6-((azidoethoxy)-carbonyl)-L-lysine (AzK) incorporation by western blotting. A distinct EGFP signal was apparent (FIG. 5B), with a shift of approximately 70% with lysate prepared from cells transfected with the synthetase plasmid, EGFP(GXC)¹⁵¹ mRNA, and tRNA^{Pyl} (GYC), and grown in medium supplemented with N6-((azidoethoxy)-carbonyl)-L-lysine (AzK). In contrast, little to no shifted band was observed in lysate prepared from cells transfected without cognate unnatural tRNAs. While the low expression level of EGFP precluded further characterization, these data strongly suggest that N6-((azidoethoxy)-carbonyl)-L-lysine (AzK) is incorporated into EGFP through decoding of the unnatural codons using tRNAs with the cognate unnatural anticodon.

### Example 2: Translation of Unnatural Codons in CHO Cells

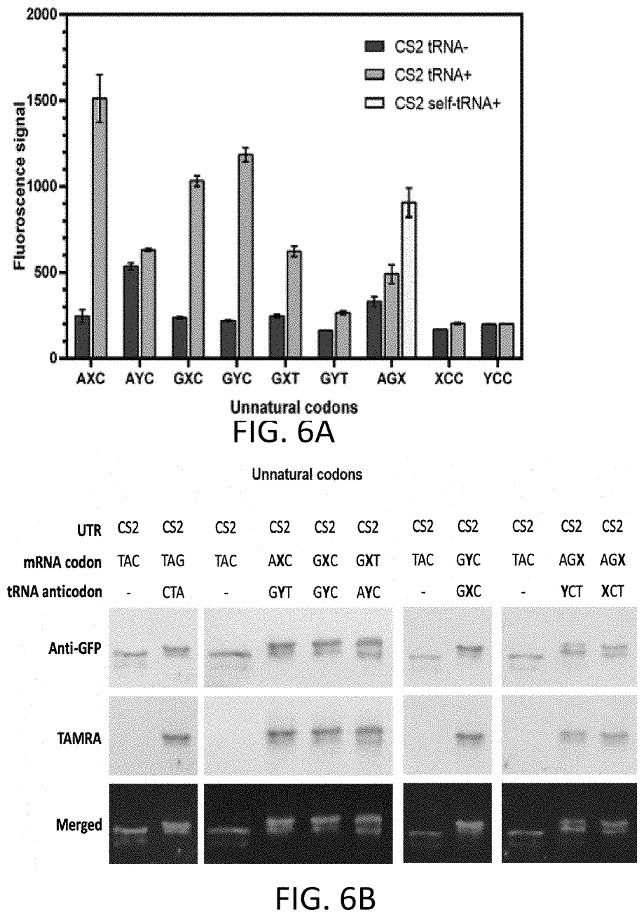

A heterogeneous CHO cell line CHO-KS3 which stably expressed ChPylRS was constructed using the FRT/Flp recombination system, thus reducing transfection to a single RNA co-transfection step. CHO-KS3 cells were transfected with EGFP(AXC)¹⁵¹, EGFP(GXC)¹⁵¹, or EGFP(GXC)¹⁵¹ mRNA, and the cognate tRNA; and N6-((azidoethoxy)-carbonyl)-L-lysine (AzK) was added to the growth medium when cells reached 80% confluence. Cells were harvested after a one-day incubation and then directly subjected to flow cytometry to detect EGFP fluorescence. Control cells not provided with a cognate unnatural tRNA showed similar low but detectable levels of EGFP signal. In contrast, cells transfected with cognate unnatural tRNAs exhibited significantly increased fluorescence, with EGFP(AXC)¹⁵¹ producing the highest fluorescence signal per cell and EGFP(GXU)¹⁵¹ producing the lowest, but fluorescence in all cases was higher than that observed with HEK293T cells (FIG. 6A-6B).

The NaM codons explored above were chosen because they are well translated by the E. coli ribosome. In contrast, the E. coli ribosome appears unable to translate codons containing TPT3. To generate comparative structure-activity relationships between the prokaryotic and eukaryotic ribosomes, EGFP(AYC)¹⁵¹, EGFP(GYC)¹⁵¹ and EGFP(GYU)¹⁵¹, as well as their cognate unnatural tRNAs tRNA^{Pyl} (GXU), tRNA^{Pyl} (GXC) and tRNA^{Pyl} (AXC), were generated and used to transfect CHO-KS3 cells. In contrast to the E. coli SSO, all three TPT3 codons resulted in increased fluorescence when CHO-KS3 cells were transfected with their cognate tRNAs compared to the controls transfected without tRNAs, and in fact, EGFP(GYU)¹⁵¹ achieved a level of fluorescence similar to that observed with the analogous NaM codon (GXU) (FIG. 6A-6B).

With higher EGFP expression levels in CHO-KS3 cells, we selected EGFP(AXC)¹⁵¹, EGFP(GXC)¹⁵¹, EGFP(GXU)¹⁵¹, and EGFP(GYC)¹⁵¹ for more quantitative characterization. EGFP was affinity-purified from cell lysates using a tandem C-terminal Strep-tag II and subjected to click chemistry with the DBCO-TAMRA dye, as described above. Purified EGFP was then analyzed by western blotting. From control cells transfected with natural EGFP mRNA, a dominant band was observed with a faster migrating, weaker band (FIG. 6B). The faster migrating band was attributed to partial Strep tag degradation (data not shown). As expected, neither band showed a TAMRA signal. With transfection of each unnatural mRNA with their cognate tRNA, a similar set of two bands was observed, but both were shifted and showed a TAMRA signal. These results suggest that in CHO cells, N6-((azidoethoxy)-carbonyl)-L-lysine (AzK) is incorporated into EGFP through decoding either NaM or TPT3 codons with cognate unnatural anticodons.

To confirm the correct encoding of N6-((azidoethoxy)-carbonyl)-L-lysine (AzK), liquid chromatography-tandem mass spectrometry (LC-MS/MS) were used to analyze protein purified from CHO-KS3 cells transfected with either EGFP(GXC)¹⁵¹ or EGFP(GYC) mRNA and their cognate tRNAs. EGFP was purified from transfected cells as described above and then subjected to copper-catalyzed click chemistry to attach a 3-butynylbenzene moiety to AzK, to facilitate MS analysis. The reaction product was purified via SDS-PAGE and excising the band between 25 kDa and 32 kDa, which based on previous gel shift assays includes both shifted and unshifted EGFP bands. Proteins recovered from the gel slices were digested with trypsin and subjected to nano-LC-MS/MS analysis. Peptide fragments containing the EGFP amino acid site 151 were detected with masses corresponding to the click reaction product, confirming the specific incorporation of N6-((azidoethoxy)-carbonyl)-L-lysine (AzK) at site 151. Unmodified peptide was not detected, and while not quantitative, this observation confirms the incorporation of N6-((azidoethoxy)-carbonyl)-L-lysine (AzK) and suggests that occurs with at least reasonable fidelity. While a more thorough sequence context analysis remains to be explored, these data demonstrate that mammalian ribosomes, unlike their E. coli counterparts, are able to decode unnatural codons containing either NaM or TPT3.

Previously, it has been shown that an E. coli SSO is also able to translate several codons with the unnatural nucleotide NaM at the third position, including the codon AGX. However, in contrast to the second position, decoding occurred with either the “hetero-pairing” tRNA^{Pyl} (YCT) or the “self-pairing” tRNA^{Pyl} (XCT) (FIG. 5). NaM-NaM self-pairing at the third position may be facilitated in a fashion similar to wobble-pairing of natural codons at the third position. To explore decoding with self-pairing cognate tRNAs in mammalian cells, the AGX codon was tested next in the same mRNA context. CHO-KS3 cells were transfected with EGFP(AGX)¹⁵¹ mRNA alone, or co-transfected along with tRNA^{Pyl} (YCT) or tRNA^{Pyl} (XCT). As with the second position unnatural codons, flow cytometry revealed a small amount of readthrough EGFP expression with cells transfected without any tRNAs. Co-transfecting with tRNA^{Pyl} (YCT) resulted in a significant increase in fluorescence, while co-transfecting with tRNA^{Pyl} (XCT), the self-pairing tRNA, resulted in an even greater increase in fluorescence (FIG. 6A). We then used the same protein shift assay described above to further assess the EGFP produced from unnatural codon AGX. Shifted bands were detected in proteins purified from cells co-transfected with either tRNA^{Pyl} (YCT) or tRNA^{Pyl} (XCT) (FIG. 6B). In both cases, the two shifted bands were again observed, with little to no unshifted band visible. These results demonstrate that at least with the AGX codon, decoding via either hetero-pairing or self-pairing is at least reasonably efficient.

The results with TPT3 codons demonstrate distinct differences between prokaryotic and eukaryotic ribosomes. To further compare these ribosomes, the translation of codons with an unnatural nucleotide in the first position, which the E. coli ribosome appears unable to decode. EGFP(XCC)¹⁵¹ and EGFP(YCC)¹⁵¹ mRNA were produced in vitro and transfected into CHO-KS3 cells without or with their cognate unnatural tRNA, tRNA^{Pyl} (GGY) or tRNA^{Pyl} (GGX), respectively. Analysis using flow cytometry indicated a small amount of readthrough when no tRNAs were added in both cases, with EGFP(YCC)¹⁵¹ resulting in a relatively higher EGFP signal than EGFP(XCC)¹⁵¹. When the corresponding tRNAs were added, a small increase of EGFP signal was observed with EGFP(XCC)¹⁵¹, but no significant increase of EGFP signal was observed with EGFP(YCC)¹⁵¹ (FIG. 6). In both cases, EGFP yields were too low for western blot analysis. These data suggest that, as with the E. coli ribosome, first position unnatural codons are not well decoded. This is likely due to the type I A-minor interaction whereby the ribosome selects for a Watson-Crick-like structure at the first position of the codon.

### Example 3: Protein Expression Ratio Between mRNA with CYBA UTRs and mRNA with CS2 UTRs

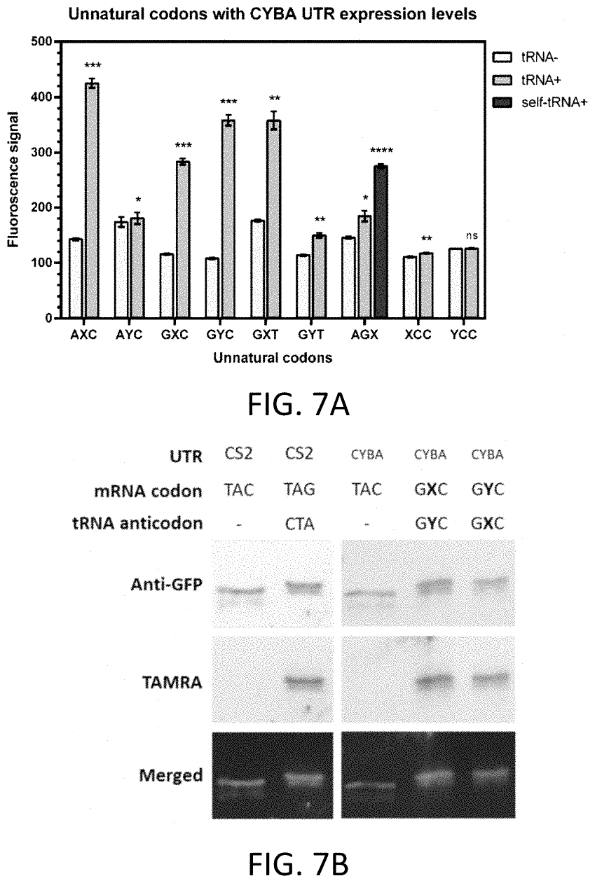

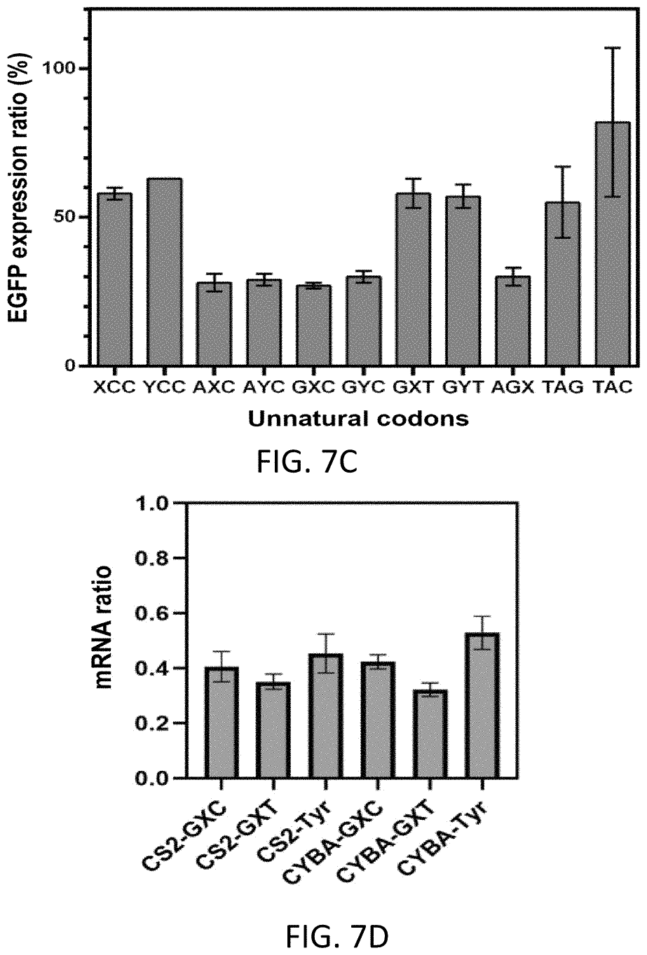

The use of alternate 5′ and 3′ UTRs was examined. The combined use of CYBA 5′ and 3′ UTRs have been reported to increase protein production while not affecting there half-life in human cells. EGFP sequences with all 9 unnatural codons tested above with the CS2 UTRs replaced with CYBA UTRs (CYBA-EGFP(NX/YN)¹⁵¹) were constructed. CHO-KS3 cells were transfected with these newly constructed mRNAs without or with a cognate unnatural tRNA. The cells were then analyzed via flow cytometry and the results were compared to their counterparts with CS2 UTRs. The flow cytometry data indicated that in all cases, less protein was produced with the CYBA UTRs than with their CS2 counterparts. For CYBA-EGFP(GXC)¹⁵¹ and CYBA-EGFP(GYC)¹⁵¹ transfected cells, we also assessed unnatural codon decoding fidelity using the gel shift assay as described above. The shifts observed were similar to those observed with the CS2 UTR counterparts (EGFP(GXC)¹⁵¹ and EGFP(GYC)¹⁵¹), respectively (FIG. 7A-B), demonstrating that the decoding fidelity is not affected significantly by changing the flanking UTRs.

While the reduced level of expression observed with the CYBA UTRs may be due to the use of hamster cells instead of human cells, we also noted that the magnitude of the effect, quite unexpectedly, was significantly different with different unnatural codons. When transfecting with their cognate unnatural tRNAs (the self-pairing tRNA was used with the AGX codon), the XCC, YCC, GXU, and GYU codons with CYBA UTRs exhibited expression levels that were ˜60% of their CS2 counterparts, while expression levels with the AXC, AYC, GXC, GYC, and AGX codons with CYBA UTRs were only ˜30% of their CS2 counterparts (FIG. 7A-D). The amber construct CYBA-EGFP(TAG)¹⁵¹ and natural construct CYBA-EGFP(TAC)¹⁵¹ were used as controls. CYBA-EGFP(TAG)¹⁵¹ and CYBA-EGFP(TAC)¹⁵¹ exhibited expression levels that was ˜60% and ˜80% of their CS2 UTR counterparts.

To test whether this unnatural codon-dependent UTR effect may have originated from differences in mRNA stability, the level of mRNA 8 h post-transfection was compared to that at 4 h post-transfection for EGFP(UAC)¹⁵¹, EGFP(GXC)¹⁵¹, EGFP(GXU)¹⁵¹, CYBA-EGFP(UAC)¹⁵¹, CYBA-EGFP(GXC)¹⁵¹ and CYBA-EGFP(GXU)¹⁵¹ using reverse transcription coupled with quantitative PCR. The differences observed in degradation among these different constructs do not account for the drastic ratio differences described above (FIG. 6), and thus other factors must be responsible. One way that UTRs are thought to affect translation is by regulating ribosome recruitment efficiency. However, it is difficult to rationalize how this could affect the translation of a codon that is far removed from either 5′ or 3′ UTR (in this case by at least 350 nts). Interestingly, multiple ribosome subpopulations are known to exist in a single cell, and may, for example, be differentiated by variable translation elongation abilities. Unlike with the translation of natural codons, this could in principle have a more significant effect on how the ribosome handles different unnatural codons, perhaps similar to our observation that ribosomes from prokaryotes and eukaryotes decode different unnatural codons differently. Further experiments are required to rest this fascinating possibility.

The results disclosed herein demonstrate that unnatural codons may be decoded with at least reasonable efficiency and fidelity in both HEK293T and CHO cells. Interestingly, recognition by the eukaryotic ribosomes shows both similarities and differences with recognition mediated by the E. coli ribosome. First position codons XCC and YCC cannot be decoded with good efficiency in either E. coli or CHO cells; second position NaM codons AXC, GXC and GXU can be decoded with good efficiency in both E. coli and CHO cells; second position codon TPT3 codons AYC, GYC, and GYU cannot be decoded in E. coli but interestingly can be decoded in CHO cells; and the third position codon AGX can be decoded in both E. coli and CHO cells by both its cognate hetero-pairing tRNA as well as its non-cognate self-pairing tRNA.

### Example 4: Methods

Materials and methods used in Examples 1-3 are as follows:

Materials. Plasmids and primers used in Examples 1-4 can be found in Tables 1 and 2. Primers and natural oligonucleotides were purchased from IDT (Coralville, Iowa). Sequencing was performed by Genewiz (San Diego, Calif.). Plasmids were purified using a commercial miniprep kit (Product #D4013, Zymo Research; Irvine, Calif.). PCR products were purified using a commercial DNA purification kit (D4054, Zymo Research) and quantified using an Infinite M200 Pro plate reader (TECAN). All experiments involving RNA species were done with RNase-free reagents, pipette tips, tubes and gloves to avoid contamination. Nucleosides of dNaM, dTPT3, NAM, TPT3, d5SICS and dMMO2bio were synthesized (WuXi AppTec; Shanghai, China) and triphosphorylated (TriLink BioTechnologies LLC; San Diego, Calif. and MyChem LLC; San Diego, Calif.) commercially. All unnatural oligonucleotides were synthesized by Biosearch Technologies (Petaluma, Calif.) with purification by HPLC.

Construction of synthetase plasmids. The chimera synthetase ChPylPS_C211 sequence was cloned from pGEX_ChPylRS, which was described in Fischer et al., Nat. Chem. Biol. 16:570-576 (2020). pcDNA3.1_C211_IRES_mCh was made by cloning ChPylRS, IRES and mCherry sequences one by one into pcDNA3.1 vector using a series of restriction enzymes.

Construction of EGFP and tRNA templates. The EGFP template plasmids, pUCCS2_EGFP(NNN) and pUCCYBA_EGFP(NNN) were made by Golden Gate assembly as described previously but with an EGFP sequence context instead of sfGFP context (see Zhang et al., Nature 551:644-647 (2017)). The inserts used in all Golden Gate assemblies were PCR products generated with synthesized dNaM-containing oligonucleotides and primers YZ73 and YZ74 (see Table 1). Plasmids pUCCS2_EGFP(NNN) and pUCCYBA_EGFP(NNN) were purified after Golden Gate assembly and quantified using Qubit (ThermoFisher). EGFP template plasmids (2 ng) were used in the template-generating PCR reaction with primers ED101 and AZ38 for pUCCS2_EGFP(NNN), and primers ED101 and AZ87 for pUCCYBA_EGFP(NNN). The PCR products were subjected to DpnI digestion and then purified to yield EGFP templates for in vitro transcription (see below). tRNA templates were made by direct PCR from synthesized dNaM-containing oligonucleotides with primers AZ01 and AZ67. The PCR products were purified to yield tRNA templates in vitro transcription.

Biotin Shift Assay. The retention of the unnatural base pair in templates of RNA species were assayed as described in previous work using d5SICSTP and dMMO2bio-TP with primers YZ73 and YZ7 (see Zhang et al., Nature 551:644-647 (2017)). Images were quantified using Image Lab (BioRad). Unnatural base pair retention was normalized by dividing the percentage raw shift of each sample by the percentage raw shift of the synthesized dNaM-containing oligonucleotide template used in the Golden Gate assembly when constructing the EGFP plasmid.

In vitro transcription of EGFP mRNAs. Templates (500-1000 ng) were used in each in vitro transcription reaction (HiScribe T7 ARCA with Tailing, E2060 S, New England Biolabs, (NEB)) with or without 1.25 mM unnatural ribonucleotriphosphate accordingly, followed by purification (D7010, Zymo Research). The mRNA products were quantified by Qubit and then stored in 5 μg aliquots at −80° C.

In vitro transcription of tRNAs. Templates (500-1000 ng) were used in each in vitro transcription reaction (T7 RNA Polymerase, E0251L, NEB) with or without 2 mM unnatural ribonucleotriphosphate accordingly, followed by purification (D7010, Zymo). The tRNA products were quantified by Qubit and then subjected to refolding (95° C. for 1 min, 37° C. for 1 min, 10° C. for 2 min). All tRNAs were stored in 1800 ng aliquots −80° C.

Construction of Stable Cell Line. The synthetase containing plasmid pcDNA3.1_FRT_HygroResist_C211_IRES_mCherry was made by replacing the kanamycin resistance cassette, KanR in pcDNA3.1_C211_IRES_mCherry with the hygromycin resistance cassette, HygroResist via blunt end ligation cloning. The CHO-KS3 heterogeneous cell line was modified to stably express ChPylRS C211 using the Flp-In™ T-REx™ system (ThermoFisher) according to the manufacturer's instructions. The original Flip-In™ CHO-K1 cells were recovered in 10% FBS, 1% PS DMEM/F12 culture. The cells were co-transfected with pOG44 and pcDNA3.1_C211_IRES_mCherry (control) or pcDNA3.1_FRT_HygroResist_C211_IRES_mCherry. The successful recombinant cells were selected with 100 μg/mL hygromycin B (Sigma Aldrich) for two weeks (refreshing the cell culture medium once every four days) until all cells in the control group were dead. Cells transfected with pcDNA3.1_FRT_HygroResist_C211_IRES_mCherry were then detached by trypsin (25200056, Life Technology Invitrogen) digestion (5 min at 37° C.) and passaged for another two rounds with cell culture medium containing 100 μg/mL hygromycin B.

Cell Transfection. Fresh cell culture containing 1 mM AzK was added to cell-culturing plates after depleting the previous medium. For RNA transfection, cells were transfected with RNA species using Lipofectamine MessengerMax (ThermoFisher) according to the reagent manual. For each transfection experiment, 300 ng mRNA and 900 ng tRNA were each mixed with 0.75 μL lipofectamine reagents and added to the cell culture (1 well of a 24-well flat-bottom polystyrene microwell plate) separately. For DNA transfection, cells were transfected with DNA species using Lipofectamine 3000 (LMRNA008, ThermoFisher) according to the reagent manual. For each transfection experiment, 500 ng of DNA plasmid was mixed with 1.5 μL lipofectamine reagents and added to the cell culture (1 well of a 24-well plate). In some cases, cells were transfected in a 12-well plate, and the volumes of the transfection reagents and RNAs were doubled.

Flow Cytometry. Cells were detached by trypsin digestion (5 min at 37° C.) and then washed with 1′ Dulbecco's phosphate buffered saline (DPBS). The cells were then collected and diluted in sorting buffer (1′ DPBS with 1% FBS) and then analyzed by flow cytometry for EGFP signal using an LSR II analytical flow cytometer (BD; EGFP signal was detected with a 488 nm laser and a 530/30 filter).

Whole Cell Lysate Preparation. Cells from transfection experiments were detached by trypsin digestion (5 min at 37° C.) followed by DPBS wash. The cells were then collected and lysed using M-PER (78503, Thermo Fisher) supplied with HALT protease inhibitor (78430, Thermo Fisher) according to the reagent manuals. Lysates were subjected to superfiltration using centrifugal filters (Amicon Ultra—0.5 mL Centrifugal Filters, 10 kDa NMWL, UFC501024, Millipore) to remove the unincorporated AzK. Lysates were washed with DPBS containing HALT (′3). Lysates were concentrated to a volume of 20 μL at the final wash step. All superfiltration was performed at 14,000 rpm for 10 min at 4° C. (5415C, Eppendorf).

Affinity Purification of EGFP. Cells collected from transfection experiments were lysed using M-PER supplied with HALT protease inhibitor according to the reagent manuals. EGFP concentration (fluorescence a.u.) in lysate samples were determined using an Infinite M200 Pro plate reader and an EGFP standard curve. Lysate containing 200 ng EGFP equivalent was diluted into 200 μL with Buffer W (50 mM HEPES pH 8, 150 mM NaCl, 1 mM EDTA) and mixed with 10 μL magnetic Strep-Tactin beads (5% (v/v) suspension of MagStrep ‘Type 3’ XT beads, product #2-4090-002, IBA Lifesciences; Goettingen, Germany). Purification was conducted according to the reagent manual with a prolonged binding time (2 h at 4° C.). EGFP was not eluted from the beads. Bead-EGFP conjugate was used directly in the following experiments.

Click Reaction on EGFP. Click reactions were done as described in previous work (see Zhang et al., Nature 551:644-647 (2017)) with modifications. Briefly, bead-EGFP conjugate from the affinity purification step was diluted in 20 μL DPBS. The mixture was incubated with 25 μM TAMRA-DBCO (Product #A131, Click Chemistry Tools; Scottsdale, Ariz.) for 1 h at 37° C. in darkness. Alternatively, bead-EGFP conjugate from the affinity purification step was diluted in 20 μL DPBS. The mixture was incubated with 2 mM tris(3-hydroxypropyltriazolylmethyl)amine (THPTA) (CAS 760952-88-3, Sigma-Aldrich), 1 mM CuSO4, 15 mM sodium ascorbate (CAS 134-03-2, Sigma-Aldrich) and 0.5 mM 4-phenyl-1-butyne (CAS 16520-62-0, Sigma-Aldrich) for 1 h at 37° C. in darkness. Click reaction of processed whole cell lysate was done by incubating 20 μL superfiltrated cell lysate with 25 μM iodoacetamide (CAS 144-48-9, Sigma-Aldrich) for 1 h at 37° C., followed by incubating the resulting mixture with 25 μM DBCO-TAMRA for 1 h at 37° C. in darkness.

Western Blot Protein Shift Assay. Western blot protein shift assay was done as described in previous work2 with some modification. Briefly, the click reaction mixture was directly boiled in 1′ protein loading dye (250 mM Tris-HCl, 30% (v/v) glycerol, 2% (w/v) SDS) at 95° C. for 15 min and products were resolved on SDS-PAGE (using a stacking gel of 5% (w/v) acrylamide:bis-acrylamide 29:1 (Fisher), 0.125 M TrisHCl and 0.1% SDS, pH 6.8 (ProtoGel Stacking Buffer, National Diagnostics)); and a resolving gel of 15% (w/v) acrylamide:bis-acrylamide 29:1 (Fisher), 0.375 M Tris-HCl and 0.1% SDS, pH 8.8 (ProtoGel Resolving Buffer, National Diagnostics); 1.5 mm spacer Mini-PROTEAN Short Plates (Bio-Rad)) with a protein ladder (Color Prestained Protein Standard, Broad Range, NEB). Gels were run at 60 V for 30 min and then at 135 V for about 3 h in SDS-PAGE buffer (25 mM Tris base, 200 mM glycine, 0.1% (w/v) SDS). Bands were then transferred to PVDF membrane (0.2 μm, Bio-Rad) by semi-dry transfer with a buffer containing 20% (v/v) MeOH, 50 mM Tris base, 400 mM glycine, 0.0373% (w/v) SDS, at 22 V for 21 min. Membranes were blocked with 5% (w/v) nonfat milk in PBS-T (PBS pH 7.4, 0.01% (v/v) Tween-20) for 1-2 h at room temperature, followed by incubation with rabbit anti-GFP antibody (product #G1544, lot 046M4871V, Sigma-Aldrich; 1:3000 in PBS-T) at 4° C. overnight. Next, membranes were washed 2′5 min with PBS-T, followed by incubation with goat anti-rabbit Alexa Fluor 647-conjugated antibody (product #A32733, lot #SD250298, Thermo Fisher Scientific; 1:20000 in PBS-T) for 1 h at room temperature. Membranes were washed 3′5 min with PBS-T and visualized by phosphorimaging (Typhoon 9410; Build S4 410 5.0.0409.0700, GE Healthcare Life Sciences) using 50-μm resolution; 532-nm laser excitation and 580/30-nm emission filter with 400 V PMT for TAMRA; 622-nm laser excitation and 670/30-nm emission filter with 500 V PMT for Alexa Fluor 647. Images were pseudocoloured and overlaid using ImageJ, bands were quantified using Image Lab (Bio-Rad).

Mass Spectrometry. Bead-EGFP conjugates clicked with 4-phenyl-1-butyne was directly boiled with 1′ protein loading dye at 95° C. for 15 min and subjected to SDS-PAGE essentially as for western blot protein shift described above with a protein ladder. Gels were run at 60 V for 30 min and then at 135 V for about 30 min in SDS-PAGE buffer. Gel bands between 25 kDa and 32 kDa were excised and collected, followed by reduction (10 mM DTT), alkylation (55 mM iodoacetamide) and digestion using trypsin. The samples were then analyzed by nano-LC-MS/MS as previously described (see Powers et al., J. Bacteriol. 193:340-348 (2011)). Briefly, data-dependent MS/MS data were obtained with a Thermo Finnigan LTQ linear ion trap mass spectrometer using a home-built nanoelectrospray source at 2 kV at the tip. One MS spectrum was followed by 4 MS/MS scans on the most abundant ions after the application of the dynamic exclusion list. Tandem mass spectra were extracted by use of Xcalibur software. All MS/MS samples were analyzed by using Mascot (version 2.1.04; Matrix Science, London, United Kingdom) with provided EGFP sequence, assuming the digestion enzyme trypsin.

Quantitative High-Resolution Mass Spectrometry of Intact Proteins. The mass spectrometry of intact proteins was conducted as previously described (see Feldman et al., J. Am. Chem. Soc. 141:10644-10653 (2019)). Purified EGFP protein (5 μg) were diluted with water (mass spec grade) and desalted by superfiltration (Amicon Ultra—0.5 mL Centrifugal Filters, 10 kDa NMWL, UFC501024, Millipore). The desalted protein was then injected (6 μL, ˜250 ng) into a Waters IClass LC connected to a Waters G2-XS TOF. Flow conditions were 0.4 mL/min of 50:50 water:acetonitrile plus 0.1% formic acid. Ionization was by ESI+, with data collected between m/z 500 and m/z 2000. A spectral combine was performed over the main portion of the peak, and combined spectrum was deconvoluted using Waters MaxEnt1.

mRNA Decay Assay. For each mRNA tested, 2 wells out of a 12-well plate of CHO-KS1 cells were transfected with 600 ng mRNA and 1800 ng of the corresponding tRNA followed by the addition of 1 mM AzK to the cell culture. After a 4-h incubation, both wells of the cells were washed twice with DPBS and then cells in 1 well were harvested using TRIzole Reagent (15596026, Thermo Fisher; 400 uL TRIzole used for each well). At the same time, the cell culture (containing transfection reagents) in the other well was depleted and fresh cell medium was added. After another 4 h (8 h in total), cells from the remaining well was washed twice with DPBS and then harvested using TRIzole. Both TRIzole solution samples were purified using a total RNA extraction kit (R1013, Zymo). Total RNA (1000 ng) from each sample was used as a template for RT-qPCR with primers AZ112 and AZ86 (suitable for both CS2 UTR and CYBA UTR), the Cq values from which were used to calculate the starting quantity of mRNA in the corresponding total RNA sample. Purified corresponding natural mRNA made from in vitro transcription were used to construct standard curves for quantification reference. The percentage by which mRNA decayed from 4 h (the end of transfection process) to 8 h was calculated by dividing the amount of mRNA difference between 4 h and 8 h by the mRNA amount at 4 h.

TABLE 1
Primers

| SEQ | | |
|---|---|---|
| ID NO | Primer | Sequence |
| 1 | AZ01 | GACAAATTAATACGACTCACTATAG |
| | | GAAACCTGATCATGTAGATCGAAC |
| 2 | AZ38 | CCCCAGGCTTTACACTTTATG |
| 3 | AZ67 | TmGGCGGAAACCCCGGGAATCTAAC |
| | | CCGGCTGAACGGATT |
| 4 | AZ86 | TCCACGCCGAACCTCCCGATC |
| 5 | AZ87 | TCCCGGCTTCGCTGCATTTATTGC |
| 6 | AZ112 | AAAATCACGGCAGACAAACAAAAG |
| | | AATGG |
| 7 | YZ73 | ATGGGTCTCACACAAACTCGA |
| | | GTACAACTTTAACTCACAC |
| 8 | YZ74 | ATGGGTCTCGATTCCATTCTTTT |
| | | GTTTGTCTGC |
| 9 | ED101 | TAATACGACTCACTATAGG |

TABLE 2
Oligonucleotides

| SEQ | | |
|---|---|---|
| ID | oligo- | oligonucleotide |
| NO | nucleotide | Sequence |
| 10 | EGFP_Y151_ | CTCGAGTACAACTTTAACTCACACAATGTATA |
| | TAC | CATCACGGCAGACAAACAAAAGAATGGAATC |
| 11 | EGFP_Y151_ | CTCGAGTACAACTTTAACTCACACAATGTAGT |
| | TAG | AATCACGGCAGACAAACAAAAGAATGGAATC |
| 12 | EGFP_Y151_ | CTCGAGTACAACTTTAACTCACACAATGTAAX |
| | AXC | CATCACGGCAGACAAACAAAAGAATGGAATC |
| 13 | EGFP_Y151_ | CTCGAGTACAACTTTAACTCACACAATGTAAY |
| | AYC | GCATCACGCAGACAAACAAAAGAATGGAATC |
| 14 | EGFP_Y151_ | CTCGAGTACAACTTTAACTCACACAATGTAGX |
| | GXC | CATCACGGCAGACAAACAAAAGAATGGAATC |
| 15 | EGFP_Y151_ | CTCGAGTACAACTTTAACTCACACAATGTAGY |
| | GYC | CATCACGGCAGACAAACAAAAGAATGGAATC |
| 16 | EGFP_Y151_ | CTCGAGTACAACTTTAACTCACACAATGTAGX |
| | GXT | TATCACGGCAGACAAACAAAAGAATGGAATC |
| 17 | EGFP_Y151_ | CTCGAGTACAACTTTAACTCACACAATGTAGY |
| | GYT | TATCACGGCAGACAAACAAAAGAATGGAATC |
| 18 | EGFP_Y151_ | CTCGAGTACAACTTTAACTCACACAATGTAAG |
| | AGX | XATCACGGCAGACAAACAAAAGAATGGAATC |
| 19 | EGFP_Y151_ | CTCGAGTACAACTTTAACTCACACAATGTAXC |
| | XCC | CATCACGGCAGACAAACAAAAGAATGGAATC |
| 20 | EGFP_Y151_ | CTCGAGTACAACTTTAACTCACACAATGTAYC |
| | YCC | CATCACGGCAGACAAACAAAAGAATGGAATC |
| 21 | Mm_tRNA_ | CCTGATCATGTAGATCGAACGGACTGTAAATC |
| | GTA | CGTTCAGCCGGGTTAGATTC |
| 22 | Mm_tRNA_ | CCTGATCATGTAGATCGAACGGACTCTAAATC |
| | CTA | CGTTCAGCCGGGTTAGATTC |
| 23 | Mm_tRNA_ | CCTGATCATGTAGATCGAACGGACTGYTAATC |
| | GYT | CGTTCAGCCGGGTTAGATTC |
| 24 | Mm_tRNA_ | CCTGATCATGTAGATCGAACGGACTGXTAATC |
| | GXT | CGTTCAGCCGGGTTAGATTC |
| 25 | Mm_tRNA_ | CCTGATCATGTAGATCGAACGGACTGYCAATC |
| | GYC | GCGTTCACCGGGTTAGATTC |
| 26 | Mm_tRNA_ | CCTGATCATGTAGATCGAACGGACTGXCAATC |
| | GXC | CGTTCAGCCGGGTTAGATTC |
| 27 | Mm_tRNA_ | CCTGATCATGTAGATCGAACGGACTAYCAATC |
| | AYC | CGTTCAGCCGGGTTAGATTC |
| 28 | Mm_tRNA_ | CCTGATCATGTAGATCGAACGGACTAXCAATC |
| | AXC | CGTTCAGCCGGGTTAGATTC |
| 29 | Mm_tRNA_ | CCTGATCATGTAGATCGAACGGACTYCTAATC |
| | YCT | CGTTCAGCCGGGTTAGATTC |
| 30 | Mm_tRNA_ | CCTGATCATGTAGATCGAACGGACTXCTAATC |
| | XCT | GCGTTCACCGGGTTAGATTC |
| 31 | Mm_tRNA_ | CCTGATCATGTAGATCGAACGGACTGGYAATC |
| | GGY | CGTTCAGCCGGGTTAGATTC |
| 32 | Mm_tRNA_ | CCTGATCATGTAGATCGAACGGACTGGXAATC |
| | GGX | CGTTCAGCCGGGTTAGATTC |

Other Sequences
IRES (SEQ ID NO: 33):
CATCTAGGGCGGCCAATTCCGCCCCTCTCCCTCCC
CCCCCCCTAACGTTACTGGCCGAAGCCGCTTGGAA
TAAGGCCGGTGTGCGTTTGTCTATATGTGATTTTC
CACCATATTGCCGTCTTTTGGCAATGTGAGGGCCC
GGAAACCTGGCCCTGTCTTCTTGACGAGCATTCCT
AGGGGTCTTTCCCCTCTCGCCAAAGGAATGCAAGG
TCTGTTGAATGTCGTGAAGGAAGCAGTTCCTCTGG
AAGCTTCTTGAAGACAAACAACGTCTGTAGCGACC
CTTTGCAGGCAGCGGAACCCCCCACCTGGCGACAG
GTGCCTCTGCGGCCAAAAGCCACGTGTATAAGATA
CACCTGCAAAGGCGGCACAACCCCAGTGCCACGTT
GTGAGTTGGATAGTTGTGGAAAGAGTCAAATGGCT
CTCCTCAAGCGTATTCAACAAGGGGCTGAAGGATG
CCCAGAAGGTACCCCATTGTATGGGATCTGATCTG
GGGCCTCGGTGCACATGCTTTACATGTGTTTAGTC
GAGGTTAAAAAAACGTCTAGGCCCCCCGAACCACG
GGGACGTGGTTTTCCTTTGAAAAACACGATGATAA
GCTTGCCAC
mCherry (SEQ ID NO: 34)
ATGGTGAGCAAGGGCGAGGAGGATAACATGGCCAT
CATCAAGGAGTTCATGCGCTTCAAGGTGCACATGG
AGGGCTCCGTGAACGGCCACGAGTTCGAGATCGAG
GGCGAGGGCGAGGGCCGCCCCTACGAGGGCACCCA
GACCGCCAAGCTGAAGGTGACCAAGGGTGGCCCCC
TGCCCTTCGCCTGGGACATCCTGTCCCCTCAGTTC
ATGTACGGCTCCAAGGCCTACGTGAAGCACCCCGC
CGACATCCCCGACTACTTGAAGCTGTCCTTCCCCG
AGGGCTTCAAGTGGGAGCGCGTGATGAACTTCGAG
GACGGCGGCGTGGTGACCGTGACCCAGGACTCCTC
CCTGCAGGACGGCGAGTTCATCTACAAGGTGAAGC
TGCGCGGCACCAACTTCCCCTCCGACGGCCCCGTA
ATGCAGAAGAAGACCATGGGCTGGGAGGCCTCCTC
CGAGCGGATGTACCCCGAGGACGGCGCCCTGAAGG
GCGAGATCAAGCAGAGGCTGAAGCTGAAGGACGGC
GGCCACTACGACGCTGAGGTCAAGACCACCTACAA
GGCCAAGAAGCCCGTGCAGCTGCCCGGCGCCTACA
ACGTCAACATCAAGTTGGACATCACCTCCCACAAC
GAGGACTACACCATCGTGGAACAGTACGAACGCGC
CGAGGGCCGCCACTCCACCGGCGGCATGGACGAGC
TGTACAAGTAA
ChPylRS_C211 (SEQ ID NO: 35)
ATGGATAAAAAACCGCTGGACGTTCTGATCTCCGC
TACGGGTCTGTGGATGAGCCGCACGGGTACGCTGC
ATAAAATCAAGCACTATGAGATTTCTCGTTCTAAA
ATCTACATCGAAATGGCGTGTGGTGACCATCTGGT
TGTGAACAACTCTCGTTCTTGTCGTCCGGCACGTG
CATTCCGTTATCATAAATACCGTAAAACCTGCAAA
CGTTGTCGTGTTTCTGACGAAGATATCAACAACTT
CCTGACCCGTTCTACCGAAGGCAAAACCTCTGTTA
AAGTTAAAGTTGTTTCTGAACCGAAAGTGAAAAAA
GCGATGCCGAAATCTGTTTCTCGTGCGCCGAAACC
GCTGGAAAATCCGGTTTCTGCGAAAGCGTCTACCG
ACACCTCTCGTTCTGTTCCGTCTCCGGCGAAATCT
ACCCCGAACTCTCCGGTTCCGACCTCTGCAAGTGC
CCCCGCACTTACGAAGAGCCAGACTGACAGGCTTG
AAGTCCTGTTAAACCCAAAAGATGAGATTTCCCTG
AATTCCGGCAAGCCTTTCAGGGAGCTTGAGTCCGA
ATTGCTCTCTCGCAGAAAAAAAGACCTGCAGCAGA
TCTACGCGGAAGAAAGGGAGAATTATCTGGGGAAA
CTCGAGCGTGAAATTACCAGGTTCTTTGTGGACAG
GGGTTTTCTGGAAATAAAATCCCCGATCCTGATCC
CTCTTGAGTATATCGAAAGGATGGGCATTGATAAT
GATACCGAACTTTCAAAACAGATCTTCAGGGTTGA
CAAGAACTTCTGCCTGAGACCCATGCTTGCTCCAA
ACCTTTACAACTACCTGCGCAAGCTTGACAGGGCC
CTGCCTGATCCAATAAAAATTTTTGAAATAGGCCC
ATGCTACAGAAAAGAGTCCGACGGCAAAGAACACC
TCGAAGAGTTTACCATGCTGAACTTCTGCCAGATG
GGATCGGGATGCACACGGGAAAATCTTGAAAGCAT
AATTACGGACTTCCTGAACCACCTGGGAATTGATT
TCAAGATCGTAGGCGATTCCTGCATGGTCTATGGG
GATACCCTTGATGTAATGCACGGAGACCTGGAACT
TTCCTCTGCAGTAGTCGGACCCATACCGCTTGACC
GGGAATGGGGTATTGATAAACCCTGGATAGGGGCA
GGTTTCGGACTCGAACGCCTTCTAAAGGTTAAACA
CGACTTTAAAAATATCAAGAGAGCTGCACGCTCGG
AATCGTATTACAACGGCATCTCAACCAATCTGTAA
CS2 5′UTR (SEQ ID NO: 36):
GAATACAAGCTACTTGTTCTTTTTGCAGGATCCGC
CACC
C52 3′UTR (SEQ ID NO: 37):
AAGCTTAATTAGCTGAGCTTGGACTCCTAAGCATG
CAAGCTTGGCGTAATCATGGTCATAGCTGTTTCCT
GTGTGAAATTGTTATCCGCTCACAATTCCACACAA
CATACGAGCCGGAAGCATAAAGTGTAAAGCCTGGG
G
CYBA 5′UTR (SEQ ID NO: 38):
CGCGCCTAGCAGTGTCCCAGCCGGGTTCGTGTCGC
C
CYBA 3′UTR (SEQ ID NO: 39):
CCTCGCCCCGGACCTGCCCTCCCGCCAGGTGCACC
CACCTGCAATAAATGCAGCGAAGCCGGGA
EGFP(Golden Gate vector)
(with 2xStrepTag)
(SEQ ID NO: 40):
ATGGTGAGCAAGGGCGAGGAGCTGTTCACCGGGGT
GGTGCCCATCCTGGTCGAGCTGGACGGCGACGTAA
ACGGCCACAAGTTCAGCGTGTCCGGCGAGGGCGAG
GGCGATGCCACCTACGGCAAGCTGACCCTGAAGTT
CATCTGCACCACCGGCAAGCTGCCCGTGCCCTGGC
CCACCCTCGTGACCACCCTGACCTACGGCGTGCAG
TGCTTCAGCCGCTACCCCGACCACATGAAGCAGCA
CGACTTCTTCAAGTCCGCCATGCCCGAAGGCTACG
TCCAGGAGCGCACCATCTTCTTCAAGGACGACGGC
AACTACAAGACCCGCGCCGAGGTGAAGTTCGAGGG
CGACACCCTGGTGAACCGCATCGAGCTGAAGGGCA
TCGACTTCAAGGAGGACGGCAACATCCTGGGGCAC
AAGAGACCCTCGAGAATATTCTCGAGGGTCTCGGA
ATCAAGGTGAACTTCAAGATCCGCCACAACATCGA
GGACGGCAGCGTGCAGCTCGCCGACCACTACCAGC
AGAACACCCCCATCGGCGACGGCCCCGTGCTGCTG
CCCGACAACCACTACCTGAGCACCCAGTCCGCCCT
GAGCAAAGACCCCAACGAGAAGCGCGATCACATGG
TCCTGCTGGAGTTCGTGACCGCCGCCGGGATCACT
CTCGGCATGGACGAGCTGTACAAGAAGCTTTGGAG
CCACCCGCAGTTCGAGAAAGGTGGAGGTTCCGGAG
GTGGATCGGGAGGTTCGGCGTGGAGCCACCCGCAG
TTCGAAAAATAA
FLP (SEQ ID NO: 41)
ATGCCACAATTTGATATATTATGTAAAACACCACC
TAAGGTGCTTGTTCGTCAGTTTGTGGAAAGGTTTG
AAAGACCTTCAGGTGAGAAAATAGCATTATGTGCT
GCTGAACTAACCTATTTATGTTGGATGATTACACA
TAACGGAACAGCAATCAAGAGAGCCACATTCATGA
GCTATAATACTATCATAAGCAATTCGCTGAGTTTG
GATATTGTCAACAAGTCACTGCAGTTTAAATACAA
GACGCAAAAAGCAACAATTCTGGAAGCCTCATTAA
AGAAATTGATTCCTGCTTGGGAATTTACAATTATT
CCTTACTATGGACAAAAACATCAATCTGATATCAC
TGATATTGTAAGTAGTTTGCAATTACAGTTCGAAT
CATCGGAAGAAGCAGATAAGGGAAATAGCCACAGT
AAAAAAATGCTTAAAGCACTTCTAAGTGAGGGTGA
AAGCATCTGGGAGATCACTGAGAAAATACTAAATT
CGTTTGAGTATACTTCGAGATTTACAAAAACAAAA
ACTTTATACCAATTCCTCTTCCTAGCTACTTTCAT
CAATTGTGGAAGATTCAGCGATATTAAGAACGTTG
ATCCGAAATCATTTAAATTAGTCCAAAATAAGTAT
CTGGGAGTAATAATCCAGTGTTTAGTGACAGAGAC
AAAGACAAGCGTTAGTAGGCACATATACTTCTTTA
GCGCAAGGGGTAGGATCGATCCACTTGTATATTTG
GATGAATTTTTGAGGAATTCTGAACCAGTCCTAAA
ACGAGTAAATAGGACCGGCAATTCTTCAAGCAACA
AGCAGGAATACCAATTATTAAAAGATAACTTAGTC
AGATCGTACAACAAAGCTTTGAAGAAAAATGCGCC
TTATTCAATCTTTGCTATAAAAAATGGCCCAAAAT
CTCACATTGGAAGACATTTGATGACCTCATTTCTT
TCAATGAAGGGCCTAACGGAGTTGACTAATGTTGT
GGGAAATTGGAGCGATAAGCGTGCTTCTGCCGTGG
CCAGGACAACGTATACTCATCAGATAACAGCAATA
CCTGATCACTACTTCGCACTAGTTTCTCGGTACTA
TGCATATGATCCAATATCAAAGGAAATGATAGCAT
TGAAGGATGAGACTAATCCAATTGAGGAGTGGCAG
CATATAGAACAGCTAAAGGGTAGTGCTGAAGGAAG
CATACGATACCCCGCATGGAATGGGATAATATCAC
AGGAGGTACTAGACTACCTTTCATCCTACATAAAT
AGACGCATATAA
FRT (SEQ ID NO: 42)
GAAGTTCCTATTCCGAAGTTCCTATTCTCTAGAAA
GTATAGGAACTTC

While preferred embodiments of the disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the invention and that methods and structures within the scope of these claims and their equivalents be covered thereby.

What is claimed is:

1. A eukaryotic cell comprising:
(a) a messenger RNA (mRNA) with a codon comprising a first unnatural base; and
(b) a transfer RNA (tRNA) with an anticodon comprising a second unnatural base,
wherein the first and second unnatural bases are capable of forming an unnatural base pair (UBP) in the eukaryotic cell, and wherein the mRNA is capable of being translated in the cell to produce a polypeptide comprising at least one unnatural amino acid.

2. The eukaryotic cell of claim 1, wherein the tRNA is charged with an unnatural amino acid.

3. The eukaryotic cell of any one of the preceding claims, further comprising a polypeptide translated from the mRNA, wherein the polypeptide comprises the unnatural amino acid, optionally wherein the polypeptide comprises a eukaryotic glycosylation pattern.

4. The eukaryotic cell of any one of the preceding claims, further comprising a tRNA synthetase, wherein the tRNA synthetase preferentially aminoacylates the tRNA with the unnatural amino acid.

5. The eukaryotic cell of any one of the preceding claims, wherein the codon of the mRNA comprises three contiguous nucleobases (N—N—N); and wherein the first unnatural base (X) is located at the first position (X—N—N) in the codon of the mRNA.

6. The eukaryotic cell of any one of the preceding claims, wherein the codon of the mRNA comprises three contiguous nucleobases (N—N—N); and wherein the first unnatural base (X) is located at the middle position (N—X—N) in the codon of the mRNA.

7. The eukaryotic cell of any one of the preceding claims, wherein the codon of the mRNA comprises three contiguous nucleobases (N—N—N); and wherein the first unnatural base (X) is located at the last position (N—N—X) in the codon of the mRNA.

8. The eukaryotic cell of any one of the preceding claims, wherein the first unnatural base and the second unnatural base are each, independently, selected from the group consisting of 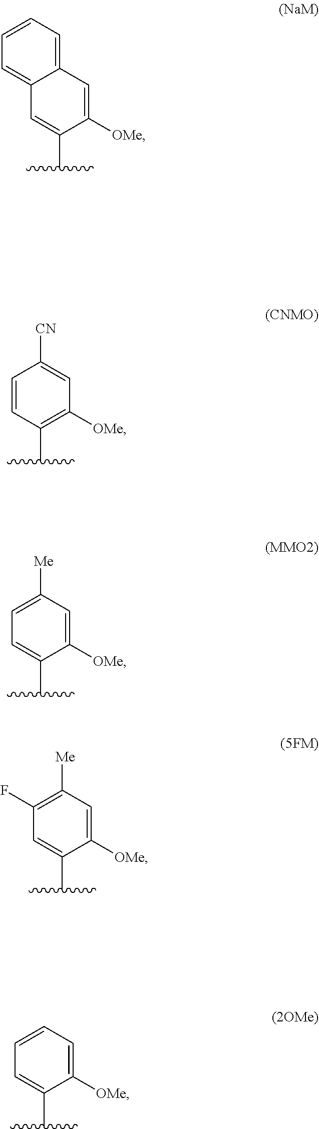 [SMILES: *c1cc2ccccc2cc1OC ; *c1ccc(C#N)cc1OC ; *c1ccc(C)cc1OC ; *c1cc(F)c(C)cc1OC ; *c1ccccc1OC] 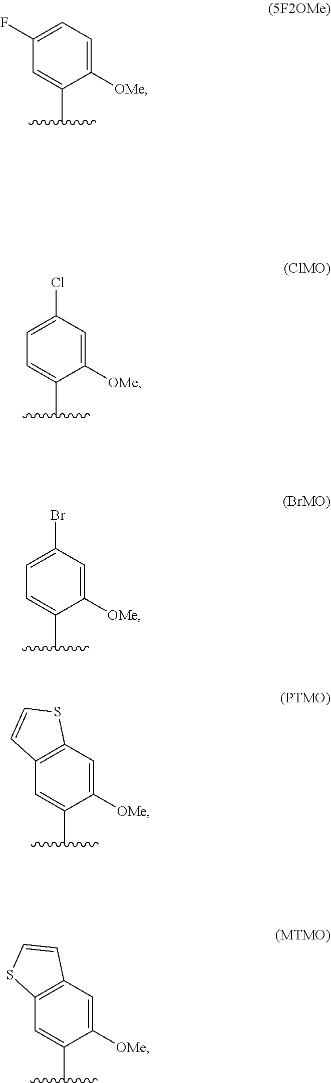 [SMILES: *c1cc(F)ccc1OC ; *c1ccc(Cl)cc1OC ; *c1ccc(Br)cc1OC ; *c1cc2ccsc2cc1OC ; *c1cc2sccc2cc1OC] 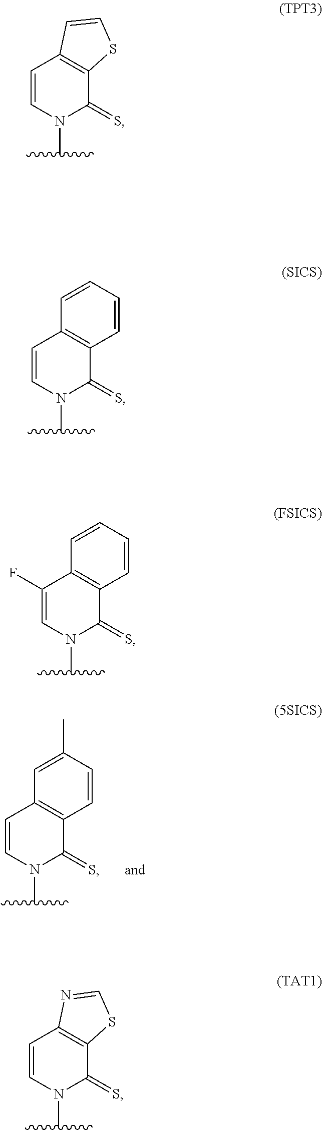 [SMILES: *n1ccc2ccsc2c1=S ; *n1ccc2ccccc2c1=S ; *n1cc(F)c2ccccc2c1=S ; *n1ccc2cc(C)ccc2c1=S ; "and" ; *n1ccc2ncsc2c1=S]
wherein the wavy line indicates a bond to a ribosyl moiety.

9. The eukaryotic cell of any one of the preceding claims, when the first unnatural base is 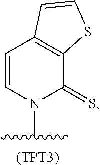
the second unnatural base is 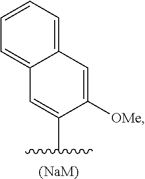
and when the first unnatural base is 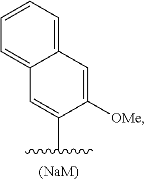
the second unnatural base is 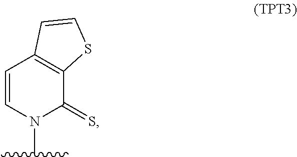 [SMILES: *n1ccc2ccsc2c1=S]
wherein the wavy line indicates a bond to a ribosyl moiety.

10. The eukaryotic cell of any one of the preceding claims, when the first unnatural base is 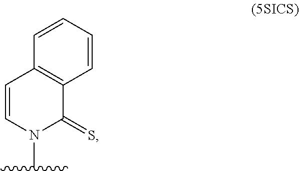 [SMILES: *n1ccc2ccccc2c1=S]
the second unnatural base is 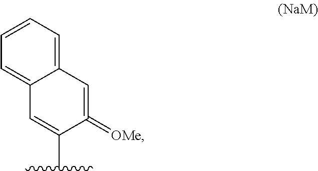
and when the first unnatural base is 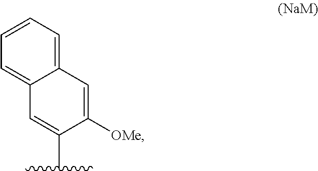 [SMILES: *c1cc2ccccc2cc1OC]
the second unnatural base is 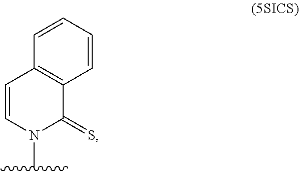 [SMILES: *n1ccc2ccccc2c1=S]
wherein the wavy line indicates a bond to a ribosyl moiety.

11. The eukaryotic cell of any one of the preceding claims, when the first unnatural base is 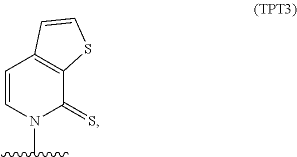 [SMILES: *n1ccc2ccsc2c1=S]
the second unnatural base is 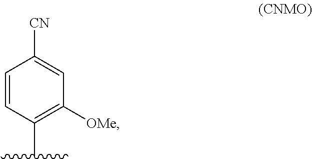 [SMILES: *c1ccc(C#N)cc1OC]
and when the first unnatural base is 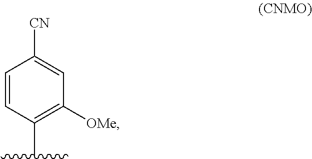 [SMILES: *c1ccc(C#N)cc1OC]
the second unnatural base is 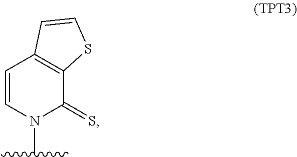 [SMILES: *n1ccc2ccsc2c1=S]
wherein the wavy line indicates a bond to a ribosyl moiety.

12. The eukaryotic cell of any one of the preceding claims, when the first unnatural base is 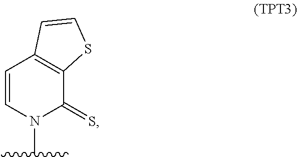 [SMILES: *n1ccc2ccsc2c1=S]
the second unnatural base is 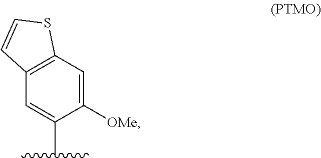 [SMILES: *c1cc2ccsc2cc1OC]
and when the first unnatural base is 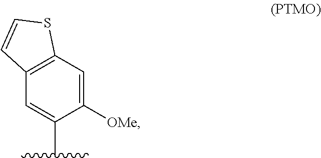 [SMILES: *c1cc2ccsc2cc1OC]
the second unnatural base is 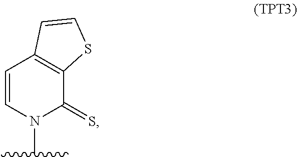 [SMILES: *n1ccc2ccsc2c1=S]
wherein the wavy line indicates a bond to a ribosyl moiety.

13. The eukaryotic cell of any one of the preceding claims, when the first unnatural base is 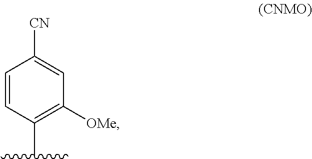 [SMILES: *c1ccc(C#N)cc1OC]
the second unnatural base is 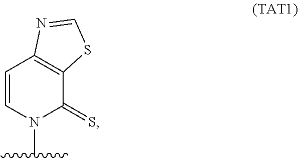 [SMILES: *n1ccc2ncsc2c1=S]
and when the first unnatural base is 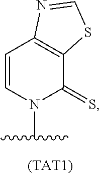
the second unnatural base is 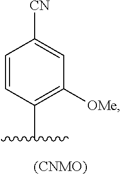
wherein the wavy line indicates a bond to a ribosyl moiety.

14. The eukaryotic cell of any one of the preceding claims, when the first unnatural base is 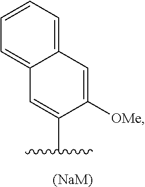
the second unnatural base is 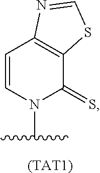
and when the first unnatural base is 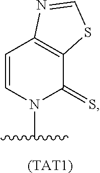
the second natural base is (NaM), wherein the wavy line indicates a bond to a ribosyl moiety.

15. The eukaryotic cell of any one of claims 3 to 14, wherein the at least one unnatural amino acid:
is a lysine analogue;
comprises an aromatic side chain;
comprises an azido group;
comprises an alkyne group; or
comprises an aldehyde or ketone group.

16. The eukaryotic cell of claim 15, wherein the at least one unnatural amino acid is selected from the group consisting of N6-((azidoethoxy)-carbonyl)-L-lysine (AzK), N6-((propargylethoxy)-carbonyl)-L-lysine (PraK), BCN-L-lysine, norbomene lysine, TCO-lysine, methyltetrazine lysine, allyloxycarbonyllysine, 2-amino-8-oxononanoic acid, 2-amino-8-oxooctanoic acid, p-acetyl-L-phenylalanine, p-azidomethyl-L-phenylalanine (pAMF), p-iodo-L-phenylalanine, m-acetylphenylalanine, 2-amino-8-oxononanoic acid, p-propargyloxyphenylalanine, p-propargyl-phenylalanine, 3-methyl-phenylalanine, L-Dopa, fluorinated phenylalanine, isopropyl-L-phenylalanine, p-azido-L-phenylalanine, p-acyl-L-phenylalanine, p-benzoyl-L-phenylalanine, p-bromophenylalanine, p-amino-L-phenylalanine, isopropyl-L-phenylalanine, O-allyltyrosine, O-methyl-L-tyrosine, O-4-allyl-L-tyrosine, 4-propyl-L-tyrosine, phosphonotyrosine, tri-O-acetyl-GlcNAcp-serine, L-phosphoserine, phosphonoserine, L-3-(2-naphthyl)alanine, 2-amino-3-((2-((3-(benzyloxy)-3-oxopropyl)amino)ethyl)selanyl)propanoic acid, 2-amino-3-(phenylselanyl)propanoic, selenocysteine, N6-(((2-azidobenzyl)oxy)carbonyl)-L-lysine, N6-(((3-azidobenzyl)oxy)carbonyl)-L-lysine, or N6-(((4-azidobenzyl)oxy)carbonyl)-L-lysine.

17. The eukaryotic cell of claim 16, wherein the at least one unnatural amino acid is N6-((azidoethoxy)-carbonyl)-L-lysine (AzK).

18. The eukaryotic cell of any one of the preceding claims, wherein the eukaryotic cell is a human cell.

19. The eukaryotic cell of the immediately preceding claim, wherein the human cell is a HEK293T cell.

20. The eukaryotic cell of any one of claims 1 to 18, wherein the cell is a mammalian cell, optionally wherein the cell is a hamster cell.

21. The eukaryotic cell of the immediately preceding claim, wherein the mammalian cell is a Chinese hamster ovary (CHO) cell.

22. The eukaryotic cell of any one of claims 18-21, further comprising a polypeptide translated from the mRNA, wherein the polypeptide comprises the unnatural amino acid and a mammalian glycosylation pattern.

23. The eukaryotic cell of any one of the preceding claims, wherein the cell is isolated.

24. A semi-synthetic organism comprising the eukaryotic cell of any one of the preceding claims.

25. A eukaryotic cell culture comprising a plurality of eukaryotic cells of any one of claims 1-24.

26. A method of delivering a cell to an organism, comprising contacting the organism with the cell of any one of claims 1-23.

27. The method of claim 26, wherein the organism is a mammal, optionally wherein the mammal is a human.

28. A method of producing a polypeptide comprising at least one unnatural amino acid in a eukaryotic cell, comprising:
(a) introducing into the cell:
(i) a messenger RNA (mRNA) with a codon comprising a first unnatural base; and
(ii) a transfer RNA (tRNA) with an anticodon comprising a second unnatural base in the eukaryotic cell, wherein the first and second unnatural bases are capable of forming an unnatural base pair (UBP) in the eukaryotic cell; and
(b) translating the polypeptide comprising the at least one unnatural amino acid from the mRNA using the tRNA.

29. The method of the preceding claim, wherein the tRNA is charged with an unnatural amino acid.

30. A method of producing a polypeptide comprising at least one unnatural amino acid in a eukaryotic cell, comprising:
(a) providing a eukaryotic cell comprising:
(i) a messenger RNA (mRNA) with a codon comprising a first unnatural base;
(ii) a transfer RNA (tRNA) with an anticodon comprising a second unnatural base, wherein the first and second unnatural bases are capable of forming an unnatural base pair (UBP) in the eukaryotic cell; and
(b) translating the polypeptide comprising the at least one unnatural amino acid from the mRNA using the tRNA by a ribosome that is endogenous to the eukaryotic cell.

31. A method of producing a polypeptide in a eukaryotic cell, wherein the polypeptide comprises at least one unnatural amino acid, the method comprising:
(a) providing a eukaryotic cell, the eukaryotic cell comprising:
(i) an mRNA comprising a codon, wherein the codon comprises a first unnatural base;
(ii) a tRNA comprising an anti-codon, wherein the anti-codon comprises a second unnatural base, and wherein the first and second unnatural bases are capable of forming a complimentary base pair; and
(iii) a tRNA synthetase, wherein the tRNA synthetase preferentially aminoacylates the tRNA with the at least one unnatural amino acid compared to a natural amino acid; and
(b) providing the one more unnatural amino acids to the eukaryotic cell, wherein the eukaryotic cell produces the polypeptide comprising the at least one unnatural amino acid.

32. The method of any one of claims 26 to 31, wherein the codon of the mRNA comprises three contiguous nucleobases (N—N—N); and wherein the first unnatural base (X) is located at the first position (X—N—N) in the codon of the mRNA.

33. The method of any one of claims 26 to 31, wherein the codon of the mRNA comprises three contiguous nucleobases (N—N—N); and wherein the first unnatural base (X) is located at the middle position (N—X—N) in the codon of the mRNA.

34. The method of any one of claims 26 to 31, wherein the codon of the mRNA comprises three contiguous nucleobases (N—N—N); and wherein the first unnatural base (X) is located at the last position (N—N—X) in the codon of the mRNA.

35. The method of any one of claims 26 to 34, wherein the one or more unnatural bases comprising the codon of the mRNA is of the formula 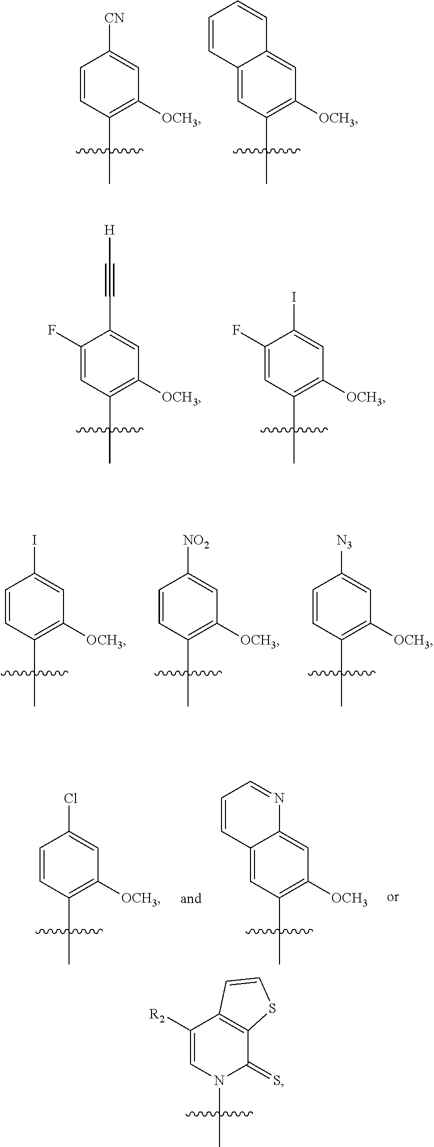
wherein R₂ is selected from the group consisting of hydrogen, alkyl, alkenyl, alkynyl, methoxy, methanethiol, methaneseleno, halogen, cyano, and azido, and the wavy line indicates a bond to a ribosyl moiety.

36. The method of any one of claims 26 to 35, wherein the first unnatural base or the second unnatural base is selected from the group consisting of 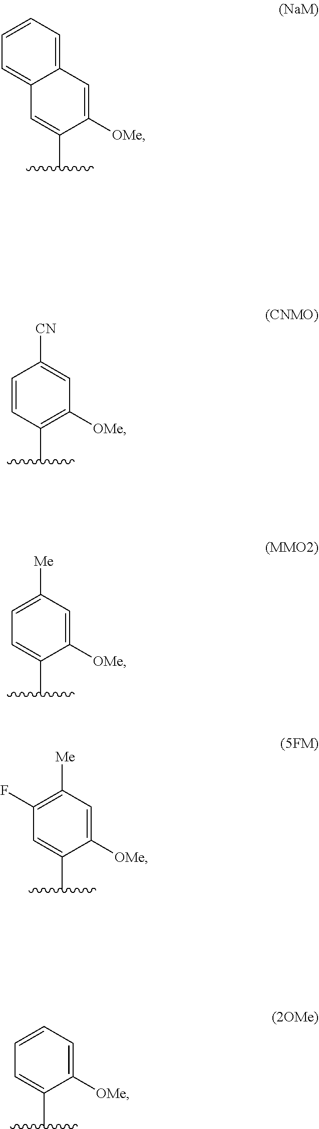 [SMILES: *c1cc2ccccc2cc1OC ; *c1ccc(C#N)cc1OC ; *c1ccc(C)cc1OC ; *c1cc(F)c(C)cc1OC ; *c1ccccc1OC] 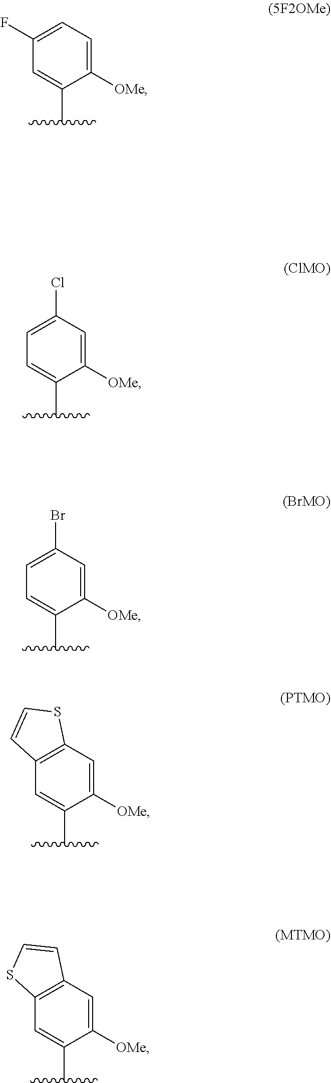 [SMILES: *c1cc(F)ccc1OC ; *c1ccc(Cl)cc1OC ; *c1ccc(Br)cc1OC ; *c1cc2ccsc2cc1OC ; *c1cc2sccc2cc1OC] 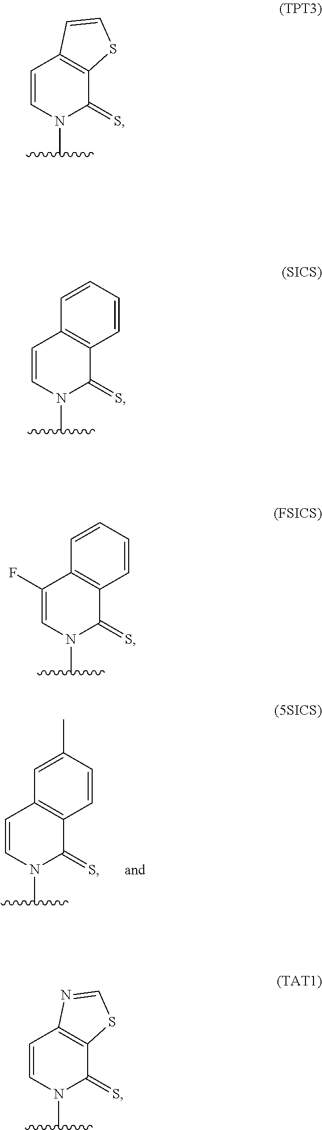 [SMILES: *n1ccc2ccsc2c1=S ; *n1ccc2ccccc2c1=S ; *n1cc(F)c2ccccc2c1=S ; *n1ccc2cc(C)ccc2c1=S ; "and" ; *n1ccc2ncsc2c1=S]
wherein the wavy line indicates a bond to a ribosyl moiety.

37. The method of claim 36, wherein the first unnatural base is 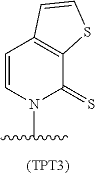
and the second unnatural base is 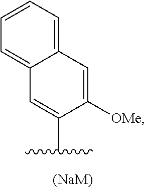
or the first unnatural base is 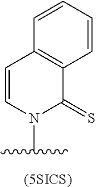
and the second unnatural base is 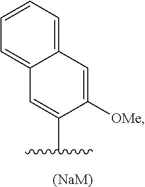
wherein the wavy line indicates a bond to a ribosyl moiety.

38. The method of claim 36, wherein the first unnatural base is 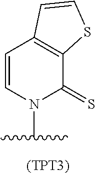
and the second unnatural base is 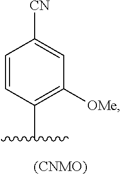
or the first unnatural base is 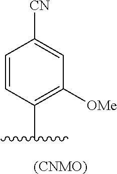
and the second unnatural base is 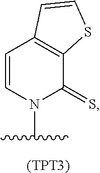
wherein the wavy line indicates a bond to a ribosyl moiety.

39. The method of claim 36, wherein the first unnatural base is 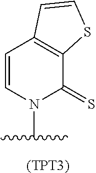
and
the second unnatural base is 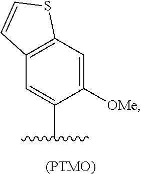
or the first unnatural base is 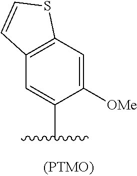 [SMILES: *c1cc2ccsc2cc1OC]
and the second unnatural base is 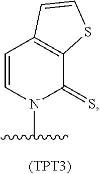
wherein the wavy line indicates a bond to a ribosyl moiety.

40. The method of claim 36, wherein the first unnatural base is 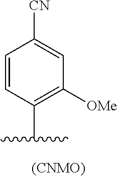
the second unnatural base is 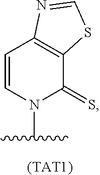
or the first unnatural base is 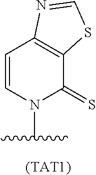
and the second unnatural base is 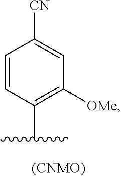
wherein the wavy line indicates a bond to a ribosyl moiety.

41. The method of claim 36, wherein the first unnatural base is 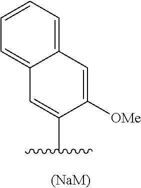
the second unnatural base is 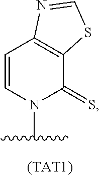
or the first unnatural base is (TAT1) and the second unnatural base is 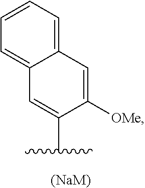
wherein the wavy line indicates a bond to a ribosyl moiety.

42. The method of any one of claims 26 to 36, wherein the codon of the mRNA comprises three contiguous nucleobases (N—N—N), wherein the first unnatural base (X) is located at the first position (X—N—N) in the codon of the mRNA, wherein the first unnatural base is selected from 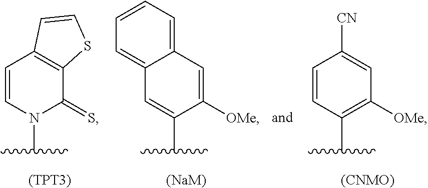
and wherein the wavy line indicates a bond to a ribosyl moiety.

43. The method of any one of claims 26 to 36, wherein the codon of the mRNA comprises three contiguous nucleobases (N—N—N), wherein the first unnatural base (X) is located at the middle position (N—X—N) in the codon of the mRNA, wherein the first unnatural base is selected from 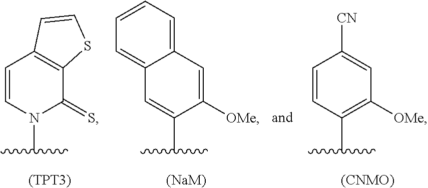
and wherein the wavy line indicates a bond to a ribosyl moiety.

44. The method of any one of claims 26 to 36, wherein the codon of the mRNA comprises three contiguous nucleobases (N—N—N), wherein the first unnatural base (X) is located at the last position —N—X in the codon of the mRNA, wherein the unnatural base is selected from 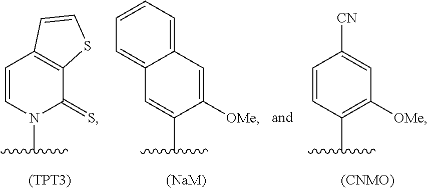
and wherein the wavy line indicates a bond to a ribosyl moiety.

45. The method of any one of claims 26 to 36, wherein the anticodon of the tRNA comprises three contiguous nucleobases (N—N—N); and wherein the second unnatural base (X) is located at the first position (X—N—N) in the anticodon of the tRNA, wherein the second unnatural base is selected from 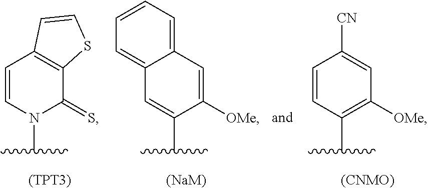
and wherein the wavy line indicates a bond to a ribosyl moiety.

46. The method of any one of claims 26 to 36, wherein the anticodon of the tRNA comprises three contiguous nucleobases (N—N—N); and wherein the second unnatural base (X) is located at the middle position (N—X—N) in the anticodon of the tRNA, wherein the second unnatural base is selected from 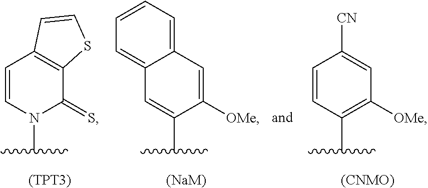
and wherein the wavy line indicates a bond to a ribosyl moiety.

47. The method of any one of claims 26 to 36, wherein the anticodon of the tRNA comprises three contiguous nucleobases (N—N—N); and wherein the second unnatural base (X) is located at the last position (N—N—X) in the anticodon of the tRNA, wherein the second unnatural base is selected from 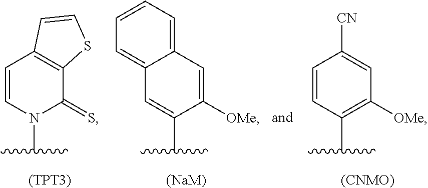
and wherein the wavy line indicates a bond to a ribosyl moiety.

48. The method of any one of claims 26 to 36, wherein the codon and the anticodon each comprise three contiguous nucleobases (N—N—N), wherein the first unnatural base (X) of the codon in the mRNA is located at a first position (X—N—N) of the codon, and the second unnatural base (Y) of the anticodon of the tRNA is located at the last position (N—N—Y) of the anticodon.

49. The method of any one of claims 26 to 36, wherein the codon and the anticodon each comprise three contiguous nucleobases (N—N—N), wherein the codon in the mRNA comprises a first unnatural base (X) located at the middle position (N—X—N) of the codon, and the anticodon in the tRNA comprises a second unnatural base (Y) located at the middle position (N—Y—N) of the anticodon.

50. The method of any one of claims 26 to 36, wherein the codon and the anticodon each comprise three contiguous nucleobases (N—N—N), wherein the codon in the mRNA comprises a first unnatural base (X) located at the last position (N—N—X) of the codon, and the anticodon in the tRNA comprises a second unnatural base (Y) located at the first position (Y—N—N) of the anticodon.

51. The method of any one of claims 48 to 50, wherein the first unnatural base (X) located in the codon of the mRNA and the second unnatural base (Y) located in the anticodon of the tRNA are the same or are different.

52. The method of any one of claims 48 to 51, wherein the first unnatural base (X) located in the codon of the mRNA and the second unnatural base (Y) located in the anticodon of the tRNA are selected from the group consisting of 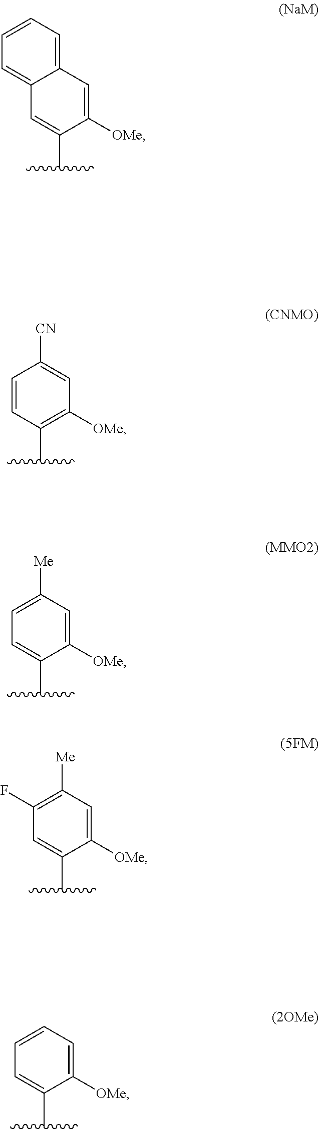 [SMILES: *c1cc2ccccc2cc1OC ; *c1ccc(C#N)cc1OC ; *c1ccc(C)cc1OC ; *c1cc(F)c(C)cc1OC ; *c1ccccc1OC] 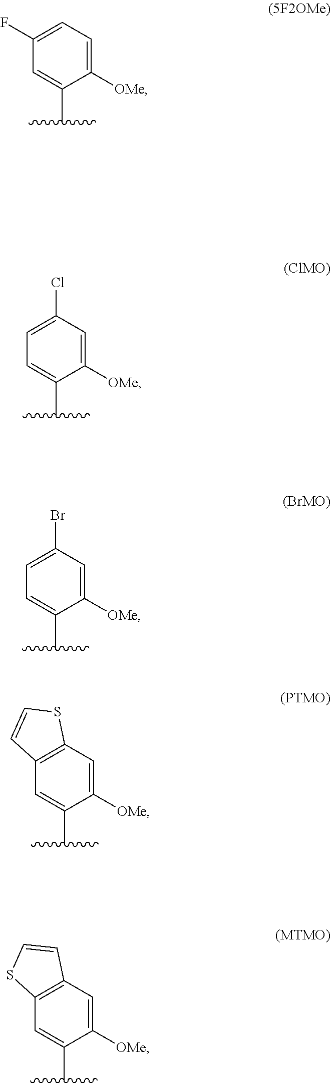 [SMILES: *c1cc(F)ccc1OC ; *c1ccc(Cl)cc1OC ; *c1ccc(Br)cc1OC ; *c1cc2ccsc2cc1OC ; *c1cc2sccc2cc1OC] 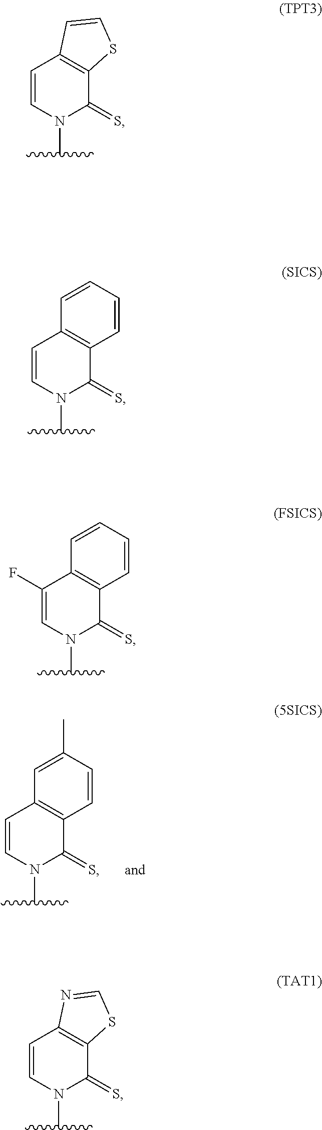 [SMILES: *n1ccc2ccsc2c1=S ; *n1ccc2ccccc2c1=S ; *n1cc(F)c2ccccc2c1=S ; *n1ccc2cc(C)ccc2c1=S ; "and" ; *n1ccc2ncsc2c1=S]
wherein the wavy line indicates a bond to a ribosyl moiety.

53. The method of claim 52, wherein the first unnatural base (X) located in the codon of the mRNA and the second unnatural base (Y) located in the anticodon of the tRNA are selected from the group consisting of 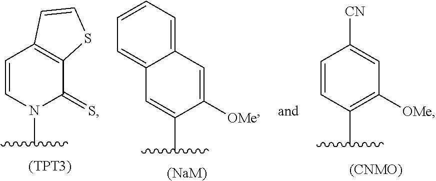
wherein the wavy line indicates a bond to a ribosyl moiety.

54. method of claim 53, wherein the first unnatural base (X) located in the codon of the mRNA and the second unnatural base (Y) located in the anticodon of the tRNA are both 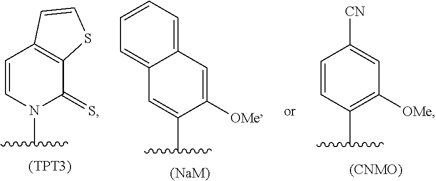
wherein the wavy line indicates a bond to a ribosyl moiety.

55. The method of claim 53, wherein the first unnatural base (X) located in the codon of the mRNA is selected from 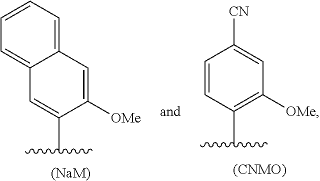
and the second unnatural base (Y) located in the anticodon of the tRNA is 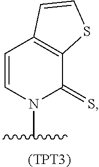
wherein in each case the wavy line indicates a bond to a ribosyl moiety.

56. The method of any one of claims 26-29, 31, 33, 35 to 41, 43, 46, and 49, wherein the codon in the mRNA is selected from AXC, GXC or GXU, wherein X is the first unnatural base.

57. The method of the immediately preceding claim, wherein the anticodon in the tRNA is selected from GYU, GYC, and AYC, and Y is a second unnatural base.

58. The method of claim 57, wherein the codon in the mRNA is AXC and the anticodon in the tRNA is GYU.

59. The method of claim 57, wherein the codon in the mRNA is GXC and the anticodon in the tRNA is GYC.

60. The method of claim 57, wherein the codon in the mRNA is GXU and the anticodon is AYC.

61. The method of any one of claims 26 to 60, wherein the first unnatural base or the second unnatural base comprise a modified sugar moiety selected from the group consisting of:
a modification at the 2′ position comprising:
OH, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N3, NH₂F, or a combination thereof;
O-alkyl, S-alkyl, N-alkyl, or a combination thereof;
O-alkenyl, S-alkenyl, N-alkenyl, or a combination thereof;
O-alkynyl, S-alkynyl, N-alkynyl, or a combination thereof;
O-alkyl-O-alkyl, 2′-F, 2′—OCH₃, 2′—O(CH₂)₂OCH₃, or a combination thereof, wherein the alkyl, alkenyl and alkynyl may be substituted or unsubstituted C₁-C₁₀, alkyl, C₂-C₁₀ alkenyl, C₂-C₁₀ alkynyl, —O[(CH₂)ₙO]ₘCH₃, —O(CH₂)ₙOCH₃, —O(CH₂)ₙNH₂, —
O(CH₂)ₙCH₃, —O(CH₂)ₙ—NH₂, and —O(CH₂)ₙON[(CH₂)ₙCH₃)]₂, wherein n and m are from 1 to about 10;
a modification at the 5′ position comprising:
5′-vinyl, 5′-methyl (R or S), or a combination thereof;
a modification at the 4′ position comprising:
4′-S, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, or a combination thereof;
or a combination thereof.

62. The method of any one of claims 26 to 61, wherein the at least one unnatural amino acid:
is a lysine analogue;
comprises an aromatic side chain;
comprises an azido group;
comprises an alkyne group; or
comprises an aldehyde or ketone group.

63. The method of any one of claims 26 to 61, wherein at least one unnatural amino acid is selected from the group consisting of N6-((azidoethoxy)-carbonyl)-L-lysine (AzK), N6-((propargylethoxy)-carbonyl)-L-lysine (PraK), BCN-L-lysine, norbomene lysine, TCO-lysine, methyltetrazine lysine, allyloxycarbonyllysine, 2-amino-8-oxononanoic acid, 2-amino-8-oxooctanoic acid, p-acetyl-L-phenylalanine, p-azidomethyl-L-phenylalanine (pAMF), p-iodo-L-phenylalanine, m-acetylphenylalanine, 2-amino-8-oxononanoic acid, p-propargyloxyphenylalanine, p-propargyl-phenylalanine, 3-methyl-phenylalanine, L-Dopa, fluorinated phenylalanine, isopropyl-L-phenylalanine, p-azido-L-phenylalanine, p-acyl-L-phenylalanine, p-benzoyl-L-phenylalanine, p-bromophenylalanine, p-amino-L-phenylalanine, isopropyl-L-phenylalanine, O-allyltyrosine, O-methyl-L-tyrosine, O-4-allyl-L-tyrosine, 4-propyl-L-tyrosine, phosphonotyrosine, tri-O-acetyl-GlcNAcp-serine, L-phosphoserine, phosphonoserine, L-3-(2-naphthyl)alanine, 2-amino-3-((2-((3-(benzyloxy)-3-oxopropyl)amino)ethyl)selanyl)propanoic acid, 2-amino-3-(phenylselanyl)propanoic, selenocysteine, N6-(((2-azidobenzyl)oxy)carbonyl)-L-lysine, N6-(((3-azidobenzyl)oxy)carbonyl)-L-lysine, and N6-(((4-azidobenzyl)oxy)carbonyl)-L-lysine.

64. The method of claim 63, wherein the unnatural amino acid is N6-((azidoethoxy)-carbonyl)-L-lysine (AzK).

65. The method of any one of claims 26 to 64, wherein the cell is a human cell.

66. The method of claim 65, wherein the human cell is a HEK293T cell.

67. The method of any one of claims 26 to 64, wherein the cell is a hamster cell.

68. The method of claim 67, wherein the hamster cell is a Chinese hamster ovary (CHO) cell.

69. The method of any one of claims 26 to 68, wherein the tRNA is derived from Methanococcus jannaschii, Methanosarcina barkeri, Methanosarcina mazei, or Methanosarcina acetivorans.

70. The method of any one of claims 26 to 69, wherein the cell comprises a tRNA synthetase derived from Methanococcus jannaschii, Methanosarcina barkeri, Methanosarcina mazei, or Methanosarcina acetivorans.

71. A system for expression of an unnatural polypeptide comprising:
(a) at least one unnatural amino acid;
(b) an mRNA encoding the unnatural polypeptide, said mRNA comprising at least one codon comprising one or more first unnatural bases;
(c) a tRNA comprising at least one anti-codon comprising one or more second unnatural bases wherein the one or more first unnatural bases and the one or more second unnatural bases are capable of forming one or more complementary base pairs; and
(d) a eukaryotic ribosome capable of translating the mRNA into a polypeptide comprising the unnatural amino acid using the tRNA and tRNA synthetase,
wherein the tRNA is charged with the unnatural amino acid, or the system further comprises a tRNA synthetase or one or more nucleic acid constructs comprising a nucleic acid sequence encoding a tRNA synthetase, wherein the tRNA synthetase preferentially aminoacylates the tRNA with the at least one unnatural amino acid.

72. The system of claim 71, wherein the at least one codon of the mRNA comprises three contiguous nucleobases (N—N—N); and wherein the one or more first unnatural bases (X) is located at the first position (X—N—N) in the at least one codon of the mRNA.

73. The system of claim 71, wherein the at least one codon of the mRNA comprises three contiguous nucleobases (N—N—N); and wherein the one or more first unnatural bases (X) is located at the middle position (N—X—N) in the codon of the mRNA.

74. The system of claim 71, wherein the at least one codon of the mRNA comprises three contiguous nucleobases (N—N—N); and wherein the one or more first unnatural bases (X) is located at the last position (N—N—X) in the at least one codon of the mRNA.

75. The system of any one of claims 71 to 74, wherein the one or more unnatural bases is of the formula 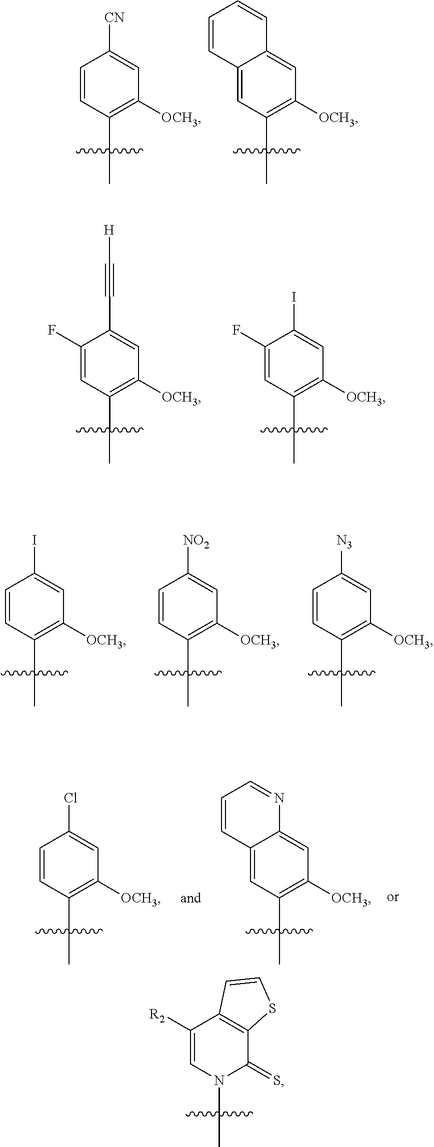
wherein R₂ is selected from the group consisting of hydrogen, alkyl, alkenyl, alkynyl, methoxy, methanethiol, methaneseleno, halogen, cyano, and azido, and the wavy line indicates a bond to a ribosyl moiety.

76. The system of any one of claims 71 to 74, wherein the one or more first unnatural bases or the one or more second unnatural bases is selected from the group consisting of 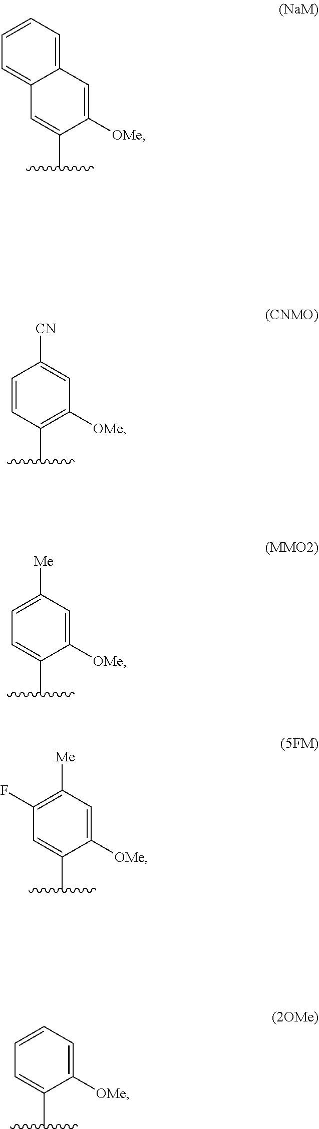 [SMILES: *c1cc2ccccc2cc1OC ; *c1ccc(C#N)cc1OC ; *c1ccc(C)cc1OC ; *c1cc(F)c(C)cc1OC ; *c1ccccc1OC] 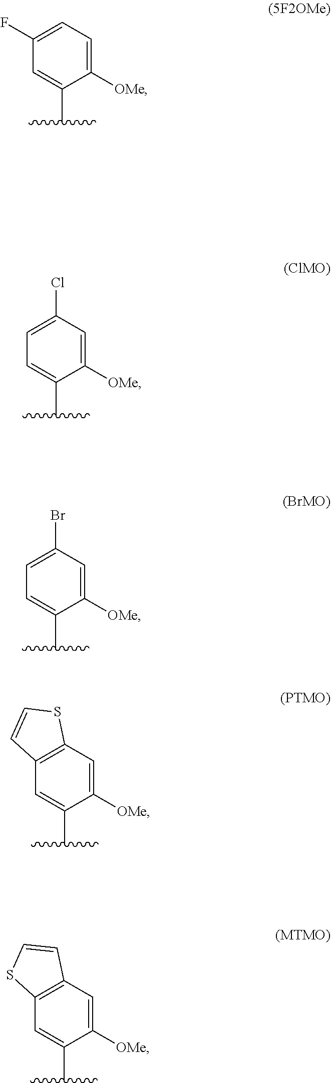 [SMILES: *c1cc(F)ccc1OC ; *c1ccc(Cl)cc1OC ; *c1ccc(Br)cc1OC ; *c1cc2ccsc2cc1OC ; *c1cc2sccc2cc1OC] 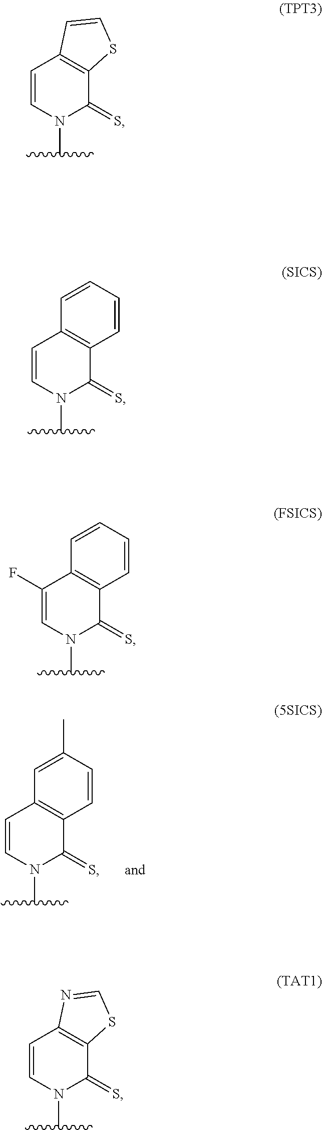 [SMILES: *n1ccc2ccsc2c1=S ; *n1ccc2ccccc2c1=S ; *n1cc(F)c2ccccc2c1=S ; *n1ccc2cc(C)ccc2c1=S ; "and" ; *n1ccc2ncsc2c1=S]
wherein the wavy line indicates a bond to a ribosyl moiety.

77. The system of claim 76, when the one or more first unnatural bases is 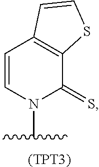
the one or more second unnatural bases is 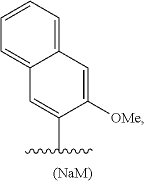
and when the one or more first unnatural bases is 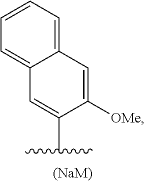
the second unnatural base is 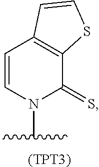
wherein the wavy line indicates a bond to a ribosyl moiety.

78. The system of claim 76, when the one or more first unnatural bases is 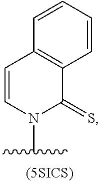
the one or more second unnatural bases is 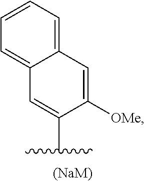
and when the one or more first unnatural base is 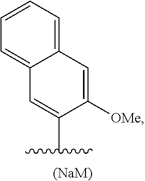
the one or more second unnatural bases is 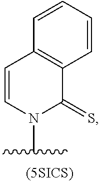
wherein the wavy line indicates a bond to a ribosyl moiety.

79. The system of claim 76, when the one or more first unnatural bases is 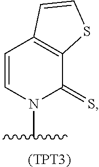
the one or more second unnatural bases is 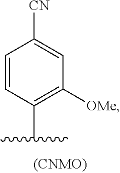 [SMILES: *c1ccc(C#N)cc1OC]
and when the one or more first unnatural is 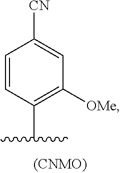 [SMILES: *c1ccc(C#N)cc1OC]
the one or more second unnatural bases is 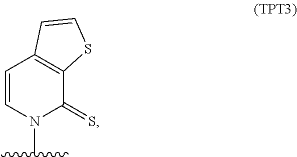 [SMILES: *n1ccc2ccsc2c1=S]
wherein the wavy line indicates a bond to a ribosyl moiety.

80. The system of claim 76, when the one or more first unnatural bases is 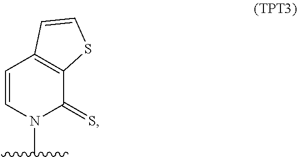 [SMILES: *n1ccc2ccsc2c1=S]
the one or more second unnatural bases is 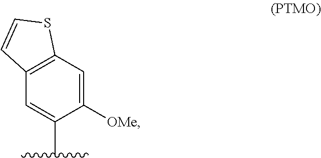 [SMILES: *c1cc2ccsc2cc1OC]
and when the one or more first unnatural bases is 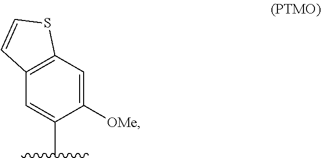 [SMILES: *c1cc2ccsc2cc1OC]
the one or more second unnatural bases is 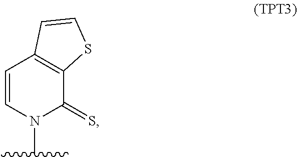 [SMILES: *n1ccc2ccsc2c1=S]
wherein the wavy line indicates a bond to a ribosyl moiety.

81. The system of claim 76, when the one or more first unnatural bases is 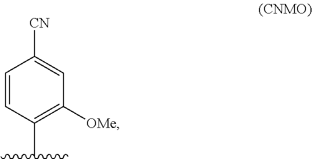 [SMILES: *c1ccc(C#N)cc1OC]
the one or more second unnatural bases is 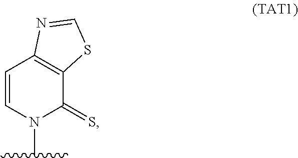 [SMILES: *n1ccc2ncsc2c1=S]
and when the one or more first unnatural bases is 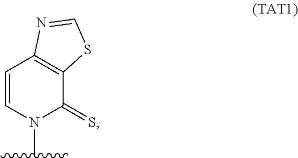 [SMILES: *n1ccc2ncsc2c1=S]
the one or more second unnatural bases is 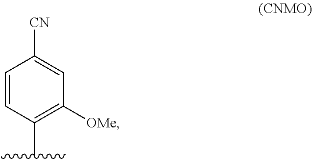 [SMILES: *c1ccc(C#N)cc1OC]
wherein the wavy line indicates a bond to a ribosyl moiety.

82. The system of claim 76, when the one or more first unnatural bases is 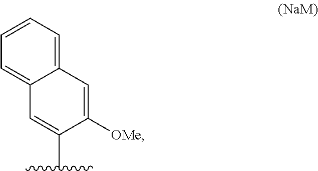 [SMILES: *c1cc2ccccc2cc1OC]
the one or more second unnatural bases is 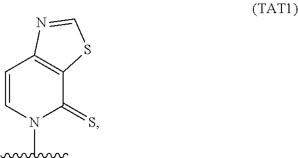 [SMILES: *n1ccc2ncsc2c1=S]
and when the one or more first unnatural bases is 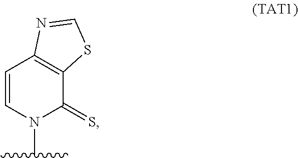 [SMILES: *n1ccc2ncsc2c1=S]
the one or more second unnatural bases is 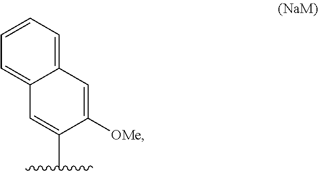 [SMILES: *c1cc2ccccc2cc1OC]
wherein the wavy line indicates a bond to a ribosyl moiety.

83. The system of claim 76, when the one or more first unnatural bases is 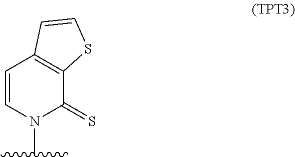 [SMILES: *n1ccc2ccsc2c1=S]
the one or more second unnatural bases is 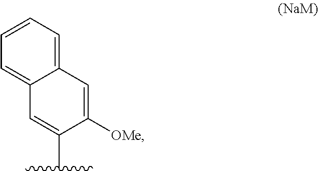 [SMILES: *c1cc2ccccc2cc1OC]
wherein the wavy line indicates a bond to a ribosyl moiety.

84. The system of any one of claims 71 to 74, wherein the one or more first unnatural bases is selected from 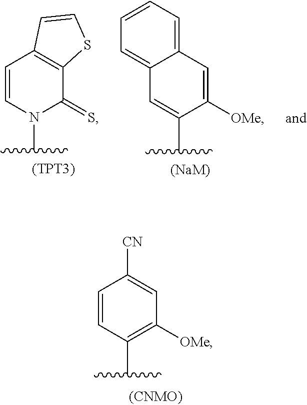
wherein the wavy line indicates a bond to a ribosyl moiety.

85. The system of claim 71, wherein the at least one codon of the mRNA comprises three contiguous nucleobases (N—N—N), wherein the one or more first unnatural bases (X) is located at the first position (X—N—N) in the codon of the mRNA, wherein the one or more first unnatural bases is selected from 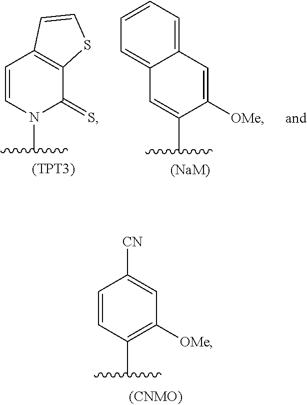
and wherein the wavy line indicates a bond to a ribosyl moiety.

86. The system of claim 71, wherein the at least one codon of the mRNA comprises three contiguous nucleobases (N—N—N), wherein the one or more first unnatural bases (X) is located at the middle position (N—X—N) in the codon of the mRNA, wherein the one or more first unnatural bases is selected from 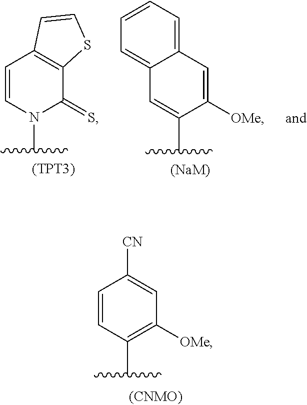
and wherein the wavy line indicates a bond to a ribosyl moiety.

87. The system of claim 71, wherein the at least one codon of the mRNA comprises three contiguous nucleobases (N—N—N), wherein the one or more first unnatural base (X) is located at the last position (N—N—X) in the codon of the mRNA, wherein the one or more first unnatural base is selected from 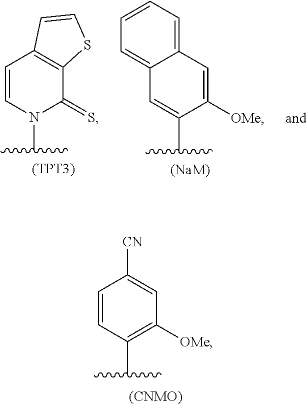
and wherein the wavy line indicates a bond to a ribosyl moiety.

88. The system of claim 71, wherein the at least one anticodon of the tRNA comprises three contiguous nucleobases (N—N—N); and wherein the one or more second unnatural base (X) is located at the first position (X—N—N) in the anticodon of the tRNA, wherein the one or more second unnatural bases is selected from 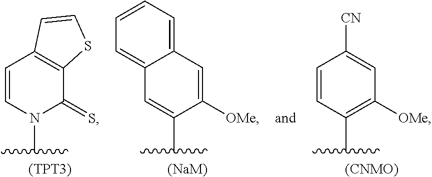
and wherein the wavy line indicates a bond to a ribosyl moiety.

89. The system of claim 71, wherein the at least one anticodon of the tRNA comprises three contiguous nucleobases (N—N—N); and wherein the one or more second unnatural bases (X) is located at the middle position (N—X—N) in the anticodon of the tRNA, wherein the one or more second unnatural bases is selected from 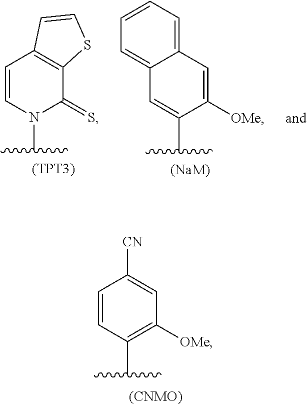
and wherein the wavy line indicates a bond to a ribosyl moiety.

90. The system of claim 71, wherein the at least one anticodon of the tRNA comprises three contiguous nucleobases (N—N—N); and wherein the one or more second unnatural bases (X) is located at the last position (N—N—X) in the anticodon of the tRNA, wherein the one or more second unnatural base is selected from 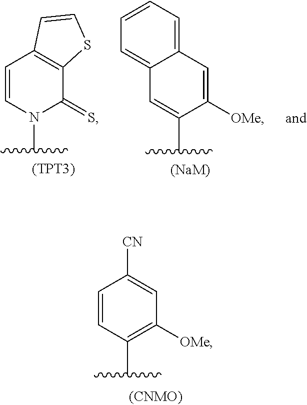
and wherein the wavy line indicates a bond to a ribosyl moiety.

91. The system of claim 71, wherein the at least one codon and the at least one anticodon each, independently, comprise three contiguous nucleobases (N—N—N), and wherein the at least one codon comprises one or more first unnatural bases (X) located at the first position (X—N—N) of the codon, and the at least one anticodons in the tRNA comprises the one or more second unnatural bases (Y) located at the last position (N—N—Y) of the anticodon.

92. The system of claim 91, wherein the one or more first unnatural bases (X) located in the codon of the mRNA and the one or more second unnatural bases (Y) located in the anticodon of the tRNA are the same or are different.

93. The system of any one of claims 91 to 92, wherein the one or more first unnatural bases (X) located in the codon of the mRNA and the one or more second unnatural bases (Y) located in the anticodon of the tRNA are selected from the group consisting of 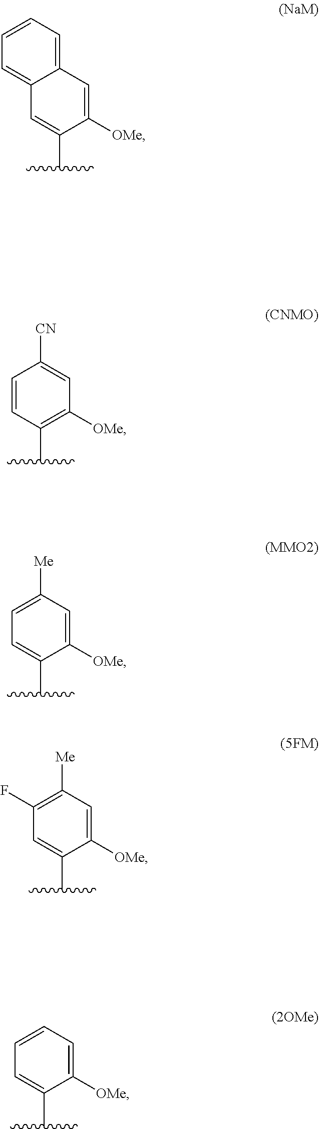 [SMILES: *c1cc2ccccc2cc1OC ; *c1ccc(C#N)cc1OC ; *c1ccc(C)cc1OC ; *c1cc(F)c(C)cc1OC ; *c1ccccc1OC] 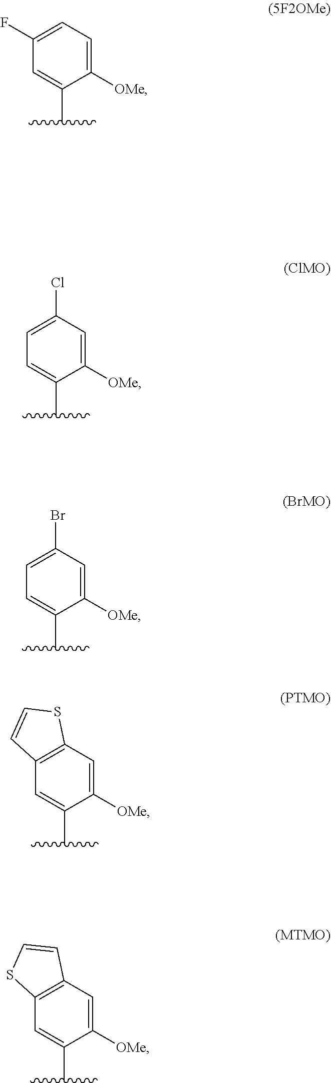 [SMILES: *c1cc(F)ccc1OC ; *c1ccc(Cl)cc1OC ; *c1ccc(Br)cc1OC ; *c1cc2ccsc2cc1OC ; *c1cc2sccc2cc1OC] 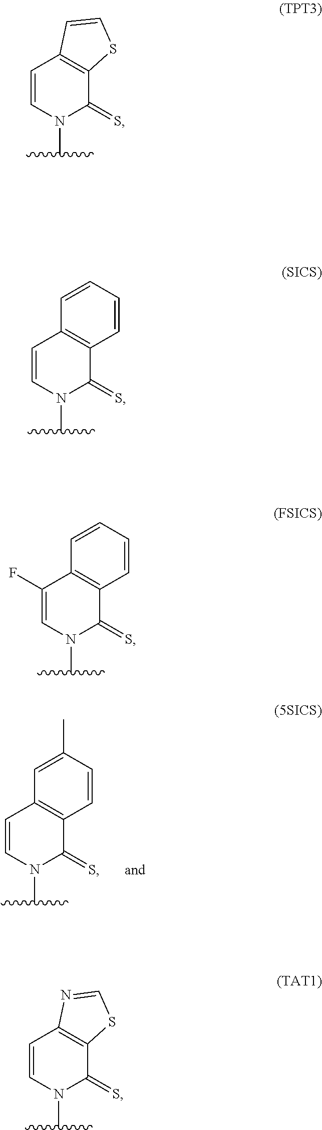 [SMILES: *n1ccc2ccsc2c1=S ; *n1ccc2ccccc2c1=S ; *n1cc(F)c2ccccc2c1=S ; *n1ccc2cc(C)ccc2c1=S ; "and" ; *n1ccc2ncsc2c1=S]
wherein the wavy line indicates a bond to a ribosyl moiety.

94. The system of claim 93, wherein the one or more first unnatural bases (X) located in the codon of the mRNA and the one or more second unnatural bases (Y) located in the anticodon of the tRNA are selected from the group consisting of 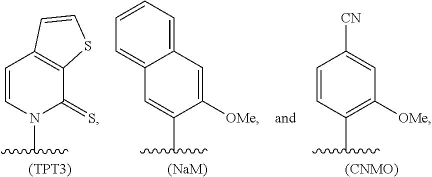
wherein the wavy line indicates a bond to a ribosyl moiety.

95. The system of claim 94, wherein the one or more first unnatural base (X) located in the codon of the mRNA is selected from 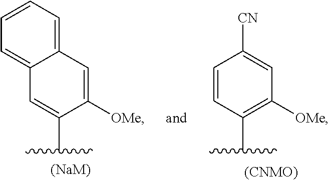
and the one or more second unnatural bases (Y) located in the anticodon of the tRNA is 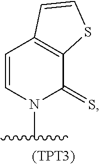
wherein in each case the wavy line indicates a bond to a ribosyl moiety.

96. The system of claim 71, wherein the at least one codon and the at least one anticodon each, independently, comprise three contiguous nucleobases (N—N—N), and wherein the at least one codon in the mRNA comprises the one or more first unnatural bases (X) located at a middle position (N—X—N) of the at least one codon, and the at least one anticodon in the tRNA comprises the one or more second unnatural bases (Y) located at a middle position (N—Y—N) of the anticodon.

97. The system of claim 96, wherein the one or more first unnatural bases (X) located in the codon of the mRNA and the one or more second unnatural bases (Y) located in the anticodon of the tRNA are the same or are different.

98. The system of any one of claims 96 to 97, wherein the one or more first unnatural bases (X) located in the codon of the mRNA and the one or more second unnatural bases (Y) located in the anticodon of the tRNA are selected from the group consisting of 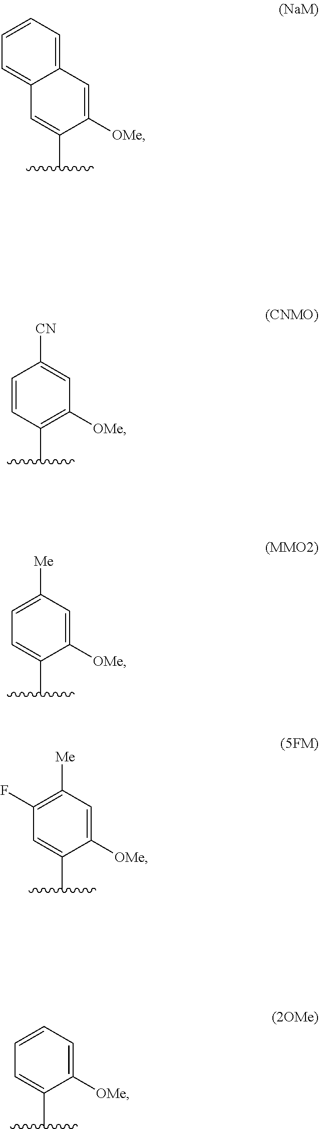 [SMILES: *c1cc2ccccc2cc1OC ; *c1ccc(C#N)cc1OC ; *c1ccc(C)cc1OC ; *c1cc(F)c(C)cc1OC ; *c1ccccc1OC] 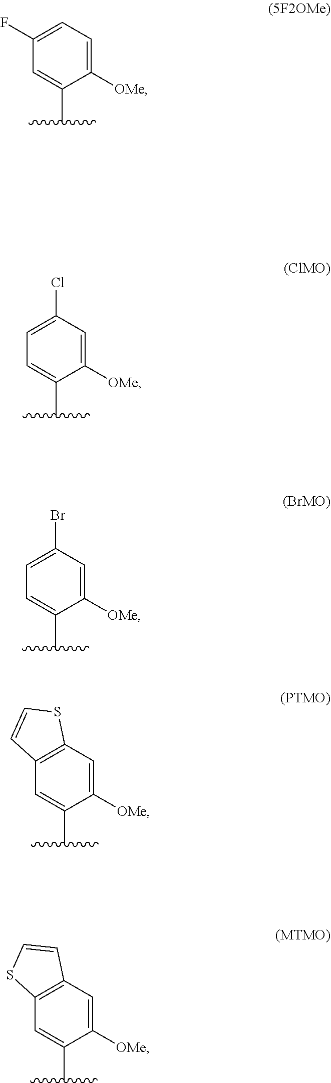 [SMILES: *c1cc(F)ccc1OC ; *c1ccc(Cl)cc1OC ; *c1ccc(Br)cc1OC ; *c1cc2ccsc2cc1OC ; *c1cc2sccc2cc1OC] 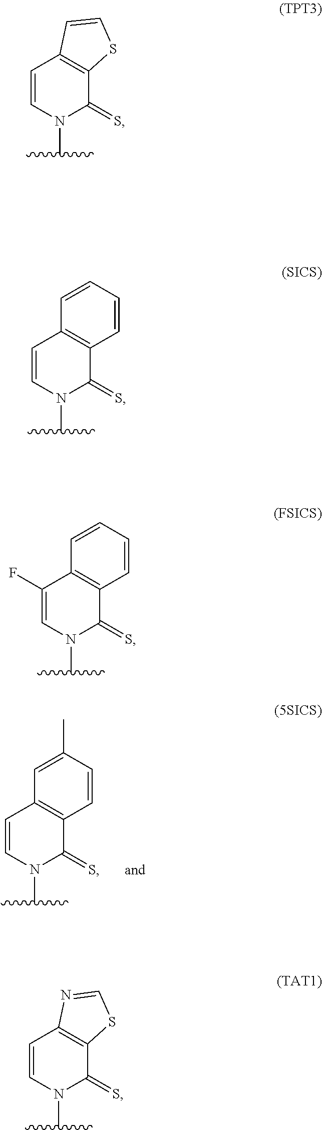 [SMILES: *n1ccc2ccsc2c1=S ; *n1ccc2ccccc2c1=S ; *n1cc(F)c2ccccc2c1=S ; *n1ccc2cc(C)ccc2c1=S ; "and" ; *n1ccc2ncsc2c1=S]
wherein the wavy line indicates a bond to a ribosyl moiety.

99. The system of claim 98, wherein the one or more first unnatural bases (X) located in the codon of the mRNA and the one or more second unnatural bases (Y) located in the anticodon of the tRNA are selected from the group consisting of 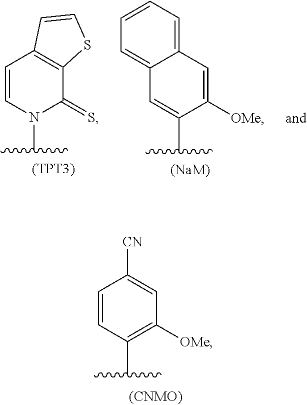
wherein the wavy line indicates a bond to a ribosyl moiety.

100. The system of claim 99, wherein the one or more first unnatural bases (X) located in the codon of the mRNA is selected from 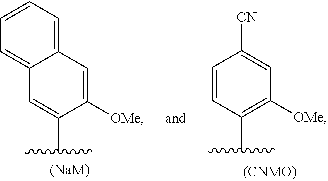
and the one or more second unnatural bases (Y) located in the anticodon of the tRNA is 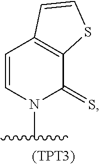
wherein in each case the wavy line indicates a bond to a ribosyl moiety.

101. The system of claim 71, wherein the at least one codon and the at least one anticodon each, independently, comprise three contiguous nucleobases (N—N—N), and wherein the at least one codon in the mRNA comprises the one or more first unnatural bases (X) located at the last position (N—N—X) of the at least one codon, and the at least one anticodon in the tRNA comprises the one or more second unnatural bases (Y) located at the first position (Y—N—N) of the anticodon.

102. The system of claim 101, wherein the one or more first unnatural bases (X) located in the codon of the mRNA and the one or more second unnatural bases (Y) located in the anticodon of the tRNA are the same or are different.

103. The system of any one of claims 101 to 102, wherein the one or more first unnatural bases (X) located in the codon of the mRNA and the one or more second unnatural bases (Y) located in the anticodon of the tRNA are selected from the group consisting of 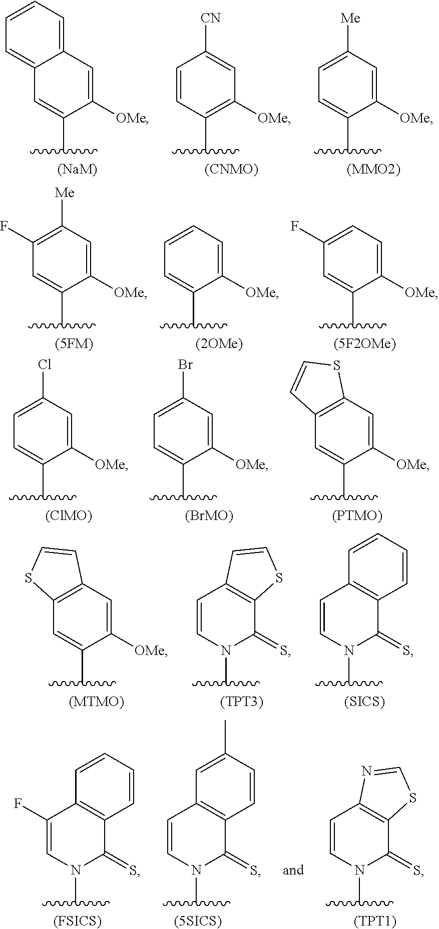
wherein the wavy line indicates a bond to a ribosyl moiety.

104. The system of claim 103, wherein the one or more first unnatural bases (X) located in the codon of the mRNA and the one or more second unnatural bases (Y) located in the anticodon of the tRNA are selected from the group consisting of 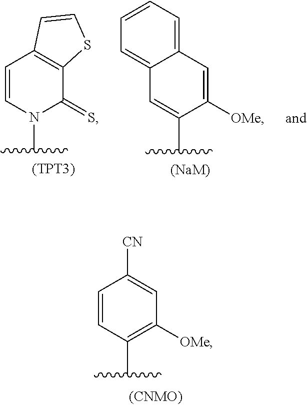
wherein the wavy line indicates a bond to a ribosyl moiety.

105. The system of claim 104, wherein the one or more first unnatural bases (X) located in the codon of the mRNA is selected from 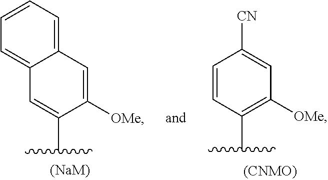
and the one or more second unnatural bases (Y) located in the anticodon of the tRNA is 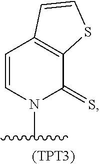
wherein in each case the wavy line indicates a bond to a ribosyl moiety.

106. The system of any one of claims 71 to 105, wherein the at least one codon in the mRNA is selected from AXC, GXC or GXU, wherein X is the one or more first unnatural bases.

107. The system of the immediately preceding claim, wherein the at least one anticodon in the tRNA is selected from GYU, GYC, and AYC, and Y is the one or more second unnatural bases.

108. The system of claim 107, wherein the at least one codon in the mRNA is AXC and the at least one anticodon in the tRNA is GYU.

109. The system of claim 107, wherein the at least one codon in the mRNA is GXC and the at least one anticodon in the tRNA is GYC.

110. The system of claim 107, wherein the at least one codon in the mRNA is GXU and the at least one anticodon is AYC.

111. The system of any one of claims 71 to 110, wherein the tRNA is derived from Methanococcus jannaschii, Methanosarcina barkeri, Methanosarcina mazei, or Methanosarcina acetivorans.

112. The system of any one of claims 71 to 111, wherein the tRNA synthetase is derived from Methanococcus jannaschii, Methanosarcina barkeri, Methanosarcina mazei, or Methanosarcina acetivorans.

113. The system of any one of claims 71 to 112, which is in vitro or cell-free.

114. The system of any one of claims 71 to 113, comprising a cell lysate.

115. The system of any one of claims 71 to 113, which is a reconstituted system of purified components.

116. The system of any one of claims 71 to 112, which is in a eukaryotic cell.

117. The system of claim 116, wherein the eukaryotic cell is a human cell.

118. The system of claim 116, wherein the eukaryotic cell is a HEK293T cell.

119. The system of claim 116, wherein the eukaryotic cell is a hamster cell.

120. The system of claim 119, wherein the hamster cell is a Chinese hamster ovary (CHO) cell.

121. The system of any one of claims 71 to 120, wherein the unnatural amino acid:
is a lysine analogue;
comprises an aromatic side chain;
comprises an azido group;
comprises an alkyne group; or
comprises an aldehyde or ketone group.

122. The system of any one of claims 71 to 121, wherein the unnatural amino acid is selected from the group consisting of N6-((azidoethoxy)-carbonyl)-L-lysine (AzK), N6-((propargylethoxy)-carbonyl)-L-lysine (PraK), BCN-L-lysine, norbomene lysine, TCO-lysine, methyltetrazine lysine, allyloxycarbonyllysine, 2-amino-8-oxononanoic acid, 2-amino-8-oxooctanoic acid, p-acetyl-L-phenylalanine, p-azidomethyl-L-phenylalanine (pAMF), p-iodo-L-phenylalanine, m-acetylphenylalanine, 2-amino-8-oxononanoic acid, p-propargyloxyphenylalanine, p-propargyl-phenylalanine, 3-methyl-phenylalanine, L-Dopa, fluorinated phenylalanine, isopropyl-L-phenylalanine, p-azido-L-phenylalanine, p-acyl-L-phenylalanine, p-benzoyl-L-phenylalanine, p-bromophenylalanine, p-amino-L-phenylalanine, isopropyl-L-phenylalanine, O-allyltyrosine, O-methyl-L-tyrosine, O-4-allyl-L-tyrosine, 4-propyl-L-tyrosine, phosphonotyrosine, tri-O-acetyl-GlcNAcp-serine, L-phosphoserine, phosphonoserine, L-3-(2-naphthyl)alanine, 2-amino-3-((2-((3-(benzyloxy)-3-oxopropyl)amino)ethyl)selanyl)propanoic acid, 2-amino-3-(phenylselanyl)propanoic, selenocysteine, N6-(((2-azidobenzyl)oxy)carbonyl)-L-lysine, N6-(((3-azidobenzyl)oxy)carbonyl)-L-lysine, and N6-(((4-azidobenzyl)oxy)carbonyl)-L-lysine.

123. The system of any one of claims 71 to 122, wherein the unnatural amino acid is N6-((azidoethoxy)-carbonyl)-L-lysine (AzK).

124. The system of any one of claims 71 to 123, wherein the tRNA is charged with the unnatural amino acid.
